SEQUENCE LISTING (289 sequences)

SEQ ID NO: 1 - protein, 58 aa, synthetic construct
VDNKFNKEETFADLEISNLPNLNFYQKWAFIRSLMDDPSQSANLLAEAKKLNDAQAPK

SEQ ID NO: 2 - protein, 58 aa, synthetic construct
VDNKFNKENQFADEEIAALPNLNFYQKWAFIRKLMDDPSQSANLLAEAKKLNDAQAPK

SEQ ID NO: 3 - protein, 121 aa, synthetic construct
GSSHHHHHHYYLEVDNKFNKEETFADLEISNLPNLNFYQKWAFIRSLMDDPSQSANLLAEAKKLNDAQAPKVDYVLAEAKVLANRELDKYGVSDYYKNLINNAKTVEGVKALIDEILAALP

SEQ ID NO: 4 - protein, 66 aa, synthetic construct
VDNKFNKEETFADLEISNLPNLNFYQKWAFIRSLMDDPSQSANLLAEAKKLNDAQAPKYYHHHHHH

SEQ ID NO: 5 - protein, 64 aa, synthetic construct
NKFNKEETFADLEISNLPNLNFYQKWAFIRSLMDDPSQSANLLAEAKKLNDAQAPKYYHHHHHH

SEQ ID NO: 6 - protein, 63 aa, synthetic construct
KFNKEETFADLEISNLPNLNFYQKWAFIRSLMDDPSQSANLLAEAKKLNDAQAPKYYHHHHHH

SEQ ID NO: 7 - protein, 62 aa, synthetic construct
FNKEETFADLEISNLPNLNFYQKWAFIRSLMDDPSQSANLLAEAKKLNDAQAPKYYHHHHHH

SEQ ID NO: 8 - protein, 61 aa, synthetic construct
NKEETFADLEISNLPNLNFYQKWAFIRSLMDDPSQSANLLAEAKKLNDAQAPKYYHHHHHH

SEQ ID NO: 9 - protein, 71 aa, synthetic construct
GSSHHHHHHYYLEVDNKFNKEATFADLEISNLPNLNFYQKWAFIRSLMDDPSQSANLLAEAKKLNDAQAPK

SEQ ID NO: 10 - protein, 71 aa, synthetic construct
GSSHHHHHHYYLEVDNKFNKEEAFADLEISNLPNLNFYQKWAFIRSLMDDPSQSANLLAEAKKLNDAQAPK

SEQ ID NO: 11 - protein, 71 aa, synthetic construct
GSSHHHHHHYYLEVDNKFNKEETAADLEISNLPNLNFYQKWAFIRSLMDDPSQSANLLAEAKKLNDAQAPK

SEQ ID NO: 12 - protein, 71 aa, synthetic construct
GSSHHHHHHYYLEVDNKFNKEETFAALEISNLPNLNFYQKWAFIRSLMDDPSQSANLLAEAKKLNDAQAPK

SEQ ID NO: 13 - protein, 71 aa, synthetic construct
GSSHHHHHHYYLEVDNKFNKEETFADAEISNLPNLNFYQKWAFIRSLMDDPSQSANLLAEAKKLNDAQAPK

SEQ ID NO: 14 - protein, 71 aa, synthetic construct
GSSHHHHHHYYLEVDNKFNKEETFADLEIANLPNLNFYQKWAFIRSLMDDPSQSANLLAEAKKLNDAQAPK

SEQ ID NO: 15 - protein, 71 aa, synthetic construct
GSSHHHHHHYYLEVDNKFNKEETFADLEISALPNLNFYQKWAFIRSLMDDPSQSANLLAEAKKLNDAQAPK

SEQ ID NO: 16 - protein, 71 aa, synthetic construct
GSSHHHHHHYYLEVDNKFNKEETFADLEISNLPNLNAYQKWAFIRSLMDDPSQSANLLAEAKKLNDAQAPK

SEQ ID NO: 17 - protein, 71 aa, synthetic construct
GSSHHHHHHYYLEVDNKFNKEETFADLEISNLPNLNFAQKWAFIRSLMDDPSQSANLLAEAKKLNDAQAPK

SEQ ID NO: 18 - protein, 71 aa, synthetic construct
GSSHHHHHHYYLEVDNKFNKEETFADLEISNLPNLNFYQAWAFIRSLMDDPSQSANLLAEAKKLNDAQAPK

SEQ ID NO: 19 - protein, 58 aa, synthetic construct
VDNKFNKEETFADLEISNLPNLNFYQKAAFIRSLMDDPSQSANLLAEAKKLNDAQAPK

SEQ ID NO: 20 - protein, 71 aa, synthetic construct
GSSHHHHHHYYLEVDNKFNKEETFADLEISNLPNLNFYQKWAFARSLMDDPSQSANLLAEAKKLNDAQAPK

SEQ ID NO: 21 - protein, 71 aa, synthetic construct
GSSHHHHHHYYLEVDNKFNKEETFADLEISNLPNLNFYQKWAFIASLMDDPSQSANLLAEAKKLNDAQAPK

SEQ ID NO: 22 - protein, 71 aa, synthetic construct
GSSHHHHHHYYLEVDNKFNKEETFADLEISNLPNLNFYQKWAFIRSLADDPSQSANLLAEAKKLNDAQAPK

SEQ ID NO: 23 - protein, 58 aa, synthetic construct
VDNKFNKEEIFADREIAFLPNLNFYQKWAFIRKLMDDPSQSANLLAEAKKLNDAQAPK

SEQ ID NO: 24 - protein, 58 aa, synthetic construct
VDNKFNKESQFADEEIEALPNLNFYQKWAFIRSLMDDPSQSANLLAEAKKLNDAQAPK

SEQ ID NO: 25 - protein, 58 aa, synthetic construct
VDNKFNKEQEFADYEIATLPNLNFYQKWAFIRSLMDDPSQSANLLAEAKKLNDAQAPK

SEQ ID NO: 26 - protein, 58 aa, synthetic construct
VDNKFNKEDSFADTEIAKLPNLNFYQKWAFIRSLMDDPSQSANLLAEAKKLNDAQAPK

SEQ ID NO: 27 - protein, 58 aa, synthetic construct
GDNKFNKEEIFADQEIEALPNLNFYQKWAFIRSLMDDPSQSANLLAEAKKLNDAQAPK

SEQ ID NO: 28 - protein, 58 aa, synthetic construct
VDNKFNKEDYFADYEIATLPNLNFYQKWAFIRSLMDDPSQSANLLAEAKKLNDAQAPK

SEQ ID NO: 29 - protein, 58 aa, synthetic construct
VDNKFNKEEQFADEEIAHLPNLNFYQKWAFIRSLMDDPSQSANLLAEAKKLNDAQAPK

SEQ ID NO: 30 - protein, 58 aa, synthetic construct
VDNKFNKESMFADIEITKLPNLNFYQKWAFIRKLMDDPSQSANLLAEAKKLNDAQAPK

SEQ ID NO: 31 - protein, 58 aa, synthetic construct
VDNKFNKEEVFADEEIASLPNLNFYQKWAFIRSLMDDPSQSANLLAEAKKLNDAQAPK

SEQ ID NO: 32 - protein, 58 aa, synthetic construct
VDNKFNKEVREADEEIAALPNLNFYQKWAFIRKLMDDPSQSANLLAEAKKLNDAQAPK

SEQ ID NO: 33 - protein, 58 aa, synthetic construct
VDNKFNKEHQFADYEIAMLPNLNFYQKWAFIRSLMDDPSQSANLLAEAKKLNDAQAPK

SEQ ID NO: 34 - protein, 58 aa, synthetic construct
VDNKFNKEHFFADKEIAKLPNLNFYQKWAFIRSLMDDPSQSANLLAEAKKLNDAQAPK

SEQ ID NO: 35 - protein, 58 aa, synthetic construct
VDNKFNKEKAFADTEIVNLPNLNFYQKWAFIRSLMDDPSQSANLLAEAKKLNDAQAPK

SEQ ID NO: 36 - protein, 58 aa, synthetic construct
VDNKFNKESEFADAEIANLPNLNFYQKWAFIRKLMDDPSQSANLLAEAKKLNDAQAPK

SEQ ID NO: 37 - protein, 58 aa, synthetic construct
GDNKFNKEENFADHEIALLPNLNFYQKWAFIRKLMDDPSQSANLLAEAKKLNDAQAPK

SEQ ID NO: 38 - protein, 58 aa, synthetic construct
VDNKFNKEDSHADEEIAALPNLNFYQKWAFIRKLMDDPSQSANLLAEAKKLNDAQAPK

SEQ ID NO: 39 - protein, 58 aa, synthetic construct
VDNKFNKESKFADEEIAKLPNLNFYQKWAFIRSLMDDPSQSANLLAEAKKLNDAQAPK

SEQ ID NO: 40 - protein, 57 aa, synthetic construct
VDNKFNKESSADLEIAHLPNLNFYQKWAFIRSLMDDPSQSANLLAEAKKLNDAQAPK

SEQ ID NO: 41 - protein, 58 aa, synthetic construct
VDNKFNKEQQFADEEIAMLPNLNFYQKWAFIRKLMDDPSQSANLLAEAKKLNDAQAPK

SEQ ID NO: 42 - protein, 58 aa, synthetic construct
VDNKFNKEHHFADIEISHLPNLNFYQKWAFIRSLMDDPSQSANLLAEAKKLNDAQAPK

SEQ ID NO: 43 - protein, 58 aa, synthetic construct
VDNKFNKESQFADQEIAALPNLNFYQKWAFIRKLMDDPSQSANLLAEAKKLNDAQAPK

SEQ ID NO: 44 - protein, 58 aa, synthetic construct
VDNKFNKEEYFADSEIAQLPNLNFYQKWAFIRKLMDDPSQSANLLAEAKKLNDAQAPK

SEQ ID NO: 45 - protein, 58 aa, synthetic construct
VDNKFNKEEKFADVEIAYLPNLNFYQKWAFIRKLMDDPSQSANLLAEAKKLNDAQAPK

SEQ ID NO: 46 - protein, 58 aa, synthetic construct
VDNKFNKETEFADSEIVRLPNLNFYQKWAFIRSLMDDPSQSANLLAEAKKLNDAQAPK

SEQ ID NO: 47 - protein, 58 aa, synthetic construct
VDNKFNKETTFADQEIASLPNLNFYQKWAFIRKLMDDPSQSANLLAEAKKLNDAQAPK

SEQ ID NO: 48 - protein, 58 aa, synthetic construct
VDNKFNKEYKFADYEIVALPNLNFYQKWAFIRKLMDDPSQSANLLAEAKKLNDAQAPK

SEQ ID NO: 49 - protein, 58 aa, synthetic construct
VDNKFNKEFQFADIEIAELPNLNFYQKWAFIRSLMDDPSQSANLLAEAKKLNDAQAPK

SEQ ID NO: 50 - protein, 58 aa, synthetic construct
VDNKFNKEQTFADQEIAELPNLNFYQKWAFIRKLMDDPSQSANLLAEAKKLNDAQAPK

SEQ ID NO: 51 - protein, 58 aa, synthetic construct
VDNKFNKETSFADKEIALLPNLNFYQKWAFIRKLMDDPSQSANLLAEAKKLNDAQAPK

SEQ ID NO: 52 - protein, 58 aa, synthetic construct
VDNKFNKERMFADIEIQQLPNLNFYQKWAFIRSLMDDPSQSANLLAEAKKLNDAQAPK

SEQ ID NO: 53 - protein, 58 aa, synthetic construct
VDNKFNKEIMFADQEIVSLPNLNFYQKWAFIRSLMDDPSQSANLLAEAKKLNDAQAPK

SEQ ID NO: 54 - protein, 58 aa, synthetic construct
VDNKFNKETQFADQEIAFLPNLNFYQKWAFIRKLMDDPSQSANLLAEAKKLNDAQAPK

SEQ ID NO: 55 - protein, 58 aa, synthetic construct
VDNKFNKELDFADQEIAYLPNLNFYQKWAFIRSLMDDPSQSANLLAEAKKLNDAQAPK

SEQ ID NO: 56 - protein, 58 aa, synthetic construct
VDNKFNKELIFADEEIVALPNLNFYQKWAFIRSLMDDPSQSANLLAEAKKLNDAQAPK

SEQ ID NO: 57 - protein, 58 aa, synthetic construct
VDNKFNKEHFFADSEIAALPNLNFYQKWAFVRSLMDDPSQSANLLAEAKKLNDAQAPK

SEQ ID NO: 58 - protein, 58 aa, synthetic construct
VDNKFNKEHNFADQEIANLPNLNFYQKWAFIRSLMDDPSQSANLLAEAKKLNDAQAPK

SEQ ID NO: 59 - protein, 58 aa, synthetic construct
VDNKFNKEMEFADIEIQSLPNLNFYQKWAFIRSLMDDPSQSANLLAEAKKLNDAQAPK

SEQ ID NO: 60 - protein, 58 aa, synthetic construct
VDNKFNKETWFADSEIAQLPNLNFYQKWAFIRSLMDDPSQSANLLAEAKKLNDAQAPK

SEQ ID NO: 61 - protein, 58 aa, synthetic construct
VDNKFNKEETFADKEIAELPNLNFYQKWAFIRKLMDDPSQSANLLAEAKKLNDAQAPK

SEQ ID NO: 62 - protein, 58 aa, synthetic construct
VDNKFNKEQEFADVEIATLPNLNFYQKWAFIRKLMDDPSQSANLLAEAKKLNDAQAPK

SEQ ID NO: 63 - protein, 58 aa, synthetic construct
VDNKFNKEEHFADIEIASLPNLNFYQKWAFIRSLMDDPSQSANLLAEAKKLNDAQAPK

SEQ ID NO: 64 - protein, 58 aa, synthetic construct
VDNKFNKEFQFADREIAVLPNLNFYQKWAFIRSLMDDPSQSANLLAEAKKLNDAQAPK

SEQ ID NO: 65 - protein, 58 aa, synthetic construct
VDNKFNKEITFADSEIASLPNLNFYQKWAFIRSLMDDPSQSANLLAEAKKLNDAQAPK

SEQ ID NO: 66 - protein, 58 aa, synthetic construct
VDNKFNKEMEFADQEIVVLPNLNFYQKWAFIRKLMDDPSQSANLLAEAKKLNDAQAPK

SEQ ID NO: 67 - protein, 58 aa, synthetic construct
VDNKFNKEKSFADNEIAELPNLNFYQKWAFIRKLMDDPSQSANLLAEAKKLNDAQAPK

SEQ ID NO: 68 - protein, 58 aa, synthetic construct
VDNKFNKETHFADKEIANLPNLNFYQKWAFIRSLMDDPSQSANLLAEAKKLNDAQAPK

SEQ ID NO: 69 - protein, 58 aa, synthetic construct
VDNKFNKEEQFADMEIASLPNLNFYQKWAFIRKLMDDPSQSANLLAEAKKLNDAQAPK

SEQ ID NO: 70 - protein, 58 aa, synthetic construct
VDNKFNKEHSFADNEIAALPNLNFYQKWAFIRSLMDDPSQSANLLAEAKKLNDAQAPK

SEQ ID NO: 71 - protein, 58 aa, synthetic construct
VDNKFNKEATFADLEIEALPNLNFYQKWAFIRKLMDDPSQSANLLAEAKKLNDAQAPK

SEQ ID NO: 72 - protein, 58 aa, synthetic construct
VDNKFNKEENFADNEIASLPNLNFYQKWAFIRSLMDDPSQSANLLAEAKKLNDAQAPK

SEQ ID NO: 73 - protein, 58 aa, synthetic construct
VDNKFNKESAFADIEIASLPNLNFYQKWAFIRKLMDDPSQSANLLAEAKKLNDAQAPK

SEQ ID NO: 74 - protein, 58 aa, synthetic construct
VDNKFNKEELFADQEIANLPNLNFYQKWAFMRSLMDDPSQSANLLAEAKKLNDAQAPK

SEQ ID NO: 75 - protein, 58 aa, synthetic construct
VDNKFNKEAVFADEEITNLPNLNFYQKWAFIRSLMDDPSQSANLLAEAKKLNDAQAPK

SEQ ID NO: 76 - protein, 58 aa, synthetic construct
VDNKFNKETLFADHEIANLPNLNFYQKWAFIRSLMDDPSQSANLLAEAKKLNDAQAPK

SEQ ID NO: 77 - protein, 58 aa, synthetic construct
VDNKFNKEKHFADEEIAKLPNLNFYQKWAFIRKLMDDPSQSANLLAEAKKLNDAQAPK

SEQ ID NO: 78 - protein, 58 aa, synthetic construct
VDNKFNKEERFADLEISVLPNLNFYQKWAFIRKLMDDPSQSANLLAEAKKLNDAQAPK

SEQ ID NO: 79 - protein, 58 aa, synthetic construct
VDNKFNKEMTFADYEISVLPNLNFYQKWAFIRSLMDDPSQSANLLAEAKKLNDAQAPK

SEQ ID NO: 80 - protein, 58 aa, synthetic construct
VDNKFNKEVQFADEEIAMLPNLNFYQKWAFIRKLMDDPSQSANLLAEAKKLNDAQAPK

SEQ ID NO: 81 - protein, 58 aa, synthetic construct
VDNKFNKEELFADEEIQALPNLNFYQKWAFIRSLMDDPSQSANLLAEAKKLNDAQAPK

SEQ ID NO: 82 - protein, 58 aa, synthetic construct
VDNKFNKEDKFADEEIAALPNLNFYQKWAFIRSLMDDPSQSANLLAEAKKLNDAQAPK

SEQ ID NO: 83 - protein, 58 aa, synthetic construct
VDNKFNKEEHFADEEIASLPNLNFYQKWAFIRKLMDDPSQSANLLAEAKKLNDAQAPK

SEQ ID NO: 84 - protein, 58 aa, synthetic construct
VDNKFNKEERFADEEIASLPNLNFYQKWAFIRKLMDDPSQSANLLAEAKKLNDAQAPK

SEQ ID NO: 85 - protein, 58 aa, synthetic construct
VDNKFNKEYRFADTEIADLPNLNFYQKWAFIRKLMDDPSQSANLLAEAKKLNDAQAPK

SEQ ID NO: 86 - protein, 58 aa, synthetic construct
VDNKFNKEQSFADEEIAFLPNLNFYQKWAFIRSLMDDPSQSANLLAEAKKLNDAQAPK

SEQ ID NO: 87 - protein, 58 aa, synthetic construct
VDNKFNKEELFADTEIAQLPNLNFYQKWAFIRSLMDDPSQSANLLAEAKKLNDAQAPK

SEQ ID NO: 88 - protein, 58 aa, synthetic construct
VDNKFNKENQFADIEIAYLPNLNFYQKWAFIRSLMDDPSQSANLLAEAKKLNDAQAPK

SEQ ID NO: 89 - protein, 58 aa, synthetic construct
VDNKFNKEHFFADTEIAFLPNLNFYQKWAFIRKLMDDPSQSANLLAEAKKLNDAQAPK

SEQ ID NO: 90 - protein, 58 aa, synthetic construct
VDNKFNKEAVFADHEIAFLPNLNFYQKWAFIRKLMDDPSQSANLLAEAKKLNDAQAPK

SEQ ID NO: 91 - protein, 58 aa, synthetic construct
VDNKFNKENEFADEEIALLPNLNFYQKWAFIRSLMDDPSQSANLLAEAKKLNDAQAPK

SEQ ID NO: 92 - protein, 58 aa, synthetic construct
VDNKFNKEDDFADEEIAALPNLNFYQKWAFIRSLMDDPSQSANLLAEAKKLNDAQAPK

SEQ ID NO: 93 - protein, 58 aa, synthetic construct
VDNKFNKETDFADVEIVALPNLNFYQKWAFIRKLMDDPSQSANLLAEAKKLNDAQAPK

SEQ ID NO: 94 - protein, 58 aa, synthetic construct
VDNKFNKEERFADMEISNLPNLNFYQKWAFIRSLMDDPSQSANLLAEAKKLNDAQAPK

SEQ ID NO: 95 - protein, 58 aa, synthetic construct
VDNKFNKEEAFADTEIAALPNLNFYQKWAFIRSLMDDPSQSANLLAEAKKLNDAQAPK

SEQ ID NO: 96 - protein, 58 aa, synthetic construct
VDNKFNKEETEADMEISNLPNLNFYQKWAFIRSLMDDPSQSANLLAEAKKLNDAQAPK

SEQ ID NO: 97 - protein, 58 aa, synthetic construct
VDNKFNKEQKFADDEIAALPNLNFYQKWAFIRSLMDDPSQSANLLAEAKKLNDAQAPK

SEQ ID NO: 98 - protein, 58 aa, synthetic construct
VDNKFNKEEAFADMEIERLPNLNFYQKWAFIRSLMDDPSQSANLLAEAKKLNDAQAPK

SEQ ID NO: 99 - protein, 58 aa, synthetic construct
VDNKFNKEINFADEEIAYLPNLNFYQKWAFIRSLMDDPSQSANLLAEAKKLNDAQAPK

SEQ ID NO: 100 - protein, 58 aa, synthetic construct
VDNKFNKEEQFADQEIALLPNLNFYQKWAFIRKLMDDPSQSANLLAEAKKLNDAQAPK

SEQ ID NO: 101 - protein, 58 aa, synthetic construct
VDNKFNKEHAFADQEIAILPNLNFYQKWAFIRSLVDDPSQSANLLAEAKKLNDAQAPK

SEQ ID NO: 102 - protein, 58 aa, synthetic construct
VDNKFNKEDTFADNEIAFLPNLNFYQKWAFIRSLMDDPSQSANLLAEAKKLNDAQAPK

SEQ ID NO: 103 - protein, 58 aa, synthetic construct
VDNKFNKEQYFADYEIAHLPNLNFYQKWAFIRKLMDDPSQSANLLAEAKKLNDAQAPK

SEQ ID NO: 104 - protein, 58 aa, synthetic construct
VDNKFNKEEHFADLEISALPNLNFYQKWAFIRSLLDDPSQSANLLAEAKKLNDAQAPK

SEQ ID NO: 105 - protein, 58 aa, synthetic construct
VDNKFNKEISFADQEIATLPNLNFYQKWAFIRSLMDDPSQSANLLAEAKKLNDAQAPK

SEQ ID NO: 106 - protein, 58 aa, synthetic construct
VDNKFNKESNFADYEIAQLPNLNFYQKWAFIRKLIDDPSQSANLLAEAKKLNDAQAPK

SEQ ID NO: 107 - protein, 58 aa, synthetic construct
VDNKFNKEYDFADEEITALPNLNFYQKWAFIRKLLDDPSQSANLLAEAKKLNDAQAPK

SEQ ID NO: 108 - protein, 58 aa, synthetic construct
VDNKFNKEAQFADIEISFLPNLNFYQKWAFIRSLVDDPSQSANLLAEAKKLNDAQAPK

SEQ ID NO: 109 - protein, 58 aa, synthetic construct
VDNKFNKEVDFADEEIAQLPNLNFYQKWAFIRSLMDDPSQSANLLAEAKKLNDAQAPK

SEQ ID NO: 110 - protein, 58 aa, synthetic construct
VDNKFNKEKEHADEEIAALPNLNFYQKWAFIRSLMDDPSQSANLLAEAKKLNDAQAPK

SEQ ID NO: 111 - protein, 58 aa, synthetic construct
VDNKFNKEEWFADNEIAELPNLNFYQKWAFIRSLMDDPSQSANLLAEAKKLNDAQAPK

SEQ ID NO: 112 - protein, 58 aa, synthetic construct
VDNKFNKEEQFADQEITNLPNLNFYQKWAFIRSLMDDPSQSANLLAEAKKLNDAQAPK

SEQ ID NO: 113 - protein, 58 aa, synthetic construct
VDNKFNKEESFADTEIANLPNLNFYQKWAFIRSLMDDPSQSANLLAEAKKLNDAQAPK

SEQ ID NO: 114 - protein, 58 aa, synthetic construct
VDNKFNKEDVEADKEIAQLPNLNFYQKWAFIRKLMDDPSQSANLLAEAKKLNDAQAPK

SEQ ID NO: 115 - protein, 58 aa, synthetic construct
VDNKFNKERYYADVEIAKLPNLNFFQKWAFIRKLMDDPSQSANLLAEAKKLNDAQAPK

SEQ ID NO: 116 - protein, 58 aa, synthetic construct
VDNKFNKENEFADEEIAELPNLNFYQKWAFIRSLMDDPSQSANLLAEAKKLNDAQAPK

SEQ ID NO: 117 - protein, 58 aa, synthetic construct
VDNKFNKEQFQADMEIAQLPNLNFYQKWAFIRSLMDDPSQSANLLAEAKKLNDAQAPK

SEQ ID NO: 118 - protein, 58 aa, synthetic construct
VDNKFNKEFAFADEEIAQLPNLNFYQKWAFIRSLMDDPSQSANLLAEAKKLNDAQAPK

SEQ ID NO: 119 - protein, 58 aa, synthetic construct
VDNKFNKEYKFADVEITDLPNLNFYQKWAFIRSLMDDPSQSANLLAEAKKLNDAQAPK

SEQ ID NO: 120 - protein, 58 aa, synthetic construct
VDNKFNKEFQADMEISHLPNLNFFYQKWAFIRSLMDDPSQSANLLAEAKKLNDAQAPK

SEQ ID NO: 121 - protein, 58 aa, synthetic construct
VDNKFNKEEETADMEIARLPNLNFYQKWAFIRSLMDDPSQSANLLAEAKKLNDAQAPK

SEQ ID NO: 122 - protein, 58 aa, synthetic construct
VDNKFNKEKQQADEEIAHLPNLNFYQKWAFIRSLMDDPSQSANLLAEAKKLNDAQAPK

SEQ ID NO: 123 - protein, 58 aa, synthetic construct
GDNKFNKEDQFADTEIAALPNLNFYQKWAFIRSLMDDPSQSANLLAEAKKLNDAQAPK

SEQ ID NO: 124 - protein, 58 aa, synthetic construct
VDNKFNKEVRFADTEIAYLPNLNFYQKWAFIRSLMDDPSQSANLLAEAKKLNDAQAPK

SEQ ID NO: 125 - protein, 58 aa, synthetic construct
VDNKFNKEDMFADMEIATLPNLNFYQKWAFIRKLMDDPSQSANLLAEAKKLNDAQAPK

SEQ ID NO: 126 - protein, 58 aa, synthetic construct
VDNKFNKEQSFADEEIAILPNLNFYQKWAFVRSLMDDPSQSANLLAEAKKLNDAQAPK

SEQ ID NO: 127 - protein, 58 aa, synthetic construct
VDNKFNKEEQYADVEIAYLPNLNFYQKWAFIRKLMDDPSQSANLLAEAKKLNDAQAPK

SEQ ID NO: 128 - protein, 58 aa, synthetic construct
VDNKFNKEESFADWEIQKLPNLNFYQKWAFIRKLMDDPSQSANLLAEAKKLNDAQAPK

SEQ ID NO: 129 - protein, 71 aa, synthetic construct
GSSHHHHHHYYLEVDNKFNKEETFADLEISNLPNLNFYQKWAFIRSLMDDPSQSANLLAEAKKLNDAQAPK

SEQ ID NO: 130 - protein, 139 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 131 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKEFWIAESEIESLPNLNIYQKWAFKYSLADDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 132 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 133 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKEQFYARDEIDLLPNLNEDQKWAFYMSLIDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 134 - protein, 192 aa, synthetic construct
VDNKFNKENQFADEEIAALPNLNFYQKWAFIRKLMDDPSQSANLLAEAKKLNDAQAPKGGGSGVDNKFNKENQFADEEIAALPNLNFYQKWAFIRKLMDDPSQSANLLAEAKKLNDAQAPKGGGSGVDNKFNKEVQMAQFEIRKLPNLNHHQSFAFIKSLMDDPSQSANLLAEAKKLNDAQAPKYYHHHHHH

SEQ ID NO: 135 - protein, 202 aa, synthetic construct
VDNKFNKENQFADEEIAALPNLNFYQKWAFIRKLMDDPSQSANLLAEAKKLNDAQAPKGGGSGGGGSGVDNKFNKENQFADEEIAALPNLNFYQKWAFIRKLMDDPSQSANLLAEAKKLNDAQAPKGGGSGGGGSGVDNKFNKEVQMAQFEIRKLPNLNHHQSFAFIKSLMDDPSQSANLLAEAKKLNDAQAPKYYHHHHHH

SEQ ID NO: 136 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 137 - protein, 212 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKYYHHHHHH

SEQ ID NO: 138 - protein, 212 aa, synthetic construct
VDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKYYHHHHHH

SEQ ID NO: 139 - protein, 131 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 140 - protein, 204 aa, synthetic construct
VDNKFNKENQFADEEIAALPNLNFYQKWAFIRKLMDDPSQSANLLAEAKKLNDAQAPKGGGSGGGGSGGGGSGVDNKFNKEFWIAESEIESLPNLNIYQKWAFKYSLADDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 141 - protein, 204 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 142 - protein, 204 aa, synthetic construct
VDNKFNKENQFADEEIAALPNLNFYQKWAFIRKLMDDPSQSANLLAEAKKLNDAQAPKGGGSGGGGSGGGGSGVDNKFNKEQFYARDEIDLLPNLNEDQKWAFYMSLIDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 143 - protein, 184 aa, synthetic construct
VDNKFNKENQFADEEIAALPNLNFYQKWAFIRKLMDDPSQSANLLAEAKKLNDAQAPKGGGSGVDNKFNKENQFADEEIAALPNLNFYQKWAFIRKLMDDPSQSANLLAEAKKLNDAQAPKGGGSGVDNKFNKEVQMAQFEIRKLPNLNHHQSFAFIKSLMDDPSQSANLLAEAKKLNDAQAPK

SEQ ID NO: 144 - protein, 194 aa, synthetic construct
VDNKFNKENQFADEEIAALPNLNFYQKWAFIRKLMDDPSQSANLLAEAKKLNDAQAPKGGGSGGGGSGVDNKFNKENQFADEEIAALPNLNFYQKWAFIRKLMDDPSQSANLLAEAKKLNDAQAPKGGGSGGGGSGVDNKFNKEVQMAQFEIRKLPNLNHHQSFAFIKSLMDDPSQSANLLAEAKKLNDAQAPK

SEQ ID NO: 145 - protein, 204 aa, synthetic construct
VDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 146 - protein, 204 aa, synthetic construct
VDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPK

SEQ ID NO: 147 - protein, 204 aa, synthetic construct
VDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPK

SEQ ID NO: 148 - protein, 270 aa, synthetic construct
MMVDNKFNKENQFADEEIAALPNLNFYQKWAFIRKLMDDPSQSANLLAEAKKLNDAQAPKGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 149 - protein, 194 aa, synthetic construct
MMVDNKFNKENQFADEEIAALPNLNFYQKWAFIRKLMDDPSQSANLLAEAKKLNDAQAPKGGGSGVDNKFNKENQFADEEIAALPNLNFYQKWAFIRKLMDDPSQSANLLAEAKKLNDAQAPKGGGSGVDNKFNKEVQMAQFEIRKLPNLNHHQSFAFIKSLMDDPSQSANLLAEAKKLNDAQAPKYYHHHHHH

SEQ ID NO: 150 - protein, 204 aa, synthetic construct
MMVDNKFNKENQFADEEIAALPNLNFYQKWAFIRKLMDDPSQSANLLAEAKKLNDAQAPKGGGSGGGGSGVDNKFNKENQFADEEIAALPNLNFYQKWAFIRKLMDDPSQSANLLAEAKKLNDAQAPKGGGSGGGGSGVDNKFNKEVQMAQFEIRKLPNLNHHQSFAFIKSLMDDPSQSANLLAEAKKLNDAQAPKYYHHHHHH

SEQ ID NO: 151 - protein, 214 aa, synthetic construct
MMVDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKYYHHHHHH

SEQ ID NO: 152 - protein, 214 aa, synthetic construct
MMVDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKYYHHHHHH

SEQ ID NO: 153 - protein, 214 aa, synthetic construct
MMVDNKFNKEVQMAQFEIRKLPNLNHHQSFAFIKSLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKGGGSGGGGSGGGGSGVDNKFNKENQFADEEIAALPNLNFYQKWAFIRKLMDDPSQSANLLAEAKKLNDAQAPKYYHHHHHH

SEQ ID NO: 154 - protein, 124 aa, synthetic construct
VDNKFNKENQFADEEIAALPNLNFYQKWAFIRKLMDDPSQSANLLAEAKKLNDAQAPKVDNKFNKEVQMAQFEIRKLPNLNHHQSFAFIKSLMDDPSQSANLLAEAKKLNDAQAPKYYHHHHHH

SEQ ID NO: 155 - protein, 124 aa, synthetic construct
VDNKFNKEVQMAQFEIRKLPNLNHHQSFAFIKSLMDDPSQSANLLAEAKKLNDAQAPKVDNKFNKENQFADEEIAALPNLNFYQKWAFIRKLMDDPSQSANLLAEAKKLNDAQAPKYYHHHHHH

SEQ ID NO: 156 - protein, 125 aa, synthetic construct
VDNKFNKENQFADEEIAALPNLNFYQKWAFIRKLMDDPSQSANLLAEAKKLNDAQAPKGVDNKFNKEVQMAQFEIRKLPNLNHHQSFAFIKSLMDDPSQSANLLAEAKKLNDAQAPKYYHHHHHH

SEQ ID NO: 157 - protein, 125 aa, synthetic construct
VDNKFNKEVQMAQFEIRKLPNLNHHQSFAFIKSLMDDPSQSANLLAEAKKLNDAQAPKGVDNKFNKENQFADEEIAALPNLNFYQKWAFIRKLMDDPSQSANLLAEAKKLNDAQAPKYYHHHHHH

SEQ ID NO: 158 - protein, 113 aa, synthetic construct
MNKENQFADEEIAALPNLNFYQKWAFIRKLMDDPSQSANLLAEAKKLNDAQGNKEVQMAQFEIRKLPNLNHHQSFAFIKSLMDDPSQSANLLAEAKKLNDAQAPKYYHHHHHH

SEQ ID NO: 159 - protein, 115 aa, synthetic construct
MNKENQFADEEIAALPNLNFYQKWAFIRKLMDDPSQSANLLAEAKKLNDAQGVDNKFNKEVQMAQFEIRKLPNLNHHQSFAFIKSLMDDPSQSANLLAEAKKLNDAQYYHHHHHH

SEQ ID NO: 160 - protein, 118 aa, synthetic construct
MNKENQFADEEIAALPNLNFYQKWAFIRKLMDDPSQSANLLAEAKKLNDAQGVDNKFNKEVQMAQFEIRKLPNLNHHQSFAFIKSLMDDPSQSANLLAEAKKLNDAQAPKYYHHHHHH

SEQ ID NO: 161 - protein, 120 aa, synthetic construct
GDNKFNKENQFADEEIAALPNLNFYQKWAFIRKLMDDPSQSANLLAEAKKLNDAQAPKGNKEVQMAQFEIRKLPNLNHHQSFAFIKSLMDDPSQSANLLAEAKKLNDAQAPKYYHHHHHH

SEQ ID NO: 162 - protein, 122 aa, synthetic construct
GDNKFNKENQFADEEIAALPNLNFYQKWAFIRKLMDDPSQSANLLAEAKKLNDAQAPKGVDNKFNKEVQMAQFEIRKLPNLNHHQSFAFIKSLMDDPSQSANLLAEAKKLNDAQYYHHHHHH

SEQ ID NO: 163 - protein, 125 aa, synthetic construct
GDNKFNKENQFADEEIAALPNLNFYQKWAFIRKLMDDPSQSANLLAEAKKLNDAQAPKGVDNKFNKEVQMAQFEIRKLPNLNHHQSFAFIKSLMDDPSQSANLLAEAKKLNDAQAPKYYHHHHHH

SEQ ID NO: 164 - protein, 59 aa, synthetic construct
VDNKFNKEETFADLEISNLPNLNFYQKWAFIRSLMDDPSQSANLLAEAKKLNDAQAPKC

SEQ ID NO: 165 - protein, 53 aa, synthetic construct
GNKENQFADEEIAALPNLNFYQKWAFIRKLNLDDPSQSANLLAEAKKLNDAQK

SEQ ID NO: 166 - protein, 133 aa, synthetic construct
MMVDNKFNKENQFADEEIAALPNLNFYQKWAFIRKLMDDPSQSANLLAEAKKLNDAQAPKGGGSGGGGSGGGGSGVDNKFNKEVQMAQFEIRKLPNLNHHQSFAFIKSLMDDPSQSANLLAEAKKLNDAQAPK

SEQ ID NO: 167 - protein, 58 aa, synthetic construct
VDNKFNKEVQMAQFEIRKLPNLNHHQSFAFIKSLMDDPSQSANLLAEAKKLNDAQAPK

SEQ ID NO: 168 - protein, 58 aa, synthetic construct
GDNKFNKENQFADEEIAALPNLNFYQKWAFIRKLMDDPSQSANLLAEAKKLNDAQAPK

SEQ ID NO: 169 - protein, 50 aa, synthetic construct
NKENQFADEEIAALPNLNFYQKWAFIRKLMDDPSQSANLLAEAKKLNDAQ

SEQ ID NO: 170 - protein, 27 aa, synthetic construct
ETFADLEISNLPNLNFYQKWAFIRSLM

SEQ ID NO: 171 - protein, 27 aa, synthetic construct
NQFADEEIAALPNLNFYQKWAFIRKLM

SEQ ID NO: 172 - protein, 27 aa, synthetic construct
EIFADREIAFLPNLNFYQKWAFIRKLM

SEQ ID NO: 173 - protein, 27 aa, synthetic construct
SQFADEEIEALPNLNFYQKWAFIRSLM

SEQ ID NO: 174 - protein, 27 aa, synthetic construct
QEFADYEIATLPNLNFYQKWAFIRSLM

SEQ ID NO: 175 - protein, 27 aa, synthetic construct
DSFADTEIAKLPNLNFYQKWAFIRSLM

SEQ ID NO: 176 - protein, 27 aa, synthetic construct
EIFADQEIEALPNLNFYQKWAFIRSLM

SEQ ID NO: 177 - protein, 27 aa, synthetic construct
DYFADYEIATLPNLNFYQKWAFIRSLM

SEQ ID NO: 178 - protein, 27 aa, synthetic construct
EQFADEEIAHLPNLNFYQKWAFIRSLM

SEQ ID NO: 179 - protein, 27 aa, synthetic construct
SMFADIEITKLPNLNFYQKWAFIRKLM

SEQ ID NO: 180 - protein, 27 aa, synthetic construct
EVFADEEIASLPNLNFYQKWAFIRSLM

SEQ ID NO: 181 - protein, 27 aa, synthetic construct
VREADEEIAALPNLNFYQKWAFIRKLM

SEQ ID NO: 182 - protein, 27 aa, synthetic construct
HQFADYEIAMLPNLNFYQKWAFIRSLM

SEQ ID NO: 183 - protein, 27 aa, synthetic construct
HFFADKEIAKLPNLNFYQKWAFIRSLM

SEQ ID NO: 184 - protein, 27 aa, synthetic construct
KAFADTEIVNLPNLNFYQKWAFIRSLM

SEQ ID NO: 185 - protein, 27 aa, synthetic construct
SEFADAEIANLPNLNFYQKWAFIRKLM

SEQ ID NO: 186 - protein, 27 aa, synthetic construct
ENFADHEIALLPNLNFYQKWAFIRKLM

SEQ ID NO: 187 - protein, 27 aa, synthetic construct
DSHADEEIAALPNLNFYQKWAFIRKLM

SEQ ID NO: 188 - protein, 27 aa, synthetic construct
SKFADEEIAKLPNLNFYQKWAFIRSLM

SEQ ID NO: 189 - protein, 27 aa, synthetic construct
QQFADEEIAMLPNLNFYQKWAFIRKLM

SEQ ID NO: 190 - protein, 27 aa, synthetic construct
HHFADIEISHLPNLNFYQKWAFIRSLM

SEQ ID NO: 191 - protein, 27 aa, synthetic construct
SQFADQEIAALPNLNFYQKWAFIRKLM

SEQ ID NO: 192 - protein, 27 aa, synthetic construct
EYFADSEIAQLPNLNFYQKWAFIRKLM

SEQ ID NO: 193 - protein, 27 aa, synthetic construct
EKFADVEIAYLPNLNFYQKWAFIRKLM

SEQ ID NO: 194 - protein, 27 aa, synthetic construct
TEFADSEIVRLPNLNFYQKWAFIRSLM

SEQ ID NO: 195 - protein, 27 aa, synthetic construct
TTFADQEIASLPNLNFYQKWAFIRKLM

SEQ ID NO: 196 - protein, 27 aa, synthetic construct
YKFADYEIVALPNLNFYQKWAFIRKLM

SEQ ID NO: 197 - protein, 27 aa, synthetic construct
FQFADIEIAELPNLNFYQKWAFIRSLM

SEQ ID NO: 198 - protein, 27 aa, synthetic construct
QTFADQEIAELPNLNFYQKWAFIRKLM

SEQ ID NO: 199 - protein, 27 aa, synthetic construct
TSFADKEIALLPNLNFYQKWAFIRKLM

SEQ ID NO: 200 - protein, 27 aa, synthetic construct
RMFADIEIQQLPNLNFYQKWAFIRSLM

SEQ ID NO: 201 - protein, 27 aa, synthetic construct
IMFADQEIVSLPNLNFYQKWAFIRSLM

SEQ ID NO: 202 - protein, 27 aa, synthetic construct
TQFADQEIAFLPNLNFYQKWAFIRKLM

SEQ ID NO: 203 - protein, 27 aa, synthetic construct
LDFADQEIAYLPNLNFYQKWAFIRSLM

SEQ ID NO: 204 - protein, 27 aa, synthetic construct
LIFADEEIVALPNLNFYQKWAFIRSLM

SEQ ID NO: 205 - protein, 27 aa, synthetic construct
HFFADSEIAALPNLNFYQKWAFVRSLM

SEQ ID NO: 206 - protein, 27 aa, synthetic construct
HNFADQEIANLPNLNFYQKWAFIRSLM

SEQ ID NO: 207 - protein, 27 aa, synthetic construct
MEFADIEIQSLPNLNFYQKWAFIRSLM

SEQ ID NO: 208 - protein, 27 aa, synthetic construct
TWFADSEIAQLPNLNFYQKWAFIRSLM

SEQ ID NO: 209 - protein, 27 aa, synthetic construct
ETFADKEIAELPNLNFYQKWAFIRKLM

SEQ ID NO: 210 - protein, 27 aa, synthetic construct
QEFADVEIATLPNLNFYQKWAFIRKLM

SEQ ID NO: 211 - protein, 27 aa, synthetic construct
EHFADIEIASLPNLNFYQKWAFIRSLM

SEQ ID NO: 212 - protein, 27 aa, synthetic construct
FQFADREIAVLPNLNFYQKWAFIRSLM

SEQ ID NO: 213 - protein, 27 aa, synthetic construct
ITFADSEIASLPNLNFYQKWAFIRSLM

SEQ ID NO: 214 - protein, 27 aa, synthetic construct
MEFADQEIVVLPNLNFYQKWAFIRKLM

SEQ ID NO: 215 - protein, 27 aa, synthetic construct
KSFADNEIAELPNLNFYQKWAFIRKLM

SEQ ID NO: 216 - protein, 27 aa, synthetic construct
THFADKEIANLPNLNFYQKWAFIRSLM

SEQ ID NO: 217 - protein, 27 aa, synthetic construct
EQFADMEIASLPNLNFYQKWAFIRKLM

SEQ ID NO: 218 - protein, 27 aa, synthetic construct
HSFADNEIAALPNLNFYQKWAFIRSLM

SEQ ID NO: 219 - protein, 27 aa, synthetic construct
ATFADLEIEALPNLNFYQKWAFIRKLM

SEQ ID NO: 220 - protein, 27 aa, synthetic construct
ENFADNEIASLPNLNFYQKWAFIRSLM

SEQ ID NO: 221 - protein, 27 aa, synthetic construct
SAFADIEIASLPNLNFYQKWAFIRKLM

SEQ ID NO: 222 - protein, 27 aa, synthetic construct
ELFADQEIANLPNLNFYQKWAFMRSLM

SEQ ID NO: 223 - protein, 27 aa, synthetic construct
AVFADEEITNLPNLNFYQKWAFIRSLM

SEQ ID NO: 224 - protein, 27 aa, synthetic construct
TLFADHEIANLPNLNFYQKWAFIRSLM

SEQ ID NO: 225 - protein, 27 aa, synthetic construct
KHFADEEIAKLPNLNFYQKWAFIRKLM

SEQ ID NO: 226 - protein, 27 aa, synthetic construct
ERFADLEISVLPNLNFYQKWAFIRKLM

SEQ ID NO: 227 - protein, 27 aa, synthetic construct
MTFADYEISVLPNLNFYQKWAFIRSLM

SEQ ID NO: 228 - protein, 27 aa, synthetic construct
VQFADEEIAMLPNLNFYQKWAFIRKLM

SEQ ID NO: 229 - protein, 27 aa, synthetic construct
ELFADEEIQALPNLNFYQKWAFIRSLM

SEQ ID NO: 230 - protein, 27 aa, synthetic construct
DKFADEEIAALPNLNFYQKWAFIRSLM

SEQ ID NO: 231 - protein, 27 aa, synthetic construct
EHFADEEIASLPNLNFYQKWAFIRKLM

SEQ ID NO: 232 - protein, 27 aa, synthetic construct
ERFADEEIASLPNLNFYQKWAFIRKLM

SEQ ID NO: 233 - protein, 27 aa, synthetic construct
YRFADTEIADLPNLNFYQKWAFIRKLM

SEQ ID NO: 234 - protein, 27 aa, synthetic construct
QSFADEEIAFLPNLNFYQKWAFIRSLM

SEQ ID NO: 235 - protein, 27 aa, synthetic construct
ELFADTEIAQLPNLNFYQKWAFIRSLM

SEQ ID NO: 236 - protein, 27 aa, synthetic construct
NQFADIEIAYLPNLNFYQKWAFIRSLM

SEQ ID NO: 237 - protein, 27 aa, synthetic construct
HFFADTEIAFLPNLNFYQKWAFIRKLM

SEQ ID NO: 238 - protein, 27 aa, synthetic construct
AVFADHEIAFLPNLNFYQKWAFIRKLM

SEQ ID NO: 239 - protein, 27 aa, synthetic construct
NEFADEEIALLPNLNFYQKWAFIRSLM

SEQ ID NO: 240 - protein, 27 aa, synthetic construct
DDFADEEIAALPNLNFYQKWAFIRSLM

SEQ ID NO: 241 - protein, 27 aa, synthetic construct
TDFADVEIVALPNLNFYQKWAFIRKLM

SEQ ID NO: 242 - protein, 27 aa, synthetic construct
ERFADMEISNLPNLNFYQKWAFIRSLM

SEQ ID NO: 243 - protein, 27 aa, synthetic construct
EAFADTEIAALPNLNFYQKWAFIRSLM

SEQ ID NO: 244 - protein, 27 aa, synthetic construct
ETEADMEISNLPNLNFYQKWAFIRSLM

SEQ ID NO: 245 - protein, 27 aa, synthetic construct
QKFADDEIAALPNLNFYQKWAFIRSLM

SEQ ID NO: 246 - protein, 27 aa, synthetic construct
EAFADMEIERLPNLNFYQKWAFIRSLM

SEQ ID NO: 247 - protein, 27 aa, synthetic construct
INFADEEIAYLPNLNFYQKWAFIRSLM

SEQ ID NO: 248 - protein, 27 aa, synthetic construct
EQFADQEIALLPNLNFYQKWAFIRKLM

SEQ ID NO: 249 - protein, 27 aa, synthetic construct
HAFADQEIAILPNLNFYQKWAFIRSLV

SEQ ID NO: 250 - protein, 27 aa, synthetic construct
DTFADNEIAFLPNLNFYQKWAFIRSLM

SEQ ID NO: 251 - protein, 27 aa, synthetic construct
QYFADYEIAHLPNLNFYQKWAFIRKLM

SEQ ID NO: 252 - protein, 27 aa, synthetic construct
EHFADLEISALPNLNFYQKWAFIRSLL

SEQ ID NO: 253 - protein, 27 aa, synthetic construct
ISFADQEIATLPNLNFYQKWAFIRSLM

SEQ ID NO: 254 - protein, 27 aa, synthetic construct
SNFADYEIAQLPNLNFYQKWAFIRKLI

SEQ ID NO: 255 - protein, 27 aa, synthetic construct
YDFADEEITALPNLNFYQKWAFIRKLL

SEQ ID NO: 256 - protein, 27 aa, synthetic construct
AQFADIEISFLPNLNFYQKWAFIRSLV

SEQ ID NO: 257 - protein, 27 aa, synthetic construct
VDFADEEIAQLPNLNFYQKWAFIRSLM

SEQ ID NO: 258 - protein, 27 aa, synthetic construct
KEHADEEIAALPNLNFYQKWAFIRSLM

SEQ ID NO: 259 - protein, 27 aa, synthetic construct
EWFADNEIAELPNLNFYQKWAFIRSLM

SEQ ID NO: 260 - protein, 27 aa, synthetic construct
EQFADQEITNLPNLNFYQKWAFIRSLM

SEQ ID NO: 261 - protein, 27 aa, synthetic construct
ESFADTEIANLPNLNFYQKWAFIRSLM

SEQ ID NO: 262 - protein, 27 aa, synthetic construct
DVEADKEIAQLPNLNFYQKWAFIRKLM

SEQ ID NO: 263 - protein, 27 aa, synthetic construct
RYYADVEIAKLPNLNFFQKWAFIRKLM

SEQ ID NO: 264 - protein, 27 aa, synthetic construct
NEFADEEIAELPNLNFYQKWAFIRSLM

SEQ ID NO: 265 - protein, 27 aa, synthetic construct
QFQADMEIAQLPNLNFYQKWAFIRSLM

SEQ ID NO: 266 - protein, 27 aa, synthetic construct
FAFADEEIAQLPNLNFYQKWAFIRSLM

SEQ ID NO: 267 - protein, 27 aa, synthetic construct
YKFADVEITDLPNLNFYQKWAFIRSLM

SEQ ID NO: 268 - protein, 27 aa, synthetic construct
EETADMEIARLPNLNFYQKWAFIRSLM

SEQ ID NO: 269 - protein, 27 aa, synthetic construct
KQQADEEIAHLPNLNFYQKWAFIRSLM

SEQ ID NO: 270 - protein, 27 aa, synthetic construct
DQFADTEIAALPNLNFYQKWAFIRSLM

SEQ ID NO: 271 - protein, 27 aa, synthetic construct
VRFADTEIAYLPNLNFYQKWAFIRSLM

SEQ ID NO: 272 - protein, 27 aa, synthetic construct
VRFADTEIAYLPNLNFYQKWAFIRSLM

SEQ ID NO: 273 - protein, 27 aa, synthetic construct
QSFADEEIAILPNLNFYQKWAFVRSLM

SEQ ID NO: 274 - protein, 27 aa, synthetic construct
EQYADVEIAYLPNLNFYQKWAFIRKLM

SEQ ID NO: 275 - protein, 27 aa, synthetic construct
ESFADWEIQKLPNLNFYQKWAFIRKLM

SEQ ID NO: 276 - protein, 58 aa, synthetic construct
VDNKFNKEQFYARDEIDLLPNLNEDQKWAFYMSLIDDPSQSANLLAEAKKLNDAQAPK

SEQ ID NO: 277 - protein, 58 aa, synthetic construct
VDNKFNKEFWIAESEIESLPNLNIYQKWAFKYSLADDPSQSANLLAEAKKLNDAQAPK

SEQ ID NO: 278 - protein, 10 aa, synthetic construct
  VAR_SEQ 1: note = X is A, C, D, E, F, G, H, I, K, L, M, N, P, Q, R, S, T, V, W, or Y
  VAR_SEQ 2: note = X is A, C, D, E, F, G, H, I, K, L, M, N, P, Q, R, S, T, V, W, or Y
  VAR_SEQ 3: note = X is E, F, H, Q, T or Y
  VAR_SEQ 6: note = X is A, C, D, E, F, G, H, I, K, L, M, N, P, Q, R, S, T, V, W, or Y
  VAR_SEQ 9: note = X is E, Q, S, T or V
  VAR_SEQ 10: note = X is A, C, D, E, F, G, H, I, K, L, M, N, P, Q, R, S, T, V, W, or Y
XXXADXEIXX

SEQ ID NO: 279 - protein, 12 aa, synthetic construct
  VAR_SEQ 2: note = X is F or Y
  VAR_SEQ 8: note = X is I, M, or V
  VAR_SEQ 10: note = X is K or S
  VAR_SEQ 12: note = X is I, L, M, or V
FXQKWAFXRXLX

SEQ ID NO: 280 - DNA, 121 bp, synthetic construct
  source 1..121: mol_type = other DNA
  misc_difference 21..23: note = nnn occupied by a codon for any one of the 20 standard amino acids, except C and P, with each codon selected with equal probability
  misc_difference 30..32: note = nnn occupied by a codon for any one of the 20 standard amino acids, except C and P, with each codon selected with equal probability
  misc_difference 33..35: note = nnn occupied by a codon for I with a 60% probability or a codon for any one of H, Y, K, or D with 10% probability each
  misc_difference 42..44: note = nnn occupied by a codon for any one of the 20 standard amino acids, except C and P, with each codon selected with equal probability
  misc_difference 45..47: note = nnn occupied by a codon for any one of the 20 standard amino acids, except C and P, with each codon selected with equal probability
  misc_difference 51..53: note = nnn occupied by a codon for any one of the 20 standard amino acids, except C and P, with each codon selected with equal probability
  misc_difference 72..74: note = nnn occupied by a codon for any one of the 20 standard amino acids, except C and P, with each codon selected with equal probability
  misc_difference 75..77: note = nnn occupied by a codon for any one of the 20 standard amino acids, except C and P, with each codon selected with equal probability
  misc_difference 84..86: note = nnn occupied by a codon for any one of the 20 standard amino acids, except C and P, with each codon selected with equal probability
  misc_difference 87..89: note = nnn occupied by a codon for any one of the 20 standard amino acids, except C and P, with each codon selected with equal probability
  misc_difference 93..95: note = nnn occupied by a codon for any one of the 20 standard amino acids, except C and P, with each codon selected with equal probability
  misc_difference 96..98: note = nnn occupied by a codon for any one of the 20 standard amino acids, except C and P, with each codon selected with equal probability
  misc_difference 99..101: note = nnn occupied by a codon for any one of the 20 standard amino acids, except C and P, with each codon selected with equal probability
gcgctttggcttgggtcatcnnntaaactnnnnnngaaggcnnnnnnttgnnnnnngttcaggttcggcagnnnnnngatctcnnnnnncgcnnnnnnnnnttctttgttgaatttgttgt

SEQ ID NO: 281 - DNA, 121 bp, synthetic construct; identical to SEQ ID NO: 280
  source 1..121: mol_type = other DNA

SEQ ID NO: 282 - DNA, 41 bp, synthetic construct
  source 1..41: mol_type = other DNA
ttagcttctgctagcaagttagcgctttggcttgggtcatc

SEQ ID NO: 283 - DNA, 121 bp, synthetic construct
  source 1..121: mol_type = other DNA
  misc_difference 21..23: note = nnn occupied by a codon for any one of the standard 20 amino acids, except C, P and G, with equal probability of each
  misc_difference 24..26: note = nnn occupied by a codon for any one of the standard 20 amino acids, except C, P and G, with equal probability of each
  misc_difference 27..29: note = nnn occupied by a codon for F with 90% probability and by a codon for any one of the remaining 19 standard amino acids, except C, P and G, with a 10% probability distributed equally between each of the remaining 17 amino acids
  misc_difference 33..35: note = nnn occupied by a codon for D with 90% probability and by a codon for any one of the remaining 19 standard amino acids, except C, P and G, with a 10% probability distributed equally between each of the remaining 17 amino acids
  misc_difference 36..38: note = nnn occupied by a codon for any one of the standard 20 amino acids, except C, P and G, with equal probability of each
  misc_difference 45..47: note = nnn occupied by a codon for A with 20% probability and by a codon for any one of the remaining 19 standard amino acids, except C, P and G, with a 80% probability distributed equally between each of the remaining 17 amino acids
  misc_difference 48..50: note = nnn occupied by a codon for A with 20% probability and by a codon for any one of the remaining 19 standard amino acids, except C, P and G, with a 80% probability distributed equally between each of the remaining 17 amino acids
  misc_difference 66..68: note = nnn occupied by a codon for F with 90% probability and by a codon for any one of the remaining 19 standard amino acids, except C, P and G, with a 10% probability distributed equally between each of the remaining 17 amino acids
  misc_difference 69..71: note = nnn occupied by a codon for Y with 90% probability and by a codon for any one of the remaining 19 standard amino acids, except C, P and G, with a 10% probability distributed equally between each of the remaining 17 amino acids
  misc_difference 75..77: note = nnn occupied by a codon for K with 90% probability and by a codon for any one of the remaining 19 standard amino acids, except C, P and G, with a 10% probability distributed equally between each of the remaining 17 amino acids
  misc_difference 78..80: note = nnn occupied by a codon for W with 90% probability and by a codon for any one of the remaining 19 standard amino acids, except C, P and G, with a 10% probability distributed equally between each of the remaining 17 amino acids
  misc_difference 87..89: note = nnn occupied by a codon for I with 90% probability and by a codon for any one of the remaining 19 standard amino acids, except C, P and G, with a 10% probability distributed equally between each of the remaining 17 amino acids
  misc_difference 90..92: note = nnn occupied by a codon for R with 90% probability and by a codon for any one of the remaining 19 standard amino acids, except C, P and G, with a 10% probability distributed equally between each of the remaining 17 amino acids
  misc_difference 93..95: note = nnn occupied by a codon for S with 90% probability and by a codon for K with a 10% probability
  misc_difference 99..101: note = nnn occupied by a codon for M with 90% probability and by a codon for any one of the remaining 19 standard amino acids, except C, P and G, with a 10% probability distributed equally between each of the remaining 17 amino acids
acaacaaattcaacaaagaannnnnnnnngcgnnnnnngagatcnnnnnnctgccgaacctgaacnnnnnncaannnnnngccttcnnnnnnnnnttannngatgacccaagccaaagcgc

SEQ ID NO: 284 - DNA, 121 bp, synthetic construct; identical to SEQ ID NO: 283
  source 1..121: mol_type = other DNA
  misc_difference 21..23: note = nnn occupied by a codon for any one of the standard 20 amino acids, except C, P and G, with equal probability of each
  misc_difference 24..26: note = nnn occupied by a codon for any one of the standard 20 amino acids, except C, P and G, with equal probability of each
  misc_difference 27..29: note = nnn occupied by a codon for F with 60% probability and by a codon for any one of the remaining 19 standard amino acids, except C, P and G, with a 40% probability distributed equally between each of the remaining 17 amino acids
  misc_difference 33..35: note = nnn occupied by a codon for D with 60% probability and by a codon for any one of the remaining 19 standard amino acids, except C, P and G, with a 40% probability distributed equally between each of the remaining 17 amino acids
  misc_difference 36..38: note = nnn occupied by a codon for any one of the standard 20 amino acids, except C, P and G, with equal probability of each
  misc_difference 45..47: note = nnn occupied by a codon for A with 20% probability and by a codon for any one of the remaining 19 standard amino acids, except C, P and G, with a 80% probability distributed equally between each of the remaining 17 amino acids
  misc_difference 48..50: note = nnn occupied by a codon for A with 20% probability and by a codon for any one of the remaining 19 standard amino acids, except C, P and G, with a 80% probability distributed equally between each of the remaining 17 amino acids
  misc_difference 66..68: note = nnn occupied by a codon for F with 60% probability and by a codon for any one of the remaining 19 standard amino acids, except C, P and G, with a 40% probability distributed equally between each of the remaining 17 amino acids
  misc_difference 69..71: note = nnn occupied by a codon for Y with 60% probability and by a codon for any one of the remaining 19 standard amino acids, except C, P and G, with a 40% probability distributed equally between each of the remaining 17 amino acids
  misc_difference 75..77: note = nnn occupied by a codon for K with 60% probability and by a codon for any one of the remaining 19 standard amino acids, except C, P and G, with a 40% probability distributed equally between each of the remaining 17 amino acids
  misc_difference 78..80: note = nnn occupied by a codon for W with 60% probability and by a codon for any one of the remaining 19 standard amino acids, except C, P and G, with a 40% probability distributed equally between each of the remaining 17 amino acids
  misc_difference 87..89: note = nnn occupied by a codon for I with 60% probability and by a codon for any one of the remaining 19 standard amino acids, except C, P and G, with a 40% probability distributed equally between each of the remaining 17 amino acids
  misc_difference 90..92: note = nnn occupied by a codon for R with 60% probability and by a codon for any one of the remaining 19 standard amino acids, except C, P and G, with a 40% probability distributed equally between each of the remaining 17 amino acids
  misc_difference 93..95: note = nnn occupied by a codon for S with 90% probability and by a codon for K with a 10% probability
  misc_difference 99..101: note = nnn occupied by a codon for M with 60% probability and by a codon for any one of the remaining 19 standard amino acids, except C, P and G, with a 40% probability distributed equally between each of the remaining 17 amino acids

SEQ ID NO: 285 - DNA, 39 bp, synthetic construct
  source 1..39: mol_type = other DNA
gatgaagccctcgaggtagacaacaaattcaacaaagaa

SEQ ID NO: 286 - DNA, 41 bp, synthetic construct; identical to SEQ ID NO: 282
  source 1..41: mol_type = other DNA

SEQ ID NO: 287 - RNA, 17 nt, synthetic construct
  source 1..17: mol_type = other RNA
gacgagtttaaaaacac

SEQ ID NO: 288 - RNA, 17 nt, synthetic construct
  source 1..17: mol_type = other RNA
gagcttaataatttctt

SEQ ID NO: 289 - RNA, 17 nt, synthetic construct
  source 1..17: mol_type = other RNA
gtgaccaattcagtgaa

(19) United States
(12) Patent Application Publication
(10) Pub. No.: US 2025/0326800 A1
(43) Pub. Date: Oct. 23, 2025

(54) NOVEL POLYPEPTIDES

(71) Applicant: Oncopeptides Innovation 1 AB, Stockholm (SE)

(72) Inventors: Per-Ake NYGREN, Stockholm (SE); Kim Anh GIANG, Stockholm (SE); Johan NILVEBRANT, Stockholm (SE); Fredrik LEHMANN, Stockholm (SE); Stefan Svensson GELIUS, Stockholm (SE)

(73) Assignee: Oncopeptides Innovation 1 AB, Stockholm (SE)

(21) Appl. No.: 18/870,324

(22) PCT Filed: May 31, 2023
(86) PCT No.: PCT/EP2023/064625

§ 371 Date: Nov. 27, 2024

(30) Foreign Application Priority Data
May 31, 2022 (GB) 2208027.9
Oct. 6, 2022 (GB) 2214718.5

(51) Int. Cl. C07K 14/00; A61K 38/00; A61K 47/64

(52) U.S. Cl. CPC C07K 14/001; A61K 47/64; A61K 38/00

(57) ABSTRACT

The invention provides a hBCMA-binding polypeptide which comprises at least one motif that binds to hBCMA, wherein said polypeptide comprises the following structure: [N-terminal portion]-[Helix 1]-[Separating portion]-[Helix 2]-[C-terminal portion] the hBCMA binding motif being the portion [Helix 1]-[Separating portion]-[Helix 2]. The invention further provides pharmaceutical compositions comprising the hBCMA-binding polypeptide, and the use of the hBCMA-binding polypeptide or pharmaceutical compositions as a medicament, particularly for use in the treatment or prophylaxis of cancers.

### FIELD OF THE INVENTION

The present invention relates to human BCMA (hBCMA)-binding polypeptides. The present invention also relates to pharmaceutical compositions comprising said hBCMA-binding polypeptides, and their use in the treatment and/or prophylaxis of cancer.

### BACKGROUND OF THE INVENTION

Immunotherapy has proven to be an effective treatment of several cancers with approved therapies constituting monoclonal and bispecific antibodies, immunomodulatory drugs, CAR-T treatments and antibody drug conjugates. Despite activity of these treatments, however, some patients exhibit very short responses or fail to respond to treatment. Side effects from some immunotherapies can be severe, especially side effects related to an exacerbated cytokine release. Indeed, cytokine release syndrome is one of the most common serious adverse effects of T cell-engaging immunotherapeutic agents (Shimabukuro-Vornhagen A et al. Cytokine release syndrome. J Immunother Cancer. 2018; 6 (1): 56. doi: 10.1186/s40425-018-0343-9). Many patients will also, eventually, become resistant to available treatments. Thus despite recent advances, there is still a need for additional treatment options in cancer immunotherapy.

One apparent obstacle with current treatment modalities is suboptimal distribution to tumorous tissue. Rates of tissue distribution are negatively correlated with molecular size, and so larger molecules such as antibodies have less efficient tumour penetration than smaller ones. Another issue is immune evasion by cancer cells, which often involves inhibitory immune signals in the tumour environment. Examples of such signals include: the production of immunosuppressive cytokines and other molecules, such as TGFβ or VEGF; cell-mediated immunosuppression, e.g. via tumour-derived regulatory T cells; modulation of antigen presentation and MHCI expression; or altered expression of other ligands, e.g. increased expression of inhibitory checkpoint ligands (such as Programmed death-ligand 1 (PD-L1) and HLA-E), which reduces tumour cell killing by CD8+ T cells and natural killer (NK) cells (Vinay D et al, Immune evasion in cancer: Mechanistic basis and therapeutic strategies, Seminars in Cancer Biology. 2015:35 (Supplement):S185, https://doi.org/10.1016/j.semcancer.2015.03.004; Ben-Shmuel A et al, Unleashing Natural Killer Cells in the Tumor Microenvironment—The Next Generation of Immunotherapy? Front Immunol. 2020; 11:275, doi: 10.3389/fimmu.2020.00275).

Multiple myeloma is a hematological malignancy that despite recent advances in immunotherapy is still incurable. Several surface proteins enriched on myeloma cells have been identified and served as targets for therapeutic interventions. One such target is BCMA (TNFRSF17), which is highly expressed on myeloma cells but to a lesser extent also expressed on non-malignant cells of the B cell compartment as well as plasmcytoid dendritic cells. Two B-cell stimulating ligands, a proliferation-inducing ligand (APRIL) and B-cell activating factor (BAFF), bind to BCMA and are implicated in autoimmune disorders as well as cancer (Bossen and Schneider (2006) BAFF, APRIL and their receptors: Structure, function and signalling, Seminars in Immunology, doi: 10.1016/j.smim.2006.04.006; Moreaux et al. (2004) BAFF and APRIL protect myeloma cells from apoptosis induced by interleukin 6 deprivation and dexamethasone, Blood 103 (8): 3148-3157; Samy et al. (2017) Targeting BAFF and APRIL in systemic lupus erythematosus and other antibody-associated diseases, International Reviews of Immunology, 36:1, 3-19).

CAR-T cell therapies and antibody-based therapies targeting BCMA have proven efficacious in clinical practice prolonging patient overall survival. Still, not all patients show satisfactory response to treatment and patients eventually relapse in their disease (see, for example, Teoh, P. J., Chng, W. J. CAR T-cell therapy in multiple myeloma: more room for improvement. Blood Cancer J. 11, 84 (2021). https://doi.org/10.1038/s41408-021-00469-5).

There therefore remains a need for improved cancer immunotherapeutics that can be delivered more efficiently to the tumour, while retaining the target specificity of antibodies and antibody-based drugs and avoiding potential side-effects such as immunogenicity.

The present invention seeks to address the afore-mentioned needs.

### SUMMARY OF THE INVENTION

The present invention provides an hBCMA-binding polypeptide which comprises at least one motif that binds to hBCMA, wherein said polypeptide comprises the following structure:
[N-terminal portion]-[Helix 1]-[Separating portion]-[Helix 2]-[C-terminal portion]
   the hBCMA binding motif being the portion [Helix 1]-[Separating portion]-[Helix 2].

In particular, the invention provides an hBCMA-binding polypeptide wherein:
i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
a) X₉ and X₁₀ are any naturally occurring amino acid; X₁₁ is E, F, H, Q, T or Y; X₁₄ is any naturally occurring amino acid; X₁₇ is A, E, Q, S, T or V; X₁₈ is any naturally occurring amino acid; X₂₅ is F or Y; X₃₁ is I, M, or V; X₃₃ is K or S; X₃₅ is I, L, M, or V;
or ii) Helix 1 and Helix 2 are defined as in i), wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue.

The invention also provides an hBCMA-binding polypeptide wherein:
i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279] wherein, independently from each other,
a) X₉ and X₁₀ are any naturally occurring amino acid; Xₙ is E, F, H, Q, T or Y; X₁₄ is any naturally occurring amino acid; X₁₇ is A, E, Q, S, T or V; X₁₈ is any naturally occurring amino acid; X₂₅ is F or Y; X₃₁ is I, M, or V; X₃₃ is K or S; X₃₅ is I, L, M, or V;
or ii) Helix 1 and Helix 2 are defined as in i), wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue;
and wherein the hBCMA binding efficacy is at least 1% of SEQ ID NO: 2.

The present inventors have surprisingly found that such hBCMA-binding polypeptides based on a non-antibody scaffold are effective in binding cancer cells and triggering cytotoxic drug-mediated or antibody-dependent cellular cytotoxicity-mediated (ADCC-mediated) cancer cell killing.

The invention therefore provides novel hBCMA-binding polypeptides that are effective in binding cancer cells (also termed ‘engagers’). They find particular use as cancer cell-binding units in conjugates and fusion proteins that can trigger cytotoxic drug-mediated or ADCC-mediated cancer cell killing and they therefore show promise in anti-cancer immunotherapeutics.

The invention further provides an hBCMA-binding oligomer, which comprises at least two hBCMA-binding polypeptides of the invention.

The invention further provides an hBCMA-binding polypeptide as disclosed herein, or hBCMA-binding oligomer as disclosed herein, which further comprises an additional functional portion (for example at least one, at least two, or at least three; for example 1, 2, 3, 4 or 5 additional functional portions).

Therefore, the invention further provides, in embodiments where the additional functional portion is an additional binding moiety, a bispecific engager or a multispecific engager comprising an additional binding moiety.

The invention further provides an hBCMA binder-drug conjugate comprising the hBCMA-binding polypeptide or hBCMA binding oligomer of the invention and an additional therapeutic agent.

The invention further provides:
a nucleic acid molecule encoding the hBCMA-binding polypeptide or hBCMA binding oligomer of the invention;
an expression vector comprising such a nucleic acid molecule;
a host cell comprising such a nucleic acid molecule or vector.

The invention further provides a method of making the hBCMA-binding polypeptide or hBCMA binding oligomer of the invention.

The invention also provides a pharmaceutical composition comprising an hBCMA-binding polypeptide, hBCMA binding oligomer, hBCMA binder-drug conjugate, nucleic acid molecule or expression vector as disclosed herein.

The invention further provides an hBCMA-binding polypeptide, hBCMA binding oligomer, hBCMA binder-drug conjugate, nucleic acid molecule, expression vector or pharmaceutical composition as disclosed herein for use in medicine, in particular in the treatment of cancer.

The invention also provides the use of an hBCMA-binding polypeptide, hBCMA binding oligomer, hBCMA binder-drug conjugate, nucleic acid molecule, expression vector or pharmaceutical composition as disclosed herein for the manufacture of a medicament for the treatment of cancer.

The invention also provides a method of treating cancer in which the method comprises administering to a patient in need thereof an active component that comprises an hBCMA-binding polypeptide or hBCMA binding oligomer as disclosed herein. For example, the hBCMA-binding polypeptide or hBCMA binding oligomer may be present as a cancer cell-binding unit in a conjugate or a fusion protein, such as one that can trigger cytotoxic drug-mediated or ADCC-mediated cancer cell killing. The invention also provides a method of treating cancer in which the method comprises administering to a patient in need thereof a nucleic acid molecule, expression vector or pharmaceutical composition as disclosed herein.

The invention further provides a kit comprising an hBCMA-binding polypeptide, hBCMA-binding oligomer, hBCMA binder-drug conjugate, nucleic acid molecule, expression vector or pharmaceutical composition as disclosed herein and, optionally, one or more further therapeutic agent(s). Such a kit finds particular use in the treatment and/or prophylaxis of cancer.

### DESCRIPTION OF THE DRAWINGS

FIG. 1 shows schematic domain organization of the hBCMA-binding polypeptides described in the present disclosure.

FIG. 2 shows the experimental set up (FIG. 2a) and the resulting sensorgram obtained after injection of the purified hBCMA binding variant His₆-Fa-G6-ABD_{WT} [SEQ ID 3], at 200 nM concentration, over a sensor chip surface on which hBCMA-Fc has been immobilized (FIG. 2b).

FIG. 3 shows sensorgrams obtained after injection of purified Fa-G6-His₆ [SEQ ID 4] and Fa-G6 truncations (-2, -3, -4 and -5 aa) [SEQ IDs 5-8], at 20 nM concentrations, over a sensor chip surface immobilized with hBCMA-Fc.

FIG. 4 shows sensorgrams obtained after injection of a set of purified His₆-Fa-G6 [SEQ ID 129] (“Fa-G6 wild-type”) and 14 Fa-G6 alanine substitution mutants [SEQ IDs 9-22], each equipped with a N-terminal His₆ tag, at 200 nM concentrations, over a sensor chip surface immobilized with hBCMA-Fc.

FIG. 5 shows sensorgrams obtained after injection of 18 purified second-generation Fa-G6 variants [SEQ IDs 2 and 23-39] equipped with a C-terminal His₆, at 100 nM, over a sensor chip surface immobilized with hBCMA-Fc.

FIG. 6 shows (a) thermal melting curves for Fa-G6-His₆ and 18 second generation variants and (b) thermal melting curves as well as determined thermal melting temperatures (Tms) for Fa-G6-His₆ [SEQ ID 4] and the second-generation clone 1-E6 [SEQ ID 2], also equipped with a C-terminal His₆.

FIG. 7 shows the receptor binding properties of 1-E6-MBP-MMAF and the non-conjugated capped 1-E6 polypeptides as observed in a BCMA-binding ELISA assay.

FIG. 8 shows the impact of 1-E6-MBP-MMAF on EJM cell viability in the presence of 5 μM 1-E6 or its corresponding non hBCMA binding counterpart (null).

FIG. 9 shows relative IFNγ secretion in cell media in cocultures of MM.1S and PBMC exposed to dual engagers for 4 h. Engagers evaluated were 1-E6-A10-His₆ [SEQ ID NO. 130], 1-E6-H09-A10-His₆ [SEQ ID NO. 131], 1-E6-A10-A10-His₆ [SEQ ID NO. 132], 1-E6-A11-A10-His₆ [SEQ ID NO. 133] and their corresponding non hBCMA binding counterparts (containing an Fa-G6 null polypeptide instead of 1-E6).

FIG. 10 shows CD 16 mediated activation in a Jurkat Lucia reporter assay relative to the maximal activation with the monoclonal SLAMF7 antibody Elotuzumab. Activation was monitored in the presence of MM.1S cells at 20 nM and 200 nM concentration of compound and in the absence of MM. 1S cells at 200 nM concentration. 1-E6-A10-His₆ [SEQ ID NO. 130], 1-E6-1E-6-A10-His₆ with three different linker lengths [SEQ ID NO. 134-136] as well as 1-E6-A10-1E6-His₆ [SEQ ID NO. 137] and A10-1-E6-1-E6-His₆ [SEQ ID NO. 138] show activation that is dependent on the presence of MM.1S. “Null” denotes a non hBCMA binding counterpart (containing an Fa-G6 null polypeptide instead of 1-E6).

FIG. 11 shows responses in a Jurkat-Lucia™ NFAT-CD16 reporter assay in the presence or absence of the BCMA positive MM.1S cell line. Responses were normalized to the maximal response of elotuzumab.

FIG. 12a shows responses in a Jurkat-Lucia™ NFAT-CD16 reporter assay in the presence or absence of the BCMA positive MM.1S cell line. Responses were normalized to the maximal response of elotuzumab.

FIG. 12b shows the enhancement of NK cell mediated cell killing of a BCMA positive MM.1S cell line. Responses were normalized to the maximal response mediated by a belantamab biosimilar.

FIG. 13a shows responses in a Jurkat-Lucia™ NFAT-CD16 reporter assay in the presence or absence of the BCMA positive MM.1S cell line. Responses were normalized to the maximal response of elotuzumab.

FIG. 13b shows the enhancement of NK cell mediated cell killing of a BCMA positive MM.1S cell line. Responses were normalized to the maximal response mediated by a belantamab biosimilar.

FIG. 14a shows results of a cell killing assay with NK cells and MM.1s cells.

FIG. 14b show results from a 8-hour flow cytometry-based killing assay with NK cells and MM.1s (E:T 5:1), treated with increasing concentrations of any of three dual engager constructs of the invention.

FIG. 15 illustrates hBCMA target specificity of the dual engager construct Example Compound131 in an in vitro multiple myeloma cell cytotoxicity assay.

FIG. 16 shows the concentration-effect curves of Fa-G6-mc-MMAF on cell viability of BCMA positive myeloma cell lines.

FIG. 17a shows the CD16a activation responses caused by the engager construct Example Compounds of SEQ IDs 156, 160 and 163 in the presence or absence of target MM.1s cells.

FIG. 17b shows the NK cell mediated killing of MM. 1S cells by the Example Compounds of SEQ IDs 156, 160 and 163 in the presence or absence of target MM.1s cells.

FIG. 18 shows relative IFN□ secretion in cell media in cocultures of MM.1S and PBMC exposed to dual engagers for 4 h. Engagers evaluated were Anti-BCMA-A10-His₆ [SEQ ID 130], and the corresponding non BCMA binding counterpart.

FIG. 19a shows responses in a Jurkat-Lucia™ NFAT-CD16 reporter assay in the presence or absence of the BCMA positive MM.1S cell line. Responses were normalized to the maximal response of elotuzumab.

FIG. 19b shows the enhancement of NK cell mediated cell killing of a BCMA positive MM.1S cell line. Responses were normalized to the maximal response mediated by a belantamab biosimilar.

### DETAILED DESCRIPTION

The present inventors have found that hBCMA-binding polypeptides as disclosed herein are surprisingly effective in binding cancer cells. They therefore find particular use in the treatment and/or prophylaxis of cancer, for example as cancer cell-binding units in conjugates and fusion proteins.

As discussed in more detail below, the present inventors have surprisingly found that hBCMA-binding polypeptides based on three-helix affibody scaffolds are effective at binding BCMA on myeloma cells, and trigger strong ADCC-mediated or cytotoxic drug-mediated responses against cancer cells. Such ADCC-mediated anti-cancer responses are beneficial in the treatment of multiple myeloma.

Notably, the inventors have found that such anti-cancer responses compare favourably with those obtained using the monoclonal antibody elotuzumab, which is approved for treatment of multiple myeloma. The inventors have further found that such anti-cancer responses compare favourably with those obtained using a biosimilar of belantamab mafodotin, which is an antibody-drug conjugate for treatment of multiple myeloma, and obtained using daratumumab, a monoclonal antibody which is approved for treatment of multiple myeloma.

In its broadest aspect, the invention provides an hBCMA-binding polypeptide which comprises at least one motif that binds to hBCMA, wherein said polypeptide comprises the following structure:
[N-terminal portion]-[Helix 1]-[Separating portion]-[Helix 2]-[C-terminal portion];
   the hBCMA binding motif being the portion [Helix 1]-[Separating portion]-[Helix 2].

The various elements of the polypeptides of the invention will now be described in further detail:

### Overall Structure

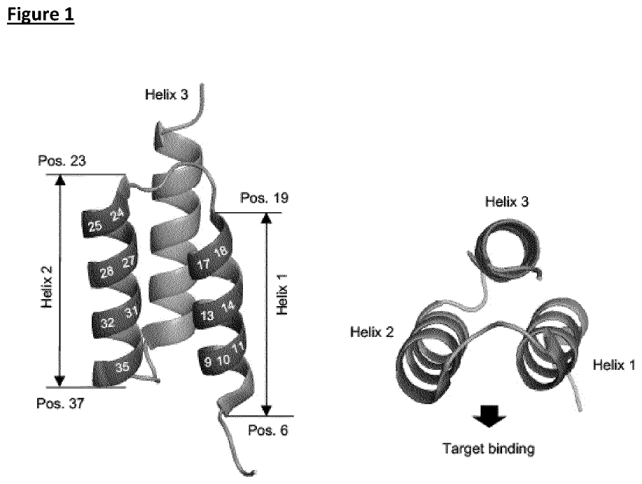

In an embodiment, the polypeptides of the present invention may be based on three-helix scaffolds, sometimes referred to as ‘affibodies’. Affibodies are small (around 6.5 kDa) engineered affinity ligands, based on the Z-domain polypeptide, which is a mutated version of the B-domain in the immunoglobulin-binding region of staphylococcal protein A (Nord K et al., Binding proteins selected from combinatorial libraries of an α-helical bacterial receptor domain, Nature Biotechnol., 1997:15:772, doi: 10.1038/nbt0897-772). In a full length affibody, the C-terminal portion includes [Second separating portion]-[Helix 3]-[C-terminal sequence]. The general structure of an affibody is shown in FIG. 1.

The portions of the molecules of the invention referred to as Helix 1 and Helix 2 (and Helix 3, when present) are generally helical in structure. In some rare embodiments, it can be found that the structure established by the sequence with particular residues may be not strictly helical. Such compounds are to be considered within the broadest aspect of the invention. More preferably, the residues in the Helix 1 and Helix 2 portions do result in those structures being helical, in the sense of being alpha-helical.

### Sequences of the Polypeptides of the Invention

The sequence of the hBCMA-binding polypeptides as disclosed herein may be expressed in terms of their constituent amino acids or in terms of nucleic acid sequences encoding polypeptides having those amino acid sequences. In the context of the present disclosure, the term “amino acid” encompasses any naturally occurring amino acid or unnatural amino acid. The term “unnatural amino acid” as used herein refers to non-proteinogenic (i.e. non-encoded) amino acids, which may either be found in nature or are chemically synthesised (for example citrulline, hydroxyproline, beta-alanine, ornithine, norleucine, 3-nitrotyrosine, pyroglutamic acid, or nitroarginine). It includes α, β, γ and δ amino acids. It includes an amino acid in any chiral configuration. The amino acid may, especially, be a naturally occurring a amino acid. The amino acid may, especially, be a naturally occurring L amino acid. The amino acid may, especially, be a naturally occurring L-α amino acid.

Within a polypeptide chain (for example a hBCMA-binding polypeptide as disclosed herein), the amino acids are linked by peptide bonds between the carboxyl group of one amino acid and the amine group of the next amino acid in the chain. An individual amino acid is called a “residue” or “amino acid residue” once it is linked in a polypeptide chain.

The amino acid sequences herein are shown with the N-terminus to the left, and where sequences are set out across multiple lines, the N-terminus is to the top left. Unless indicated otherwise, the amino acid residues in the sequences are L-amino acids.

The amino acid sequences listed in the application are shown using standard letter abbreviations for amino acids.

The specific sequences given herein relate to specific embodiments of the invention.

The present disclosure also includes derivatives of all the sequences described herein (for example, derivatives of each of the hBCMA-binding polypeptide and hBCMA-binding oligomer sequences described here). Derivatives of the sequences described herein are preferably derivatives wherein from 1 to 5 (for example 1, 2 or 3) amino acid residues may be replaced by an alternative residue, for example a different naturally occurring amino acid or a different unnatural amino acid; or a different naturally occurring amino acid excluding methionine or a different unnatural amino acid. Preferably, an unnatural amino acid according to the present invention is one that is isosteric with a naturally occurring amino acid, for example norleucine.

The present disclosure also includes derivatives of the sequences described herein wherein from 1 to 5 (for example 1, 2 or 3) amino acid residues may be replaced by an alternative residue that is a conservative replacement, that is to say that a residue is replaced with another residue in the same class, for example:

An aliphatic residue (Glycine (G), Alanine (A), Valine (V), Leucine (L) or Isoleucine (I)) may be replaced with another aliphatic residue. A hydroxyl-, sulphur- or selenium-containing residue (Serine (S), Cysteine (C), Selenocysteine (U), Threonine (T) or Methionine (M)) may be replaced with another hydroxyl-, sulphur- or selenium-containing residue. An aromatic residue (Phenylalanine (F), Tyrosine (Y) or Tryptophan (W)) may be replaced with another aromatic residue. A basic residue (Histidine (H), Lysine (K), Arginine (R)) may be replaced with another basic residue. An acidic residue or amide (Aspartate (D), Glutamate (E), Asparagine (N), Glutamine (Q)) may be replaced with another acidic residue or amide.

In certain embodiments, the sequences described herein (for example, hBCMA-binding polypeptide and hBCMA-binding oligomer sequences described here) do not comprise methionine. In certain embodiments, in the sequences described herein (for example, the hBCMA-binding polypeptide and hBCMA-binding oligomer sequences described here), at a position at which a methionine residue is recited, the polypeptide has the sequence with the methionine residue independently substituted for a different residue, for example a different naturally occurring amino acid or unnatural amino acid. In certain embodiments, in the sequences described herein, at a position at which a methionine residue is recited, the polypeptide has the sequence with the methionine residue independently substituted for an amino acid selected from isoleucine (I), leucine (L), glutamine (Q), and norleucine; for example isoleucine and norleucine.

In another embodiment, when one or more methionine is present in a hBCMA-binding polypeptides or CD16a-binding oligomers as defined herein, one, some or all of the methionine residues may be replaced by an alternative amino acid, for example a different naturally occurring amino acid or unnatural amino acid, such as an amino acid selected from isoleucine (I), leucine (L), glutamine (Q), or norleucine; and especially isoleucine (I) and norleucine.

For example, in one embodiment, one or more (for example each) methionine residues of the sequences described herein may be replaced by a different naturally occurring amino acid or unnatural amino acid, such as an amino acid selected from isoleucine, leucine, glutamine, and norleucine; and especially isoleucine and norleucine. For example, from 1 to 5 methionine residues, from 1 to 3 methionine residues (for example 1, 2 or 3 methionine residues), or 1 or 2 methionine residues, or 1 methionine residue, when present, may be replaced by a different naturally occurring amino acid or unnatural amino acid, such as an amino acid selected from isoleucine, leucine, glutamine, and norleucine; and especially isoleucine and norleucine.

For example, in embodiments wherein X₉ may be or is methionine, the residue at X₉ may be replaced by a different naturally occurring amino acid or unnatural amino acid, such as an amino acid selected from isoleucine, leucine, glutamine, and norleucine; and especially isoleucine and norleucine. For example, in embodiments wherein X₁₀ may be or is methionine, the residue at X₁₀ may be replaced by a different naturally occurring amino acid or unnatural amino acid, such as an amino acid selected from isoleucine, leucine, glutamine, and norleucine; and especially isoleucine and norleucine. For example, in embodiments wherein X₁₄ may be or is methionine, the residue at X₁₄ may be replaced by a different naturally occurring amino acid or unnatural amino acid, such as an amino acid selected from isoleucine, leucine, glutamine, and norleucine; and especially isoleucine and norleucine. For example, in embodiments wherein X₁₈ may be or is methionine, the residue at X₁₈ may be replaced by a different naturally occurring amino acid or unnatural amino acid, such as an amino acid selected from isoleucine, leucine, glutamine, and norleucine; and especially isoleucine and norleucine. For example, in embodiments wherein X₃₁ may be or is methionine, the residue at X₃₁ may be replaced by a different naturally occurring amino acid or unnatural amino acid, such as an amino acid selected from isoleucine, leucine, glutamine, and norleucine; and especially isoleucine and norleucine. For example, in embodiments wherein X₃₅ may be or is methionine, the residue at X₃₅ may be replaced by a different naturally occurring amino acid or unnatural amino acid, such as an amino acid selected from isoleucine, leucine, glutamine, and norleucine; and especially isoleucine and norleucine. In particular, in embodiments wherein X₃₁ and X₃₅ may be or are methionine, the residues at X₃₁ and X₃₅ may be replaced by a different naturally occurring amino acid or unnatural amino acid, such as an amino acid selected from isoleucine, leucine, glutamine, and norleucine; and especially isoleucine and norleucine.

Alternatively, or additionally, in certain embodiments, the sequences described herein (for example, hBCMA-binding polypeptide and hBCMA-binding oligomer sequences described here) may contain amino acid substitutions wherein one or more residues is replaced by an unnatural amino acid.

For example, in one embodiment, one or more residues of the sequences described herein (for example, the hBCMA-binding polypeptide and hBCMA-binding oligomer sequences described here) may be replaced by an unnatural amino acid, for example norleucine. For example, from 1 to 15 residues may be replaced by unnatural amino acid(s), for example from 1 to 10 residues (for example 1, 2, 3, 4, 5, 6, 7, 8, 9 or 10 residues), from 1 to 5 residues (for example 1, 2, 3, 4 or 5), from 1 to 3 residues (for example 1, 2, or 3) or 1 residue may be replaced by unnatural amino acid(s) (for example norleucine).

For example, in one embodiment, one or more leucine residues of the sequences described herein (for example, the hBCMA-binding polypeptide and hBCMA-binding oligomer sequences described here) may be replaced by an unnatural amino acid, and preferably norleucine. For example, from 1 to 5 leucine residues, from 1 to 3 leucine residues (for example 1, 2 or 3 leucine residues), or 1 or 2 leucine residues, or 1 leucine residue, when present, may be replaced by unnatural amino acid (for example norleucine). For example, in certain embodiments, in the sequences described herein (for example, the hBCMA-binding polypeptide and hBCMA-binding oligomer sequences described here), at a position at which a leucine residue is recited, the polypeptide has the sequence with the leucine residue independently substituted for an unnatural amino acid, and preferably norleucine.

Alternatively, or additionally, in one embodiment, one or more methionine residues of the sequences described herein (for example, the hBCMA-binding polypeptide and hBCMA-binding oligomer sequences described here) may be replaced by an unnatural amino acid, and preferably norleucine. For example, from 1 to 5 methionine residues, from 1 to 3 methionine residues (for example 1, 2 or 3 methionine residues), or 1 or 2 methionine residues, or 1 methionine residue, when present, may be replaced by unnatural amino acid (for example norleucine). For example, in certain embodiments, in the sequences described herein (for example, the hBCMA-binding polypeptide and hBCMA-binding oligomer sequences described here), at a position at which a methionine residue is recited, the polypeptide has the sequence with the methionine residue independently substituted for an unnatural amino acid, and preferably norleucine.

For example, in embodiments wherein X₉ may be or is methionine, the residue at X₉ may be replaced by an unnatural amino acid(s) (for example norleucine). For example, in embodiments wherein X₁₀ may be or is methionine, the residue at X₁₀ may be replaced by an unnatural amino acid(s) (for example norleucine). For example, in embodiments wherein X₁₄ may be or is methionine, the residue at X₁₄ may be replaced by an unnatural amino acid(s) (for example norleucine). For example, in embodiments wherein X₁₈ may be or is methionine, the residue at X₁₈ may be replaced by an unnatural amino acid(s) (for example norleucine). For example, in embodiments wherein X₃₁ may be or is methionine, the residue at X₃₁ may be replaced by an unnatural amino acid(s) (for example norleucine). For example, in embodiments wherein X₃₅ may be or is methionine, the residue at X₃₅ may be replaced by an unnatural amino acid(s) (for example norleucine). In particular, in embodiments wherein X₃₁ and X₃₅ may be or are methionine, the residues at X₃₁ and X₃₅ may be replaced by an unnatural amino acid(s) (for example norleucine).

In certain embodiments, one or more methionine residues of the sequences described herein (for example, the hBCMA-binding polypeptide and hBCMA-binding oligomer sequences described here) may be oxidised, i.e. in the form of methionine sulfoxide (“Met(O)”). For example, from 1 to 5 methionine residues, from 1 to 3 methionine residues (for example 1, 2 or 3 methionine residues), or 1 or 2 methionine residues, or 1 methionine residue, when present, may be oxidised (for example may be Met(O)). For example, in embodiments wherein X₉ may be or is methionine, when present the methionine at X₉ may be oxidised (for example Met(O)). For example, in embodiments wherein X₁₀ may be or is methionine, when present the methionine at X₁₀ may be oxidised (for example Met(O)). For example, in embodiments wherein X₁₄ may be or is methionine, when present the methionine at X₁₄ may be oxidised (for example Met(O)). For example, in embodiments wherein X₁₈ may be or is methionine, when present the methionine at X₁₈ may be oxidised (for example. Met(O)). For example, in embodiments wherein X₃₁ may be or is methionine, when present the methionine at X₃₁ may be oxidised (for example Met(O)). For example, in embodiments wherein X₃₅ may be or is methionine, when present the methionine at X₃₅ may be oxidised (for example Met(O)). In particular, in embodiments wherein X₃₁ and X₃₅ may be or are methionine, when present the methionines at X₃₁ and X₃₅ may be oxidised (for example Met(O)).

Alternatively, or additionally, in certain embodiments, the sequences described herein (for example, the hBCMA-binding polypeptide and hBCMA-binding oligomer sequences described here) comprise a peptide purification tag or moiety (for example a histidine-tag (for example a polyhistidine tag) or a methionine-tag (for example a single methionine tag or a polymethionine tag)), a signalling tag or moiety (for example a glycine residue, or a signal peptide, for example selected from signal peptides of OmpA, DsbA, PhoA, and PelB), a fluorophore tag (for example Alexa448), or a tag or moiety to assist conjugation (a cysteine tag (for example a single cysteine at the C or N terminal)). Such tags and/or moieties may preferably be present at the N-terminal and/or the C-terminal of the BCMA-binding polypeptide and BCMA-binding oligomer sequences described herein.

Therefore, the sequences described herein (for example, the hBCMA-binding polypeptide and hBCMA-binding oligomer sequences described here) may further comprise an additional sequence of at least 1 histidine residue (and optionally at least 1 tyrosine residue) and/or at least 1 methionine residue; for example at least 4, at least 5, or at least 6 histidine residues (and optionally at least 1 tyrosine residue, for example 1, 2 or 3 tyrosine residues) and/or at least 1 methionine residue. In one embodiment, the sequences described herein (for example, the hBCMA-binding polypeptide and hBCMA-binding oligomer sequences described here) may further comprise an additional sequence of at least 6 histidine residues and optionally at least 1 tyrosine residue (for example 6 histidine residues (e.g. HHHHHH) or 6 histidine residues and two tyrosine residues (e.g. YYHHHHHH)) and/or at least 1 methionine residue (for example 1 or 2 methionine residues). A peptide purification tag or moiety, for example a histidine-tag or a methionine-tag as described above, may preferably be present at the N-terminal and/or the C-terminal of the hBCMA-binding polypeptide and hBCMA-binding oligomer sequences described herein. For example, an additional sequence of at least 6 histidine residues (for example 6 histidine residues; or 6 histidine residues and two tyrosine residues) and/or at least 1 methionine residue (for example 1 or 2 methionine residues) may be present at the N-terminal and/or the C-terminal of the hBCMA-binding polypeptide and hBCMA-binding oligomer sequences described herein.

The sequences described herein (for example, the hBCMA-binding polypeptide and hBCMA-binding oligomer sequences described here) may further comprise an additional sequence of at least one cysteine (for example one cysteine) at the N-terminal or the C-terminal. The sequences described herein (for example, the hBCMA-binding polypeptide and hBCMA-binding oligomer sequences described here) may further comprise a fluorophore tag (for example a 448Alexa tag) at the N-terminal or the C-terminal. The sequences described herein (for example, the hBCMA-binding polypeptide and hBCMA-binding oligomer sequences described here) may further comprise a signal peptide, for example selected from OmpA, DsbA, PhoA, and PelB, athe the N-terminal or the C-terminal, preferably the N-terminal. The sequences described herein (for example, the hBCMA-binding polypeptide and hBCMA-binding oligomer sequences described here) may further comprise an additional sequence of at least one glycine (for example one glycine) at the N-terminal or the C-terminal.

In certain embodiments, the hBCMA-binding polypeptide is one wherein the hBCMAbinding efficacy is at least 1%, at least 5%, or preferably at least 10% (more preferably at least 15%, 20% 25% or 50%) of SEQ ID NO: 2. When a hBCMA-binding polypeptide is described herein as having hBCMA binding efficacy that is at least X % of a specific peptide (e.g. SEQ ID NO: 2), it is understood that the IC₅₀ concentration of the polypeptide for binding to the hBCMA receptor is no more than 100/X times the IC₅₀ concentration for the specific peptide (SEQ ID NO: 2) to the hBCMA receptor, when measured under the same conditions.

For example, if the binding efficacy of a hBCMA-binding polypeptide is at least 5%, and more preferably at least 10%, 20%, 25% or 50% of the hBCMA-binding efficacy of the specific peptide (e.g. SEQ ID NO: 2), that is to say that the IC₅₀ concentration of the alternative polypeptide for binding to the hBCMA receptor is no more than 20 times and more preferably 10 times, 5 times, 4 times or 2 times, respectively, the IC₅₀ concentration for the specific peptide (e.g. SEQ ID NO: 2) to the hBCMA receptor, when measured under the same conditions.

In certain embodiments, alternatively, or additionally, the hBCMA-binding polypeptide is one that competes for binding at hBCMA with the peptide of SEQ ID NO: 2.

The present invention provides an hBCMA-binding polypeptide which comprises at least one motif that binds to hBCMA, wherein said polypeptide comprises the following structure:
[N-terminal portion]-[Helix 1]-[Separating portion]-[Helix 2]-[C-terminal portion]
the hBCMA-binding motif being the portion [Helix 1]-[Separating portion]-[Helix 2]

Preferably, the hBCMA-binding polypeptide is one wherein:
i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are any naturally occurring amino acid; Xₙ is E, F, H, Q, T or Y; X₁₄ is any naturally occurring amino acid; X₁₇ is A, E, Q, S, T or V; X₁₈ is any naturally occurring amino acid; X₂₅ is F or Y; X₃₁ is I, M, or V; X₃₃ is K or S; X₃₅ is I, L, M, or V;
or ii) Helix 1 and Helix 2 are defined as in i), wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue.

In an embodiment, Helix 1 comprises the sequence KEX₉X₁₀X₁₁ADX₁₄EIX₁₇X₁₈ and Helix 2 comprises the sequence FX₂₅QKWAFX₃₁RX₃₃LX₃₅D, for example Helix 1 comprises the sequence NKEX₉X₁₀X₁₁ADX₁₄EIX₁₇X₁₈L and Helix 2 comprises the sequence NFX₂₅QKWAFX₃₁RX₃₃LX₃₅DD, wherein X₉, X₁₀, X₁₁, X₁₄, X₁₇, X₁₈, X₂₅, X₃₁, X₃₃, and X₃₅ are as defined above.

Preferably, such an hBCMA-binding polypeptide is one wherein the hBCMA binding efficacy is at least 1% of SEQ ID NO: 2. Preferably, the binding efficacy is at least 5%, for example at least 10%, 20%, 25% or 50% of the hBCMA binding efficacy of the specific peptide (e.g. SEQ ID NO: 2).

In certain embodiments, alternatively, or additionally, the hBCMA-binding polypeptide is one that competes with SEQ ID NO: 2 for binding of hBCMA.

In an embodiment, the hBCMA-binding polypeptide is one wherein i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are any naturally occurring amino acid;
X₁₁ is E, F, H, Q, T or Y;
X₁₄ is any naturally occurring amino acid;
X₁₇ is A, E, Q, S, T or V;
X₁₈ is any naturally occurring amino acid;
X₂₅ is F or Y;
X₃₁ is I, M, or V;
X₃₃ is K or S;
X₃₅ is I, L, M, or V;
or ii) Helix 1 and Helix 2 are defined as in i), wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue;
   and wherein the hBCMA binding efficacy is at least 1% of SEQ ID NO: 2.

Preferably, the binding efficacy is at least 5%, for example at least 10%, 20%, 25% or 50% of the hBCMA binding efficacy of the specific peptide (e.g. SEQ ID NO: 2).

In certain embodiments, alternatively, or additionally, the hBCMA-binding polypeptide is one that competes with SEQ ID NO: 2 of hBCMA.

As mentioned above, full identity with the sequences set out for (i) is not required (though in one preferred embodiment of the invention, the peptide does have the exact recited sequence). Of the residues denoted with an Xₙ label, at least 1 and no more than 8 of the residues may be replaced with an alternative residue. The replaced residues may be all in Helix 1 or in Helix 2, or there may be 1 replaced residue in one of them and 1, 2, 3, 4, 5, 6 or 7 replaced residues in the other, or there may be 2 replaced residues in one of them and 1, 2, 3, 4, 5, or 6 replaced residues in the other, or there may be 3 replaced residues in one of them and 1, 2, 3, 4, or 5 replaced residues in the other, or there may be 4 replaced residues in one of them and 1, 2, 3 or 4 replaced residue in the other, or there may be 5 replaced residues in one of them and 1, 2 or 3 replaced residues in the other, or there may be 6 replaced residues in one of them and 1 or 2 replaced residues in the other, or there may be 7 replaced residues in one of them and 1 replaced residue in the other. For example, there may be 1, 2, 3, 4 or 5 replaced residues in the residues denoted with an Xₙ label, for example 1, 2 or 3, for example 1. As there are 10 residues with an Xₙ label, a peptide with 5 residues replaced has 50% sequence identity with the recited sequence. For replacement of 2 residues, it is 80% and for replacement of 1 residue it is 90% sequence identity.

In another embodiment, of the residues not labelled as an Xₙ residue, at least 1 and no more than 5 of the residues may be replaced with an alternative residue. The replaced residues may be all in Helix 1 or in Helix 2, or there may be 1 replaced residue in one of them and 1, 2, 3, 4 or 5 replaced residues in the other, or there may be 2 replaced residues in one of them and 1, 2, or 3 replaced residues in the other, or there may be 3 replaced residues in one of them and 1 or 2 replaced residues in the other, or there may be 4 replaced residues in one of them and 1 replaced residue in the other. For example, there may be 1, 2, 3, 4 or 5 replaced residues in the residues not denoted with an Xₙ label, for example 1, 2 or 3, for example 1. As there are 12 residues without an Xₙ label, a peptide with 5 residues replaced has 58% sequence identity with the recited sequence. For replacement of 2 residues, it is 83% and for replacement of 1 residue it is 92% sequence identity.

In another embodiment, of the residues not labelled as an Xₙ residue, at least 1 and no more than 3 of the residues may be replaced with an alternative residue. The replaced residues may be all in Helix 1 or in Helix 2, or there may be 1 replaced residue in one of them and 1 or 2 replaced residues in the other, or there may be 2 replaced residues in one of them and 1 replaced residues in the other. For example, there may be 1, 2, or 3 replaced residues in the residues not denoted with an Xₙ label, for example 1 or 2, for example 1. As there are 12 residues without an Xₙ label, a peptide with 3 residues replaced has 75% sequence identity with the recited sequence. For replacement of 2 residues, it is 83% and for replacement of 1 residue it is 92% sequence identity.

In an embodiment, the total number of residues in the Helix 1 and Helix 2 portions that are replaced is at least 1 and no more than 13, at least 1 and no more than 12, at least 1 and no more than 11, at least 1 and no more than 10, for example at least 1 and no more than 9, at least 1 and no more than 8, for example at least 1 and no more than 7, for example at least 1 and no more than 6, for example at least 1 and no more than 5, for example at least 1 and no more than 4, for example at least 1 and no more than 3, for example at least 1 and no more than 2. Particularly, there may be 1, 2, 3, 4, 5, 6, 7, 8, 9, or 10 replacement residues in total in those portions.

An amino acid residue replacement for a residue denoted as Xₙ or a residue not denoted as Xₙ may be a conservative replacement. That is to say that a residue is replaced with another residue in the same class, for example:

An aliphatic residue (Glycine (G), Alanine (A), Valine (V), Leucine (L) or Isoleucine (I)) may be replaced with another aliphatic residue. A hydroxyl-, sulphur- or selenium-containing residue (Serine (S), Cysteine (C), Selenocysteine (U), Threonine (T) or Methionine (M)) may be replaced with another hydroxyl-, sulphur- or selenium-containing residue. An aromatic residue (Phenylalanine (F), Tyrosine (Y) or Tryptophan (W)) may be replaced with another aromatic residue. A basic residue (Histidine (H), Lysine (K), Arginine (R)) may be replaced with another basic residue. An acidic residue or amide (Aspartate (D), Glutamate (E), Asparagine (N), Glutamine (Q)) may be replaced with another acidic residue or amide.

Alternatively, an amino acid residue replacement for a residue denoted as Xₙ or a residue not denoted as Xₙ may be a non-conservative replacement, i.e. a residue is replaced with another residue in a different class, for example an aliphatic residue (Glycine (G), Alanine (A), Valine (V), Leucine (L) or Isoleucine (I)) may be replaced with an aromatic residue (Phenylalanine (F), Tyrosine (Y) or Tryptophan (W)), or for example replaced by an amino acid with opposite characteristics, for example replacement of a Lysine (K) residue with an Aspartic acid (D) residue.

Alternatively, an amino acid residue replacement for a residue denoted as Xₙ or a residue not denoted as Xₙ may be a non-canonical (also known as unnatural) amino acid, i.e. an amino acid that is not found in natural polypeptide chains. As mentioned above, the term “unnatural amino acid” as used herein refers to non-proteinogenic (i.e. non-encoded) amino acids, which may either be found in nature or are chemically synthesised (for example citrulline, hydroxyproline, beta-alanine, ornithine, norleucine, 3-nitrotyrosine, pyroglutamic acid, or nitroarginine). These can be produced as secondary metabolites or synthesised chemically. Preferably, an unnatural amino acid according to the present invention is one that is isosteric with a naturally occurring amino acid, for example norleucine. Therefore, an amino acid residue replacement for a residue denoted as Xₙ or a residue not denoted as Xₙ may be an unnatural amino acid according to the present invention is one that is isosteric with a naturally occurring amino acid, and preferably norleucine.

In another embodiment, the hBCMA-binding polypeptide is one wherein:
i) Helix 1 comprises the sequence X₉X₁₀X₁₁ADX₁₄EIX₁₇X₁₈ [SEQ ID NO. 278] and Helix 2 comprises the sequence FYQKWAFIRX₃₃LM, wherein, independently from each other,
X₉ is D, E, H, K, N, Q, S, or V; X₁₀ is A, E, F, I, K, M, N, Q, R, S, T, Y, or V; Xu is E, F, or H; X₁₄ is A, E, H, I, K, L, Q, R, T, or Y; X₁₇ is A, E S, T, or V; X₁₈ is A, F, H, K, L, M, N, T, or S; X₃₃ is K or S;
or ii) Helix 1 and Helix 2 are defined as in i), wherein within Helix 1 and Helix 2, at least 1 and no more than 3 (for example 1, 2 or 3) of the Xₙ residues are replaced by an alternative residue, and/or at least 1 and no more than 3 (for example 1, 2 or 3) of the residues not labelled as Xₙ are replaced by an alternative residue.

Preferably, such an hBCMA-binding polypeptide is one wherein the hBCMA binding efficacy is at least 1% of SEQ ID NO. 2. For example, the binding efficacy is at least 5%, for example at least 10%, 20%, 25% or 50% of the hBCMA binding efficacy of the specific peptide (e.g. SEQ ID NO: 2).

In certain embodiments, alternatively, or additionally, the hBCMA-binding polypeptide is one that competes with SEQ ID NO: 2 for binding of hBCMA.

As mentioned above, full identity with the sequences set out for (i) is not required (though in one preferred embodiment of the invention, the peptide does have the exact recited sequence). Of the residues denoted with an Xₙ label, at least 1 and no more than 3 (for example 1, 2 or 3) of the residues may be replaced with an alternative residue. The replaced residues may be all in Helix 1 or in Helix 2, or there may be 1 replaced residue in one of them and 1 or 2 replaced residues in the other. For example, there may be 1, 2, or 3 replaced residues in the residues denoted with an Xₙ label, for example 1 or 2, for example 1. As there are 7 residues with an Xₙ label, a peptide with 3 residues replaced has 57% sequence identity with the recited sequence. For replacement of 2 residues, it is 71% and for replacement of 1 residue it is 86% sequence identity.

Of the residues not labelled as an Xₙ residue, at least 1 and no more than 3 (for example 1, 2 or 3) of the residues may be replaced with an alternative residue. The replaced residues may be all in Helix 1 or in Helix 2, or there may be 1 replaced residue in one of them and 1 or 2 replaced residues in the other. For example, there may be 1, 2, or 3 replaced residues in the residues not denoted with an Xₙ label, for example 1 or 2, for example 1. As there are 15 residues without an Xₙ label, a peptide with 3 residues replaced has 80% sequence identity with the recited sequence. For replacement of 2 residues, it is 87% and for replacement of 1 residue it is 93% sequence identity.

In an embodiment, the total number of residues in the Helix 1 and Helix 2 portions that are replaced is at least 1 and no more than 6, for example at least 1 and no more than 5, at least 1 and no more than 4, for example at least 1 and no more than 3, for example at least 1 and no more than 3. Particularly, there may be 1, 2, 3, 4, 5, or 6 replacement residues in total in those portions.

An amino acid residue replacement for a residue denoted as Xₙ or a residue not denoted as Xₙ may be a conservative or a non-conservative replacement, or a non-canonical (also known as unnatural) amino acid, as described herein above.

In a further embodiment, the hBCMA-binding polypeptide is one which does not have a methionine residue at positions X₉, X₁₀, X₁₄, X₁₈, X₃₁ or X₃₅, for example an hBCMA-binding polypeptide wherein Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are an unnatural amino acid (for example norleucine) or any naturally occurring amino acid excluding methionine;
X₁₁ is E, F, H, Q, T or Y;
X₁₄ is an unnatural amino acid (for example norleucine) or any naturally occurring amino acid excluding methionine;
X₁₇ is A, E, Q, S, T or V;
X₁₈ is an unnatural amino acid (for example norleucine) or any naturally occurring amino acid excluding methionine;
X₂₅ is F or Y;
X₃₁ is I, V, L, glutamine, or an unnatural amino acid (for example norleucine) (preferably I, L, V, glutamine, or norleucine);
X₃₃ is K or S;
X₃₅ is I, L, V, glutamine, or an unnatural amino acid (for example norleucine) (preferably I, L, V, glutamine, or norleucine); or

In a further embodiment, the hBCMA-binding polypeptide is one which does not have a methionine residue at positions X₉, X₁₀, X₁₄, X₁₈, X₃₁ or X₃₅, for example an hBCMA-binding polypeptide wherein Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other, X₉ and X₁₀ are norleucine or any naturally occurring amino acid excluding methionine;
X₁₁ is E, F, H, Q, T or Y;
X₁₄ is norleucine or any naturally occurring amino acid excluding methionine;
X₁₇ is A, E, Q, S, T or V;
X₁₈ is norleucine, or any naturally occurring amino acid excluding methionine;
X₂₅ is F or Y;
X₃₁ is I, V, L, glutamineor norleucine (for example I, V or norleucine; preferably I or norleucine);
X₃₃ is K or S;
X₃₅ is I, L, V glutamine or norleucine (for example I, V or norleucine; preferably I or norleucine); or

In a further embodiment, the hBCMA-binding polypeptide is one which does not have a methionine residue at positions X₉, X₁₀, X₁₄, X₁₈, X₃₁ or X₃₅, for example an hBCMA-binding polypeptide wherein Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are any naturally occurring amino acid excluding methionine;
X₁₁ is E, F, H, Q, T or Y;
X₁₄ is any naturally occurring amino acid excluding methionine;
X₁₇ is A, E, Q, S, T or V;
X₁₈ is any naturally occurring amino acid excluding methionine;
X₂₅ is F or Y;
X₃₁ is I or V;
X₃₃ is K or S;
X₃₅ is I, L or V.

In a further embodiment, the hBCMA-binding polypeptide is one which does not have a methionine residue at positions X₉, X₁₀, X₁₄, X₁₈, X₃₁ or X₃₅, for example an hBCMA-binding polypeptide wherein Helix 1 comprises the sequence X₉X₁₀X₁₁ADX₁₄EIX₁₇X₁₈ [SEQ ID NO. 278] and Helix 2 comprises the sequence FYQKWAFIRX₃₃LX₃₅, wherein, independently from each other,
X₉ is D, E, H, K, N, Q, S, or V;
X₁₀ is A, E, F, I, K, N, Q, R, S, T, Y, V, L or an unnatural amino acid (for example norleucine);
X₁₁ is E, F, or H;
X₁₄ is A, E, H, I, K, L, Q, R, T, or Y;
X₁₇ is A, E S, T, or V;
X₁₈ is A, F, H, K, L, N, T, S, I, Q or an unnatural amino acid (for example norleucine);
X₃₃ is K or S;
X₃₅ is a naturally occurring amino acid excluding methionine (preferably a naturally occurring amino acid that is isosteric with methionine, for example isoleucine) or an unnatural amino acid (preferably a naturally occurring amino acid that is isosteric with methionine, for example norleucine); or;

In a further embodiment, the hBCMA-binding polypeptide is one which does not have a methionine residue at positions X₉, X₁₀, X₁₄, X₁₈, X₃₁ or X₃₅, for example an hBCMA-binding polypeptide wherein Helix 1 comprises the sequence X₉X₁₀X₁₁ADX₁₄EIX₁₇X₁₈ [SEQ ID NO. 278] and Helix 2 comprises the sequence FYQKWAFIRX₃₃LX₃₅, wherein, independently from each other,
X₉ is D, E, H, K, N, Q, S, or V;
X₁₀ is A, E, F, I, K, N, Q, R, S, T, Y, V, L or norleucine;
X₁₁ is E, F, or H;
X₁₄ is A, E, H, I, K, L, Q, R, T, or Y;
X₁₇ is A, E S, T, or V;
X₁₈ is A, F, H, K, L, N, T, S, I, Q or norleucine;
X₃₃ is K or S;
X₃₅ is a naturally occurring amino acid that is isosteric with methionine (for example isoleucine), or norleucine; or;

In a further embodiment, the hBCMA-binding polypeptide is one which does not have a methionine residue at positions X₉, X₁₀, X₁₄, X₁₈, X₃₁ or X₃₅, for example an hBCMA-binding polypeptide wherein Helix 1 comprises the sequence X₉X₁₀X₁₁ADX₁₄EIX₁₇X₁₈ [SEQ ID NO. 278] and Helix 2 comprises the sequence FYQKWAFIRX₃₃L X₃₅, wherein, independently from each other,
X₉ is D, E, H, K, N, Q, S, or V;
X₁₀ is A, E, F, I, K, N, Q, R, S, T, Y, or V;
X₁₁ is E, F, or H;
X₁₄ is A, E, H, I, K, L, Q, R, T, or Y;
X₁₇ is A, E S, T, or V;
X₁₈ is A, F, H, K, L, N, T, or S;
X₃₃ is K or S;
X₃₅ is isoleucine or norleucine;

Preferably, in the embodiments described above, the hBCMA binding efficacy is at least 1% at least 5%, or at least 10% (for example at least 15%, 20% 25% or 50%) of the binding efficacy of the peptide of SEQ ID NO: 2 (i.e. binding efficacy of the peptide of SEQ ID NO: 2 to hBCMA, when measured under the same conditions).

Preferably, in the embodiments described above, alternatively, or additionally, the hBCMA-binding polypeptide is one that competes with SEQ ID NO: 2.

In advantageous embodiments of hBCMA-binding polypeptides of the invention, Proline (P) and Cysteine (C) are not present in Helix 1 or Helix 2 of a hBCMA-binding polypeptides of the invention. Advantageously Glycine (G) is also not present in Helix 1 or Helix 2 of a hBCMA-binding polypeptides of the invention.

In certain embodiments of the invention, such as the embodiments described above, the hBCMA-binding polypeptide is one wherein:
Helix 1 comprises the sequence X₆X₇X₈X₉X₁₀X₁₁ADX₁₄EIX₁₇X₁₈X₁₉ and/or
Helix 2 comprises the sequence X₂₃FX₂₅QKWAFX₃₁RX₃₃LX₃₅X₃₆X₃₇,

In such embodiments, X₆ may be any naturally occurring amino acid or is absent; X₇ may be any naturally occurring amino acid or is absent; X₈ may be any naturally occurring amino acid or is absent; X₁₉ may be any naturally occurring amino acid or is absent; X₂₃ may be any naturally occurring amino acid or is absent; X₃₆ may be any naturally occurring amino acid or is absent; and X₃₇ may be any naturally occurring amino acid or is absent.

More preferably, X₆ may be any naturally occurring amino acid; X₇ may be any naturally occurring amino acid; X₈ may be any naturally occurring amino acid; X₁₉ may be any naturally occurring amino acid; X₂₃ may be any naturally occurring amino acid; X₃₆ may be any naturally occurring amino acid; and X₃₇ may be any naturally occurring amino acid.

In another preferred embodiment, X₆ may be D, E, N or Q or is absent; X₇ may be H, K or R or is absent; X₈ may be D, E, N or Q or is absent; X₁₉ may be G, A, V, L or I or is absent; X₂₃ may be D, E, N or Q or is absent; X₃₆ may be D, E, N or Q or is absent; and X₃₇ may be D, E, N or Q or is absent;

In another preferred embodiment, X₆ may be D, E, N or Q; X₇ may be H, K or R; X₈ may be D, E, N or Q; X₁₉ may be G, A, V, L or I; X₂₃ may be D, E, N or Q; X₃₆ may be D, E, N or Q; and X₃₇ may be D, E, N or Q.

In another preferred embodiment, X₆ may be N or is absent; X₇ may be K or is absent; X₈ may be E or is absent; X₁₉ may be L or is absent; X₂₃ may be N or is absent; X₃₆ may be D or is absent; and X₃₇ may be D or is absent.

In an especially preferred embodiment, X₆ is N; X₇ is K; and X₈ is E. In another especially preferred embodiment, X₃₆ is D; and X₃₇ is D.

In a very especially preferred embodiment, X₆ is N; X₇ is K; X₈ is E; X₁₉ is L; X₂₃ is N; X₃₆ is D; and X₃₇ is D. In such embodiments,
Helix 1 comprises the sequence NKEX₉X₁₀X₁₁ADX₁₄EIX₁₇X₁₈L and/or Helix 2 comprises the sequence NFX₂₅QKWAFX₃₁RX₃₃LX₃₅DD,

In advantageous embodiments of hBCMA-binding polypeptides of the invention, Proline (P) and Cysteine (C) are not present in Helix 1 or Helix 2. Advantageously Glycine (G) is also not present in Helix 1 or Helix 2.

Preferably, such an hBCMA-binding polypeptide has an hBCMA binding efficacy that is at least 1% of SEQ ID NO. 2.

In certain embodiments, alternatively, or additionally, the hBCMA-binding polypeptide is one that competes with SEQ ID NO: 2 for being of hBCMA.

In another preferred embodiment, the hBCMA-binding polypeptide is one wherein:
i) Helix 1 comprises the sequence NKEETFADLEISNL and Helix 2 comprises the sequence NFYQKWAFIRSLMDD,
or ii) Helix 1 and Helix 2 are defined as in i), wherein within Helix 1 and Helix 2, at least 1 and no more than 2 (for example 1 or 2) residues are replaced by an alternative residue,
or (iii) at least 1 and no more than 3 (for example 1, 2, or 3) residues in the sequence of Helix 1 and/or Helix 2 are replaced by an alternative residue (for example replaced by an alternative residue that is a conservative replacement).

Preferably, such an hBCMA-binding polypeptide is one wherein the hBCMA binding efficacy is at least 1% of SEQ ID NO: 1.

In certain embodiments, alternatively, or additionally, the hBCMA-binding polypeptide is one that competes with SEQ ID NO: 1.

In another preferred embodiment, the hBCMA-engaging polypeptide is one wherein:
i) Helix 1 comprises the sequence NKENQFADEEIAAL and Helix 2 comprises the sequence NFYQKWAFIRKLMDD,
or ii) Helix 1 and Helix 2 are defined as in i), wherein within Helix 1 and Helix 2, at least 1 and no more than 2 (for example 1 or 2) residues are replaced by an alternative residue
or (iii) at least 1 and no more than 3 (for example 1, 2, or 3) residues in the sequence of Helix 1 and/or Helix 2 are replaced by an alternative residue (for example replaced by an alternative residue that is a conservative replacement).

Preferably, such a hBCMA-binding polypeptide is one wherein the hBCMA binding efficacy is at least 1% of SEQ ID NO: 2.

In certain embodiments, alternatively, or additionally, the hBCMA-binding polypeptide is one that competes with SEQ ID NO: 2.

In one embodiment, the hBCMA-binding polypeptide is one wherein:
a) Helix 1 comprises the sequence ETFADLEISN and Helix 2 comprises the sequence FYQKWAFIRSLM. For example, Helix 1 comprises the sequence NKEETFADLEISNL and Helix 2 comprises the sequence NFYQKWAFIRSLMDD.

In a further embodiment, the hBCMA-binding polypeptide is one wherein
b) Helix 1 comprises the sequence NQFADEEIAA and Helix 2 comprises the sequence FYQKWAFIRKLM. For example, Helix 1 comprises the sequence NKENQFADEEIAAL and Helix 2 comprises the sequence NFYQKWAFIRKLMDD

As described herein above, a number of residues may each be substituted by an alternative residue. For example, at least 1 and no more than 3, at least 1 and no more than 2, or 1 residue may be substituted by an alternative residue, for example 3, 2, or 1 residues may be substituted by an alternative residue. An amino acid residue replacement may be a conservative or a non-conservative replacement, or a non-canonical (also known as unnatural) amino acid, as described herein above.

In advantageous embodiments of hBCMA-binding polypeptides of the invention, Proline (P) and Cysteine (C) are not present in Helix 1 or Helix 2. Advantageously Glycine (G) is also not present in Helix 1 or Helix 2.

In one embodiment, the hBCMA binding motif, being the portion [Helix 1]-[Separating portion]-[Helix 2], is (i.e. has a sequence):

X₆X₇X₈X₉X₁₀X₁₁ADX₁₄EIX₁₇X₁₈X₁₉X₂₀X₂₁X₂₂X₂₃FX₂₅QKWA
FX₃₁RX₃₃LX₃₅X₃₆X₃₇

wherein
X₂₀ is any naturally occurring amino acid, X₂₁ is any naturally occurring amino acid; and X₂₂ is any naturally occurring amino acid, and wherein optionally one or two (for example optionally 1) of X₂₀, X₂₁ or X₂₂ are absent; and the other residues are as defined above.

More preferably, X₆ may be D, E, N or Q or is absent; X₇ may be H, K or R or is absent; X₈ may be D, E, N or Q or is absent; X₉, X₁₀, X₁₁, X₁₄, X₁₇ and X₁₈ are as defined above; X₁₉ may be G, A, V, L or I or is absent; X₂₀ may be S, T, M, P, F, Y or W (for example P); X₂₁ may be D, E, N or Q; X₂₂ may be G, A, V, L, I or is absent; X₂₃ may be D, E, N or Q or is absent; X₂₅, X₃₁, X₃₃ and X₃₅ are as defined above; X₃₆ may be D, E, N or Q or is absent; and X₃₇ may be D, E, N or Q or is absent.

For example, X₆ may be D, E, N or Q; X₇ may be H, K or R; X₈ may be D, E, N or Q; X₉, X₁₀, X₁₁, X₁₄, X₁₇ and X₁₈ are as defined above; X₁₉ may be G, A, V, L or I; X₂₀ may be S, T, M, P, F, Y or W (for example P); X₂₁ may be D, E, N or Q; X₂₂ may be G, A, V, L, I; X₂₃ may be D, E, N or Q; X₂₅, X₃₁, X₃₃ and X₃₅ are as defined above; X₃₆ may be D, E, N or Q; and X₃₇ may be D, E, N or Q.

In an especially preferred embodiment, X₆ is N; X₇ is K; X₈ is E; X₉, X₁₀, X₁₁, X₁₄, X₁₇ and X₁₈ are as defined above; X₁₉ is L; X₂₀ is S, T, M, P, F, Y or W (for example P), X₂₁ is D, E, N or Q (for example N); X₂₂ is G, A, V, L or, I (for example L); X₂₃ is N; X₂₅, X₃₁, X₃₃ and X₃₅ are as defined above; X₃₆ is D; and X₃₇ is D.

For example, the X₉ to X₃₅ portion of the hBCMA binding motif is selected from the group consisting of:

| | | | Motif is |
|---|---|---|---|
| | | | found in |
| | | | SEQ ID |
| Sequence | SEQ ID | Name | No. |
| ETFADLEISNLPNLNFYQKWAFIRSLM | 170 | Fa-G6 | 1 |
| NQFADEEIAALPNLNFYQKWAFIRKLM | 171 | 1-E6 | 2 |
| EIFADREIAFLPNLNFYQKWAFIRKLM | 172 | 1-E1 | 23 |
| SQFADEEIEALPNLNFYQKWAFIRSLM | 173 | 1-A5 | 24 |
| QEFADYEIATLPNLNFYQKWAFIRSLM | 174 | 1-A7 | 25 |
| DSFADTEIAKLPNLNFYQKWAFIRSLM | 175 | 1-F9 | 26 |
| EIFADQEIEALPNLNFYQKWAFIRSLM | 176 | 1-F10 | 27 |
| DYFADYEIATLPNLNFYQKWAFIRSLM | 177 | 2-B1 | 28 |
| EQFADEEIAHLPNLNFYQKWAFIRSLM | 178 | 2-C4 | 29 |
| SMFADIEITKLPNLNFYQKWAFIRKLM | 179 | 2-D3 | 30 |
| EVFADEEIASLPNLNFYQKWAFIRSLM | 180 | 2-H2 | 31 |
| VREADEEIAALPNLNFYQKWAFIRKLM | 181 | 2-A7 | 32 |
| HQFADYEIAMLPNLNFYQKWAFIRSLM | 182 | 2-E5 | 33 |
| HFFADKEIAKLPNLNFYQKWAFIRSLM | 183 | 2-E6 | 34 |
| KAFADTEIVNLPNLNFYQKWAFIRSLM | 184 | 2-G5 | 35 |
| SEFADAEIANLPNLNFYQKWAFIRKLM | 185 | 2-A10 | 36 |
| ENFADHEIALLPNLNFYQKWAFIRKLM | 186 | 2-B10 | 37 |
| DSHADEEIAALPNLNFYQKWAFIRKLM | 187 | 2-C5 | 38 |
| SKFADEEIAKLPNLNFYQKWAFIRSLM | 188 | 2-D10 | 39 |
| QQFADEEIAMLPNLNFYQKWAFIRKLM | 189 | 1-A2 | 41 |
| HHFADIEISHLPNLNFYQKWAFIRSLM | 190 | 1-B1 | 42 |
| SQFADQEIAALPNLNFYQKWAFIRKLM | 191 | 1-B2 | 43 |
| EYFADSEIAQLPNLNFYQKWAFIRKLM | 192 | 1-B3 | 44 |
| EKFADVEIAYLPNLNFYQKWAFIRKLM | 193 | 1-C1 | 45 |
| TEFADSEIVRLPNLNFYQKWAFIRSLM | 194 | 1-C2 | 46 |
| TTFADQEIASLPNLNFYQKWAFIRKLM | 195 | 1-C3 | 47 |
| YKFADYEIVALPNLNFYQKWAFIRKLM | 196 | 1-D1 | 48 |
| FQFADIEIAELPNLNFYQKWAFIRSLM | 197 | 1-D2 | 49 |
| QTFADQEIAELPNLNFYQKWAFIRKLM | 198 | 1-D3 | 50 |
| TSFADKEIALLPNLNFYQKWAFIRKLM | 199 | 1-E3 | 51 |
| RMFADIEIQQLPNLNFYQKWAFIRSLM | 200 | 1-F1 | 52 |
| IMFADQEIVSLPNLNFYQKWAFIRSLM | 201 | 1-F2 | 53 |
| TQFADQEIAFLPNLNFYQKWAFIRKLM | 202 | 1-F3 | 54 |
| LDFADQEIAYLPNLNFYQKWAFIRSLM | 203 | 1-G1 | 55 |
| LIFADEEIVALPNLNFYQKWAFIRSLM | 204 | 1-G2 | 56 |
| HFFADSEIAALPNLNFYQKWAFVRSLM | 205 | 1-H1 | 57 |
| HNFADQEIANLPNLNFYQKWAFIRSLM | 206 | 1-H2 | 58 |
| MEFADIEIQSLPNLNFYQKWAFIRSLM | 207 | 1-A4 | 59 |
| TWFADSEIAQLPNLNFYQKWAFIRSLM | 208 | 1-A6 | 60 |
| ETFADKEIAELPNLNFYQKWAFIRKLM | 209 | 1-B4 | 61 |
| QEFADVEIATLPNLNFYQKWAFIRKLM | 210 | 1-B6 | 62 |
| EHFADIEIASLPNLNFYQKWAFIRSLM | 211 | 1-C4 | 63 |
| FQFADREIAVLPNLNFYQKWAFIRSLM | 212 | 1-C5 | 64 |
| ITFADSEIASLPNLNFYQKWAFIRSLM | 213 | 1-C6 | 65 |
| MEFADQEIVVLPNLNFYQKWAFIRKLM | 214 | 1-D4 | 66 |
| KSFADNEIAELPNLNFYQKWAFIRKLM | 215 | 1-D5 | 67 |
| THFADKEIANLPNLNFYQKWAFIRSLM | 216 | 1-E4 | 68 |
| EQFADMEIASLPNLNFYQKWAFIRKLM | 217 | 1-E5 | 69 |
| HSFADNEIAALPNLNFYQKWAFIRSLM | 218 | 1-F4 | 70 |
| ATFADLEIEALPNLNFYQKWAFIRKLM | 219 | 1-F5 | 71 |
| ENFADNEIASLPNLNFYQKWAFIRSLM | 220 | 1-G3 | 72 |
| SAFADIEIASLPNLNFYQKWAFIRKLM | 221 | 1-G4 | 73 |
| ELFADQEIANLPNLNFYQKWAFMRSLM | 222 | 1-G5 | 74 |
| AVFADEEITNLPNLNFYQKWAFIRSLM | 223 | 1-H3 | 75 |
| TLFADHEIANLPNLNFYQKWAFIRSLM | 224 | 1-H5 | 76 |
| KHFADEEIAKLPNLNFYQKWAFIRKLM | 225 | 1-A8 | 77 |
| ERFADLEISVLPNLNFYQKWAFIRKLM | 226 | 1-B7 | 78 |
| MTFADYEISVLPNLNFYQKWAFIRSLM | 227 | 1-B8 | 79 |
| VQFADEEIAMLPNLNFYQKWAFIRKLM | 228 | 1-C7 | 80 |
| ELFADEEIQALPNLNFYQKWAFIRSLM | 229 | 1-C8 | 81 |
| DKFADEEIAALPNLNFYQKWAFIRSLM | 230 | 1-D6 | 82 |
| EHFADEEIASLPNLNFYQKWAFIRKLM | 231 | 1-D7 | 83 |
| ERFADEEIASLPNLNFYQKWAFIRKLM | 232 | 1-D8 | 84 |
| YRFADTEIADLPNLNFYQKWAFIRKLM | 233 | 1-E7 | 85 |
| QSFADEEIAFLPNLNFYQKWAFIRSLM | 234 | 1-E8 | 86 |
| ELFADTEIAQLPNLNFYQKWAFIRSLM | 235 | 1-F8 | 87 |
| NQFADIEIAYLPNLNFYQKWAFIRSLM | 236 | 1-G6 | 88 |
| HFFADTEIAFLPNLNFYQKWAFIRKLM | 237 | 1-H6 | 89 |
| AVFADHEIAFLPNLNFYQKWAFIRKLM | 238 | 1-H7 | 90 |
| NEFADEEIALLPNLNFYQKWAFIRSLM | 239 | 1-H8 | 91 |
| DDFADEEIAALPNLNFYQKWAFIRSLM | 240 | 1-A9 | 92 |
| TDFADVEIVALPNLNFYQKWAFIRKLM | 241 | 1-A10 | 93 |
| ERFADMEISNLPNLNFYQKWAFIRSLM | 242 | 1-C9 | 94 |
| EAFADTEIAALPNLNFYQKWAFIRSLM | 243 | 1-C11 | 95 |
| ETEADMEISNLPNLNFYQKWAFIRSLM | 244 | 1-D10 | 96 |
| QKFADDEIAALPNLNFYQKWAFIRSLM | 245 | 1-D11 | 97 |
| EAFADMEIERLPNLNFYQKWAFIRSLM | 246 | 1-E9 | 98 |
| INFADEEIAYLPNLNFYQKWAFIRSLM | 247 | 1-E10 | 99 |
| EQFADQEIALLPNLNFYQKWAFIRKLM | 248 | 1-H10 | 100 |
| HAFADQEIAILPNLNFYQKWAFIRSLV | 249 | 2-B2 | 101 |
| DTFADNEIAFLPNLNFYQKWAFIRSLM | 250 | 1-B12 | 102 |
| QYFADYEIAHLPNLNFYQKWAFIRKLM | 251 | 2-D1 | 103 |
| EHFADLEISALPNLNFYQKWAFIRSLL | 252 | 1-D12 | 104 |
| ISFADQEIATLPNLNFYQKWAFIRSLM | 253 | 1-F11 | 105 |
| SNFADYEIAQLPNLNFYQKWAFIRKLI | 254 | 2-G1 | 106 |
| YDFADEEITALPNLNFYQKWAFIRKLL | 255 | 1-G11 | 107 |
| AQFADIEISFLPNLNFYQKWAFIRSLV | 256 | 2-A4 | 108 |
| VDFADEEIAQLPNLNFYQKWAFIRSLM | 257 | 2-B3 | 109 |
| KEHADEEIAALPNLNFYQKWAFIRSLM | 258 | 2-B4 | 110 |
| EWFADNEIAELPNLNFYQKWAFIRSLM | 259 | 2-C3 | 111 |
| EQFADQEITNLPNLNFYQKWAFIRSLM | 260 | 2-D4 | 112 |
| ESFADTEIANLPNLNFYQKWAFIRSLM | 261 | 2-E3 | 113 |
| DVEADKEIAQLPNLNFYQKWAFIRKLM | 262 | 2-G2 | 114 |
| RYYADVEIAKLPNLNFFQKWAFIRKLM | 263 | 2-G3 | 115 |
| NEFADEEIAELPNLNFYQKWAFIRSLM | 264 | 2-H4 | 116 |
| QFQADMEIAQLPNLNFYQKWAFIRSLM | 265 | 2-A5 | 117 |
| FAFADEEIAQLPNLNFYQKWAFIRSLM | 266 | 2-A6 | 118 |
| YKFADVEITDLPNLNFYQKWAFIRSLM | 267 | 2-B7 | 119 |
| EETADMEIARLPNLNFYQKWAFIRSLM | 268 | 2-F6 | 121 |
| KQQADEEIAHLPNLNFYQKWAFIRSLM | 269 | 2-D9 | 122 |
| DQFADTEIAALPNLNFYQKWAFIRSLM | 270 | 2-H8 | 123 |
| VRFADTEIAYLPNLNFYQKWAFIRSLM | 271 | 2-E10 | 124 |
| DMFADMEIATLPNLNFYQKWAFIRKLM | 272 | 2-H10 | 125 |
| QSFADEEIAILPNLNFYQKWAFVRSLM | 273 | 2-C11 | 126 |
| EQYADVEIAYLPNLNFYQKWAFIRKLM | 274 | 2-G12 | 127 |
| ESFADWEIQKLPNLNFYQKWAFIRKLM | 275 | 2-F11 | 128 |

In a polypeptide of the table above, optionally from 1 to 5 residues (for example 1, 2, 3, 4 or 5), preferably 1, 2 or 3 residues, in the sequence are replaced by an alternative residue. Preferably, in embodiments wherein optionally from 1 to 5 residues (for example 1, 2, 3, 4 or 5), preferably 1, 2 or 3 residues, in the sequence are replaced by 5 an alternative residue, the replacement residue is a conservative replacement. In a preferred embodiment, the sequence is the one of SEQ ID 170-275. In a preferred embodiment, the hBCMA binding motif is selected from the group consisting of:

| | | | Motif is |
|---|---|---|---|
| | | | found in |
| | | | SEQ ID |
| Sequence | SEQ ID | Name | No. |
| ETFADLEISNLPNLNFYQKWAFIRSLM | 170 | Fa-G6 | 1 |
| NQFADEEIAALPNLNFYQKWAFIRKLM | 171 | 1-E6 | 2 |
| EIFADREIAFLPNLNFYQKWAFIRKLM | 172 | 1-E1 | 23 |
| SQFADEEIEALPNLNFYQKWAFIRSLM | 173 | 1-A5 | 24 |
| QEFADYEIATLPNLNFYQKWAFIRSLM | 174 | 1-A7 | 25 |
| DSFADTEIAKLPNLNFYQKWAFIRSLM | 175 | 1-F9 | 26 |
| EIFADQEIEALPNLNFYQKWAFIRSLM | 176 | 1-F10 | 27 |
| DYFADYEIATLPNLNFYQKWAFIRSLM | 177 | 2-B1 | 28 |
| EQFADEEIAHLPNLNFYQKWAFIRSLM | 178 | 2-C4 | 29 |
| SMFADIEITKLPNLNFYQKWAFIRKLM | 179 | 2-D3 | 30 |
| EVFADEEIASLPNLNFYQKWAFIRSLM | 180 | 2-H2 | 31 |
| VREADEEIAALPNLNFYQKWAFIRKLM | 181 | 2-A7 | 32 |
| HQFADYEIAMLPNLNFYQKWAFIRSLM | 182 | 2-E5 | 33 |
| HFFADKEIAKLPNLNFYQKWAFIRSLM | 183 | 2-E6 | 34 |
| KAFADTEIVNLPNLNFYQKWAFIRSLM | 184 | 2-G5 | 35 |
| SEFADAEIANLPNLNFYQKWAFIRKLM | 185 | 2-A10 | 36 |
| ENFADHEIALLPNLNFYQKWAFIRKLM | 186 | 2-B10 | 37 |
| DSHADEEIAALPNLNFYQKWAFIRKLM | 187 | 2-C5 | 38 |
| SKFADEEIAKLPNLNFYQKWAFIRSLM | 188 | 2-D10 | 39 |

In a polypeptide of the table above, optionally from 1 to 5 residues (for example 1, 2, 3, 4 or 5), preferably 1, 2 or 3 residues, in the sequence are replaced by an alternative residue. Preferably, in embodiments wherein optionally from 1 to 5 residues (for example 1, 2, 3, 4 or 5), preferably 1, 2 or 3 residues, in the sequence are replaced by an alternative residue, the replacement residue is a conservative replacement.

In preferred embodiments, the hBCMA binding motif sequence additionally has the residues NKE at positions X₆X₇X₈ (i.e. X₆ is N; X₇ is K; X₈ is E). In preferred embodiments, the motif sequence additionally has the residues DD at its positions X₃₆X₃₇ (i.e. X₃₆ is D; and X₃₇ is D). For example, the motif sequences have NKE at positions X₆X₇X₈ and DD at positions X₃₆X₃₇ (i.e. X₆ is N; X₇ is K; X₈ is E; X₃₆ is D; and X₃₇ is D).

Therefore, in preferred embodiments, the hBCMA binding motif sequence may be selected from:

NKEX₉X₁₀X₁₁ADX₁₄EIX₁₇X₁₈X₁₉X₂₀X₂₁X₂₂X₂₃FX₂₅QKWAF
X₃₁RX₃₃LX₃₅X₃₆X₃₇;
X₆X₇X₈X₉X₁₀X₁₁ADX₁₄EIX₁₇X₁₈X₁₉X₂₀X₂₁X₂₂X₂₃FX₂₅QK
WAFX₃₁RX₃₃LX₃₅DD;
and;
NKEX₉X₁₀X₁₁ADX₁₄EIX₁₇X₁₈X₁₉X₂₀X₂₁X₂₂X₂₃FX₂₅QKWAF
X₃₁RX₃₃LX₃₅DD.

As mentioned above, in embodiments of the invention a number of residues may each be substituted by an alternative residue, as described herein. For example, in the embodiments described above, even when not explicitly mentioned, optionally 1 to 5 (for example 1, 2, 3, 4 or 5), and preferably optionally 1 to 3 (for example 1, 2, or 3), residues in the sequence of Helix 1 and/or Helix 2 defined above are replaced by an alternative residue (for example replaced by an alternative residue that is a conservative replacement). In any such alternative polypeptides of the invention with alternative residues in place, binding to hBCMA is maintained. For example, the hBCMA binding efficacy is at least 1% of the binding efficacy of the peptide of SEQ ID NO: 2 (i.e. binding efficacy of the peptide of SEQ ID NO: 2 to hBCMA when measured under the same conditions (as described above)). Alternatively, or additionally, for example, in any such alternative polypeptides of the invention with alternative residues in place, the hBCMA-binding polypeptide is one that competes with SEQ ID NO: 2 for hBCMA binding.

More preferably, the binding efficacy is at least 5%, and more preferably at least 10%, 20%, 25% or 50% of the binding efficacy of the peptide of SEQ ID NO: 2 ((i.e. binding efficacy of the peptide of SEQ ID NO: 2 to hBCMA, when measured under the same conditions).

In advantageous embodiments of hBCMA-binding polypeptides of the invention, Proline (P) and Cysteine (C) are not present in the hBCMA binding motif. Advantageously Glycine (G) is also not present in the hBCMA binding motif.

As described herein above, the hBCMA-binding polypeptide of the invention has the overall structure [N-terminal portion]-[Helix 1]-[Separating portion]-[Helix 2]-[C-terminal portion].

The separating portion may be a sequence of 1 to 5 (preferably 1, 2, 3, 4 or 5) naturally occurring amino acids. Preferably, the separating portion is a sequence of from 2 to 5 (for example 2, 3, 4 or 5) naturally occurring amino acids. For example, the separating portion is a sequence of from 3 to 5 (for example 3, 4 or 5) naturally occurring amino acids. Preferably the separating portion is a sequence of 3 amino acids.

In certain embodiments, the separating portion has the sequence X₂₀X₂₁X₂₂, wherein X₂₀ is any naturally occurring amino acid, X₂₁ is any naturally occurring amino acid; and X₂₂ is any naturally occurring amino acid; and wherein optionally one or two of X₂₀, X₂₁ or X₂₂ are absent. For example, none of X₂₀, X₂₁ or X₂₂ is absent, one of X₂₀, X₂₁ or X₂₂ is absent, or two of X₂₀, X₂₁ or X₂₂ are absent. More preferably, none of X₂₀, X₂₁ or X₂₂ is absent or one of X₂₀, X₂₁ or X₂₂ is absent. Most preferably none of X₂₀, X₂₁ or X₂₂ is absent, i.e. the separating portion has the sequence X₂₀X₂₁X₂₂, wherein X₂₀ is any naturally occurring amino acid, X₂₁ is any naturally occurring amino acid; and X₂₂ is any naturally occurring amino acid.

Preferably, X₂₀ is S, T, M, P, F, Y or W (for example P or T), X₂₁ is D, E, N, Q, and X₂₂ is G, A, V, L, I; wherein optionally one or two (for example one) of X₂₀, X₂₁ or X₂₂ are absent. In one embodiment, X₂₀ is P, S, C, U, T, or M, X₂₁ is D, E, N or Q, and X₂₂ is G, A, V, L, I. More preferably, X₂₀ is P or T; X₂₁ is N; and X₂₂ is L. For example, the separating portion has the sequence PNL or TNL.

The N-terminal portion may be absent or may be a sequence of 1 to 15 naturally occurring amino acids. For example, the N-terminal portion may be absent or may be a sequence of from 1 to 10, from 1 to 8, from 1 to 6, or from 1 to 5 naturally occurring amino acids, for example 1, 2, 3, 4 or 5 naturally occurring amino acids.

In certain embodiments, the N-terminal portion has the sequence XₐX_{b}X₁X₂X₃X₄X₅, wherein Xₐ is any naturally occurring amino acid (for example M) or is absent; X_{b} is any naturally occurring amino acid (for example M) or is absent; X₁ is any naturally occurring amino acid or is absent; X₂ is any naturally occurring amino acid or is absent; X₃ is any naturally occurring amino acid or is absent; X₄ is any naturally occurring amino acid or is absent; and X₅ is any naturally occurring amino acid or is absent.

Preferably, the N-terminal portion has the sequence XₐX_{b}X₁X₂X₃X₄X₅, wherein Xₐ is M or is absent; X_{b} is M or is absent; X₁ is G, A, V, L, I or is absent, X₂ is D, E, N or Q or is absent, X₃ is D, E, N or Q or is absent, X₄ is H, K, R or is absent, and X₅ is F, Y, W or is absent. More preferably, Xₐ is M or is absent; X_{b} is M or is absent; X₁ is V, G or absent (for example V or absent); X₂ is D or absent; X₃ is N or absent; X₄ is K or absent; and X₅ is F or absent. More preferably, Xₐ is M or is absent; X_{b} is M or is absent; X₁ is V or absent; X₂ is D or absent; X₃ is N or absent; X₄ is K or absent; and X₅ is F or absent.

In certain embodiments, preferably none of Xₐ, X_{b}, X₁, X₂, X₃, X₄ and X₅ is absent; or Xₐ, and/or X_{b} are absent and the other residues are not absent; or Xₐ, X_{b}, and X₁ are absent and the other residues are not absent; or Xₐ, X_{b}, X₁ and X₂ are absent and the other residues are not absent; Xₐ, X_{b}, X₁, X₂ and X₃ are absent and the other residues are not absent; Xₐ, X_{b}, X₁, X₂, X₃ and X₄ are absent and the other residue is not absent; or all of Xₐ, X_{b}, X₁, X₂, X₃, X₄ and X₅ are absent.

In certain embodiments, preferably none of X₁, X₂, X₃, X₄ and X₅ is absent; X₁ is absent and the other residues are not absent; X₁ and X₂ are absent and the other residues are not absent; X₁, X₂ and X₃ are absent and the other residues are not absent; X₁, X₂, X₃ and X₄ are absent and the other residue is not absent; or all of X₁, X₂, X₃, X₄ and X₅ are absent. In such embodiments, optionally Xₐ, and/or X_{b} are absent.

In certain especially preferred embodiments, the N-terminal portion has the sequence XₐX_{b}X₁X₂X₃X₄X₅, wherein: Xₐ, and X_{b} are M, X₁ is V or G (preferably V), X₂ is D, X₃ is N, X₄ is K, and X₅ is F; or Xₐ, and X_{b} are absent, X₁ is V or G (preferably V), X₂ is D, X₃ is N, X₄ is K, and X₅ is F; or Xₐ, and X_{b} are absent, X₁ is absent, X₂ is D, X₃ is N, X₄ is K, and X₅ is F; or Xₐ, and X_{b} are absent, X₁ is absent, X₂ is absent, X₃ is N, X₄ is K, and X₅ is F; or Xₐ, and X_{b} are absent, X₁ is absent, X₂ is absent, X₃ is absent, X₄ is K, and X₅ is F; Xₐ, and X_{b} are absent, X₁ is absent, X₂ is absent, X₃ is absent, X₄ is absent, and X₅ is F; or Xₐ, and X_{b} are absent, X₁ is absent, X₂ is absent, X₃ is absent, X₄ is absent, and X₅ is absent.

In certain embodiments, the N-terminal portion has the sequence X₁X₂X₃X₄X₅, wherein: X₁ is any naturally occurring amino acid (preferably G, A, V, L or I; more preferably V or G) or is absent; X₂ is any naturally occurring amino acid (preferably D, E, N or Q; more preferably D) or is absent; X₃ is any naturally occurring amino acid (preferably D, E, N or Q; more preferably N) or is absent; X₄ is any naturally occurring amino acid (preferably H, K or R; more preferably K) or is absent; X₅ is any naturally occurring amino acid (preferably F, Y or W; more preferably F) or is absent.

In certain embodiments, preferably none of X₁, X₂, X₃, X₄ and X₅ is absent; or Xₐ, and/or X_{b} are absent and the other residues are not absent; or Xₐ, X_{b}, and X₁ are absent and the other residues are not absent; or Xₐ, X_{b}, X₁ and X₂ are absent and the other residues are not absent; Xₐ, X_{b}, X₁, X₂ and X₃ are absent and the other residues are not absent; Xₐ, X_{b}, X₁, X₂, X₃ and X₄ are absent and the other residue is not absent; or all of Xₐ, X_{b}, X₁, X₂, X₃, X₄ and X₅ are absent.

For example, the N-terminal portion has the sequence X₁X₂X₃X₄X₅, wherein: X₁ is V or G (preferably V), X₂ is D, X₃ is N, X₄ is K, and X₅ is F; or X₁ is absent, X₂ is D, X₃ is N, X₄ is K, and X₅ is F; or X₁ is absent, X₂ is absent, X₃ is N, X₄ is K, and X₅ is F; or X₁ is absent, X₂ is absent, X₃ is absent, X₄ is K, and X₅ is F; or X₁ is absent, X₂ is absent, X₃ is absent, X₄ is absent, and X₅ is F; or X₁ is absent, X₂ is absent, X₃ is absent, X₄ is absent, and X₅ is absent. For example, X₁ is V or G (preferably V), X₂ is D, X₃ is N, X₄ is K, and X₅ is F; or X₁ is absent, X₂ is absent, X₃ is absent, X₄ is absent, and X₅ is absent, i.e. the N-terminal portion has the sequence VDNKF, GDNKF or is absent; and more preferably has the sequence VDNKF or is absent.

In one preferred embodiment, X₁ is V or G (preferably V), X₂ is D, X₃ is N, X₄ is K, and X₅ is F, i.e. the N-terminal portion has the sequence VDNKF or GDNKF; and more preferably the N-terminal portion has the sequence VDNKF.

In one preferred embodiment, X₁ is absent, X₂ is absent, X₃ is absent, X₄ is absent, and X₅ is absent, i.e. the N-terminal portion is absent.

The C-terminal portion may be absent or may be a sequence of 1 to 50 naturally occurring amino acids. For example, the C-terminal portion may be absent or may be a sequence of from 1 to 40, 1 to 35, 1 to 30, from 1 to 25, from 1 to 22, or from 1 to 21 naturally occurring amino acids. Preferably, the C-terminal portion may be absent or may be a sequence of from 10 to 35, 10 to 30, from 15 to 25, from 18 to 22, from 1 to 21 naturally occurring amino acids, for example 18, 19, 20, 21 or 22 naturally occurring amino acids. Preferably, the C-terminal portion has a sequence such that it enhances target binding by the hBCMA-binding motif of Helix 1 and Helix 2.

In certain embodiments, the C-terminal portion is absent or has the sequence X₃₈X₃₉QSANLLAEAKKLNDAQX₅₆X₅₇X₅₈, wherein X₃₈ is a sequence of 1 to 14 naturally occurring amino acids, X₃₉ is any naturally occurring amino acid, X₅₆ is any naturally occurring amino acid or is absent, X₅₇ is any naturally occurring amino acid or is absent; and X₅₈ is any naturally occurring amino acid or is absent, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In certain embodiments, X₃₈ is a sequence of 1 to 9, 1 to 7 or 1 to 5 naturally occurring amino acids. More preferably X₃₈ is a sequence of 1 to 4, for example 1, 2, 3 or 4. In one especially preferred embodiment, X₃₈ is any naturally occurring amino acid (i.e. any single (one) naturally occurring amino acid residue).

In one preferred embodiment, the C-terminal portion has the sequence X₃₈X₃₉QSANLLAEAKKLNDAQX₅₆X₅₇X₅₈, wherein X₃₈ is P, X₃₉ is S, T, M, P, F, Y or W, X₅₆ is G, A, V, L, I or is absent, X₅₇ is P or is absent, and X₅₈ is H, K, R or is absent, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. In another preferred embodiment, the C-terminal portion has the sequence PSQSANLLAEAKKLNDAQX₅₆X₅₇X₅₈, wherein X₅₆ is G, A, V, L, I or is absent, X₅₇ is P or is absent, and X₅₈ is H, K, R or is absent, and optionally wherein from 1 to 5 (for example 1, 2, 3, 4 or 5; preferably 1, 2 or 3) of the residues in the sequence PSQSANLLAHAKKINDAQ are replaced by an alternative residue, for example replaced by an alternative residue that is a conservative replacement.

In an especially preferred embodiment, the C-terminal portion has the X₃₈X₃₉QSANLLAEAKKLNDAQX₅₆X₅₇X₅₈, wherein X₃₈ is P, X₃₉ is S, X₅₆ is A or is absent, X₅₇ is P or is absent, and X₅₈ is K or is absent, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. In another especially preferred embodiment, the C-terminal portion has the PSQSANLLAEAKKLNDAQX₅₆X₅₇X₅₈, wherein X₅₆ is A or is absent, X₅₇ is P or is absent, and X₅₈ is K or is absent, and optionally wherein from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

For example, in certain embodiments the C-terminal portion has the sequence X₃₈X₃₉QSANLLAEAKKLNDAQX₅₆X₅₇X₅₈, wherein X₃₈ is P, and X₃₉ is S; and:
X₅₆ is A, X₅₇ is P, and X₅₈ is K; or X₅₆ is A, or X₅₇ is P, and X₅₈ is absent; or X₅₆ is A, X₅₇ is absent, and X₅₈ is absent; or X₅₆ is absent, X₅₇ is absent, and X₅₈ is absent. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. Or, for example, the C-terminal portion has the sequence PSQSANLLAEAKKLNDAQX₅₆X₅₇X₅₈, wherein X₅₆ is A, X₅₇ is P, and X₅₈ is K; or X₅₆ is A, or X₅₇ is P, and X₅₈ is absent; or X₅₆ is A, X₅₇ is absent, and X₅₈ is absent; or X₅₆ is absent, X₅₇ is absent, and X₅₈ is absent. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one preferred the C-terminal portion has the sequence X₃₈X₃₉QSANLLAEAKKLNDAQX₅₆X₅₇X₅₈, wherein X₃₈ is P, and X₃₉ is S; and X₅₆ is A, X₅₇ is P, and X₅₈ is K. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. Or, for example,

For example, the C-terminal portion has the sequence PSQSANLLAEAKKLNDAQX₅₆X₅₇X₅₈, wherein X₅₆ is A, X₅₇ is P, and X₅₈ is K. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In an alternative preferred the C-terminal portion has the sequence X₃₈X₃₉QSANLLAEAKKLNDAQX₅₆X₅₇X₅₈, wherein X₃₈ is P, and X₃₉ is S; and:
X₅₆ is absent, X₅₇ is absent, and X₅₈ is absent. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement. Or, for example, For example, the C-terminal portion has the sequence PSQSANLLAEAKKLNDAQX₅₆X₅₇X₅₈, X₅₆ is absent, X₅₇ is absent, and X₅₈ is absent. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one embodiment, the N-terminal portion has the sequence XₐX_{b}X₁X₂X₃X₄X₅ wherein Xₐ is M, X_{b} is M, X₁ is V or G (preferably V), X₂ is D, X₃ is N, X₄ is K, and X₅ is F; Xₐ is absent, X_{b} is M, X₁ is V or G (preferably V), X₂ is D, X₃ is N, X₄ is K, and X₅ is F; Xₐ is absent, X_{b} is absent, X₁ is V or G (preferably V), X₂ is D, X₃ is N, X₄ is K, and X₅ is F; Xₐ is absent, X_{b} is absent, X₁ is absent, X₂ is D, X₃ is N, X₄ is K, and X₅ is F; Xₐ is absent, X_{b} is absent, X₁ is absent, X₂ is absent, X₃ is N, X₄ is K, and X₅ is F; Xₐ is absent, X_{b} is absent, X₁ is absent, X₂ is absent, X₃ is absent, X₄ is K, and X₅ is F; Xₐ is absent, X_{b} is absent, X₁ is absent, X₂ is absent, X₃ is absent, X₄ is absent, and X₅ is F; or Xₐ is absent, X_{b} is absent, X₁ is absent, X₂ is absent, X₃ is absent, X₄ is absent, and X₅ is absent (for example, Xₐ is M, X_{b} is M, X₁ is V or G (preferably V), X₂ is D, X₃ is N, X₄ is K, and X₅ is F; or Xₐ is absent, X_{b} is absent, X₁ is V or G (preferably V), X₂ is D, X₃ is N, X₄ is K, and X₅ is F; or Xₐ is absent, X_{b} is absent, X₁ is absent, X₂ is absent, X₃ is absent, X₄ is absent, and X₅ is absent); and the C-terminal portion has the sequence X₃₈X₃₉QSANLLAEAKKLNDAQX₅₆X₅₇X₅₈, wherein X₃₈ is P, X₃₉ is S, T, M, P, F, Y or W, X₅₆ is G, A, V, L, I or is absent, X₅₇ is P, and X₅₈ is H, K, or R, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement; and more preferably X₃₈ is P, X₃₉ is S, X₅₆ is A, X₅₇ is P, and X₅₈ is K, and optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In another embodiment, the N-terminal portion has the sequence XₐX_{b}X₁X₂X₃X₄X₅ wherein Xₐ is M, X_{b} is M, X₁ is V or G (preferably V), X₂ is D, X₃ is N, X₄ is K, and X₅ is F; Xₐ is absent, X_{b} is absent, X₁ is absent, X₂ is D, X₃ is N, X₄ is K, and X₅ is F; Xₐ is absent, X_{b} is absent, X₁ is absent, X₂ is absent, X₃ is N, X₄ is K, and X₅ is F; Xₐ is absent, X_{b} is absent, X₁ is absent, X₂ is absent, X₃ is absent, X₄ is K, and X₅ is F; Xₐ is absent, X_{b} is absent, X₁ is absent, X₂ is absent, X₃ is absent, X₄ is absent, and X₅ is F; or Xₐ is absent, X_{b} is absent, X₁ is absent, X₂ is absent, X₃ is absent, X₄ is absent, and X₅ is absent (for example, Xₐ is M, X_{b} is M, X₁ is V or G (preferably V), X₂ is D, X₃ is N, X₄ is K, and X₅ is F, or Xₐ is absent, X_{b} is absent, X₁ is V or G (preferably V), X₂ is D, X₃ is N, X₄ is K, and X₅ is F, or Xₐ is absent, X_{b} is absent, X₁ is absent, X₂ is absent, X₃ is absent, X₄ is absent, and X₅ is absent); and the C-terminal portion has the sequence PSQSANLLAEAKKLNDAQX₅₆X₅₇X₅₈, wherein X₅₆ is G, A, V, L, I or is absent, X₅₇ is P, and X₅₈ is H, K, or R, and optionally wherein from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement; and more preferably X₅₆ is A, X₅₇ is P, and X₅₈ is K, and optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one embodiment, the N-terminal portion has the sequence XₐX_{b}X₁X₂X₃X₄X₅ wherein Xₐ is M, X_{b} is M, X₁ is G, A, V, L, or I, X₂ is D, E, N, or Q, X₃ is D, E, N, or Q, X₄ is H, K, or R, and X₅ is F, Y, or W (for example, Xₐ is M, X_{b} is M, X₁ is V or G (preferably V), X₂ is D, X₃ is N, X₄ is K, and X₅ is F); and the C-terminal portion has the sequence X₃₈X₃₉QSANLLAEAKKLNDAQX₅₆X₅₇X₅₈, wherein X₃₈ is P, X₃₉ is S, T, M, P, F, Y or W (preferably S); and X₅₆ is G, A, V, L or I (preferably A), X₅₇ is P, and X₅₈ is H, K, or R (preferably K); X₅₆ is G, A, V, L or I (preferably A); X₅₇ is P, and X₅₈ is absent; X₅₆ is G, A, V, L or I (preferably A), X₅₇ is absent, and X₅₈ is absent; or X₅₆ is absent, X₅₇ is absent, and X₅₈ s absent. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In one embodiment, the N-terminal portion has the sequence XₐX_{b}X₁X₂X₃X₄X₅ wherein Xₐ is M, X_{b} is M, X₁ is G, A, V, L, or I, X₂ is D, E, N, or Q, X₃ is D, E, N, or Q, X₄ is H, K, or R, and X₅ is F, Y, or W (for example, Xₐ is M, X_{b} is M, X₁ is V or G (preferably V), X₂ is D, X₃ is N, X₄ is K, and X₅ is F); and the C-terminal portion has sequence PSQSANLLAEAKKLNDAQX₅₆X₅₇X₅₈, wherein X₅₆ is G, A, V, L or I (preferably A), X₅₇ is P, and X₅₈ is H, K, or R (preferably K); X₅₆ is G, A, V, L or I (preferably A); X₅₇ is P, and X₅₈ is absent; X₅₆ is G, A, V, L or I (preferably A), X₅₇ is absent, and X₅₈ is absent; or X₅₆ is absent, X₅₇ is absent, and X₅₈ s absent. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In another embodiment, the N-terminal portion has the sequence X₁X₂X₃X₄X₅ wherein X₁ is V or G (preferably V), X₂ is D, X₃ is N, X₄ is K, and X₅ is F; X₁ is absent, X₂ is D, X₃ is N, X₄ is K, and X₅ is F; X₁ is absent, X₂ is absent, X₃ is N, X₄ is K, and X₅ is F; X₁ is absent, X₂ is absent, X₃ is absent, X₄ is K, and X₅ is F; X₁ is absent, X₂ is absent, X₃ is absent, X₄ is absent, and X₅ is F; or Xₐ is absent, X_{b} is absent, X₁ is absent, X₂ is absent, X₃ is absent, X₄ is absent, and X₅ is absent (for example, X₁ is V or G (preferably V), X₂ is D, X₃ is N, X₄ is K, and X₅ is F, or X₁ is absent, X₂ is absent, X₃ is absent, X₄ is absent, and X₅ is absent); and the C-terminal portion has the sequence X₃₈X₃₉QSANLLAEAKKLNDAQX₅₆X₅₇X₅₈, wherein X₃₈ is P, X₃₉ is S, T, M, P, F, Y or W, X₅₆ is G, A, V, L, I or is absent, X₅₇ is P, and X₅₈ is H, K, or R, and optionally wherein from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement; and more preferably X₃₈ is P, X₃₉ is S, X₅₆ is A, X₅₇ is P, and X₅₈ is K, and optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In another embodiment, the N-terminal portion has the sequence X₁X₂X₃X₄X₅ wherein X₁ is V or G (preferably V), X₂ is D, X₃ is N, X₄ is K, and X₅ is F; X₁ is absent, X₂ is D, X₃ is N, X₄ is K, and X₅ is F; X₁ is absent, X₂ is absent, X₃ is N, X₄ is K, and X₅ is F; X₁ is absent, X₂ is absent, X₃ is absent, X₄ is K, and X₅ is F; X₁ is absent, X₂ is absent, X₃ is absent, X₄ is absent, and X₅ is F; or Xₐ is absent, X_{b} is absent, X₁ is absent, X₂ is absent, X₃ is absent, X₄ is absent, and X₅ is absent (for example, X₁ is V or G (preferably V), X₂ is D, X₃ is N, X₄ is K, and X₅ is F, or X₁ is absent, X₂ is absent, X₃ is absent, X₄ is absent, and X₅ is absent); and the C-terminal portion has the sequence PSQSANLLAEAKKLNDAQX₅₆X₅₇X₅₈, wherein X₅₆ is G, A, V, L, I or is absent, X₅₇ is P, and X₅₈ is H, K, or R, and optionally wherein from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement; and more preferably X₅₆ is A, X₅₇ is P, and X₅₈ is K, and optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In another embodiment, the N-terminal portion has the sequence X₁X₂X₃X₄X₅, wherein X₁ is G, A, V, L, or I, X₂ is D, E, N, or Q, X₃ is D, E, N, or Q, X₄ is H, K, or R, and X₅ is F, Y, or W (for example, X₁ is V or G (preferably V), X₂ is D, X₃ is N, X₄ is K, and X₅ is F); and the C-terminal portion has sequence X₃₈X₃₉QSANLLAEAKKLNDAQX₅₆X₅₇X₅₈, wherein X₃₈ is P, X₃₉ is S, T, M, P, F, Y or W (preferably S); and X₅₆ is G, A, V, L or I (preferably A), X₅₇ is P, and X₅₈ is H, K, or R (preferably K); X₅₆ is G, A, V, L or I (preferably A); X₅₇ is P, and X₅₈ is absent; X₅₆ is G, A, V, L or I (preferably A), X₅₇ is absent, and X₅₈ is absent; or X₅₆ is absent, X₅₇ is absent, and X₅₈ s absent. More preferably, X₅₆ is G, A, V, L or I (preferably A), X₅₇ is P, and X₅₈ is H, K, or R (preferably K); or X₅₆ is absent, X₅₇ is absent, and X₅₈ is absent. In such embodiments optionally from 1 to 5 (for example 1, 2, 3, 4 or 5; preferably 1, 2 or 3) of the residues in the sequence QSANLLAEAKKLNDAQ are replaced by an alternative residue, for example replaced by an alternative residue that is a conservative replacement.

In another embodiment, the N-terminal portion has the sequence X₁X₂X₃X₄X₅, wherein X₁ is G, A, V, L, or I, X₂ is D, E, N, or Q, X₃ is D, E, N, or Q, X₄ is H, K, or R, and X₅ is F, Y, or W (for example, X₁ is V or G (preferably V), X₂ is D, X₃ is N, X₄ is K, and X₅ is F); and the C-terminal portion has sequence PSQSANLLAEAKKLNDAQX₅₆X₅₇X₅₈, wherein X₅₆ is G, A, V, L or I (preferably A), X₅₇ is P, and X₅₈ is H, K, or R (preferably K); X₅₆ is G, A, V, L or I (preferably A); X₅₇ is P, and X₅₈ is absent; X₅₆ is G, A, V, L or I (preferably A), X₅₇ is absent, and X₅₈ is absent; or X₅₆ is absent, X₅₇ is absent, and X₅₈ s absent. More preferably, X₅₆ is G, A, V, L or I (preferably A), X₅₇ is P, and X₅₈ is H, K, or R (preferably K); or X₅₆ is absent, X₅₇ is absent, and X₅₈ is absent. In such embodiments optionally from 1 to 5 (for example 1, 2, 3, 4 or 5; preferably 1, 2 or 3) of the residues in the sequence PSQSANLLAEAKKLNDAQ are replaced by an alternative residue, for example replaced by an alternative residue that is a conservative replacement.

In one very preferred embodiment, X₁ is V or G (preferably V), X₂ is D, X₃ is N, X₄ is K, and X₅ is F; and X₅₆ is absent, X₅₇ is absent, and X₅₈ is absent.

In an alternative very preferred embodiment, X₁ is V or G (preferably V), X₂ is D, X₃ is N, X₄ is K, and X₅ is F; X₅₆ is G, A, V, L or I (preferably A), X₅₇ is P, and X₅₈ is H, K, or R (preferably K).

In an alternative very preferred embodiment Xₐ is absent, X_{b} is absent, X₁ is absent, X₂ is absent, X₃ is absent, X₄ is absent, and X₅ is absent; X₅₆ is G, A, V, L or I (preferably A), X₅₇ is P, and X₅₈ is H, K, or R (preferably K).

In certain embodiments, (i) the separating portion has the sequence X₂₀X₂₁X₂₂; and/or the N-terminal portion has the sequence XₐX_{b}X₁X₂X₃X₄X₅; and/or the C-terminal portion has the sequence PSQSANLLAEAKKLNDAQX₅₆X₅₇X₅₈;
wherein, in said separating portion, X₂₀ is P; X₂₁ is N; X₂₂ is L;
wherein in said N-terminal portion, Xₐ is M or absent; X_{b} is M or absent; X₁ is V or G (preferably V), or absent; X₂ is D or absent; X₃ is N, or absent; X₄ is K or absent; X₅ is F or absent;
and wherein in said C-terminal portion, X₅₆ is A or absent; X₅₇ is P or absent; X₅₈ is K or absent;
or (ii) the separating portion, N-terminal portion, and C-terminal portion are as defined in (i), wherein optionally
(a) within each portion 1, 2 or 3 residues are replaced by an alternative residue; or
(b) within those portions taken together at least 1 and no more than 10 (for example, not more than 5, for example 1, 2, 3, 4, or 5) residues are replaced by an alternative residue.

In such embodiments, preferably the separating portion has the sequence PNL. Alternatively, or additionally, in such embodiments, the N-terminal portion has the sequence MMVDNKF or VDNKF.

In another embodiment, the N-terminal portion may comprise the sequence X₁X₂X₃X₄X₅, wherein: X₁ is G, V, or absent; X₂ is D or absent; X₃ is N or absent; X₄ is K or absent; X₅ is F or absent. For example, the N-terminal portion may comprise the sequence VDNKF, the sequence DNKF, the sequence NKF, the sequence KF, or the sequence F.

The separating portion may comprise the sequence PNL.

The C-terminal portion may have the structure [Second separating portion]-[Helix 3]-[C-terminal sequence]. For example:
the Second separating portion comprises the sequence PS;
the Helix 3 portion comprises the sequence QSANLLAEAKKLNDAQ;
and the C-terminal sequence comprises the sequence APK.

For example, the C-terminal portion may comprise the sequence PSQSANLLAEAKKLNDAQAPK.

Within those five portions (N-terminal portion, separating portion, second separating portion, Helix 3, and C-terminal sequence) taken together, at least 1 and no more than 5 of the residues may be replaced by an alternative residue. For example, the number of replaced residues is at least 1 and no more than 4, for example at least 1 and no more than 3, for example at least 1 and no more than 2. For example, there may be 1, 2, 3, 4 or 5 replacement residues in total in those portions.

In advantageous embodiments of hBCMA-binding polypeptides of the invention, where the C-terminal portion comprises a helical or substantially helical region (for example Helix 3 as described immediately above), Proline (P) and Cysteine (C) are not present in that helical or substantially helical region. In another embodiment, additionally Glycine (G) is also not present in that helical or substantially helical region.

Thus, across the entire hBCMA-binding polypeptide of the invention (i.e. across the entire overall structure [N-terminal portion]-[Helix 1]-[Separating portion]-[Helix 2]-[C-terminal portion]), at least 1 and no more than 18 of the residues may be replaced by an alternative residue. For example, the number of replaced residues is at least 1 and no more than 18, at least 1 and no more than 17, at least 1 and no more than 16, at least 1 and no more than 15, at least 1 and no more than 14, at least 1 and no more than 13, at least 1 and no more than 12, at least 1 and no more than 11, at least 1 and no more than 10, at least 1 and no more than 9, at least 1 and no more than 8, at least 1 and no more than 7, or at least 1 and no more than 6. Particularly, there may be 1, 2, 3, 4, 5, 6, 7, 8, 9, 10, 11, 12, 13, 14 15, 16, 17, or 18 replacement residues in total across the entire overall structure [N-terminal portion]-[Helix 1]-[Separating portion]-[Helix 2]-[C-terminal portion]. An amino acid residue replacement may be a conservative or a non-conservative replacement, or a non-canonical (also known as unnatural) amino acid, as described herein above.

Preferably, across the entire hBCMA-binding polypeptide of the invention (i.e. across the entire overall structure [N-terminal portion]-[Helix 1]-[Separating portion]-[Helix 2]-[C-terminal portion]), at least 1 and no more than 15 of the residues may be replaced by an alternative residue. For example, the number of replaced residues is at least 1 and no more than 13, at least 1 and no more than 12, at least 1 and no more than 11, at least 1 and no more than 10, at least 1 and no more than 9, at least 1 and no more than 8, at least 1 and no more than 7, or at least 1 and no more than 6. Particularly, there may be 1, 2, 3, 4, 5, 6, 7, 8, 9, 10, 11, 12, 13, 14 or 15 replacement residues in total across the entire overall structure [N-terminal portion]-[Helix 1]-[Separating portion]-[Helix 2]-[C-terminal portion]. An amino acid residue replacement may be a conservative or a non-conservative replacement, or a non-canonical (also known as unnatural) amino acid, as described herein above.

In one aspect of the invention, the hBCMA-binding polypeptide comprises a sequence selected from SEQ ID NOs. 1, 2, 23-39, 41-119, or 121-128 as shown in Table 1. In such sequences, optionally from 1 to 5 (preferably 1, 2 or 3) residues in the sequence are replaced by an alternative residue, and preferably a residue that is a conservative replacement. In one embodiment, the hBCMA binding polypeptide comprises a sequence selected from SEQ ID NOs. 1, 2, 23-39, 41-119, or 121-128. In one embodiment, the hBCMA binding polypeptide has a sequence selected from the group consisting of SEQ ID NOs. 1, 2, 23-39, 41-119, or 121-128.

In one aspect of the invention, the hBCMA-binding polypeptide comprises a sequence selected from SEQ ID NOs. 2 and 23-39, as shown in Table 1. In such sequences, optionally from 1 to 5 (preferably 1, 2 or 3) residues in the sequence are replaced by an alternative residue, and preferably a residue that is a conservative replacement. In one embodiment, the hBCMA binding polypeptide comprises a sequence selected from SEQ ID NOs. 2 and 23-39. In one embodiment, the hBCMA binding polypeptide has a sequence selected from the group consisting of SEQ ID NOs. 2 and 23-39.

In one aspect of the invention, the hBCMA-binding polypeptide comprises the sequence:

a)
[SEQ ID NO: 1]
VDNKFNKEETFADLEISNLPNLNFYQKWAFIRSLMDDPSQSANLLAE
AKKLNDAQAPK;

In a further aspect of the invention, the hBCMA-binding polypeptide comprises the sequence:

b)
[SEQ ID NO: 2]
VDNKFNKENQFADEEIAALPNLNFYQKWAFIRKLMDDPSQSANLLA
EAKKLNDAQAPK.

In a further aspect of the invention, the hBCMA-binding polypeptide comprises the sequence

c)
[SEQ ID NO: 23]
VDNKFNKEEIFADREIAFLPNLNFYQKWAFIRKLMDDPSQSANLLAE
AKKLNDAQAPK.

In a further aspect of the invention, the hBCMA-binding polypeptide comprises the sequence

c)
[SEQ ID NO: 33]
VDNKFNKEHQFADYEIAMLPNLNFYQKWAFIRSLMDDPSQSANLLA
EAKKLNDAQAPK.

As described herein above, a number of residues in the polypeptides of the invention may be substituted by an alternative residue. For example, in a polypeptide as set out in a), b), c) or d) immediately above, at least 1 and no more than 5 of the residues may be replaced by an alternative residue. For example, the number of replaced residues is at least 1 and no more than 4, for example at least 1 and no more than 3, for example 1 at least 1 and no more than 2. Particularly, there may be 1, 2, 3, 4 or 5 replacement residues in total in those portions. As there are 58 residues in polypeptides of this aspect of the invention, a peptide with 5 residues replaced has 91% sequence identity (91.4%) with the recited sequence. For replacement of 4 residues, it is 93% (93.1%), for replacement of 3 residues, it is 95% (94.8%), for replacement of 2 residues it is 97% (96.6%) and for replacement of 1 residue it is 98% sequence identity (98.3%).

As disclosed herein above, in an embodiment, Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other, X₉ and X₁₀ are any naturally occurring amino acid; X₁₁ is E, F, H, Q, T or Y; X₁₄ is any naturally occurring amino acid; X₁₇ is A, E, Q, S, T or V; X₁₈ is any naturally occurring amino acid; X₂₅ is F or Y; X₃₁ is I, M, or V; X₃₃ is K or S; X₃₅ is I, L, M, or V. In this embodiment, as in other embodiments, a number of residues may each be substituted by an alternative residue, as described herein. In any such alternative polypeptide of the invention with alternative residues in place, binding to hBCMA is maintained. For example, the hBCMA binding efficacy is at least 0.1% of the binding efficacy of the peptide of SEQ ID NO: 2 to hBCMA, when measured under the same conditions.

With 0.1% binding efficacy, it is understood that the EC₅₀ concentration of the alternative polypeptide for binding to hBCMA is no more than 1000 times the EC₅₀ concentration for the peptide of SEQ ID NO: 2 to hBCMA, when measured under the same conditions.

More preferably, the binding efficacy is at least 0.5%, 1%, 2%, 4%, 5%, 10%, 20%, 25%, or 50% of the binding efficacy of the peptide of SEQ ID NO: 2 to hBCMA, when measured under the same conditions. That is to say that the EC₅₀ concentration of the alternative polypeptide for binding to hBCMA is no more than 500 times, 100 times, 50 times, 25 times, 20 times, 10 times, 5 times, 4 times or 2 times the EC₅₀ concentration for the peptide of SEQ ID NO: 2 to hBCMA, when measured under the same conditions.

As disclosed herein above, in another embodiment, Helix 1 comprises the sequence X₉X₁₀X₁₁ADX₁₄EIX₁₇X₁₈ [SEQ ID NO. 278] and Helix 2 comprises the sequence FYQKWAFIRX₃₃LM, wherein, independently from each other, X₉ is D, E, H, K, N, Q, S, or V; X₁₀ is A, E, F, I, K, M, N, Q, R, S, T, Y, or V; Xₙ is E, F, or H; X₁₄ is A, E, H, I, K, L, Q, R, T, or Y; X₁₇ is A, E S, T, or V; X₁₈ is A, F, H, K, L, M, N, T, or S; X₃₃ is K or S. In this embodiment, as in other embodiments, a number of residues may each be substituted by an alternative residue, as described herein. In any such alternative polypeptides of the invention with alternative residues in place, binding to hBCMA is maintained. For example, the hBCMA binding efficacy is at least 0.1% of the binding efficacy of the peptide of SEQ ID NO: 2 to hBCMA, when measured under the same conditions.

With 0.1% binding efficacy, it is understood that the EC₅₀ concentration of the alternative polypeptide for binding to hBCMA is no more than 1000 times the EC₅₀ concentration for the peptide of SEQ ID NO: 2 to hBCMA, when measured under the same conditions.

More preferably, the binding efficacy is at least 0.5%, 1%, 2%, 4%, 5%, 10%, 20%, 25%, or 50% of the binding efficacy of the peptide of SEQ ID NO: 2 to hBCMA, when measured under the same conditions. That is to say that the EC₅₀ concentration of the alternative polypeptide for binding to hBCMA is no more than 500 times, 100 times, 50 times, 25 times, 20 times, 10 times, 5 times, 4 times or 2 times the EC₅₀ concentration for the peptide of SEQ ID NO: 2 to hBCMA, when measured under the same conditions.

As disclosed herein above, in another embodiment, Helix 1 comprises the sequence ETFADLEISN and Helix 2 comprises the sequence FYQKWAFIRSLM. In this embodiment, as in other embodiments, a number of residues may each be substituted by an alternative residue, as described herein. In any such alternative polypeptides of the invention with alternative residues in place, binding to hBCMA is maintained. For example, the hBCMA binding efficacy is at least 0.1% of the binding efficacy of the peptide of SEQ ID NO: 2 to hBCMA, when measured under the same conditions. With 0.1% binding efficacy, it is understood that the EC₅₀ concentration of the alternative polypeptide for binding to hBCMA is no more than 1000 times the EC₅₀ concentration for the peptide of SEQ ID NO: 2 to hBCMA, when measured under the same conditions.

More preferably, the binding efficacy is at least 0.5%, 1%, 2%, 4%, 5%, 10%, 20%, 25%, or 50% of the binding efficacy of the peptide of SEQ ID NO: 2 to hBCMA, when measured under the same conditions. That is to say that the EC₅₀ concentration of the alternative polypeptide for binding to hBCMA is no more than 500 times, 100 times, 50 times, 25 times, 20 times, 10 times, 5 times, 4 times or 2 times the EC₅₀ concentration for the peptide of SEQ ID NO: 2 to hBCMA, when measured under the same conditions.

As also disclosed herein above, in another embodiment, Helix 1 comprises the sequence NQFADEEIAA and Helix 2 comprises the sequence FYQKWAFIRKLM. In this embodiment, as in other embodiments, a number of residues may each be substituted by an alternative residue, as described herein. In any such alternative polypeptides of the invention with alternative residues in place, binding to hBCMA is maintained. For example, the hBCMA binding efficacy is at least 0.1% of the binding efficacy of the peptide of SEQ ID NO: 2 to hBCMA, when measured under the same conditions.

With 0.1% binding efficacy, it is understood that the EC₅₀ concentration of the alternative polypeptide for binding to hBCMA is no more than 1000 times the EC₅₀ concentration for the peptide of SEQ ID NO: 2 to hBCMA, when measured under the same conditions.

More preferably, the binding efficacy is at least 0.5%, 1%, 2%, 4%, 5%, 10%, 20%, 25%, or 50% of the binding efficacy of the peptide of SEQ ID NO: 2 to hBCMA, when measured under the same conditions. That is to say that the EC₅₀ concentration of the alternative polypeptide for binding to hBCMA is no more than 500 times, 100 times, 50 times, 25 times, 20 times, 10 times, 5 times, 4 times or 2 times the EC₅₀ concentration for the peptide of SEQ ID NO: 2 to hBCMA, when measured under the same conditions.

The invention further provides an hBCMA-binding polypeptide, wherein the hBCMA-binding polypeptide consists of one motif that binds to hBCMA, wherein said polypeptide consists of the following structure:
[N-terminal portion]-[Helix 1]-[Separating portion]-[Helix 2]-[C-terminal portion]
the hBCMA binding motif being the portion [Helix 1]-[Separating portion]-[Helix 2] (and optionally the peptide further comprises one or more additional functional portions as described below (for example 1, 2, 3, 4, 5, 6 ore more); for example one, two or three additional functional portions).

The invention further provides a hBCMA-binding polypeptide, which consists of a CD16a-binding polypeptide of the invention; and optionally comprising an additional binding moiety. The invention further provides a hBCMA-binding polypeptide of the invention, wherein the hBCMA-binding polypeptide consists of the hBCMA-binding polypeptide (and optionally further comprises one or more additional functional portions as described below (for example 1, 2, 3, 4, 5, 6 ore more); for example one, two or three additional functional portions).

In one embodiment the hBCMA-binding polypeptide (for example a hBCMA-binding polypeptide of the invention described above or below) consists of one motif that binds to hBCMA, wherein said polypeptide consists of the following structure:
[N-terminal portion]-[Helix 1]-[Separating portion]-[Helix 2]-[C-terminal portion]
   the hBCMA-binding motif being the portion [Helix 1]-[Separating portion]-[Helix 2] (and optionally further comprises one or more additional functional portions as described below (for example 1, 2, 3, 4, 5, 6 ore more); for example one, two or three additional functional portions).

In one embodiment the hBCMA-binding polypeptide consists of a hBCMA-binding polypeptide of the present invention (and optionally further comprises one or more additional functional portions as described below (for example 1, 2, 3, 4, 5, 6 ore more); for example one, two or three additional functional portions).

For the avoidance of doubt, in embodiments wherein the hBCMA-binding motif and/or a hBCMA-binding polypeptide consists of a hBCMA-binding motif and/or a hBCMA-binding polypeptide of the invention, the hBCMA-binding polypeptide is not connected to a further hBCMA-binding polypeptide, i.e. the hBCMA-binding polypeptide is not a portion of a hBCMA-binding oligomer of the invention.

In another embodiment the hBCMA-binding polypeptide (for example a hBCMA-binding polypeptide of the invention described above or below) consists of one motif that binds to hBCMA, wherein said polypeptide consists of the following structure:
[N-terminal portion]-[Helix 1]-[Separating portion]-[Helix 2]-[C-terminal portion]
   the hBCMA-binding motif being the portion [Helix 1]-[Separating portion]-[Helix 2], and wherein the polypeptide further comprises one or more additional functional portions as described below (for example 1, 2, 3, 4, 5, 6 ore more); for example one, two or three additional functional portions). In another very preferred embodiment the hBCMA-binding polypeptide consists of a hBCMA-binding polypeptide of the invention, and further comprises one or more additional functional portions as described below (for example 1, 2, 3, 4, 5, 6 or more); for example 1, 2 or 3 additional functional portions (preferably 1 or 2 additional functional portions).
   Multimeric hBCMA-Binding Polypeptides: hBCMA-Binding Oligomers

The present invention further provides hBCMA-binding oligomers which comprise at least two (i.e. two, or more than two, for example 2, 3, 4, 5, 6 or more; preferably 2, 3 or 4) hBCMA-binding polypeptides of the present invention. In one preferred embodiment, an hBCMA-binding oligomer of the present invention comprises two hBCMA-binding polypeptides of the present invention. In another embodiment, a hBCMA-binding oligomer of the present invention comprises at least three, at least four, at least 5 or at least 6 hBCMA-binding polypeptides of the present invention, for example 3, 4, 5 or 6 or more hBCMA-binding polypeptides of the present invention.

This aspect of the invention may also be defined such that the hBCMA-binding polypeptide constitutes two or more hBCMA-binding moieties or hBCMA-binding peptides optionally connected via one or more linkers, for example two, three, four, five, six or more hBCMA-binding moieties or hBCMA-binding peptides optionally connected via one or more linkers, for example two, three, or four hBCMA-binding moieties or hBCMA-binding peptides optionally connected via one or more linkers, for example two hBCMA-binding moieties or hBCMA-binding peptides optionally connected via one or more linkers. This definition is used in the numbered embodiments of the invention below to refer to the hBCMA-binding oligomers aspects of the invention.

An hBCMA-binding oligomer of the present invention comprises, at least, a first hBCMA-binding polypeptide which is an hBCMA-binding polypeptide of the present invention, and a second hBCMA-binding polypeptide which is a hBCMA-binding polypeptide of the present invention. The first and second hBCMA-binding polypeptides may have the same sequence. Alternatively, the first and second hBCMA-binding polypeptides may have different sequences. A hBCMA-binding oligomer of the present invention may optionally further comprise a third hBCMA-binding polypeptide which is a hBCMA-binding polypeptide of the present invention. The third hBCMA-binding polypeptides may have the same sequence as the first and/or second hBCMA-binding polypeptides sequences. Alternatively, the third hBCMA-binding polypeptide may have a different sequence to the first and second hBCMA-binding polypeptides. A hBCMA-binding oligomer of the present invention may optionally further comprise a fourth hBCMA-binding polypeptide which is a hBCMA-binding polypeptide of the present invention. The fourth hBCMA-binding polypeptides may have the same sequence as the first and/or second and/or third hBCMA-binding polypeptides sequences. Alternatively, the fourth hBCMA-binding polypeptide may have a different sequence to the first, second and third hBCMA-binding polypeptides.

In one preferred embodiment, an hBCMA-binding oligomer of the present invention comprises a first hBCMA-binding polypeptide that comprises a first binding motif selected from SEQ ID NOs. 170 to 275 (preferably from SEQ ID NOs. 170-188) (and wherein optionally from 1 to 5 (preferably 1, 2 or 3) residues in the sequence are replaced by an alternative residue, and preferably a residue that is a conservative replacement); and a second hBCMA-binding polypeptide that comprises a second binding motif selected from SEQ ID NOs. 170 to 275 (preferably from SEQ ID NOs. 170-188) (and wherein optionally from 1 to 5 (preferably 1, 2 or 3) residues in the sequence are replaced by an alternative residue, and preferably a residue that is a conservative replacement). The first and second hBCMA-binding motifs may have the same sequence or a different sequence.

In one preferred embodiment, an hBCMA-binding oligomer of the present invention comprises a first hBCMA-binding polypeptide that has a sequence selected from SEQ ID NOs. 1, 2, 23-39, 41-119, or 121-128 (preferably SEQ ID NOs. 2 and 23-39) (and wherein optionally from 1 to 5 (preferably 1, 2 or 3) residues in the sequence are replaced by an alternative residue, and preferably a residue that is a conservative replacement); and a second hBCMA-binding polypeptide that has a sequence selected from SEQ ID NOs. 1, 2, 23-39, 41-119, or 121-128 (preferably SEQ ID NOs. 2 and 23-39) (and wherein optionally from 1 to 5 (preferably 1, 2 or 3) residues in the sequence are replaced by an alternative residue, and preferably a residue that is a conservative replacement). The first and second hBCMA-binding polypeptide may have the same sequence or a different sequence.

The hBCMA-binding polypeptides in a hBCMA-binding oligomer of the present invention may be separated by a linker. For example, each hBCMA-binding polypeptide in a hBCMA-binding oligomer of the present invention may be separated by a linker. Preferably, the linker is a linker as defined herein, for example a flexible amino acid linker, a rigid amino acid linker or cleavable amino acid linker or non-amino acid linker. Where a hBCMA-binding oligomer of the present invention comprises more than one linker, the linkers may be the same, or may be different. Preferably, a linker for a hBCMA-binding oligomer of the present invention comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for a hBCMA-binding oligomer of the present invention is G or comprises or has the sequence GGGSG, GGGGS, GGSGG, GSGGG and/or SGGGG; for example the linker is G or comprises or has the sequence

GGGSG, GGGSGGGGSG, GGGSGGGGSGGGGSG,
GGGSGGGGSGGGGSGGGGSG, GGGGS, GGGGGGGGS,
GGGGSGGGGSGGGGS, GGGGSGGGGSGGGGSGGGGS, GGSGG,
GGSGGGGSGG, GGSGGGGSGGGGSGG,
GGSGGGGSGGGGSGGGGSGGGGSGG, GSGGG, GSGGGGSGGG,
GSGGGGSGGGGSGGG, GSGGGGSGGGGSGGGGSGGG, SGGGG,
SGGGGSGGGG, SGGGGSGGGGSGGGG, or
SGGGGSGGGGSGGGGSGGGG.

In one preferred embodiment, a linker for an hBCMA-binding oligomer of the present invention is G or comprises or has sequence GGGSG; for example the linker comprises or has the sequence GGGSG, GGGSGGGGSG, GGGSGGGGSGGGGSG or GGGSGGGGSGGGGSGGGGSG. In another embodiment, a linker for an hBCMA-binding oligomer of the present invention comprises or has the sequence GGGGS; for example the linker comprises or has the sequence GGGGS, GGGGSGGGGS, GGGGSGGGGSGGGGS or GGGGSGGGGSGGGGSGGGGS.

Alternatively, hBCMA-binding polypeptides in a hBCMA-binding oligomer of the present invention may not be separated by a linker (i.e. they may be directly attached to each other).

In one preferred embodiment, a hBCMA-binding oligomer does not comprise a linker.

In one embodiment, the hBCMA-binding oligomer of the present invention comprises at least 2 (for example 2) hBCMA-binding polypeptides, and the hBCMA-binding oligomer comprises the following structure:
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion];
   wherein each N-terminal portion in the oligomer may have the same sequence or have different sequences; each C-terminal portion in the oligomer may have the same sequence or have different sequences; each separating portion in the oligomer may have the same sequence or have different sequences; each Helix 1 portion in the oligomer may have the same sequence or have different sequences; and each Helix 2 portion in the oligomer may have the same sequence or have different sequences.

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

Alternatively, the linker may be absent, i.e. the hBCMA-binding oligomer comprises the following structure:
[N-terminal portion]-[Helix 1]-[Separating portion]-[Helix 2]-[C-terminal portion]-[N-terminal portion]-[Helix 1]-[Separating portion]-[Helix 2]-[C-terminal portion];

In one embodiment, the hBCMA-binding oligomer of the present invention comprises at least 3 (for example 3) hBCMA-binding polypeptides, and the hBCMA-binding oligomer comprises the following structure
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion];
   and wherein each linker portion in the oligomer may have the same sequence or have different sequences; each N-terminal portion in the oligomer may have the same sequence, have different sequences, or two may have the same sequence and one may have a different sequence; each C-terminal portion may have the same sequence, have different sequences, or two may have the same sequence and one may have a different sequence; each separating portion in the oligomer may have the same sequence, have different sequences, or two may have the same sequence and one may have a different sequence; each Helix 1 portion in the oligomer may have the same sequence, have different sequences, or two may have the same sequence and one may have a different sequence; and each Helix 2 portion in the oligomer may have the same sequence, have different sequences, or two may have the same sequence and one may have a different sequence.

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG. Also, for example, the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

Alternatively, one or both linkers may be absent, i.e. the hBCMA-binding oligomer comprises the following structure:
[N-terminal portion]-[Helix 1]-[Separating portion]-[Helix 2]-[C-terminal portion]-[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion];
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[N-terminal portion]-[Helix 1]-[Separating portion]-[Helix 2]-[C-terminal portion]; or
[N-terminal portion]-[Helix 1]-[Separating portion]-[Helix 2]-[C-terminal portion]-[N-terminal portion]-[Helix 1]-[Separating portion]-[Helix 2]-[C-terminal portion]-[N-terminal portion]-[Helix 1]-[Separating portion]-[Helix 2]-[C-terminal portion].

In some aspects of the invention, the hBCMA-binding oligomer constitutes two or more hBCMA-binding polypeptides optionally connected via one or more linkers, for example two, three, four, five, six or more hBCMA-binding polypeptides optionally connected via one or more linkers, for example two, three, or four hBCMA-binding polypeptides optionally connected via one or more linkers, for example two hBCMA-binding polypeptides optionally connected via one or more linkers.

For example, such an oligomer may comprise two hBCMA-binding polypeptides, each of which has a sequence in which Helix 1 comprises the sequence NKEETFADLEISNL and Helix 2 comprises the sequence NFYQKWAFIRSLMDD.

As set out elsewhere, such portions may have a number of residues substituted by an alternative residue. For example, each of the two polypeptides may have the sequence:

[SEQ ID NO: 1]
VDNKFNKEETFADLEISNLPNLNFYQKWAFIRSLMDDPSQSANLLAEAK
KLNDAQAPK.

In an alternative example, such an oligomer may comprise two hBCMA-binding polypeptides, each of which has a sequence in which Helix 1 comprises the sequence NKENQFADEEIAAL and Helix 2 comprises the sequence NFYQKWAFIRKLMDD. As set out elsewhere, such portions may have a number of residues substituted by an alternative residue. For example, each of the two polypeptides may have the sequence:

[SEQ ID NO: 2]
VDNKFNKENQFADEEIAALPNLNFYQKWAFIRKLMDDPSQSANLLAEAK
KLNDAQAPK.

In an alternative example, such an oligomer may comprise two hBCMA-binding polypeptides, one of which has a sequence in which Helix 1 comprises the sequence NKEETFADLEISNL and Helix 2 comprises the sequence NFYQKWAFIRSLMDD, and the other of which has a sequence in which Helix 1 comprises the sequence NKENQFADEEIAAL and Helix 2 comprises the sequence NFYQKWAFIRKLMDD. As set out elsewhere, such portions may have a number of residues substituted by an alternative residue. For example, one of the two polypeptides may have the sequence:

[SEQ ID NO: 1]
VDNKFNKEETFADLEISNLPNLNFYQKWAFIRSLMDDPSQSANLLAEAK
KLNDAQAPK.

and

The other may have the sequence:

[SEQ ID NO: 2]
VDNKFNKENQFADEEIAALPNLNFYQKWAFIRKLMDDPSQSANLLAEAK
KLNDAQAPK.

### Linkers

Where present, a linker connects together two or more functional portions (defined further hereinbelow) of the hBCMA-binding polypeptides of the invention (for example in an hBCMA-binding oligomer of the invention), or a linker may connect together an hBCMA binding polypeptide of the present invention and an additional functional portion of the present invention, or a linker may connect together two or more functional portions of the hBCMA binding polypeptide-drug conjugates of the invention. A linker may also connect together an additional functional of the present invention and an additional functional portion of the present invention in embodiments where more than one additional functional portion is present. The skilled person is aware that different kinds of linkers with varying properties are known in the art, and is able to select the appropriate linker(s) depending on the properties desired. Types of linkers include, for example, flexible amino acid linkers, rigid amino acid linkers and cleavable amino acid linkers; non-amino acid linkers may also be used. For example, linkers may be selected to increase stability or improve folding, to increase expression, improve biological activity, enable targeting, or alter pharmacokinetics. A non-amino acid linker may be referred to as a synthetic linker.

For the avoidance of doubt, in certain embodiments a linker may not connect together two hBCMA binding polypeptides of the present invention (for example in a hBCMA-binding oligomer of the invention), and/or a linker may not connect together a hBCMA-binding polypeptides of the present invention and an additional functional portion of the present invention; and/or a linker may not connect together an additional functional of the present invention and an additional functional portion of the present invention in embodiments where more than one additional functional portion is present. For example, in certain embodiments two hBCMA-binding polypeptides of the present invention (for example in a hBCMA-binding oligomer of the invention) may be directly connected; and/or a hBCMA-binding polypeptide or oligomer of the present invention may be directly connected to an additional functional portion of the present invention; and/or an additional functional portion of the present invention may be directly connected to an additional functional portion of the present invention.

In one embodiment, the hBCMA-binding polypeptide, hBCMA-binding oligomer, or hBCMA-binder-drug conjugate according to any aspect disclosed herein further comprises at least one linker, such as at least one linker selected from flexible amino acid linkers, rigid amino acid linkers, cleavable amino acid linkers and synthetic linkers. In one embodiment, said linker is between two or more hBCMA-binding polypeptides, for example in an hBCMA binding oligomer of the present invention, or between an hBCMA-binding polypeptide or hBCMA-binding oligomer and an additional functional portion, for example an immune signalling molecule or an additional binding moiety (for example as described in further detail below). In another embodiment, said linker is between an hBCMA-binding polypeptide or hBCMA-binding oligomer of the present invention and a therapeutic agent (for example as described in further detail below).

In one embodiment, the hBCMA binding oligomer according to any aspect disclosed herein comprises at least one linker, such as at least one linker selected from flexible amino acid linkers, rigid amino acid linkers and cleavable amino acid linkers. The linker is between two or more hBCMA-binding polypeptides. A further linker between a hBCMA-binding polypeptide or a hBCMA-binding oligomer and an additional functional portion, for example an immune signalling molecule or an additional binding moiety (for example as described in further detail below), may also be present in a hBCMA binding oligomer according to any aspect disclosed herein. A further linker between an additional functional portion (for example an immune signalling molecule or an additional binding moiety (for example as described in further detail below) and an additional functional portion (for example an immune signalling molecule or an additional binding moiety (for example as described in further detail below), may also be present in a hBCMA binding oligomer according to any aspect disclosed herein.

In embodiments and aspects of the present invention that comprise more than one linker, each linker may be the same, or may be different, or some linkers may be the same, and some may be different (for example in embodiments having 3 or more linkers (for example 3, 4, 5, 6, 7, 8 or more linkers).

Flexible amino acid linkers may be used when the linked domains require some distance and conformational freedom, and may be advantageous in some embodiments of the invention. Such linkers are generally composed of small, non-polar (for example G) or polar (for example S or T) amino acids. Some flexible linkers primarily consist of stretches of G and S residues, for example (GGGGS)p or (GGGSG)p. Other examples include (GGSGG)p, (GSGGG)p or (SGGGG)p. Adjusting the copy number “p” allows optimization of the linker in order to achieve appropriate separation between the functional moieties, or to maintain necessary inter-moiety interaction. In an embodiment, the linker is (GGGSG)p, for example (GGGSG)₁, (GGGSG)₂ or (GGGSG)₃, for example (GGGSG)₃. In an embodiment, the linker is (GGGGS)p, for example (GGGGS)₁, (GGGGS)₂ or (GGGGS)₃, for example (GGGGS)₃. In an embodiment, the linker is (GGSGG)p, for example (GGSGG)₁, (GGSGG)₂ or (GGSGG)₃, for example (GGSGG)₃. In an embodiment, the linker is (GSGGG)p, for example (GSGGG)₁, (GSGGG)₂ or (GSGGG)₃, for example (GSGGG)₃. In an embodiment, the linker is (SGGGG)p, for example (SGGGG)₁, (SGGGG)₂ or (SGGGG)₃, for example (SGGGG)₃. In another embodiment, the linker is G.

In one embodiment, a linker of the present invention comprises or has a sequence of 1 to 50 (for example 1 to 40, 1 to 30, or 1 to 25, for example 1, 2, 3, 4, 5 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T. In one embodiment, a linker of the present invention comprises or has a sequence of 1 to 50 (for example 1 to 25, for example 15, 16, 17, 18, 19, 20, 21, 22, 23, 24 or 25; or 1 to 20, for example 1, 5, 10, 15 or 20; or 1 to 15, for example 1, 5, 10 or 15) naturally occurring amino acids, for example selected from the group consisting of G and S. For example, a linker of the present invention is G or comprises or has sequence GGGSG; for example the linker comprises or has the sequence GGGSG, GGGSGGGGSG, GGGSGGGGSGGGGSG or GGGSGGGGSGGGGSGGGGSG.

Also, for example, a linker of the present invention is G or comprises or has sequence GGGSG, GGGGS, GGSGG, GSGGG and/or SGGGG. For example, the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGGGGGSGGG, SGGGG, SGGGGSGGGG, SGGGGSGGGGSGGGG, or SGGGGSGGGGSGGGGSGGGG.

In one preferred embodiment, a linker of the present invention is G or comprises or has sequence GGGSG; for example the linker comprises or has the sequence GGGSG, GGGSGGGGSG, GGGSGGGGSGGGGSG or GGGSGGGGSGGGGSGGGGSG. In another embodiment, a linker for a hBCMA-binding oligomer of the present invention comprises or has sequence GGGGS; for example the linker comprises or has the sequence GGGGS, GGGGSGGGGS, GGGGSGGGGSGGGGS or GGGGSGGGGSGGGGSGGGGS.

Apart from G and S linkers, other flexible linkers are known in the art, such as G and S linkers containing additional amino acid residues, such as T and A, to maintain flexibility, as well as polar amino acid residues to improve solubility. In general, it is known in the art that linker sequence and length may affect the characteristics of the linked moieties, and so the skilled person will be able to select an appropriate linker for use in the binding polypeptides as described herein.

Other types of linkers, such as rigid and/or cleavable linkers, can also be used to connect domains in multidomain constructs to improve or control their biological activity. Such linkers are known in the art (Chen X et al, Fusion protein linkers: Property, design and functionality, Adv Drug Deliv Rev 2013:65:10:1357, doi: 10.1016/j.addr.2012.09.039).

Alternatively, the different moieties of the binding polypeptide or oligomer as described herein (for example two or more hBCMA-binding polypeptides, an hBCMA-binding polypeptide and an additional functional portion, for example an immune signalling molecule or an additional binding moiety, or an hBCMA-binding polypeptide or hBCMA binding oligomer and a therapeutic agent) may be covalently linked by a chemical linker. Such a chemical linker may be produced by, for example, maleimido or ‘click’ chemistry. Such a chemical linker may be cleavable, for example an MC-Val-Ala-PAB linker, or non-cleavable, for example an MPB linker. The skilled person will be aware of other linkers suitable for use in the binding polypeptides as described herein.

With regard to the description above of binding polypeptides comprising an hBCMA-binding polypeptide according to the disclosure, it is to be noted that the designation of first, second and further moieties or functional portions is made for clarity reasons to distinguish between hBCMA-binding polypeptide or polypeptides according to the invention on the one hand, and binding moieties or functional portions exhibiting other functions on the other hand. These designations are not intended to refer to the actual order of the different regions of the binding polypeptide. Similarly, the designations first and second moiety or functional portion (or monomer unit) are made for clarity reasons to distinguish between said units. Thus, for example, said first moiety or functional portion (or monomer unit) may without restriction appear at the N-terminal end, in the middle, or at the C-terminal end of the binding polypeptide.

The hBCMA-binding polypeptides in a hBCMA-binding oligomer of the present invention may be separated by a linker. For example, each hBCMA-binding polypeptides in a hBCMA-binding oligomer of the present invention may be separated by a linker. Preferably, the linker is a linker as defined herein. Where a hBCMA-binding oligomer of the present invention comprises more than one linker, the linkers may be the same, or may be different.

Preferably, a linker for an hBCMA-binding oligomer of the present invention comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for an hBCMA-binding oligomer of the present invention is G or comprises or has sequence GGGSG, GGGGS, GGSGG, GSGGG and/or SGGGG; for example the linker comprises or has the sequence GGGSG, GGGSGGGGSG, GGGSGGGGSGGGGSG or GGGSGGGGSGGGGSGGGGSG. In another embodiment, a linker for a hBCMA-binding oligomer of the present invention comprises or has sequence GGGGS; for example the linker comprises or has the sequence GGGGS, GGGGSGGGGS, GGGGSGGGGSGGGGS or GGGGGGGGSGGGGSGGGGS.

In embodiments of the present invention comprising an additional functional portion, the hBCMA-binding polypeptide(s) and additional functional portion(s) in an hBCMA-binding polypeptide or an hBCMA-binding oligomer of the present invention may be separated by a linker. For example, each additional functional portion and hBCMA-binding polypeptide in a hBCMA-binding polypeptide or hBCMA-binding oligomer of the present invention may be separated by a linker. Preferably, the linker is a linker as defined herein. In embodiments of the present invention comprising two or more additional functional portions, the additional functional portions may be separated by a linker. Preferably, the linker is a linker as defined herein.

Where a hBCMA-binding polypeptide or a hBCMA-binding oligomer of the present invention comprises more than one linker (i.e, wherein there are at least two hBCMA-binding polypeptides and at least one additional functional portion; or wherein there is at least one hBCMA-binding polypeptides and at least two additional functional portion), the linkers may be the same, or may be different. Preferably, a linker for a hBCMA-binding polypeptide or a hBCMA-binding oligomer comprising an additional functional portion comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for a hBCMA-binding polypeptide or a hBCMA-binding oligomer comprising an additional functional portion is G or comprises or has sequence GGGSG, GGGGS, GGSGG, GSGGG and/or SGGGG; for example the linker comprises or has the sequence GGGSG, GGGSGGGGSG, GGGSGGGGSGGGGSG or GGGSGGGGSGGGGSGGGGSG. In another embodiment, a linker for a hBCMA-binding oligomer of the present invention comprises or has sequence GGGGS; for example the linker comprises or has the sequence GGGGS, GGGGSGGGGS, GGGGSGGGGSGGGGS or GGGGGGGGSGGGGSGGGGS.

### Additional Functional Portions

An hBCMA binding polypeptide as disclosed herein may be attached to one or more additional functional portions, for example a binding partner recognising an immune cell surface molecule. An hBCMA binding oligomer as disclosed herein may be attached to one or more functional portions, for example a binding partner recognising an immune cell surface molecule. Therefore, in an embodiment, the at least one hBCMA binding polypeptide (or hBCMA binding oligomer) is attached to one or more additional functional portions, optionally via a linker as described above.

A ‘functional portion’, as used herein, refers to a component or ‘moiety’ with a specific desired biological activity.

The one or more additional component (i.e. the one or more additional functional portion) may for example be a signalling molecule or a derivative thereof. Examples of suitable signalling molecules include immune signalling molecules or derivatives thereof, for example cytokines or derivatives thereof, for example IL-15 and derivatives thereof.

The one or more additional component(s) (i.e. the one or more additional functional portion) may be one or more additional binding moiety(ies), for example one or more binding partner(s) recognising a cell surface protein or antigen, for example an immune cell surface protein or a cell surface tumour antigen (also referred to as a cancer cell surface antigen or cancer cell surface target). Cell surface tumour antigens may for example be tumour-associated antigens or tumour-specific antigens.

Such additional binding moieties may for example be specific for an immune cell surface protein, for example an NK cell activating receptor, for example CD16a. In particular, such a CD16a-binding moiety may for example be an immune cell-engaging polypeptide as disclosed in UK patent application no. 2208027.9 (which may be obtained on publication or from the publicly available file on publication of other patent applications, for example international patent applications claiming priority from it). Such ‘dual engager’ binding polypeptides may for example comprise at least one hBCMA binding polypeptide as disclosed herein, for example one, two, three or more hBCMA binding polypeptides as disclosed herein, or at least one hBCMA binding oligomer as disclosed herein. The at least one (for example one, two, three or more) hBCMA binding polypeptide(s) as disclosed herein or at least one hBCMA binding oligomer as disclosed herein may for example be attached to at least one CD16a-binding polypeptide as described above, for example one, two, three, or more CD16a-binding polypeptides as described above. In such a ‘dual engager’ binding polypeptide, the additional binding moiety (for example a binding moiety specific for an immune cell surface molecule such as CD16a, for example the at least one CD16a-binding polypeptide as described above) may be attached at the N-terminal end or the C-terminal end of the hBCMA binding moiety (for example, the at least one hBCMA binding polypeptide as disclosed herein or the at least one hBCMA binding oligomer as disclosed herein), optionally via one or more linkers as described above. The additional binding moiety (for example a binding moiety specific for an immune cell surface molecule such as CD16a, for example the at least one CD16a-binding polypeptide as described above) may, alternatively, be attached between two hBCMA-binding moieties, optionally separated by one or more linkers as described above. Non-limiting examples of such ‘dual engager’ binding polypeptides, comprising one hBCMA-binding polypeptide and one or more CD16a-binding polypeptides are given in Table 1; sequences of these dual engagers comprising a C-terminal His₆ tag are given as [SEQ ID NOs. 130-133] and sequences without a C-terminal His₆ tag are given as [SEQ ID NOs 139-142]. Additional non-limiting examples of ‘dual engager’ polypeptides comprising two hBCMA-binding polypeptides and a moiety targeting CD16a are given in Table 1; sequences of these dual engagers comprising a C-terminal His₆ tag are given as [SEQ ID NOs. 134-138] and sequences without a C-terminal His₆ tag are given as [SEQ ID NOs 143-147]. Non-limiting examples of such ‘dual engager’ binding polypeptides, comprising one hBCMA-binding polypeptide and one or more CD16a-binding polypeptides are also provided; sequences of these dual engagers comprising a C-terminal His₆ tag are given as SEQ ID NOs 154-163, and a sequence without a C-terminal His₆ tag is given as SEQ ID NOs 166. Further non-limiting examples of ‘dual engager’ polypeptides comprising two hBCMA-binding polypeptides and a moiety targeting CD16a are also provided; sequences of such dual engagers comprising a C-terminal His₆ tag include SEQ ID NOs 149, 150, 151, 152, 153, and sequences without a C-terminal His₆ tag include SEQ ID NO 167.

In certain embodiments, a hBCMA-binding polypeptide of the invention (for example an hBCMA-binding polypeptide incorporated into a ‘dual engager’ polypeptide, for example a dual engager polypeptide further comprising at least one moiety targeting an NK cell surface target, for example CD16a) is especially active in functional assays, e.g. a CD16 reporter assay and/or a cell killing assay. Exemplary sequences of such dual engager polypeptides include SEQ ID NOs 149, 150, 151, 158, 160, and 161 (also disclosed in the Examples hereinbelow).

The skilled person will understand that such non-limiting examples of dual engagers (as well as other dual engagers comprising at least one hBCMA-binding polypeptide or at least one hBCMA binding oligomer disclosed herein attached to a CD16a-binding moiety, and other molecules comprising at least one hBCMA-binding polypeptide or at least one hBCMA binding oligomer attached to different functional portions (for example additional binding moieties as described below)) may optionally comprise one or more additional N-terminal, C-terminal or other modification, for example a C-terminal His₆ tag, depending on the intended use of the engager or requirements for expression, purification or the like. Non-limiting examples of such N-terminal, C-terminal or other modifications may include a peptide purification tag or moiety (for example a histidine-tag (for example a polyhistidine tag) or a methionine-tag (for example a single methionine tag or a polymethionine tag)), a signalling tag or moiety (for example a glycine residue, or a signal peptide, for example selected from OmpA, DsbA, PhoA, and PelB), a fluorophore tag (for example Alexa448), or a tag or moiety to assist conjugation (a cysteine tag (for example a single cysteine at the C or N terminal)). Such tags and/or moieties may preferably be present at the N-terminal and/or the C-terminal of the dual engagers comprising at least one hBCMA-binding polypeptide or at least one hBCMA binding oligomer as described herein.

Further examples of additional components (i.e. additional functional portions) include additional binding moieties that are binding partners recognising B-cell maturation antigen (BCMA), cytotoxic T-lymphocyte-associated protein 4 (CTLA-4), Programmed cell death protein 1 (PD-1), disintegrin and metalloprotease 17 (ADAM17), Programmed death-ligand 1 (PD-L1), SLAM family member 7 (SLAMF7), Epithelial Cell Adhesion Molecule (EPCAM), Epidermal growth factor receptor (EGFR/ErbB-1), Epidermal growth factor receptor variant 3(EGFRvIII), erb-b2 tyrosine kinase 2 (ERBB2/HER2/CD340), prostate-specific membrane antigen (PSMA), Claudin8.2 (CLDN18.2), delta like protein 3 (DLL3), mucin 16 (MUC16), mucin 17 (MUC17), mucin 1 (MUC1), Trophoblast glycoprotein (TPBG/5T4/WAIF1), V-set domain-containing T-cell activation inhibitor 1 (B7-H4/VTCN1/B7x/B7S1), cluster of differentiate 20 (CD20), B-Lymphocyte Surface Antigen B4 (CD19), Sialic Acid-Binding Ig-Like Lectin 2 (CD22), TNF receptor superfamily member 8 (CD30), Natural cytotoxicity triggering receptor 1 (NKp46), and NKG2D.

Examples of additional components (i.e. additional functional portions) include additional binding moieties that are binding partners recognising a cell surface tumour antigen or cancer cell surface target selected from the group consisting of BCMA, ADAM17, SLAMF7, PD-L1, EPCAM, EGFR/ErbB-1, EGFRVIII, ERBB2/HER2/CD340, PSMA, CLDN18.2, DLL3, MUC16, MUC17, MUC1, TPBG/5T4/WAIF1 and B7-H4/VTCN1/B7x/B7S1.

Examples of additional components (i.e. additional functional portions) include additional binding moieties that are binding partners recognising an immune cell surface protein or immune cell surface target selected from the group consisting of CTLA-4, PD-1, CD20, CD19, CD22 and CD30. Examples of additional components (i.e. additional functional portions) include additional binding moieties that are binding partners recognising a cell surface tumour antigen or cancer cell surface target expressed in haematological malignancies, for example BCMA, CD20, CD19, CD22 or CD30.

Alternatively or additionally, such additional binding moieties may for example be specific for a different target on multiple myeloma cells that is not hBCMA. For example, an additional binding moiety may bind to a different target on the same multiple myeloma cell.

For the avoidance of doubt, such examples of additional binding moieties, for example binding partners recognising cell surface proteins or antigens, are non-limiting and are specified here by way of illustration. The additional binding moiety as referred to in this context is not an hBCMA binding polypeptide of the present invention.

In one embodiment, an hBCMA binding polypeptide or hBCMA-binding oligomer of the present invention is attached to one or more additional functional portion (for example one or more additional binding moiety(ies) and/or signalling molecule), optionally via a linker as described above. For example, the at least one hBCMA binding polypeptide or hBCMA-binding oligomer is attached to one, two, three, four or more additional functional portions (for example one, two, three, four or more additional binding moieties and/or signalling molecules). In certain embodiments, at least one hBCMA binding polypeptide or hBCMA-binding oligomer is attached to one or two additional functional portions (for example one or two additional binding moieties or signalling molecules). In certain embodiments, at least one hBCMA binding polypeptide or hBCMA-binding oligomer is attached to one additional functional portion (for example one additional binding moiety or signalling molecule).

An hBCMA-binding polypeptide or hBCMA-binding oligomer of the present invention comprising one or more (for example 1, 2, 3, 4, 5 or 6 or more) additional function portions may be referred to as heteromultimeric. An hBCMA-binding polypeptide of the present invention consisting of one hBCMA-binding polypeptide and one additional functional portion only and no other functional portions (i.e. no further hBCMA-binding polypeptides functional portions or additional functional portions) may be referred to as heterodimeric. An hBCMA-binding polypeptide of the present invention consisting of one hBCMA-binding polypeptide and two additional function portions only and no other functional portions may be referred to as heterotrimeric. An hBCMA-binding oligomer of the present invention consisting of two hBCMA-binding polypeptide functional portions and one additional function portion only and no other functional portions may be referred to as a heterotrimeric.

In certain preferred embodiments, an additional function portion is a signalling molecule. A signalling molecule, for example an immune signalling molecule such as a cytokine, for example IL-15 or derivatives thereof, may be attached at the N-terminal end or the C-terminal end of an hBCMA-binding polypeptide or an hBCMA-binding oligomer, optionally via a linker as described above. One or more signalling molecule(s) may, alternatively, be attached between two hBCMA-binding polypeptides in an hBCMA-binding oligomer, optionally separated by one or more linkers or linking sequences as described above. Preferably, the signalling molecule(s) may be attached at the N-terminal end or the C-terminal end of an hBCMA-binding polypeptide or a hBCMA-binding oligomer, optionally via a linker as described above. Preferably, the signalling molecule(s) may be attached at the C-terminal end of a hBCMA-binding polypeptide or a hBCMA-binding oligomer, optionally via a linker as described above.

In certain preferred embodiments, an additional functional portion is an additional binding moiety. For example, an additional functional portion is an additional binding moiety that is a binding partner recognising one of the following: CTLA-4, PD-1, BCMA, ADAM17, PD-L1, SLAMF7, EPCAM, EGFR/ErbB-1, EGFRVIII, ERBB2/HER2/CD340, PSMA, CLDN18.2, DLL3, MUC16, MUC17, MUC1, TPBG/5T4/WAIF1, B7-H4/VTCN1/B7x/B7S1, CD20, CD19, CD22 or CD30. For example, an additional functional portion is an additional binding moiety that is specific for one of the following: CTLA-4, PD-1, BCMA, ADAM17, PD-L1, SLAMF7, EPCAM, EGFR/ErbB-1, EGFRvIII, ERBB2/HER2/CD340, PSMA, CLDN18.2, DLL3, MUC16, MUC17, MUC1, TPBG/5T4/WAIF1, B7-H4/VTCN1/B7x/B7S1, CD20, CD19, CD22 or CD30.

In certain preferred embodiments, an additional binding moiety is specific for a cancer cell surface target (for example a myeloma cell surface antigen, for example BCMA). In certain preferred embodiments, an additional binding moiety is specific for an immune cell surface protein (for example a NK cell surface protein, for example CD16a, NKp46, NKG2D, PD-1, especially CD16a).

As mentioned above, an additional binding moiety, for example a binding partner recognising the NK cell surface protein CD16a, may be attached at the N-terminal end or the C-terminal end of a hBCMA-binding polypeptide or a hBCMA-binding oligomer, optionally via a linker as described above. The one or more additional binding moiety(ies) may, alternatively, be attached between two hBCMA-binding polypeptides in a hBCMA-binding oligomer, optionally separated by one or more linking sequences as described above. Preferably, the additional binding moiety(ies), for example a binding partner recognising the NK cell surface protein CD16a, may be attached at the N-terminal end or the C-terminal end of an hBCMA-binding polypeptide or a hBCMA-binding oligomer, optionally via a linker as described above. More preferably, the additional binding moiety(ies), for example a binding partner recognising the NK cell surface protein CD16a, may be attached at the C-terminal end of an hBCMA-binding polypeptide or an hBCMA-binding oligomer, optionally via a linker as described above.

The present inventors have surprisingly found that a ‘dual engager’ polypeptide comprising a hBCMA binding polypeptide or a hBCMA binding oligomer as disclosed herein can retain its hBCMA binding ability when fused to an additional binding moiety targeting the NK cell surface protein CD16a. This ‘dual engager’ polypeptide comprising a hBCMA binding polypeptide as disclosed herein fused to a CD16a binding moiety is also surprisingly capable of activating NK cells in the presence of BCMA-expressing tumour cells.

In one preferred embodiment, the hBCMA-binding polypeptide or hBCMA-binding oligomer of the present invention further comprises one or more additional functional portions, for example at least one, at least two, or at least three or at least four additional functional portions. For example, in particular embodiments, the hBCMA-binding polypeptide or hBCMA-binding oligomer of the present invention further comprises 1, 2, 3, 4 or 5 additional functional portions.

In embodiments wherein the hBCMA-binding polypeptide or hBCMA-binding oligomer comprises at least two, at least three or at least four additional functional portions (for example 2, 3, 4 or 5 additional functional portions), each additional functional portion may be the same, or may be different, or some additional functional portions may be the same, and some additional functional portions be different (for example in embodiments having at least 3 or at least 4 additional functional portions (for example 3, 4, 5, 6 or more additional functional portions)).

In embodiments wherein the hBCMA-binding polypeptide or hBCMA-binding oligomer comprises at least two, at least three or at least four additional functional portions (for example 2, 3, 4 or 5 additional functional portions), each additional functional portion may have the same function, or may have different functions, or some additional functional portions may have the same function, and some may have different functions (for example in embodiments having at least 3 or at least 4 additional functional portions (for example 3, 4, 5, 6 or more additional functional portions)).

In embodiments wherein the hBCMA-binding polypeptide or hBCMA-binding oligomer comprises at least two, at least three or at least four additional functional portions (for example 2, 3, 4 or 5) additional functional portions, a first additional functional portion may comprise an additional binding moiety (for example an additional binding moiety specific for a cancer cell surface target, for example a myeloma cell surface antigen, for example a myeloma cell surface antigen that is not hBCMA, or an additional binding moiety specific for an immune cell surface target, for example a NK cell surface protein, for example CD16a); and a second additional functional portion may comprise an immune signalling molecule, for example a cytokine, for example IL-15 or derivatives thereof. For example, in one preferred embodiment, a first additional functional portion may comprise an additional binding moiety specific for a cancer cell surface target (for example a myeloma cell surface antigen, for example a myeloma cell surface antigen that is not BCMA) and a second additional functional portion may comprise a cytokine, for example IL-15 or derivatives thereof. In particular, a first additional functional portion may comprise an additional binding moiety specific for a myeloma cell surface antigen that is not BCMA; and the second additional functional portion may comprise a cytokine, for example IL-15 or derivatives thereof. Alternatively, for example, in one preferred embodiment, a first additional functional portion may comprise an additional binding moiety specific for a immune cell target (for example an NK cell target, for example CD16a) and a second additional functional portion may comprise a cytokine, for example IL-15 or derivatives thereof. In particular, a first additional functional portion may comprise an additional binding moiety specific for an NK cell target (for example CD16a); and second additional functional portion may comprise a cytokine, for example IL-15 or derivatives thereof.

In another preferred embodiment, a first additional functional portion may comprise an additional binding moiety (for example an additional binding moiety specific for a cancer cell surface target, for example a myeloma cell surface antigen, for example a myeloma cell surface antigen that is not BCMA, or is specific for an immune cell target, for example a NK cell target, for example CD16a); and a second additional functional portion may comprise an additional binding moiety (for example an additional binding moiety specific for a cancer cell surface target, for example a myeloma cell surface antigen, for example a myeloma cell surface antigen that is not BCMA, or is specific for an immune cell target, for example a NK cell target, for example CD16a). For example, a first additional functional portion may comprise an additional binding moiety specific for a cancer cell surface target (for example a myeloma cell surface antigen, for example a myeloma cell surface antigen that is not BCMA); and a second additional functional portion may comprise an additional binding moiety specific for a cancer cell surface target (for example a myeloma cell surface antigen, for example a myeloma cell surface antigen that is not BCMA). In particular, a first additional functional portion may comprise an additional binding moiety specific for a myeloma cell surface antigen that is not BCMA; and a second additional functional portion may comprise an additional binding moiety specific for a myeloma cell surface antigen that is not BCMA.

Alternatively, for example, a first additional functional portion may comprise an additional binding moiety specific for a cancer cell surface target (for example a myeloma cell surface antigen, for example a myeloma cell surface antigen that is not BCMA); and a second additional functional portion may comprise an additional binding moiety specific for an immune cell surface target (for example a NK cell target, for example CD16a). In particular, a first additional functional portion may comprise an additional binding moiety specific for a myeloma cell surface antigen that is not BCMA; and a second additional functional portion may comprise an additional binding moiety specific for a NK cell target (for example CD16a).

Alternatively, in another embodiment, a first additional functional portion may comprise an immune signalling molecule, for example a cytokine, for example IL-15 or derivatives thereof; and a second additional functional portion may comprise an immune signalling molecule, for example a cytokine, for example IL-15 or derivatives thereof.

When present, (for example in embodiments wherein the hBCMA-binding polypeptide or hBCMA-binding oligomer comprises at least three or at least four additional functional portions (for example 3, 4 or 5)) a third additional functional portion may comprises an additional binding moiety (for example an additional binding moiety specific for a cancer cell surface target, for example a myeloma cell surface antigen, for example a myeloma cell surface antigen that is not BCMA, or is specific for an immune cell target, for example a NK cell target, for example CD16a), or comprises an immune signalling molecule, for example a cytokine, for example IL-15 or derivatives thereof.

When present, (for example in embodiments wherein the hBCMA-binding polypeptide or hBCMA-binding oligomer comprises at least four additional functional portions (for example 4 or 5)) a fourth additional functional portion may comprises an additional binding moiety (for example an additional binding moiety specific for a cancer cell surface target, for example a myeloma cell surface antigen, for example a myeloma cell surface antigen that is not BCMA, or is specific for an immune cell target, for example a NK cell target, for example CD16a), or comprises an immune signalling molecule, for example a cytokine, for example IL-15 or derivatives thereof.

When present, (for example in embodiments wherein the hBCMA-binding polypeptide or hBCMA-binding oligomer comprises at least five additional functional portions (for example 5 or 6)) a fifth additional functional portion may comprises an additional binding moiety (for example an additional binding moiety specific for a cancer cell surface target, for example a myeloma cell surface antigen, for example a myeloma cell surface antigen that is not BCMA, or is specific for an immune cell target, for example a NK cell target, for example CD16a), or comprises an immune signalling molecule, for example a cytokine, for example IL-15 or derivatives thereof.

In one preferred embodiment, the hBCMA-binding polypeptide or hBCMA-binding oligomer comprises at least three (for example 3, 4 or 5) additional functional portions, a first additional functional portion may comprise an additional binding moiety (for example an additional binding moiety specific for a cancer cell surface target, for example a myeloma cell surface antigen, for example a myeloma cell surface antigen that is not BCMA, or is specific for an immune cell target, for example a NK cell target, for example CD16a); and a second additional functional portion may comprise an immune signalling molecule, for example a cytokine, for example IL-15 or derivatives thereof; and a third additional functional portion may comprises an additional binding moiety (for example an additional binding moiety specific for a cancer cell surface target, for example a myeloma cell surface antigen, for example a myeloma cell surface antigen that is not BCMA, or is specific for an immune cell target, for example a NK cell target, for example CD16a).

For example In one embodiment, a first additional functional portion may comprise an additional binding moiety specific for a cancer cell surface target (for example a myeloma cell surface antigen, for example BCMA); and a second additional functional portion may comprise a cytokine, for example IL-15 or derivatives thereof; and a third additional functional portion may comprises an additional binding moiety specific for a cancer cell surface target (for example a myeloma cell surface antigen, for example BCMA) or a immune cell target (for example a NK cell target). In particular, a first additional functional portion may comprise an additional binding moiety specific for BCMA; and a second additional functional portion may comprise a cytokine, for example IL-15 or derivatives thereof; and a third additional functional portion may comprise an additional binding moiety specific for a cancer cell surface target (for example a myeloma cell surface antigen, for example BCMA). In another embodiment, a first additional functional portion may comprise an additional binding moiety specific for BCMA; and a second additional functional portion may comprise a cytokine, for example IL-15 or derivatives thereof; and a third additional functional portion may comprise an additional binding moiety specific for an immune cell target (for example a NK cell target).

For example, a first additional functional portion may comprise an additional binding moiety specific for a cancer cell surface target (for example a myeloma cell surface antigen, for example a myeloma cell surface antigen that is not BCMA); and a second additional functional portion may an additional binding moiety specific for a cancer cell surface target (for example a myeloma cell surface antigen, for example a myeloma cell surface antigen that is not BCMA); and a third additional functional portion may comprises an additional binding moiety specific for a cancer cell surface target (for example a myeloma cell surface antigen, for example a myeloma cell surface antigen that is not BCMA) or a immune cell target (for example a NK cell target, for example CD16a). In particular, a first additional functional portion may comprise an additional binding moiety specific for a myeloma cell surface antigen that is not BCMA; and a second additional functional portion may comprise an additional binding moiety specific for a myeloma cell surface antigen that is not BCMA; and a third additional functional portion may comprise an additional binding moiety specific for a cancer cell surface target (for example a myeloma cell surface antigen, for example a myeloma cell surface antigen that is not BCMA). In another embodiment, a first additional functional portion may comprise an additional binding moiety specific for a myeloma cell surface antigen that is not BCMA; and a second additional functional portion may comprise an additional binding moiety specific for a myeloma cell surface antigen that is not BCMA; and a third additional functional portion may comprise an additional binding moiety specific for an immune cell target (for example a NK cell target, for example CD16a).

Alternatively, a first additional functional portion may comprise an additional binding moiety specific for a immune cell target (for example a NK cell target, for example CD16a); and a second additional functional portion may an additional binding moiety specific for immune cell target (for example a NK cell target, for example CD16a); and a third additional functional portion may comprises an additional binding moiety specific for a cancer cell surface target (for example a myeloma cell surface antigen, for example a myeloma cell surface antigen that is not BCMA) or a immune cell target (for example a NK cell target, for example CD16a). In particular, a first additional functional portion may comprise an additional binding moiety specific for a immune cell target (for example a NK cell target, for example CD16a); and a second additional functional portion may comprise an additional binding moiety specific for immune cell target (for example a NK cell target, for example CD16a); and a third additional functional portion may comprise an additional binding moiety specific for a cancer cell surface target (for example a myeloma cell surface antigen, for example immune cell target (for example a NK cell target, for example CD16a). In another embodiment, a first additional functional portion may comprise an additional binding moiety specific for immune cell target (for example a NK cell target, for example CD16a); and a second additional functional portion may comprise an additional binding moiety specific for immune cell target (for example a NK cell target, for example CD16a); and a third additional functional portion may comprise an additional binding moiety specific for myeloma cell surface antigen that is not BCMA.

In one embodiment, a hBCMA-binding polypeptide or hBCMA-binding oligomer comprising an additional binding moiety of the present invention has an additional binding moiety separated from the hBCMA-binding polypeptide or the hBCMA-binding oligomer by a linker. A linker may be any linker defined hereinabove. For example, a linker selected from flexible amino acid linkers, rigid amino acid linkers, cleavable amino acid linkers and non-amino acid linkers.

Preferably, a linker for a hBCMA-binding polypeptide or hBCMA-binding oligomer comprising an additional binding moiety comprises or has a sequence of 1 to 50 (for example 1 to 25, for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S).

In one preferred embodiment, a linker for a hBCMA-binding polypeptide or hBCMA-binding oligomer comprising an additional binding moiety of the present invention is G or comprises or has sequence GGGSG, GGGGS, GGSGG, GSGGG and/or SGGGG; for example the linker is G or comprises or has the sequence GGGSG, GGGSGGGGSG, GGGSGGGGSGGGGSG or GGGSGGGGSGGGGSGGGGSGGGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

In one preferred embodiment, a linker for a hBCMA-binding oligomer of the present invention is G or comprises or has sequence GGGSG; for example the linker comprises or has the sequence GGGSG, GGGSGGGGSG, GGGSGGGGSGGGGSG or GGGSGGGGSGGGGSGGGGSG. In another embodiment, a linker for a hBCMA-binding oligomer of the present invention comprises or has sequence GGGGS; for example the linker comprises or has the sequence GGGGS, GGGGSGGGGS, GGGGSGGGGSGGGGS GGGGSGGGGSGGGGSGGGGS.

Alternatively, in one embodiment, a hBCMA-binding polypeptide, or hBCMA-binding oligomer, comprising an additional binding moiety of the present invention has an additional binding moiety that is not separated from the hBCMA-binding polypeptide or the hBCMA-binding oligomer by a linker (i.e. the hBCMA-binding polypeptide or CD16a-binding oligomer is directly attached to an additional binding moiety).

In one preferred embodiment, a hBCMA-binding polypeptide or hBCMA-binding oligomer comprising an additional binding moiety of the present invention does not comprise a linker. In one preferred embodiment, the hBCMA-binding polypeptide or CD16a-binding oligomer is directly attached to an additional binding moiety.

In certain embodiments, an hBCMA-binding polypeptide of the present invention comprising an additional functional portion comprises the following structure:
[N-terminal portion]-[Helix 1]-[Separating portion]-[Helix 2]-[C-terminal portion]-[linker]-[additional functional portion];
[additional functional portion]-[linker]-[N-terminal portion]-[Helix 1]-[Separating portion]-[Helix 2]-[C-terminal portion]
[N-terminal portion]-[Helix 1]-[Separating portion]-[Helix 2]-[C-terminal portion]-[linker]-[additional functional portion]-[additional functional portion];
[additional functional portion]-[additional functional portion]-[linker]-[N-terminal portion]-[Helix 1]-[Separating portion]-[Helix 2]-[C-terminal portion]; or
[additional functional portion]-[linker]-[N-terminal portion]-[Helix 1]-[Separating portion]-[Helix 2]-[C-terminal portion]-[linker]-[additional functional portion];
   wherein, when more than one additional functional portion is present, each additional functional may be the same, or may be different.

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG; for example the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

Alternatively, the linker may be absent, i.e. the CD16a-binding polypeptide or CD16a-binding oligomer comprises the following structure:
[N-terminal portion]-[Helix 1]-[Separating portion]-[Helix 2]-[C-terminal portion]-[additional functional portion];
[additional functional portion]-[N-terminal portion]-[Helix 1]-[Separating portion]-[Helix 2]-[C-terminal portion]
[N-terminal portion]-[Helix 1]-[Separating portion]-[Helix 2]-[C-terminal portion]-[additional functional portion]-[additional functional portion];
[additional functional portion]-[additional functional portion]-[N-terminal portion]-[Helix 1]-[Separating portion]-[Helix 2]-[C-terminal portion]; or
[additional functional portion]-[N-terminal portion]-[Helix 1]-[Separating portion]-[Helix 2]-[C-terminal portion]-[additional functional portion].

In such embodiments, more preferably the hBCMA-binding polypeptide comprises the following structure:
[N-terminal portion]-[Helix 1]-[Separating portion]-[Helix 2]-[C-terminal portion]-[linker]-[additional functional portion];
[N-terminal portion]-[Helix 1]-[Separating portion]-[Helix 2]-[C-terminal portion]-[linker]-[additional functional portion]-[additional functional portion] (or
[N-terminal portion]-[Helix 1]-[Separating portion]-[Helix 2]-[C-terminal portion]-[additional functional portion];
[N-terminal portion]-[Helix 1]-[Separating portion]-[Helix 2]-[C-terminal portion]-[additional functional portion]-[additional functional portion]).

In such embodiments, preferably each additional functional portion is an additional binding moiety specific for a NK cell target, for example CD16a. Most preferably each additional functional portion is an additional binding moiety specific for CD16a,

In certain embodiments, a hBCMA-binding oligomer of the present invention comprising an additional functional portion comprises the following structure:
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[linker]-[additional functional portion];
[additional functional portion]-[linker]-[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion];
[N-terminal portion]-[Helix 1]-[Separating portion]-[Helix 2]-[C-terminal portion]-[linker]-[additional functional portion]-[linker]-[N-terminal portion]-[Helix 1]-[Separating portion]-[Helix 2]-[C-terminal portion];
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[linker]-[additional functional portion]-[additional functional portion];
[additional functional portion]-[additional functional portion]-[linker]-[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion];
[additional functional portion]-[linker]-[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[-linker]-[additional functional portion];
[additional functional portion]-[linker]-[N-terminal portion]-[Helix 1]-[Separating portion]-[Helix 2]-[C-terminal portion]-[linker]-[additional functional portion]-[linker]-[N-terminal portion]-[Helix 1]-[Separating portion]-[Helix 2]-[C-terminal portion];
[N-terminal portion]-[Helix 1]-[Separating portion]-[Helix 2]-[C-terminal portion]-[linker]-[additional functional portion]-[linker]-[N-terminal portion]-[Helix 1]-[Separating portion]-[Helix 2]-[C-terminal portion]-[linker]-[additional functional portion]; or
[N-terminal portion]-[Helix 1]-[Separating portion]-[Helix 2]-[C-terminal portion]-[linker]-[additional functional portion]-[additional functional portion]-[linker]-[N-terminal portion]-[Helix 1]-[Separating portion]-[Helix 2]-[C-terminal portion]
   wherein each linker portion in the oligomer may have the same sequence or have different sequences; each N-terminal portion in the oligomer may have the same sequence or have different sequences; each C-terminal portion in the oligomer may have the same sequence or have different sequences; each separating portion in the oligomer may have the same sequence or have different sequences; each Helix 1 portion in the oligomer may have the same sequence or have different sequences; and each Helix 2 portion in the oligomer may have the same sequence or have different sequences.

In such embodiments, preferably each additional functional portion is an additional binding moiety specific for a NK cell target, for example CD16a. Most preferably each additional functional portion is an additional binding moiety specific for CD16a,

In such embodiments, the linker preferably comprises or has a sequence of 1 to 25 (for example 1, 2, 3, 4, 5, 6, 7, 8, 9, 10, 11, 12, 13, 14, 15, 16, 17, 18, 19, 20, 21, 22, 23, 24 or 25) naturally occurring amino acids; and preferably 1 to 20 (for example 1, 5, 10, 15, or 20; and more preferably 1 to 15) naturally occurring amino acids, for example selected from the group consisting of G, S and T (preferably G and S). For example, the linker is G or comprises or has sequence GGGSG, GGGGS, GGSGG, GSGGG and/or SGGGG. Also, for example, the linker is G or comprises or has the sequence GGGSG, GGGSGGGGSG, GGGSGGGGSGGGGSG, GGGSGGGGSGGGGSGGGGSG, GGGGS, GGGGSGGGGS, GGGGSGGGGSGGGGS, GGGGSGGGGSGGGGSGGGGS, GGSGG, GGSGGGGSGG, GGSGGGGSGGGGSGG, GGSGGGGSGGGGSGGGGSGGGGSGG, GSGGG, GSGGGGSGGG, GSGGGGSGGGGSGGG, GSGGGGSGGGGSGGGGSGGG, SGGGG, SGGGGSGGGG, SGGGGSGGGGSGGGG, or SGGGGSGGGGSGGGGSGGGG.

In such embodiments, one or more linker may be absent.

In such embodiments, more preferably the hBCMA-binding oligomer comprises the following structure:
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[linker]-[additional functional portion]; or
[N-terminal portion]-[Helix 1]-[Separating portion]-[Helix 2]-[C-terminal portion]-[linker]-[additional functional portion]-[linker]-[N-terminal portion]-[Helix 1]-[Separating portion]-[Helix 2]-[C-terminal portion]; or
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[linker]-[additional functional portion]-[additional functional portion];
[N-terminal portion]-[Helix 1]-[Separating portion]-[Helix 2]-[C-terminal portion]-[linker]-[additional functional portion]-[linker]-[N-terminal portion]-[Helix 1]-[Separating portion]-[Helix 2]-[C-terminal portion]-[linker]-[additional functional portion]; or
[N-terminal portion]-[Helix 1]-[Separating portion]-[Helix 2]-[C-terminal portion]-[linker]-[additional functional portion]-[additional functional portion]-[linker]-[N-terminal portion]-[Helix 1]-[Separating portion]-[Helix 2]-[C-terminal portion]
   and more preferably
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[linker]-[additional functional portion]; or
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[linker]-[additional functional portion]-[additional functional portion].

In such embodiments, preferably each additional functional portion is an additional binding moiety specific for a NK cell target, for example CD16a. Most preferably each additional functional portion is an additional binding moiety specific for CD16a,

In such embodiments, one or more linker may be absent.

In one especially preferred embdoiemnt, the hBCMA-binding oligomer comprises the following structure:
[N-terminal portion]-[Helix 1]-[Separating portion]-[Helix 2]-[C-terminal portion]-[linker]-[N-terminal portion]-[Helix 1]-[Separating portion]-[Helix 2]-[C-terminal portion]-[linker]-[additional functional portion].

In such embodiments, preferably each additional functional portion is an additional binding moiety specific for a NK cell target, for example CD16a. Most preferably each additional functional portion is an additional binding moiety specific for CD16a.

In certain embodiments, a hBCMA-binding oligomer of the present invention comprising an additional functional portion comprises at least three hBCMA-binding polypeptides, and comprises the following structure:
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[linker]-[additional functional portion];
[additional functional portion]-[linker]-[N-terminal portion]-[Helix 1]-[Separating portion]-[Helix 2]-[C-terminal portion]-[Linker]-[N-terminal portion]-[Helix 2]-[Separating portion]-[Helix 2]-[C-terminal portion]-[Linker]-[N-terminal portion]-[Helix 1]-[Separating portion]-[Helix 2]-[C-terminal portion];
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[linker]-[additional functional portion]-[additional functional portion];
[additional functional portion]-[additional functional portion]-[linker]-[N-terminal portion]-[Helix 1]-[Separating portion]-[Helix 2]-[C-terminal portion]-[Linker]-[N-terminal portion]-[Helix 2]-[Separating portion]-[Helix 2]-[C-terminal portion]-[Linker]-[N-terminal portion]-[Helix 1]-[Separating portion]-[Helix 2]-[C-terminal portion];
   wherein each linker portion in the oligomer may have the same sequence, have different sequences, or two may have the same sequence and one may have a different sequence; each N-terminal portion in the oligomer may have the same sequence, have different sequences, or two may have the same sequence and one may have a different sequence; each C-terminal portion may have the same sequence, have different sequences, or two may have the same sequence and one may have a different sequence; each separating portion in the oligomer may have the same sequence, have different sequences, or two may have the same sequence and one may have a different sequence; each Helix 1 portion in the oligomer may have the same sequence, have different sequences, or two may have the same sequence and one may have a different sequence; and each Helix 2 portion in the oligomer may have the same sequence, have different sequences, or two may have the same sequence and one may have a different sequence.

In such embodiments, preferably each additional functional portion is an additional binding moiety specific for a NK cell target, for example CD16a. Most preferably each additional functional portion is an additional binding moiety specific for CD16a,

In such embodiments, the linker is preferably one as defined above. In such embodiments, one or more linker may be absent.

In such embodiments, more preferably the hBCMA-binding oligomer comprises the following structure:
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[linker]-[additional functional portion]; or
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[linker]-[additional functional portion]-[additional functional portion].

In such embodiments, more preferably each additional functional portion is an additional binding moiety specific for a NK cell target, for example CD16a. Most preferably each additional functional portion is an additional binding moiety specific for CD16a,

In the above embodiments defining the structure of the CD16a-binding polypeptide or CD16a-binding oligomer comprising an additional functional portion, each additional function portion may be any one described herein. For example, each additional functional portion may be independently selected from
an additional binding moiety (for example an additional binding moiety specific for a cancer cell surface target, for example a myeloma cell surface antigen, for example a myeloma cell surface antigen that is not BCMA, or an immune cell target, for example a NK cell target, for example CD16a); and
an immune signalling molecule, for example a cytokine, for example IL-15 or derivatives thereof.

For example, each additional functional portion may be independently selected from
an additional binding moiety specific for a cancer cell surface target (for example a myeloma cell surface antigen, for example a myeloma cell surface antigen that is not BCMA);
an additional binding moiety specific for an immune cell target (for example a NK cell target, for example CD16a); and
an immune signalling molecule, for example a cytokine, for example IL-15 or derivatives thereof.

For example, each additional functional portion may be independently selected from
an additional binding moiety specific for myeloma cell surface antigen that is not BCMA;
an additional binding moiety specific for a NK cell target, for example CD16a; and
an immune signalling molecule, for example a cytokine, for example IL-15 or derivatives thereof.

For example, each additional functional portion may be independently selected from
an additional binding moiety specific for a myeloma cell surface antigen that is not BCMA; and
an additional binding moiety specific for a NK cell target, for example CD16a; and
a a cytokine, for example IL-15 or derivatives thereof.

For example, each additional functional portion may be independently selected from
an additional binding moiety specific for a myeloma cell surface antigen that is not BCMA; and
an additional binding moiety specific for a NK cell target, for example CD16a; and
IL-15 or derivatives thereof.

In one preferred embodiment, each additional functional portion may be independently selected from
an additional binding moiety specific for a myeloma cell surface antigen that is not BCMA; and
an additional binding moiety specific for CD16a; and IL-15 or derivatives thereof.

In one preferred embodiment, each additional functional portion may be independently selected from
an additional binding moiety specific for CD16a; and
a cytokine, for example IL-15 or derivatives thereof.

In one preferred embodiment, each additional functional portion may be independently selected from
an additional binding moiety specific for a myeloma cell surface antigen that is not BCMA; and
an additional binding moiety specific for CD16a.

In one preferred embodiment, each additional functional portion is an additional binding moiety specific for CD16a.

In the above embodiments defining the structure of the hBCMA-binding polypeptide or hBCMA-binding oligomer comprising an additional functional portion, each additional function portion may be an additional binding moiety (for example an additional binding moiety specific for immune cell surface molecule, for example an NK cell surface target, for example CD16a). For example, each additional function portion may be an additional binding moiety for CD16a.

Further non-limiting examples of additional binding moieties include aptamers, monobodies, nanobodies, small molecules, and antibodies. In particular, additional binding moieties may be antibodies, for example monoclonal antibodies, for example monoclonal antibodies specific for hBCMA, for example monoclonal antibodies specific for CD3 (for example foralumab, otelixizumab, teplizumab, visilizumab, OKT3, UCHT1, SP34 or F2B), monoclonal antibodies specific for CD38 (for example daratumumab or isatuximab), or for example monoclonal antibodies specific for SLAMF7 (for example elotuzumab). Additional binding moieties may also be T cell engagers.

The additional binding moiety may be attached at the N-terminal end or the C-terminal end of the at least one hBCMA-binding polypeptide, optionally via a linker as described above. The additional binding moiety may, alternatively, be attached between two hBCMA-binding polypeptides, optionally separated by a linker as described above.

### HBCMA Binder-Drug Conjugates

Alternatively or additionally, an hBCMA binding polypeptide or hBCMA-binding oligomer as disclosed herein may be attached to a therapeutic agent to form an hBCMA binder-drug conjugate. Therefore, in an embodiment, the at least one hBCMA binding polypeptide or hBCMA-binding oligomer is typically covalently attached to one or more therapeutic agent(s), optionally via a linker or linkers as described above. Non-limiting examples of such therapeutic agents include cytotoxic drugs, for example mitomycin C, desmethyltopotecan, SN-38, MMAE, MMAF, doxorubicin, pyrrolobenzodiazepine, amanitin, maytansinoids (for example maytansinoid DM1 or maytansinoid DM4), or duostatins (for example duostatin 5.2). Further non-limiting examples of therapeutic agents include radioisotopes or radiolabelled compounds, which may be attached to an hBCMA binding polypeptide or hBCMA-binding oligomer of the invention (for example to form a radioligand). An hBCMA binding polypeptide or hBCMA oligomer of the invention attached to a radioisotope or radiolabelled compound finds use in the diagnosis (e.g. via imaging) or treatment (e.g. via radiotherapy) of cancer.

For the avoidance of doubt, during the generation of an hBCMA binder-drug conjugate according to the invention, the one or more therapeutic agent(s) (for example MMAF) is necessarily modified by the reaction between the functional group at the point of attachment (for example —NH, —OH or —SH (for example in a Cys)) and any attachment groups or linkers used. The skilled person will therefore understand that the therapeutic agent in an hBCMA binder-drug conjugate (for example an hBCMA binder-MMAF conjugate) comprises such a modification.

### Production of Polypeptides

The hBCMA-binding polypeptides of the invention can be manufactured using methods known in the art. For example, they can be prepared by chemical synthesis methods, by recombinant protein production techniques in, for example, bacterial, yeast, insect, fungal, plant or mammalian cells, or by cell-free in vitro protein expression methods using cell lysates and/or purified cell extracts. For example, hBCMA-binding polypeptides used in hBCMA binder-drug conjugates of the invention may be produced either by chemical synthesis or recombinant protein production methods.

hBCMA-binding polypeptides of the invention may also be fused, via recombinant or chemical synthesis techniques as described above, to a different molecule with therapeutic potential, for example an immunoglobulin or a polypeptide with therapeutic potential.

### Modulation of Polypeptide Properties

The pharmacokinetic properties of the hBCMA-binding polypeptides of the invention can be modulated by methods known in the art. For example, they can be linked to a moiety extending the plasma half-life, such as a polyethylene glycol polymer, an unstructured polypeptide (such as XTEN or PAS), an FcRn binding ligand such as serum albumin or the Fc domain of an immunoglobulin or a polypeptide capable of binding to a serum protein of long in vivo half-life including for example serum albumin or immunoglobulins.

### Formulations

An hBCMA-binding polypeptide or hBCMA binding oligomer (in particular an hBCMA binder-drug conjugate) according to the invention may be present in a formulation and particularly in a pharmaceutical formulation.

In certain embodiments, the invention provides a nucleic acid molecule encoding the hBCMA-binding polypeptide or hBCMA-binding oligomer of the invention. A nucleic acid molecule encoding the hBCMA-binding polypeptide or hBCMA-binding oligomer of the invention finds use as a medicament, for example for use in the treatment of cancer. The nucleic acid molecule may, for example, be a DNA or an RNA molecule, for example a mRNA molecule. Nucleic acid molecules according to the invention may be present in a formulation and particularly in a pharmaceutical formulation. As such, the present invention further provides a formulation and particularly in a pharmaceutical formulation of a nucleic acid molecule (for example, a DNA or RNA, and in particular a mRNA molecule) encoding the hBCMA-binding polypeptide or hBCMA-binding oligomer of the invention.

Pharmaceutical formulations include for example those suitable for oral, parenteral (including subcutaneous, intradermal, intraosseous infusion, intramuscular, intravascular (bolus or infusion), and intramedullary), or intraperitoneal administration, although the most suitable route may depend upon, for example, the condition and disorder of the subject under treatment.

In one embodiment of the invention, an hBCMA-binding polypeptide or hBCMA binding oligomer (in particular an hBCMA binder-drug conjugate) or nucleic acids (for example DNA or RNA molecules of the present invention, for example an mRNA molecule) according to the invention is administered as a pharmaceutical formulation suitable for oral or parenteral (including subcutaneous, intradermal, intraosseous infusion, intramuscular, intravascular (bolus or infusion), and intramedullary) administration.

Pharmaceutical formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. Peptides of the invention may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Preferably, the formulations may be presented in unit dosage or divided dosage containers, for example sealed ampoules and vials. The formulation may be stored in a freeze-dried (lyophilised) condition requiring only the addition of the sterile liquid carrier, for example saline, a physiologically acceptable solution or water-for-injection, immediately prior to use.

Extemporaneous injection and infusion solutions and suspensions may be prepared from sterile powders, granules or other dry composition. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

An hBCMA-binding polypeptide or hBCMA binding oligomer (in particular an hBCMA binder-drug conjugate) of the invention, and a pharmaceutical formulation comprising such a polypeptide, oligomer or binder-drug conjugate, or nucleic acid molecules of the invention (for example, DNA or RNA molecules of the present invention, for example a mRNA molecule of the present invention), and pharmaceutical formulations comprising those nucleic acid molecules, find use in the treatment and/or prophylaxis of cancer, for example multiple myeloma.

The amount of an hBCMA-binding polypeptide or hBCMA binding oligomer (in particular a hBCMA binder-drug conjugate) or nucleic acid molecule, required to achieve a therapeutic effect will vary with the particular route of administration and the characteristics of the subject under treatment, for example the species, age, weight, sex, medical conditions, the particular disease and its severity, and other relevant medical and physical factors. An ordinarily skilled physician can readily determine and administer the effective amount of the binding polypeptide, oligomer, binder-drug conjugate and/or composition comprising the same, or nucleic acid molecule and/or composition comprising the same, required for treatment and/or prophylaxis of cancer.

An hBCMA-binding polypeptide, hBCMA binding oligomer (in particular an hBCMA binder-drug conjugate) or nucleic acid molecule of the invention, or a pharmaceutical formulation thereof, may for example be administered daily, weekly, every second, third or fourth week or even as a high single dose depending on the subject and severity of the cancer to be treated.

An hBCMA-binding polypeptide, hBCMA binding oligomer (in particular an hBCMA binder-drug conjugate) or nucleic acid molecule of the invention, or a pharmaceutical formulation thereof, may for example be administered as a parenteral or oral dosage. Parenteral administration includes intravenous (into a vein, for example a central or a peripheral vein, bolus or infusion), intra-arterial (into an artery, for example a central or a peripheral artery), intraosseous infusion (into the bone marrow), intra-muscular (into muscle), intradermal (into the dermis), and subcutaneous (under the skin) administration. In one preferred embodiment, the dosage of the present invention is administered intravenously or intra-arterially, and more preferably by intravenous infusion (for example central intravenous infusion or peripheral intravenous infusion). In another preferred embodiment, the dosage of the present invention is administered by subcutaneous injection. As such, pharmaceutical formulations especially useful for the present invention are those suitable for intravenous administration, more especially intravenous infusion, or subcutaneous administration.

An hBCMA-binding polypeptide, hBCMA binding oligomer (in particular an hBCMA binder-drug conjugate) or nucleic acid molecule of the invention, or a pharmaceutical formulation thereof, may be administered as part of a treatment cycle. In a treatment cycle, said polypeptide may be administered on day 1 of the cycle, wherein the cycle lasts X days, with no further administration of the polypeptide of the invention for the next X-1 days. X may be, for example, from 1 to 42, for example from 2 to 28 days, for example from 2 to 21 days, for example from 2 to 14 days. Alternatively, the binding polypeptides may be administered as a split dose, for example on for example on days 1 and 2 of the cycle.

The cycle may be repeated one or several times depending on the category, class or stage of the cancer to be treated. For example, the cycle may be repeated from 1 to 100 times, for example from 2 to 50 times, for example 8 to 40 times, for example 8 or 16 times. For example, the hBCMA-binding polypeptide (for example in the form of an hBCMA binder-drug conjugate) may be administered for 8 repeats of a 7 day cycle, followed by 16 repeats of a 14 day cycle, optionally followed by further repeats of a 28 day cycle. For example, it may be administered in 21 day cycles (i.e. once every 3 weeks) until disease progression or unacceptable toxicity. An ordinarily skilled physician or clinician can readily determine the number of cycles of hBCMA-binding polypeptide (for example hBCMA binder-drug conjugate) required to prevent, counter or arrest the progress of the cancer.

### Combination Treatments

Whilst an hBCMA-binding polypeptide or hBCMA binding oligomer (in particular an hBCMA binder-drug conjugate) or nucleic acid molecules disclosed herein may be used as the sole active ingredient in the present invention, it is also possible for it to be used in combination with one or more further therapeutic agent(s), and the use of such combinations provides one embodiment of the invention. Such further therapeutic agents may be agents useful in the treatment and/or prophylaxis of cancer, or other pharmaceutically active materials. Such agents are known in the art.

Non-limiting examples of further therapeutic agents for use in the present invention may include proteasome inhibitors (PIs) (for example carfilzomib, bortezomib or ixazomib), immunomodulatory agents (IMiDs) (for example lenalidomide, thalidomide or pomalidomide), alkylators (for example cyclophosphamide, melphalan, melflufen or bendamustine), anthracyclines (for example doxorubicin), steroids (for example dexamethasone, prednisone or prednisolone), BCL-2 inhibitors (for example venetoclax), histone deacetylase (HDAC) inhibitors (for example panobinostat), anti-CD38 agents (for example daratumumab or isatuximab), anti-SLAMF7 agents (for example elotuzumab), immune checkpoint inhibitors (for example a CTLA-4 inhibitor, a PD-1 inhibitor, or a PD-L1 inhibitor), or ADAM17 inhibitors. For example, further therapeutic agents may be selected from proteasome inhibitors (for example carlfizomib or bortezomib), immunomodulatory agents (for example lenalidomide or thalidomide), alkylators (for example melphalan or melflufen), steroids (for example dexamethasone or prednisone), anti-CD38 agents (for example daratumumab), immune checkpoint inhibitors (for example a CTLA-4 inhibitor, a PD-1 inhibitor, or a PD-L1 inhibitor), and an ADAM17 inhibitor.

The one or more further therapeutic agent(s) may be used simultaneously, sequentially or separately with/from the administration of the dosage of hBCMA binding polypeptides, hBCMA binding oligomers, hBCMA binder-drug conjugates, or nucleic acid molecules of the invention. The individual components of such combinations can be administered separately at different times during the course of therapy or concurrently in divided or single combination forms.

In an embodiment of the invention, the one or more further therapeutic agent(s) are selected from a PI, an IMiD, and a steroid. For example, the one or more further therapeutic agents are a PI (for example bortezomib or carfilzomib), an IMiD (for example lenalidomide, thalidomide and pomalidomide), and a steroid (for example, prednisone, prednisolone and dexamethasone). For example, the one or more therapeutic agents are bortezomib, thalidomide, and dexamethasone.

In an embodiment of the invention, the one or more further therapeutic agent(s) are selected from a PI, an alkylator, and a steroid. For example, the one or more further therapeutic agents are a PI (for example bortezomib or carfilzomib), an alkylator (for example cyclophosphamide, melphalan, melflufen or bendamustine), and a steroid (for example, prednisone, prednisolone and dexamethasone). For example, the one or more therapeutic agents are bortezomib, melphalan or melflufen, and prednisone.

In an embodiment of the invention, the one or more further therapeutic agent(s) are selected from a PI and a steroid. For example, the one or more therapeutic agents are a PI (for example bortezomib or carfilzomib), and a steroid (for example, prednisone, prednisolone and dexamethasone). For example, the PI is bortezomib and the steroid is dexamethasone.

In an embodiment of the invention, the one or more further therapeutic agent(s) are selected from an IMiD and a steroid. For example, the one or more therapeutic agents are an IMiD (for example lenalidomide, thalidomide and pomalidomide), and a steroid (for example, prednisone, prednisolone and dexamethasone). For example, the IMiD is lenalidomide and the steroid is dexamethasone.

In another embodiment of the invention, the one or more therapeutic agent(s) may be selected from an NK cell-based or T cell-based therapy. For example, in the case where an hBCMA-binding polypeptide of the invention is attached to an additional binding moiety specific for an NK cell surface protein (such as a NK cell-binding polypeptide), allogeneic NK cells may be administered. For example, those NK cells may be co-injected with the polypeptide.

Alternatively, an hBCMA-binding polypeptide or hBCMA binding oligomer (in particular an hBCMA binder-drug conjugate) or nucleic acid molecule according to the invention may be combined with a therapeutic procedure such as a stem cell transplantation procedure, for example an autologous stem cell transplant or an allogenic stem cell transplant. Therefore, in an embodiment of the invention, an hBCMA-binding polypeptide disclosed herein may be combined with an autologous stem cell transplantation procedure. In another embodiment of the invention, an hBCMA-binding polypeptide disclosed herein may be combined with an allogenic stem cell transplantation procedure.

The simultaneous, sequential or separate administration of one or more further therapeutic agent(s) or therapeutic procedure with the hBCMA binding polypeptides, hBCMA binding oligomers, hBCMA binder-drug conjugates or nucleic acid molecules of the invention further enhances their effectiveness in the treatment and/or prophylaxis of cancer.

### Kits

The present invention provides a kit comprising an hBCMA-binding polypeptide, hBCMA binding oligomer (in particular an hBCMA binder-drug conjugate) or nucleic acid molecule as disclosed herein, and one or more further therapeutic agents that are useful in the treatment and/or prophylaxis of cancer.

Non-limiting examples of further therapeutic agents for use in a kit of the present invention may include proteasome inhibitors (PIS) (for example carfilzomib, bortezomib or ixazomib), immunomodulatory agents (IMiDs) (for example lenalidomide, thalidomide or pomalidomide), alkylators (for example cyclophosphamide, melphalan, melflufen, or bendamustine), anthracyclines (for example doxorubicin), steroids (for example dexamethasone, prednisone or prednisolone), BCL-2 inhibitors (for example venetoclax), histone deacetylase (HDAC) inhibitors (for example panobinostat), anti-CD38 agents (for example daratumumab or isatuximab), anti-SLAMF7 agents (for example elotuzumab), immune checkpoint inhibitors (for example a CTLA-4 inhibitor, a PD-1 inhibitor, or a PD-L1 inhibitor), or ADAM17 inhibitors.

For example, the one or more further therapeutic agents may be selected from proteasome inhibitors (for example carlfizomib or bortezomib), immunomodulatory agents (for example lenalidomide or thalidomide), alkylators (for example melphalan or melflufen), steroids (for example dexamethasone or prednisone), anti-CD38 agents (for example daratumumab), immune checkpoint inhibitors (for example a CTLA-4 inhibitor, a PD-1 inhibitor, or a PD-L1 inhibitor), and an ADAM17 inhibitor.

The one or more further therapeutic agents may for example be selected from proteasome inhibitors (for example carfilzomib or bortezomib), immunomodulatory agents (for example lenalidomide or thalidomide), alkylators (for example melphalan or melflufen), and steroids (for example dexamethasone or prednisone).

In one embodiment of the invention, the kit of the present invention finds use in the treatment and/or prophylaxis of cancer.

For the avoidance of doubt, an hBCMA-binding polypeptide, hBCMA binding oligomer (in particular an hBCMA binder-drug conjugate) or nucleic acid molecule as disclosed herein is present in a kit according to the present invention in a form and quantity suitable for use according to the present invention. Suitable pharmaceutical formulations are described herein. The skilled person can readily determine a quantity of the hBCMA binding polypeptide or oligomer (for example in the form of an hBCMA binder-drug conjugate) or nucleic acid molecule as disclosed herein suitable for the use according to the present invention.

### Uses of the Polypeptides of the Invention

hBCMA-binding polypeptides, hBCMA binding oligomers (in particular hBCMA binder-drug conjugates) or nucleic acid molecules of the present invention, as well as pharmaceutical formulations or kits of the present invention comprising said hBCMA-binding polypeptides, hBCMA binding oligomers, hBCMA binder-drug conjugates or nucleic acid molecules, find use in medicine, for example in the treatment and/or prophylaxis of cancer in a subject. Non-limiting examples of cancers include lung cancer (for example non-small cell lung cancer), breast cancer, or blood cancers. Blood cancers may include leukaemias (for example acute myeloid leukaemia, acute lymphoblastic leukaemia, chronic myeloid leukaemia, or chronic lymphocytic leukaemia), lymphomas (for example Hodgkin lymphoma, non-Hodgkin lymphoma, cutaneous T-cell lymphoma, small lymphocytic lymphoma, and other high-grade B-cell lymphomas), or plasma cell neoplasms and myelomas (for example, MGUS, plasmacytoma, smouldering myeloma, multiple myeloma, light chain myeloma, or non-secretory myeloma).

Preferably, hBCMA-binding polypeptides or hBCMA binding oligomers of the present invention (in particular hBCMA binder-drug conjugates) or nucleic acid molecules of the present invention, as well as pharmaceutical formulations or kits of the present invention comprising said hBCMA-binding polypeptides, oligomers, binder-drug conjugates or nucleic acid molecules, find use in the treatment and/or prophylaxis of myelomas (for example, MGUS, plasmacytoma, smouldering myeloma, multiple myeloma, light chain myeloma, or non-secretory myeloma). Most preferably, hBCMA-binding polypeptides, hBCMA-binding oligomers, hBCMA binder-drug conjugates or nucleic acid molecules of the present invention, as well as pharmaceutical formulations or kits of the present invention comprising said hBCMA-binding polypeptides, hBCMA-binding oligomers, hBCMA binder-drug conjugates or nucleic acid molecules, find use in the treatment and/or prophylaxis of multiple myeloma.

Alternatively or additionally, hBCMA-binding polypeptides, hBCMA binding oligomers (in particular hBCMA binder-drug conjugates) or nucleic acid molecules of the present invention, as well as pharmaceutical formulations or kits of the present invention comprising said hBCMA-binding polypeptides, hBCMA-binding oligomers, hBCMA binder-drug conjugates or nucleic acid molecules, may also find use in the treatment and/or prophylaxis of an autoimmune disorder in a subject. Non-limiting examples of autoimmune disorders include Addison's disease, coeliac disease, dermatomyositis, Graves disease, Hashimoto's thyroiditis, multiple sclerosis and optic neuritis, myasthenia gravis, pernicious anemia, reactive arthritis or rheumatoid arthritis, Sjögren's syndrome, systemic lupus erythematosus, or Type I diabetes, in particular autoimmune disorders in which hBCMA signalling is implicated, for example systemic lupus erythematosus, rheumatoid arthritis, multiple sclerosis and optic neuritis and Sjögren's syndrome.

hBCMA-binding polypeptides, hBCMA binding oligomers (in particular hBCMA binder-drug conjugates) or nucleic acid molecules of the present invention also find use in biotechnology and research applications, for example the detection of hBCMA in biological samples. hBCMA-binding polypeptides, hBCMA-binding oligomers, hBCMA binder-drug conjugates or nucleic acid molecules of the invention may for example be used in enzyme-linked immunosorbent assays (ELISAs) or attached to reporters such as fluorophores, for example for use in flow cytometry or immunohistochemistry.

### Examples

The following Examples illustrate the invention.

Preparative Example 1: Selection of Binders to hBCMA Using Phage Display

In this Example, human BCMA (hBCMA) was used as the target in phage display selections using a phage-based affibody library. Individual clones obtained after four selection cycles were assayed for binding to hBCMA in a monoclonal phage-ELISA (enzyme-linked immunosorbent assay) and ELISA-positive clones were DNA sequenced.

### Material and Methods

### Affibody Library.

An M13 phage display library of affibody molecules was prepared based on the phagemid vector pAffi-1 (Grönwall et al. (2007) J. Biotechnol. 128:162-183), This phagemid, containing a lac promoter and an OmpA signal peptide, is designed for phage display of encoded affibody library members as in-frame fusions to an albumin binding domain (ABD_{WT}), an amber stop codon and a truncated form (residues 249-406) of the M13 phage coat protein 3. A synthetic 121 bases long oligonucleotide (5′-GCGCTTTGGCTTGGGTCATCXXXTAAACTXXXYYYGAAGGCXXXXXXTTG XXXXXXGTTCAGGTTCGGCAGXXXXXXGATCTCXXXXXXCGCXXXXXXX XXTTCTTTGTTGAATTTGTTGT-3)′ [SEQ ID NO. 280] encoding amino acids 3-41 (reverse complementary strand) of the Z domain (Nilsson et al. (1987) Protein Eng. 1:107-113), in which wildtype codons for 14 positions in the domain had been randomized using mixtures of trinucleotide codons building blocks (XXX=equal mixture of codons for all 20 amino acids except Cys and Pro, YYY=60% Ile and 10% each of His, Tyr, Lys and Asp) during the synthesis was used as template for PCR amplification using primers

Forward:
[SEQ ID NO. 281]
(5′-GATGAAGCCCTCGAGGTAGACAACAAATTCAACAAAGAA-3′)
and
Reverse:
[SEQ ID NO. 282]
(5′-TTAGCTTCTGCTAGCAAGTTAGCGCTTTGGCTTGGGTCATC-3′).

Approximately 6.4 μg of XhoI and NheI double-cleaved and gel-purified (Qiagen, Germany) PCR product was ligated to 35 μg of XhoI and NheI double-cleaved and gel-purified pAffi-1 phagemid vector using T4 DNA ligase. The resulting ligation mixture was desalted using column-purification (Qiagen, Germany), divided into 24 portions and used to electroporate (0.1 cm BioRad cuvettes) 25 μl electrocompetent ER2738 E. coli cells (F′, glnV amber suppressor) (Lucigen, USA). 970 μl of Recovery medium (2% tryptone, 0.5% yeast extract, 10 mM NaCl, 2.5 mM KCl, 10 mM MgCl₂, 10 mM MgSO₄, and 20 mM glucose) was added to electroporated cells which were subsequently pooled (six electroporations per pool) and incubated at 37° C. for 1 h under shaking, after which pools of cells were titrated via spreading of dilution series on Amp (ampicillin)plates and transferred to four 5 litre shakeflasks, each containing one litre of Tryptic Soy Broth+Yeast extract medium (30 g/l tryptic soy broth, 5 g/l yeast extract; TSB+Y) supplemented with 2% glucose and 100 μg/ml Amp. Overnight cultures of cells were pelleted by centrifugation and resuspended in 50 ml of cold 40% glycerol, followed by distribution into 29 tubes of ca. 3.5 ml cell/glycerol solution per tube. From the post-electroporation titrations and the OD₆₀₀ measurements after the overnight cultivation it was calculated that the library size (diversity) was ca. 3×10¹⁰ and that each 3.5 ml aliquot of cells contained a number of cells corresponding to ca. 10,9× the library size. The tubes with cells were stored at −80° C. until used for phage stock preparation using M13KO7 helper phage.

### Naïve Phage Library Stock Production.

For the production of an affibody-displaying phage stock, 2 ml library glycerol stock was distributed and inoculated into three E-flasks containing 750 ml TSB+Y, 1% (w/v) glucose, 10 μg/ml Tet (tetracycline) and 100 μg/ml Carb (carbenicillin) and grown at 37° C. with shaking at 150 rpm until the cultures reached an OD₆₀₀=1. Cells were infected with 600 μl (multiplicity of infection (MOI) of 5) of M13KO7 helper phage (New England Biolabs)per E-flask, gently swirled and incubated without shaking for 15 min at 37° C., followed by 70 rpm at 37° C. for 15 min. The cells of each culture were centrifuged and resuspended in 670 ml TSB+Y, 100 μg/ml Carb and 1 mM IPTG (Isopropyl β-D-1-thiogalactopyranoside). 25 μg/ml Kan (kanamycin) was added 2 h after inoculation. Cultures were incubated at 37° C. with shaking at 200 rpm for 16-18 h. The phage library stock was harvested by precipitation with 20% (w/v) PEG6000/2.5 M NaCl twice. The titer of the stock was measured by spot titration and polymerase chain reaction-screening was used to analyze the percentage of phage particles carrying the phagemid with affibody insert.

Phage Display Selection of hBCMA Binding Candidates.

Four cycles of panning were performed using biotinylated hBCMA-Fc (human TNFRSF17/BCMA/CD269 Protein (His & human IgG1 Fc tag)), Biotinylated, (Sino Biological Inc., Eschborn, Germany, cat. no. 10620-H03H-B, corresponding to residues 1-54 of Uniprot entry Q02223) at concentrations 150 nM (cycle 1), 100 nM (cycle 2) and 50 nM (cycles 3-4). SA (streptavidin)-coated paramagnetic beads (Dynabeads M-280 Streptavidin, cat. no. 11205D, Invitrogen, Waltham, Massachusetts, USA) were washed twice with PBS (150 mM NaCl, 8 mM Na₂HPO₄, 2 mM NaH₂PO₄·H₂O, pH 7.4). To avoid selection of unspecific binders, all tubes used in the biopanning were pre-treated with 1% (w/v) bovine serum albumin (BSA) in PBS-T (PBS supplemented with 0.05% (v/v) Tween-20, pH 7.4). Further, in cycles 2-4, negative selection was performed by pre-incubating phage stock in PBS-T with 0.1% (w/v) BSA and SA-beads containing biotinylated trastuzumab mAb (contains Fc region) (Herceptin, Roche, Basel, Germany) for 30 min at room temperature (RT) under constant end-over-end (eoe) rotation, to remove phages carrying binders against SA and Fc. The amount of phage stock used was approximately 8×10¹¹ colony forming units (cfu) in cycle 1 and in subsequent cycles Biotinylated target protein was immobilised on 1.5 mg beads for 1 h at RT and eoe. Target-containing beads were incubated with 1% (w/v) BSA in PBS-T for 30 min at RT and eoe and then washed with PBS-T, before the addition of pre-incubated phage stock to perform the selection. The selection step was performed at RT and the incubation time for selection was 3 h (cycle 1) followed by wash 1, 3, 6 or 10 times with PBS-T eoe at RT, in subsequent cycles. The final wash volume was transferred to new 1% (w/v) BSA pre-treated tubes to remove sticky binders attached to the tube walls. Antigen-binding phages were eluted by incubation with 0.3 M acetic acid, pH 2.8 for 15 min eoe at RT, followed by transfer of the eluate to new pre-treated tubes and neutralization with equal volume 1 M Tris-HCl, pH 8.

### Phage Stock Amplifications.

New phage stocks were generated by infecting Escherichia coli XL-1 Blue cells (Agilent) grown in TSB+Y with 10 μg/ml Tet at 37° C. with shaking at 150 rpm until OD₆₀₀=0.5-0.8 with phage eluate (total volume after cycle 1, half volume after cycles 2-3). 15 ml bacterial culture was used in cycles 1-2 and 7.5 ml in cycles 3-4. The infected culture was gently swirled, incubated without shaking for 25 min at 37° C., followed by shaking at 70 rpm at 37° C. for 15 min. The culture was then centrifuged and resuspended in TSB+Y, before plating on blood agar plates (40 g/l blood agar) with 100 μg/ml Carb and 1% (w/v) glucose, and incubation for 16-18 h at 37° C. Bacterial colonies were collected to TSB+Y by scraping and inoculated to 200 ml TSB+Y with 100 g/ml Carb and grown at 37° C. with shaking at 150 rpm until OD₆₀₀=0.5-0.8. 30 ml after cycle 1, 20 ml after cycle 2 or 10 ml after cycle were superinfected with M13KO7 helper phage (MOI of ca. 10), gently swirled, incubated without shaking for 25 min at 37° C., followed by shaking at 70 rpm at 37° C. for 15 min. The culture was centrifuged and resuspended in 150 ml TSB+Y with 100 μg/ml Carb and 1 mM IPTG. 25 μg/ml Kan was added 2 h after inoculation. The culture was incubated at 37° C. with shaking at 150 rpm for 16-18 h. The phage library stock was harvested by precipitation with 20% (w/v) PEG6000/2.5 M NaCl twice. The titre of the stock was measured by spot titration on Carb plates and polymerase chain reaction-screening was used to analyse the percentage of colonies carrying phagemids with correctly-sized affibody inserts.

### Monoclonal Phage Supernatant Preparation.

Following cycle four, 48 bacterial colonies generating correctly sized PCR products were individually grown in 500 μl TSB+Y/Carb in 96-well deep-well plates at 30° C. with shaking at 250 rpm for 16-18 h. 30 μl of overnight culture was inoculated to 720 μl TSB+Y/Carb in a new 96-well deep-well plate and incubated for 2 h at 37° C., 250 rpm, before superinfection by addition of excess M13K07 helper phage in 100 μl TSB+Y/Carb per well. Plates were incubated for 30 min at 37° C. without shaking. Finally, 150 μl TSB+Y/Carb/IPTG/Kan was added per well. Final concentrations were 100 μg/ml Carb, 1 mM IPTG and 25 μg/ml Kan. The deep-well plates were incubated at 37° C., 250 rpm, for 16-18 h. Phage supernatants were collected by centrifugation the next day.

### Monoclonal Phage-ELISA Screening of Candidates.

A MaxiSorp ELISA plate (Clear Flat-Bottom Immuno Nonsterile 384-Well Plates, cat. no. 464718, Thermo Fisher Scientific) was coated at 4° C. with slow shaking for 16-18 h with 30 μl of 5 μg/ml hBCMA-Fc (Sino Biological, cat. No. 10620-H15H corresponding to residues 1-54 of NCBI entry NP_001183.2 fused to a rabbit Fc), 15 μg/ml human serum albumin (HSA; Sigma product no. SRP6182) (for assessment of proper display of the expression cassette containing a tripartite fusion protein including an affibody, an affibody albumin binding domain and the truncated protein 3), 10 μg/ml trastuzumab (as a control for binding to the Fc tag) or 5 μg/ml unrelated control protein (Human CD38, His Tag, Acro Biosystems, cat. no. CD8-H5224 corresponding to residues 43-300 of NCBI entry NP_001766.2) in 100 mM sodium carbonate buffer pH 8.5 per well (¼ of the wells with each coated protein). The coating solution was removed and the plate was washed two times with PBS-T, and blocked with PBS supplemented with 1% (w/v) BSA for 1 h at RT with slow shaking. The blocking solution was discarded, and the wells were incubated with 10 μl phage supernatant diluted 1:3 with 20 μl PBS-T for 1 h at RT with slow shaking. The supernatant was removed and the plate washed three times with PBS-T. Following addition of 30 μl 1:5000 α-M13-HRP (Sigma-Aldrich, Stockholm, Sweden) in PBS-T and incubated for 30 min at RT with slow shaking, the plate was washed twice with PBS-T and once with PBS. 30 μl TMB substrate (TMB Substrate Kit, Thermo Fisher Scientific) was added to each well. The reactions were stopped by adding 30 μl 2 M H₂SO₄ after 15-25 min. Absorbance at 450 nm was measured using a CLARIOstar microplate reader (BMG Labtech, Ortenberg, Germany). Candidates that were considered as ELISA-positive clones had high HSA signals and relatively high signals to hBCMA-Fc compared to signals observed to trastuzumab (Fc control) and other unrelated target controls.

DNA sequencing. Following ELISA screening, six ELISA-positive clones were sent for DNA sequencing by Sanger sequencing (Microsynth Seqlab Sanger Sequencing Service, Microsynth, Balgach, Switzerland).

### Results

To identify polypeptides binding to hBCMA, an affibody phage library was constructed and used as input in the first cycle in a four-cycle phage display selection campaign as described in Material and methods (above). After the fourth cycle of selections, the phage eluates from the acid and trypsin selection tracks were used to infect E. coli cells to obtain individual colonies on Carbenicillin plates. 48 randomly picked colonies (clones) with correctly sized affibody gene inserts from each track were subjected to binding analyses using a phage-ELISA experiment using microtiter plates onto which four test antigens (HSA, hBCMA-Fc, trastuzumab (Fc) and CD38) had been coated. One clone, which showed strong ELISA signals to HSA and hBCMA-Fc and low signals for the trastuzumab (Fc) and CD38 controls, was subsequently subjected to DNA sequencing. This clone was denoted Fa-G6 and its deduced amino acid sequence is listed in Table 1 and in the sequence listing as SEQ ID NO. 1. Table 1 also lists the remaining sequences of the present disclosure, with the corresponding SEQ ID references. Throughout this Examples section, an Example Compound with number X is a polypeptide with the amino sequence of the same number, ie SEQ ID X.

Biological Example 2: Initial Biosensor Characterisation of a Candidate hBCMA Binding Clone Fa-G6

In this Example, the hBCMA-binding polypeptide Fa-G6 corresponding to SEQ ID NO: 1 was subcloned, expressed, purified as an His₆-affibody-ABD_{WT} fusion protein [SEQ ID NO: 3] and initially analysed by surface plasmon resonance for binding to hBCMA-Fc.

### Materials and Methods

Subcloning of the Fa-G6 polypeptide variant.

A DNA fragment encoding the Fa-G6-binding polypeptide candidate variant [SEQ ID 1] was amplified by PCR from the library phagemid vector pAffi-1 with specific primers introducing restriction sites for XhoI and AscI. The fragment was cleaved and ligated to a T7 promoter-based E. coli expression vector prepared using the same restriction enzymes. This generated a monomeric polypeptide construct with an N-terminal His₆ tag and a C-terminal ABD_{WT}. The DNA construct was sequence verified using Sanger sequencing (Microsynth). The amino acid sequence of this affibody variant is listed in Table 1 and in the sequence listing as SEQ ID NO. 3.

### Expression and Purification.

E. coli BL21 (DE3) cells were transformed with plasmid containing the expression DNA construct and cultivated in 10 ml TSB+Y with 25 μg/ml Kan at 37° C. with shaking at 150 rpm for 16-18 h. The culture was inoculated 1:100 in 200 ml TSB+Y with 25 μg/ml Kan and grown at 37° C. with shaking at 150 rpm until OD₆₀₀=0.6-1. IPTG was added to final concentration 1 mM to induce protein expression. The cultivation was incubated at 25° C. with shaking at 150 rpm for 16-18 h and the cells were harvested by centrifugation. The cell pellet was resuspended in denaturing lysis buffer (7 M guanidinium chloride, 47 mM Na₂HPO₄, 2.65 mM NaH₂PO₄, 10 mM TRIS-HCl, 100 mM NaCl, pH 8). After incubation at 37° C. for 2 h with shaking at 150 rpm, the cells were centrifuged and the denatured protein from the supernatant of the cell lysate was added to a tube containing 3 ml HisPur Cobalt IMAC Resin (cat. no. 89966, Thermo Scientific). Supernatant was incubated with resin for 30 min at RT with end-over-end rotation. Contaminants were removed by washing three times with wash buffer (7 M guanidium chloride, 46.6 mM Na₂HPO₄, 3.4 mM NaH₂PO₄, 300 mM NaCl, 10 mM imidazole, pH 8) and hBCMA binding affibody Fa-G6-ABD_{WT} fusion protein was subsequently eluted with elution buffer (6 M urea, 50 mM NaH₂PO₄, 100 mM NaCl, 30 mM glacial acetic acid, 70 mM sodium acetate, pH 5) by incubation at RT for 10 min with end-over-end rotation. Following IMAC purification, the protein buffer was exchanged to PBS using PD-10 desalting columns (cat. no. 17085101, Cytiva, Uppsala, Sweden). SDS-PAGE analysis was performed to confirm the purity and concentration, which were measured by absorbance at 280 nm using extinction coefficients calculated from the amino acid sequence, of the purified protein (NuPAGE, 4 to 12%, Bis Tris, Invitrogen, Waltham, Massachusetts, USA). The low molecular weight marker was from Cytiva (Art. no. 17044601).

Biosensor Analyses of the hBCMA Binding Variant Fa-G6 with an N-Terminal His₆ tag and a C-Terminal ABD_{WT}.

A Biacore T200 instrument (Cytiva) was used to analyse the real-time interaction of the His₆-Fa-G6-ABD_{WT} fusion protein [SEQ ID NO. 3] with hBCMA. The protein ligand hBCMA-Fc (human TNFRSF17/BCMA/CD269 Protein (Fc tag), Sino Biological Inc., cat. no. 10620-H15H, corresponding to residues 1-54 of Uniprot entry Q02223) and HSA as a positive control were diluted in 10 mM NaOAc, pH 4.5 and immobilised on a Series S CM5 sensor chip (Cytiva) by amine coupling, using the manufacturer's instructions. One flow cell was activated and deactivated to be used as a reference cell. The His₆-Fa-G6-ABD_{WT} fusion protein [SEQ ID NO: 3] was diluted in the running buffer PBS-T before binding analysis was performed at 25° C. and a flow rate of 30 μl/min. After each injection series the flow cells were regenerated by the injection of 10 mM HCl. In a first experiment, the hBCMA binding variant was injected over the hBCMA-Fc surface and a trastuzumab (containing Fc) control surface at a 200 nM concentration.

### Results

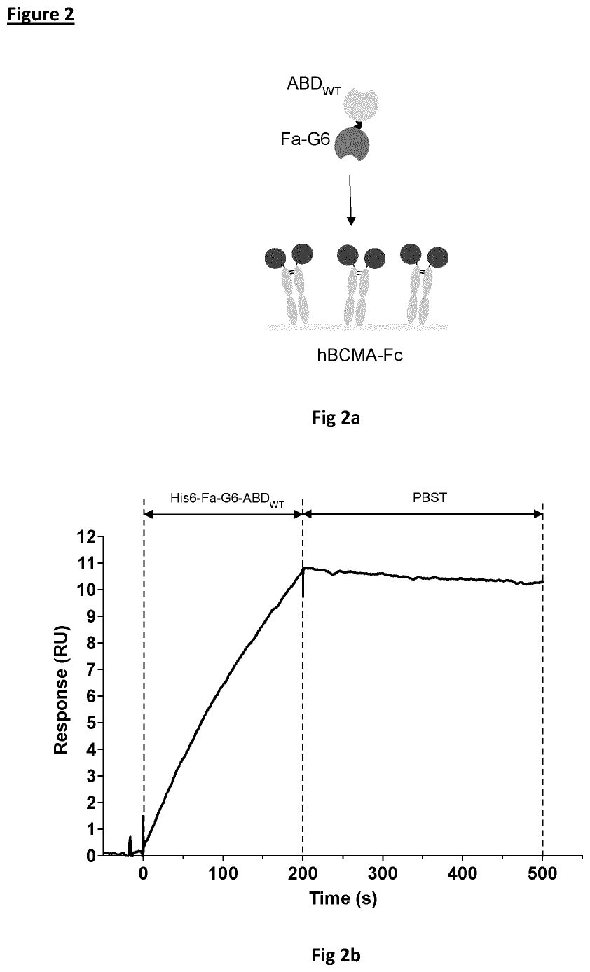

To investigate if the anti-hBCMA-binding polypeptide candidate clone Fa-G6 identified via the phage-ELISA and DNA sequencing [SEQ ID NO. 1] showed target binding when expressed as a soluble protein, it was subcloned, expressed in E. coli cytoplasm, and purified as a His₆-polypeptide-ABD_{WT} fusion protein [SEQ ID NO. 3]. The ABD_{WT} moiety is a small serum albumin binding protein that can be used for affinity chromatography purification and/or immobilization using human serum albumin as ligand, if wanted. Immobilised metal ion affinity chromatography (IMAC)-purified fusion protein, employing the hexahistidyl (His₆) gene fusion partner in the protein, was analysed by SDS-PAGE which showed that the protein was of high purity and had an approximate molecular weight in accordance with its amino acid sequences. Injection of the fusion protein at a 200 nM concentration over a sensor chip surface containing immobilised hBCMA-Fc, showed that the fusion protein bound to hBCMA-Fc. In FIG. 2a there is shown the general binding experiment in cartoon form. The binding experiment trace obtained in shown in FIG. 2b. No binding to a trastuzumab (Fc) control was observed (data not shown).

Biological Example 3: Biosensor Evaluation of N-Terminally Truncated Variants of the hBCMA Binding Clone Fa-G6

In this Example, residues 1-5 at the N-terminus of the hBCMA binding clone Fa-G6 [SEQ ID NO: 1] were successively truncated to investigate the effect on the binding interaction between Fa-G6 and hBCMA.

### Materials and Methods

Cloning of the hBCMA Binding Clone to Construct the Truncation Mutants.

The DNA fragment encoding the hBCMA-binding polypeptide candidate [SEQ ID NO. 1] was amplified from the library vector pAffi-1 with specific primers, designed to introduce overhangs to the full encoding sequence, or the encoding sequence with deletions spanning up to residue five of the N-terminus, with complementary ends to ends of linearised expression vectors (one for each truncated variant). In-Fusion HD Cloning Kit (Takara Bio, Gothenburg, Sweden) was used to clone five monomeric affibody constructs (the wildtype Fa-G6 variant and four truncation mutants with deletions truncations corresponding to -2, -3, -4 and -5 residues of the affibody N-terminus, respectively) with a C-terminal His₆ tag. The DNA constructs were sequence verified using Sanger sequencing (Microsynth). The amino acid sequences of these five hBCMA-binding polypeptide variants are listed in Table 1 and in the sequence listing as SEQ ID NOs 4-8.

### Expression and Purification of Truncation Variants.

E. coli BL21 (DE3) cells were transformed with plasmids containing the five constructs all with a C-terminal His₆ tag, and cultivated in 10 ml TSB+Y with 25 μg/ml Kan at 37° C. with shaking at 150 rpm for 16-18 h. The cultures were inoculated 1:100 in 200 ml TSB+Y with 25 g/ml Kan and grown at 37° C. with shaking at 150 rpm until OD₆₀₀=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in denaturing lysis buffer. After incubation at 37° C. for 2 h with shaking at 150 rpm, the cells were centrifuged and the denatured protein from the supernatant of the cell lysates was added to tubes containing 3 ml HisPur Cobalt IMAC Resin (cat. no. 89966, Thermo Scientific). Supernatant was incubated with resin for 30 min at RT with end-over-end rotation. Contaminants were removed by washing three times with wash buffer and h the Fa-G6 affibody variants were subsequently eluted with elution buffer by incubation at RT for 10 min with end-over-end rotation. Following IMAC purification, the protein buffer was exchanged to PBS using PD-10 desalting columns (cat. no. 17085101, Cytiva). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen). The low molecular weight marker was from Cytiva (Art. no. 17044601).

### Biosensor Analyses of the Truncation Mutants.

A Biacore T200 instrument (Cytiva) was used to analyse in real-time the interactions between four truncation mutants [Example Compounds 5-8 with SEQ ID NOs: 5-8] and hBCMA, and in comparison to the wildtype hBCMA binder Fa-G6 [SEQ ID NO: 4]. The protein ligand hBCMA-Fc (Sino Biological Inc., cat. no. 10620-H15H) was immobilised (1,000 RUs) on a Series S CM5 sensor chip (Cytiva) by amine coupling, using the manufacturer's instructions. One flow cell was activated and deactivated to be used as a reference cell. Analytes were diluted in the running buffer PBS-T before binding analysis was performed at 25° C. and a flow rate of 30 μl/min. After each injection series the flow cells were regenerated by the injection of 10 mM HCl. The wildtype Fa-G6-His₆ construct and the four truncation mutants were injected over the hBCMA-Fc surface at a concentration of 20 nM.

### Results

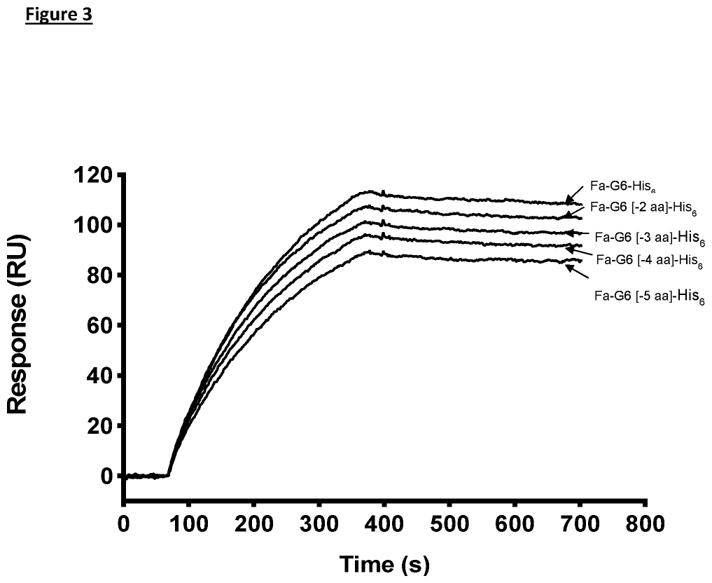

To investigate if truncation of N-terminal residues has an effect on the binding of the Fa-G6 clone to hBCMA, truncations corresponding to -2, -3, -4 and -5 residues of the polypeptide N-terminus was performed to obtain four truncation mutants which, together with the wildtype Fa-G6, were expressed in E. coli cytoplasm and purified as His₆ fusion proteins [SEQ ID NOs: 4-8]. IMAC-purified fusion proteins, employing the His₆ gene fusion partner in the proteins, were analysed by SDS-PAGE which showed that all five proteins were of high purity and had approximate molecular weights in accordance with their amino acid sequences. Injection of the four truncation mutants with successively smaller molecular weights and the wildtype binder over the sensor chip surfaces containing immobilised hBCMA-Fc showed that truncations up to residues five of the N-terminus did not impair the binding response profile to hBCMA-Fc, compared to the parental full-length Fa-G6 (FIG. 3).

Biological Example 4: Alanine Scan of the hBCMA Binding Clone Fa-G6

The hBCMA binding Fa-G6 clone indicated relatively low solubility and/or thermostability properties, prompting the development of second generation hBCMA binders based on the parental Fa-G6 binder, with potentially different biophysical properties compared to Fa-G6. In this Example, an alanine scan of Fa-G6 was performed to investigate which positions are important for the binding interaction of Fa-G6 with hBCMA, to be employed as the basis for the design of second-generation libraries based on Fa-G6.

### Materials and Methods

Site-Directed Mutagenesis of the hBCMA Binding Clone Fa-G6.

The DNA fragment encoding the Fa-G6-binding polypeptide candidate variant [SEQ ID 1] was amplified by PCR from the library phagemid vector pAffi-1 with specific primers introducing restriction sites. The fragment was cleaved and ligated to a T7 promoter-based E. coli expression vector prepared using the same restriction enzymes. This generated a monomeric polypeptide construct with an N-terminal His₆ tag. This expression plasmid containing the His₆-Fa-G6 construct (SEQ ID NO. 129) was sequence verified using Sanger sequencing (Microsynth) and then used as the DNA template in individual mutagenesis PCR reactions to substitute the original codons in the 14 variable positions to alanine codons, using primers designed for each position. The 14 alanine substitution variants were sequence verified using Sanger sequencing (Microsynth) and their amino acid sequences are listed in Table 1 and in the sequence listing as SEQ ID NOs 9-22.

### Expression and Purification of Alanine Variants.

E. coli BL21 (DE3) cells were separately transformed with individual plasmids encoding for the 14 alanine variants [SEQ ID NOs 9-22], each equipped with a N-terminal His₆ tag, as well as the wildtype His₆-Fa-G6 construct [SEQ ID NO. 129], and cultivated in 10 ml TSB+Y with 25 μg/ml Kan at 37° C. with shaking at 150 rpm for 16-18 h. The cultures were inoculated at a 1:100 dilution in 200 ml TSB+Y with 25 μg/ml Kan and grown at 37° C. with shaking at 150 rpm until OD₆₀₀=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in denaturing lysis buffer. After incubation at 37° C. for 2 h with shaking at 150 rpm, the cells were centrifuged and the denatured protein from the supernatant of the cell lysates was added to tubes containing 3 ml HisPur Cobalt IMAC Resin (cat. no. 89966, Thermo Scientific). Supernatant was incubated with resin for 30 min at RT with end-over-end rotation. Contaminants were removed by washing three times with wash buffer and hFa-G6-His₆ variants were subsequently eluted with elution buffer by incubation at RT for 10 min with end-over-end rotation. Following IMAC purification, the protein buffer was exchanged to PBS using PD-10 desalting columns (cat. no. 17085101, Cytiva). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen). The low molecular weight marker was from Cytiva (Art. no. 17044601).

### Biosensor Analyses of the Alanine Variants.

A Biacore T200 instrument (Cytiva) was used to analyse the interactions between the His₆ tagged wild-type Fa-G6 polypeptide [SEQ ID NO. 129] and 14 alanine variants [SEQ IDs 9-22] and hBCMA in real-time. The protein ligand hBCMA-Fc (Sino Biological Inc., cat. no. 10620-H15H) was immobilised (3500 RU) on a Series S CM5 sensor chip (Cytiva) by amine coupling, using the manufacturer's instructions. One flow cell was activated and deactivated to be used as a reference cell. Analytes were diluted in the running buffer PBS-T before binding analysis was performed at 25° C. and a flow rate of 30 μl/min. After each injection series the flow cells were regenerated by an injection of 10 mM HCl. The wildtype His₆-Fa-G6 construct and the 14 alanine variants were injected over the hBCMA-Fc surface at a common concentration of 200 nM.

### Results

To investigate which of the amino acids occupying the 14 variable positions in the Fa-G6 clone are most important for the binding interaction to hBCMA, for the ensuing design of second-generation libraries based on Fa-G6, a site-directed alanine scanning mutagenesis was performed to obtain 14 alanine variants which, together with the wildtype Fa-G6 (His₆-Fa-G6, [SEQ ID 129]), were expressed in E. coli cytoplasm and purified as His₆-affibody fusion proteins [SEQ IDs 9-22]. IMAC-purified fusion proteins, employing the His₆ gene fusion partner in the proteins, were analysed by SDS-PAGE which showed that all 15 proteins were of high purity and had approximate molecular weights in accordance with their amino acid sequences. FIG. 4 shows the injections of the 14 alanine variants and the wild-type Fa-G6 binder over the sensor chip surface containing immobilised hBCMA-Fc. The resulting sensorgrams showed that substitution to alanine in positions 9, 10, 11, 14 and 18 reduced the binding of these variants to hBCMA, whereas substitution to alanine in positions 13, 24, 25, 27, 28, 31, 32 and 35 impaired the binding. Substitution to alanine in position 17 suggested improved hBCMA-Fc binding.

### Preparative Example 5: Second-Generation Library Construction, Selection and Phage ELISA

In this Example, two different polypeptide libraries were constructed based on the re-randomization of certain variable positions, and to various degrees, of the Fa-G6 clone. The two libraries were used for the selection of new hBCMA binders, with diversified biophysical properties and potential to be used for different application purposes compared to the existing Fa-G6. Individual clones obtained after three phage display selection cycles were assayed for binding to hBCMA in a monoclonal phage-ELISA and ELISA-positive clones were DNA sequenced.

### Materials and Methods

hBCMA Affibody Fa-G6 Second Generation Libraries.

Two M13 phage display second-generation selection libraries were prepared, based on the Fa-G6 clone. The phagemid vectors used were pAffi-1 (Grönwall et al. (2007) J. Biotechnol. 128:162-183) and a variant thereof denoted pAffi-100. The pAffi-1 phagemid, containing a lac promoter and an OmpA signal peptide, is designed for phage display of encoded affibody library members as in-frame fusions to an albumin binding domain (ABD), an amber stop codon and a truncated form (residues 249-406) of the M13 phage coat protein 3. The pAffi-100 phagemid also contains a lac promoter, an OmpA signal peptide, affibody library members as in-frame fusions to ABD, but additionally contains a trypsin protease cleavage site before the amber stop codon, followed by a full-length form of the M13 phage coat protein 3.

Two synthetic 121 bases long oligonucleotide based on the Fa-G6 sequence, Oligo A (5′-ACAACAAATTCAACAAAGAA X01 X01 X03 GCG X04 X01 GAGATC X06 X06 CTGCCGAACCTGAAC X03 X09 CAA X10 X11 GCCTTC X12 X13 X14 TTA X15 GATGACCCAAGCCAAAGCGC-3′) [SEQ ID NO. 283] and Oligo B (5′-ACAACAAATTCAACAAAGAA X01 X01 Y03 GCG Y04 X01 GAGATC X06 X06 CTGCCGAACCTGAAC Y03 Y09 CAA Y10 Y11 GCCTTC Y12 Y13 X14 TTA Y15 GATGACCCAAGCCAAAGCGC-3′) [SEQ ID NO. 284], encoding amino acid positions 3-41 (reverse complementary strand) corresponding to the Z domain numbering (Nilsson et al. (1987) Protein Eng. 1:107-113), in which wildtype codons for 14 positions in the domain had been randomized using mixtures of trinucleotide codons building blocks (amino acid codons for Cys, Pro and Gly were excluded; X01=equal mixture of codons for all 17 amino acids, X03=90% Phe and the remaining 10% equally distributed between the remaining amino acids, X04=90% Asp and the remaining 10% equally distributed between the remaining amino acids, X06=20% Ala and the remaining 80% equally distributed between the remaining amino acids, X09=90% Tyr and the remaining 10% equally distributed between the remaining amino acids, X10-90% Lys and the remaining 10% equally distributed between the remaining amino acids, X11-90% Trp and the remaining 10% equally distributed between the remaining amino acids, X12-90% Ile and the remaining 10% equally distributed between the remaining amino acids, X13=90% Arg and the remaining 10% equally distributed between the remaining amino acids, X14=90% Ser and 10% Lys, X15=90% Met and the remaining 10% equally distributed between the remaining amino acids, Y03=60% Phe and the remaining 40% equally distributed between the remaining amino acids, Y04-60% Asp and the remaining 40% equally distributed between the remaining amino acids, Y09-60% Tyr and the remaining 40% equally distributed between the remaining amino acids, Y10=60% Lys and the remaining 40% equally distributed between the remaining amino acids, Y11=60% Trp and the remaining 40% equally distributed between the remaining amino acids, Y12-60% Ile and the remaining 40% equally distributed between the remaining amino acids, Y13=60% Arg and the remaining 40% equally distributed between the remaining amino acids, Y15-60% Met and the remaining 40% equally distributed between the remaining amino acids); during the synthesis was used as template for PCR amplification using primers

Forward
[SEQ ID NO. 285]
(5′-GATGAAGCCCTCGAGGTAGACAACAAATTCAACAAAGAA-3′)
and
Reverse
(SEQ ID NO. 286]
[5′-TTAGCTTCTGCTAGCAAGTTAGCGCTTTGGCTTGGGTCATC-3′).

Approximately 5.4 μg of XhoI and NheI double-cleaved and gel-purified (Qiagen, Germany) PCR products of Oligo A and Oligo B, respectively, were ligated to 30 μg of XhoI and NheI double-cleaved and gel-purified pAffi-1 and pAffi-100 phagemid vector, respectively, using T4 DNA ligase.

Each resulting ligation mixture was desalted using column-purification (Qiagen, Germany), divided into 24 portions and used to electroporate (0.1 cm BioRad cuvettes)₂₅ μl electrocompetent ER2738 E. coli cells (F′, glnV amber suppressor) (Lucigen, USA). 970 μl of Recovery medium (2% tryptone, 0.5% yeast extract, 10 mM NaCl, 2.5 mM KCl, 10 mM MgCl₂, 10 mM MgSO₄, and 20 mM glucose) was added to electroporated cells which were subsequently pooled (six electroporations per pool) and incubated at 37° C. for 1 h under shaking, after which pools of cells were titrated via spreading of dilution series on ampicillin plates and transferred to four 5 litre shake flasks, each containing 500 ml of Tryptic Soy Broth+Yeast extract medium (30 g/l tryptic soy broth, 5 g/l yeast extract; TSB+Y) supplemented with 1.5% glucose and 100 μg/ml ampicillin. Overnight cultures of cells were pelleted by centrifugation and resuspended in 50 ml of cold 40% glycerol, followed by distribution into approximately 29 tubes of ca. 2 ml cell/glycerol solution per tube. From the post-electroporation titrations and the OD₆₀₀ measurements after the overnight cultivation it was calculated that the library size (diversity) of Oligo A in pAffi-1 was ca. 3.4×10⁷ and that each 2 ml aliquot of cells contained a number of cells corresponding to ca. 147× the library size. For Oligo B in pAffi-100 the calculated library size (diversity) was ca. 5.9×10⁷ and each 2 ml aliquot of cells contained a number of cells corresponding to ca. 84× the library size. The tubes with cells were stored at −80° C. until used for phage stock preparation using M13KO7 helper phage.

### Phage Stock Preparations.

For the production of an affibody-displaying phage stock, 0.5 ml of each library glycerol stock was distributed and inoculated into one non-baffled E-flask each, containing 500 ml TSB+Y, 1% (w/v) glucose, 10 μg/ml Tet (tetracycline) and 100 μg/ml Carb (carbenicillin), and grown at 37° C. with shaking at 150 rpm until the cultures reached an OD₆₀₀=1.25 ml cells per culture were infected with a multiplicity of infection (MOI) of 5 of M13KO7 helper phage (New England Biolabs), gently swirled and incubated without shaking for 15 min at 37° C., followed by 70 rpm at 37° C. for 15 min. The cells of each culture were centrifuged and resuspended in two baffled E-flasks for each library, containing 700 ml TSB+Y, 100 μg/ml Carb and 1 mM IPTG (Isopropyl β-D-1-thiogalactopyranoside). 25 μg/ml Kan (kanamycin) was added 2 h after inoculation. Cultures were incubated at 37° C. with shaking at 200 rpm for 16-18 h. The phage library stock was harvested by precipitation with 20% (w/v) PEG6000/2.5 M NaCl twice. The titer of the stock was measured by spot titration and polymerase chain reaction-screening was used to analyse the percentage of clones carrying a phagemid with a correctly sized affibody gene insert.

Selections from Secondary Libraries of the BCMA Binding Clone Fa-G6.

Three rounds of panning were performed with the two optimisation selection library stocks using recombinant, biotinylated hBCMA-Fc at concentrations 140 nM in cycle 1, 40 nM in cycle 2 and 25 nM in cycle 3, in six separate selection tracks. In three additional selection tracks, where competition with non-biotinylated target was included, the concentrations of biotinylated hBCMA-Fc were 30 nM in cycle 1 and 10 nM cycle 2-3 and the concentrations of recombinant, non-biotinylated hBCMA (BCMA-His, 10620-H08H, Sino Biological) were 3 μM in cycle 1 and 1 μM in cycles 2-3. Trastuzumab containing human IgG1 Fc was used for simultaneous negative selection at 300 nM in all selection cycles. SA (streptavidin)-coated paramagnetic beads (Dynabeads M-280 Streptavidin, cat. no. 11205D, Invitrogen, Waltham, Massachusetts, USA) were washed twice with PBS (150 mM NaCl, 8 mM Na₂HPO₄, 2 mM NaH₂PO₄·H₂O, pH 7.4). To avoid unspecific binders, all tubes used in the biopanning were pre-treated with 1% (w/v) bovine serum albumin (BSA) in PBS-T (PBS supplemented with 0.05% (v/v) Tween-20, pH 7.4). Furthermore, phage stock in PBS-T was pre-incubated for 30 min at room temperature (RT) under constant end-over-end (eoe) rotation with 0.1% (w/v) BSA and beads, to remove phages carrying binders against SA. Amount of phage stock used was 10¹² colony forming units (cfu) in cycle 1, 10¹³ cfu in cycle 2 and 10¹¹⁻¹³ cfu in cycle 3, depending on the track.

For three tracks with solid-phase selection, biotinylated target protein was immobilised on 0.5 mg beads, for 1 h at RT and end-over-end (eoe). Target-containing beads were incubated with 1% (w/v) BSA in PBS-T for 30 min at RT and eoe and then washed with PBS-T, before the addition of pre-incubated phage stock and Trastuzumab to perform the selection for 2 h at RT and eoe. For six tracks with liquid-phase selection, pre-incubated phage stock and trastuzumab was added to biotinylated target protein to perform the selection in solution. The selection step was performed at RT and the incubation time for selection was 2 h in cycles 1 and 3 or 1.5 h in cycle 2. Three of these tracks included competition with non-biotinylated target (off-rate selection), which was added after the aforementioned incubation and incubated for an additional 1 h. Phage antigen complexes were captured by incubation with 0.5 mg SA-beads, for 30 min at RT and eoe.

SA-bead captured phage antigen complexes were washed with PBS-T eoe at RT for a total of 5 min, 10 min or 20 min, for each subsequent cycle. For three tracks including competition with non-biotinylated target, the washing in cycles 2-3 was performed using 100 nM non-biotinylated hBCMA in PBS-T for 4 min at RT and eoe. The final wash volumes were transferred to new 1% (w/v) BSA pre-treated tubes to remove sticky binders attached to the tube walls. For six of the selection tracks, antigen-binding phages were eluted by incubation with 0.5 M acetic acid, pH 2.8 for 15 min eoe at RT, followed by transfer of the eluate to new pre-treated tubes and neutralization with equal volume 1 M Tris-HCl, pH 8. For the remaining three selection tracks, antigen-binding phages were eluted by incubating with 0.25 mg/ml trypsin (Gibco Life Technologies) in TBS-T (TRIS buffered saline, 0.1% (v/v) Tween-20) supplemented with 1 mM CaCl₂ for 30 min eoe at RT before transferring the eluate to new pre-treated tubes. Following cycles 1-2, new phage stocks were generated by growing Escherichia coli XL-1 Blue cells (Agilent) in TSB+Y with 10 μg/ml Tet at 37° C. with shaking at 150 rpm until OD₆₀₀=0.5-0.8 and then infecting them (100 ml after cycle 1 and 50 ml after cycle 2) with phage eluate (total volume after cycle 1, half volume after cycle 2). Infected cultures were gently swirled, incubated without shaking for 25 min at 37° C., followed by shaking at 70 rpm at 37° C. for 15 min. The cultures were then centrifuged and resuspended in TSB+Y, before plating on blood agar plates (40 g/l blood agar) with 100 μg/ml Carb and 1% (w/v) glucose, and incubation for 16-18 h at 37° C. Bacterial colonies were collected to TSB+Y by scraping and inoculated to 200 ml TSB+Y with 100 μg/ml Carb and grown at 37° C. with shaking at 150 rpm until OD₆₀₀=0.5. 30 ml were superinfected with M13KO7 helper phage (MOI 8) or KM13 helper phage (MOI of 33 in cycle 1 and MOI of 5 in cycle 2), gently swirled, incubated without shaking for 25 min at 37° C., followed by shaking at 70 rpm at 37° C. for 15 min. The cultures were centrifuged and resuspended in 150 ml TSB+Y with 100 μg/ml Carb and 1 mM IPTG. 25 μg/ml Kan was added 2 h after inoculation. Cultures were incubated at 37° C. with shaking at 150 rpm for 16-18 h. The phage library stock was harvested by precipitation with 20% (w/v) PEG6000/2.5 M NaCl twice. The titre of the stock was measured by spot titration and polymerase chain reaction-screening was used to analyse the percentage of phage particles carrying phagemids with the affibody insert.

### Monoclonal Phage ELISA.

Following cycle 3, 20-21 bacterial colonies generating correctly sized PCR products and equally representing the nine selection tracks were individually grown in 500 μl TSB+Y/Carb in 96-well deep-well plates at 30° C. with shaking at 250 rpm for 16-18 h. 30 μl of overnight culture was inoculated to 720 μl TSB+Y/Carb in a new 96-well deep-well plate and incubated for 2 h at 37° C., 250 rpm, before superinfection by addition of M13K07 helper phage (MOI 8) in 100 μl TSB+Y/Carb per well. Plates were incubated for 30 min at 37° C. without shaking. Finally, 150 μl TSB+Y/Carb/IPTG/Kan was added per well. Final concentrations were 100 μg/ml Carb, 1 mM IPTG and 25 μg/ml Kan. The deep-well plates were incubated at 37° C., 250 rpm, for 16-18 h. Phage supernatants were collected by centrifugation the next day.

### Monoclonal Phage-ELISA Screening of Optimised Candidates.

A MaxiSorp ELISA plate (Clear Flat-Bottom Immuno Nonsterile 384-Well Plates, cat. no. 464718, Thermo Fisher Scientific) was coated at 4° C. with slow shaking for 16-18 h with 30 μl 1 μg/ml biotinylated hBCMA-Fc, 20 μg/ml HSA (for assessment of proper display of the expression cassette containing a tripartite fusion protein including an affibody, an albumin binding domain and the truncated (pAffi-1) or full-length (pAffi-100)protein 3), 10 μg/ml SA or 10 μg/ml trastuzumab (as a control for binding to the Fc tag) in 100 mM sodium carbonate buffer pH 9.6 per well (¼ of the wells with each coated protein). The coating solution was removed and the plate was washed two times with PBS-T, and blocked with PBS supplemented with 1% (w/v) BSA for 1 h at RT with slow shaking. The blocking solution was discarded, and the wells were incubated with 10 μl phage supernatant diluted 1:3 with 20 μl PBS-T for 1 h at RT with slow shaking. The supernatant was removed and the plate washed three times with PBS-T. Following addition of 30 μl 1:5000 α-M13-HRP (Sigma-Aldrich, Stockholm, Sweden) in PBS-T and incubated for 30 min at RT with slow shaking, the plate was washed twice with PBS-T and once with PBS. 30 μl TMB substrate (TMB Substrate Kit, Thermo Fisher Scientific) was added to each well. The reactions were stopped by adding 30 μl 2 M H₂SO₄ after 15-25 min. Absorbance at 450 nm was measured using a CLARIOstar microplate reader (BMG Labtech, Ortenberg, Germany). Candidates that were considered as ELISA-positive clones had high HSA signal and relatively high signal to hBCMA compared to the SA and trastuzumab controls.

DNA sequencing. Following ELISA screening, 120 ELISA-positive clones were sent for sequencing by Sanger sequencing (Microsynth).

### Results

To isolate second-generation polypeptides binding to hBCMA, two affibody phage libraries were constructed primarily based on the hBCMA binding clone Fa-G6 and the results from the alanine scan. The two libraries were used as the input in the first cycle in a three-cycle selection campaign as described in Material and methods (above). After the third cycle of selections, the eluates from the nine selection tracks were used to infect E. coli cells to obtain individual colonies on Carb plates. 20-21 clones from each track were randomly picked for binding analyses using a phage-ELISA experiment. The majority of the analysed clones showed strong ELISA signals to HSA and hBCMA, and low signals for the controls used, and were subjected to DNA sequencing. The DNA sequencing of the phage ELISA positive clones identified 107 unique variants. The amino acid sequences of these 107 hBCMA-binding polypeptide variants are listed in Table 1 and in the sequence listing as SEQ ID NOs 2 (clone 1-E6) and 23-128.

Biological Example 6: Characterisation of Second-Generation Candidate hBCMA Binding Polypeptides

In this Example, a subset of 18 second-generation polypeptide variants (named 1-E6, 1-E1, 1-A5, 1-A7, 1-F9, 1-F10, 2-B1, 2-C4, 2-D3, 2-H2, 2-A7, 2-E5, 2-E6, 2-G5, 2-A10, 2-B10, 2-C5 and 2-D10) corresponding to SEQ IDs 2 and 23-39 were subcloned, expressed, purified as affibody-His₆ fusion proteins and initially assessed for their binding to hBCMA-Fc by surface plasmon resonance. Circular dichroism (CD) spectroscopy was used to assess the secondary structure contents and their thermal denaturation profiles.

### Materials and Methods

Cloning of the Second-Generation hBCMA Binding Clones.

The DNA fragments encoding a subset of 18 new candidate hBCMA binders [SEQ IDs 2 and 23-39], equally representing the nine selection tracks, were amplified from the library vector pAffi-1 with specific primers, designed to introduce overhangs complementary to ends of linearised expression vectors. In-Fusion HD Cloning Kit (Takara Bio, Gothenburg, Sweden) was used to clone 18 monomeric polypeptide constructs with a C-terminal His₆ tag. The 18 DNA constructs containing SEQ IDs 2 and 23-39 in the format [polypeptide sequence]-YYHHHHHH were sequence verified using Sanger sequencing (Microsynth).

### Expression and Purification.

E. coli BL21 (DE3) cells were transformed with plasmids containing DNA constructs encoding for the 18 polypeptide variants, each equipped with a C-terminal His₆ tag, as well as the parental Fa-G6-His₆ construct, and cultivated in 10 ml TSB+Y with 25 μg/ml Kan at 37° C. with shaking at 150 rpm for 16-18 h. The cultures were inoculated 1:100 in 200 ml TSB+Y with 25 μg/ml Kan and grown at 37° C. with shaking at 150 rpm until OD₆₀₀=0.6-1. IPTG was added to final concentration 1 mM to induce protein expression. The cultivations were incubated at 25° C. with shaking at 150 rpm for 16-18 h and the cells were harvested by centrifugation. The cell pellets were resuspended in denaturing lysis buffer. After incubation at 37° C. for 2 h with shaking at 150 rpm, the cells were centrifuged and the denatured protein from the supernatant of the cell lysates was added to tubes containing 3 ml HisPur Cobalt IMAC Resin (cat. no. 89966, Thermo Scientific). Supernatant was incubated with resin for 30 min at RT with end-over-end rotation. Contaminants were removed by washing three times with wash buffer and hBCMA binding polypeptides were subsequently eluted with elution buffer by incubation at RT for 10 min with end-over-end rotation. Following IMAC purification, the protein buffer was exchanged to PBS using PD-10 desalting columns (cat. no. 17085101, Cytiva). SDS-PAGE analysis was performed to confirm the purity and concentrations, which were measured by absorbance at 280 nm using extinction coefficients calculated from amino acid sequences, of the purified proteins (NuPAGE, 4 to 12%, Bis Tris, Invitrogen). The low molecular weight marker was from Cytiva (Art. no. 17044601).

Biosensor analyses of second generation Fa-G6 variants. A Biacore T200 instrument (Cytiva) was used to analyse real-time interaction of the 18 second-generation variants, expressed and purified as polypeptide-His₆ (SEQ IDs 2 and 23-39 in the format [polypeptide sequence]-YYHHHHHH) with hBCMA. The protein ligand hBCMA-Fc (Sino Biological Inc., cat. no. 10620-H15H) was immobilised on a Series S CM5 sensor chip (Cytiva) by amine coupling (3,500 RUs, using the manufacturer's instructions. One flow cell was activated and deactivated to be used as a reference cell. Analytes were diluted in the running buffer PBS-T before binding analysis was performed at 25° C. and a flow rate of 30 μl/min. After each injection series the flow cells were regenerated by the injection of 10 mM HCl. The 18 second-generation variants and the parental Fa-G6 binder were injected at 100 nM over the hBCMA-Fc surface.

CD analyses. A Chirascan CD Spectrometer (Applied Photophysics, Leatherhead, United Kingdom) was used to determine the secondary structure content of the Fa-G6 binder and second-generation binders, by recoding the CD spectra at wavelength range 195-260 nm at 20° C., as well as their thermal denaturation profiles, by recording the CD ellipticity at 221 nm during heating from 20 to 92° C.

### Results

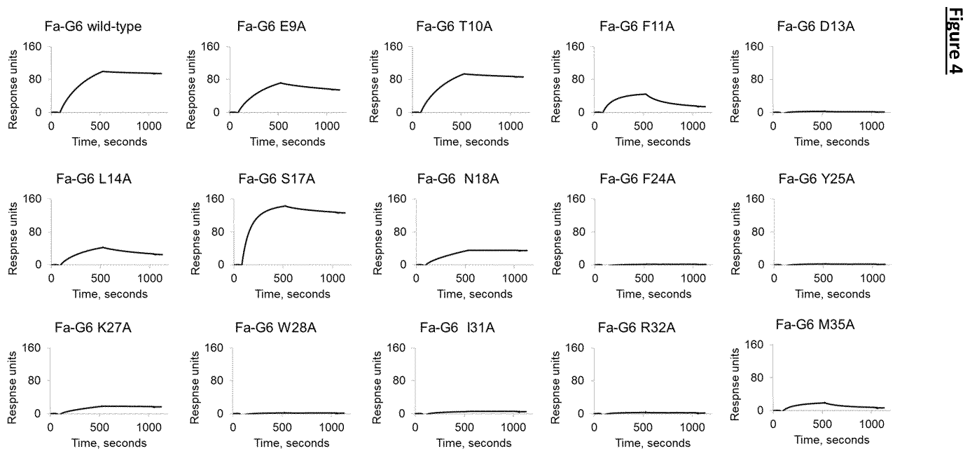

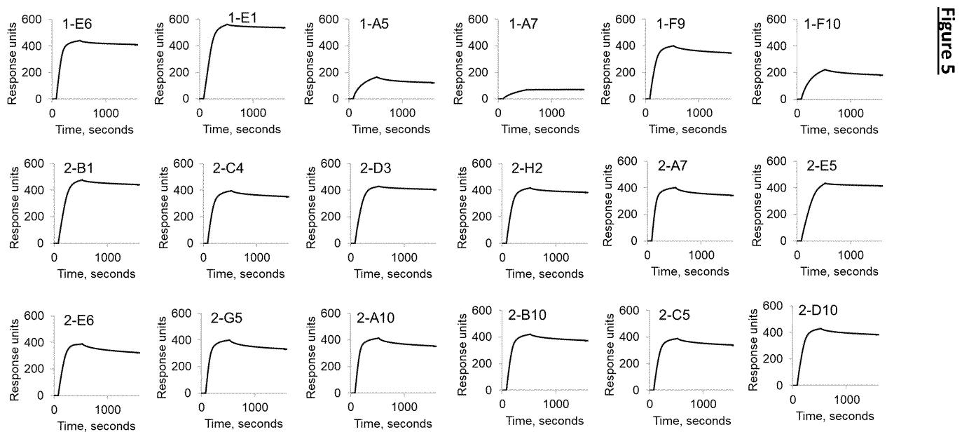

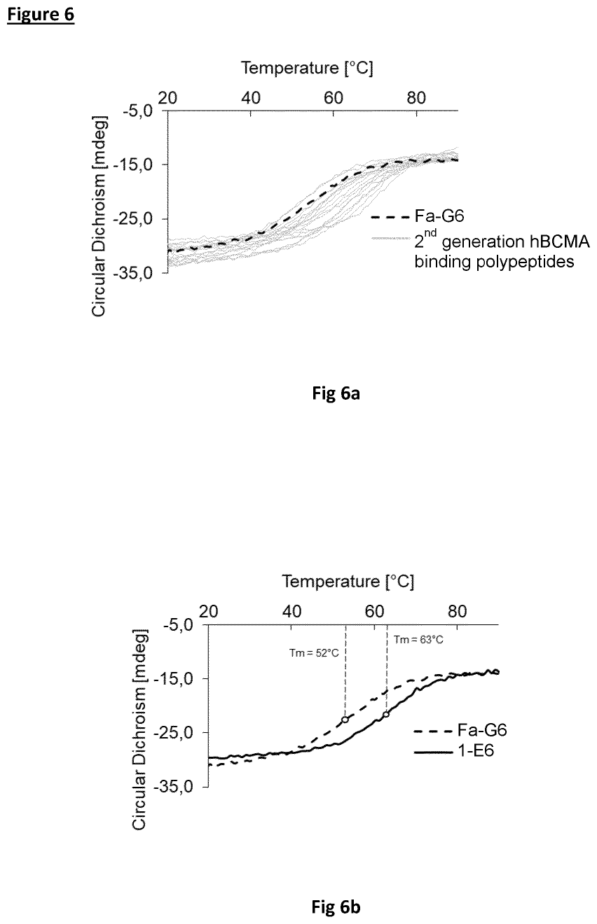

To investigate if a subset of new candidate hBCMA binding variants emerging from the selections from the second-generation libraries and identified via the phage-ELISA and DNA sequencing showed hBCMA target binding when expressed as soluble proteins, 18 new variants [SEQ IDs 2 and 23-39] chosen to equally represent the nine selection tracks were subcloned, cytoplasmically expressed in E. coli, and purified as affibody-His₆ fusion proteins. IMAC-purified fusion proteins, employing the His₆ gene fusion partner in the proteins, were analysed by SDS-PAGE which showed that all 18 proteins were of high purity and had approximate molecular weights in accordance with their amino acid sequences. The 18 candidate optimised variants were separately injected over a sensor chip immobilised with hBCMA-Fc at a single concentration of 100 nM. The resulting sensorgrams (FIG. 5) showed that the 18 new variants all bound to hBCMA. The CD spectra obtained showed that the analysed binders all had a high α-helix secondary structure content at 20° C., and from the thermal denaturation profiles (FIG. 6a) of the second-generation hBCMA binders an improved resistance to heat denaturation could be observed for the majority of these binders, compared to the parental Fa-G6 clone, also included in the analysis. One of the new variants, clone 1-E6, showed a melting temperature (Tm) of 63° C., determined by variable temperature measurement, which is 11° C. higher than that observed for the parental Fa-G6 clone (Tm of 52° C.). This is shown in FIG. 6b. Taken together, the results showed that a pool of new hBCMA binding polypeptides based on the parental Fa-G6 binder had been generated, with diversified biophysical properties compared to Fa-G6.

### Biological Example 7: Anti-hBCMA Drug Conjugates are Cytotoxic to BCMA⁺ Multiple Myeloma Cells

In this Example, an hBCMA binder-drug conjugate was synthesised and its cell binding and cancer cell killing abilities tested using multiple myeloma cells.

MMAF Conjugation to Produce an hBCMA Binder-Drug Conjugate

The hBCMA binder, 1-E6 SEQ ID 2 (6756.5 Da), was synthetically produced at Almac Science Limited (Scotland). The protein was comprised of 59 natural amino acids including the free thiol-bearing terminal cysteine residue. The toxin of choice was MMAF with a non-cleavable MPB linker. The unbound cysteine-residue thiol functionality was to be exploited by thiol-Michael addition reaction to the linker's electrophilic maleimide moiety. Buffer exchange and purifications were performed in Amicon Ultra centrifugal filters (3 kDa cut-off). HPLC-MS on reaction progress was performed using an Agilent 1100 series Liquid Chromatograph/Mass Selective Detector (MSD) (Single Quadrupole) equipped with an electrospray interface and a UV diode array detector. Analyses were performed by using an ACE 3 C8 (3.0×50 mm) column with a gradient of acetonitrile in 0.1% aqueous TFA over 3 min and a flow of 1 mL/min. Purity and ID analysis was performed using Agilent 1290 Ultra Performance Liquid Chromatograph/Quadrople Time of Flight (Q-ToF) mass spectrometry detector. Analyses were performed by using an ACE 3 C8 5μ (3.0×50 mm) with UV detection at 280 nm and gradient of acetonitrile in 0.1% aqueous formic acid and flow of 0.8 mL/min. An Fa-G6 null variant of the polypeptide devoid of binding ability was also synthetically synthesized using the same methods as above. TCEP HCl (8.47 mM) Tris(2-carboxyethyl)phosphineHCl (4.24 mg, 14.8 μM) was dissolved in PBS pH 7.4 (2 mL, 10 mM) and gently vortexed. MPB-MMAF solution (2.06 mM) MPB-MMAF (4.00 mg, 4.11 μmol) was dissolved in dry DMF (2.0 mL) and gently vortexed. Both solutions were prepared freshly before use. hBCMA binder 1-E6 (10 mg, 1.48 μmol) was weighed into a glass vial and dissolved in PBS buffer (4 mL, pH 7.4, 10 mM) and TCEP (2.0 mL, 10 molar equivalents) was carefully added. The reaction mixture was gently agitated at room temperature for 30 min. Then the solution was divided into two centrifuge filtration units and centrifuged at 5000 rpm (6° C.) for 25 min. Each concentrate (0.5 mL) was diluted with PBS pH 7.4 (3.5 mL) and the centrifugation step was repeated. The reaction mixture was transferred from the filter units to a 10 mL glass vial, then PBS pH 7.4 (4 mL) and MPB-MMAF in dry DMF (2 mL, 2.78 molar equivalents) were added. The reaction mixture was gently agitated at room temperature for 2 h. The reaction mixture was diluted with 10% DMF in PBS (2.0 mL, pH 7.4) and the resulting solution was centrifuged in the filter units at 5000 rpm for 25 min. Eight cycles of washing by this filtration method were performed, whereupon no trace of nonconjugated toxin was detected by LCMS. Then, the buffer was exchanged to arginine containing buffer pH 7.8 (50 mM Arg, 75 mM NaCl, 2% (w/w) sucrose, 0.01% (w/w)polysorbate 20. The buffer exchange was performed in centrifugal filter units at 5000 rpm (6° C.) for 25 min. The buffer exchange step was repeated 7 times to give a material with UPLC UV purity 87% and HRMS (deconvoluted): 7729.

### Capping of 1-E6 and Fa-G6 Null Variant Polypeptides

The protocol to produce a capped non-toxic version of the polypeptide was essentially the same as for the MMAF conjugation with the only major difference that the free thiol-bearing terminal cysteine residue was reacted with 1-ethylpyrrole-2,5-dione in the molar equivalents of 1.50 compared to the linker drug. The procedure gave the capped versions with UPLC UV purity 99% and HRMS (deconvoluted): 6681 for 1-E6 and 6637 for Fa-G6.

### BCMA Binding ELISA

96 well assay plate (3690, Costar) was coated with 1 μg/ml BCMA (193-BC-050, R&D) and incubated overnight at 4° C. After blocking, serial dilutions of 1-E6-capped and 1-E6-MPB-MMAF in blocking buffer (37528, Thermo Scientific) was added and incubated. Then anti-affibody antibody (20.1000.01.0005, Affibody AB) and anti-goat Antibody-HRP (PA1-28664, Invitrogen) in blocking buffer was used. The reaction was developed using 1-Step™ Ultra TMB-ELISA Substrate Solution (34028, Thermo Scientific)), and stopped by adding 2M sulfuric acid. The absorbance was read at 450 nm using SpectraMax plate reader. All incubation steps were performed at room temperature for 1 hour, and the assay plate was washed 4 times with PBST after each incubation.

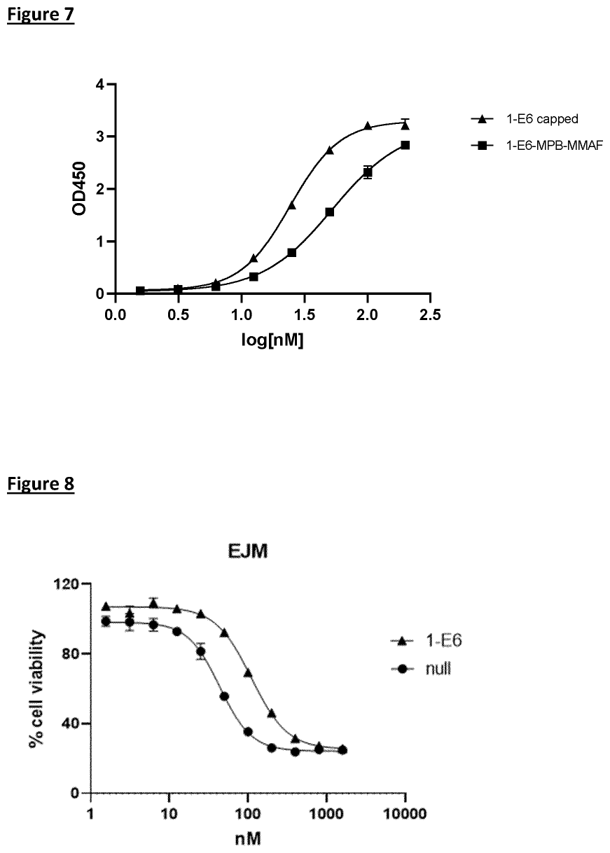

MMAF conjugation reduced the affinity of 1E6 to BCMA to some extent (FIG. 7). Still, it could be demonstrated that 1-E6-MPB-MMAF showed binding affinity to BCMA.

### Cell Viability of BCMA+ MM.1S Cells

4×10⁵ MM.1S cells (CRL-2974, ATCC) were treated with 1-E6-MPB-MMAF at various concentrations for 72 hours in the presence of 5 μM 1-E6 or Fa-G6_W24A, F28A capped. DMSO treated and 200 μM Hydrochloride Chlorpromazine (C8138, Sigma-Aldrich) treated wells served as the controls of living and dead cells, respectively. After incubation, CellTiter-Glo (G924B, Promega) was dispensed into the cells, and luminescence was read using PerkinElmer En Vision Multilabel Plate Reader.

1-E6-MPB-MMAF was cytotoxic to the BCMA+ cells in a dose dependent manner (FIG. 8). The potency was lower in the presence of a competing 1-E6 as compared to the non-competitive Fa-G6 null variant of the polypeptide. Thus, it could be demonstrated that a drug conjugate of 1-E6 could be useful to target proliferation of BCMA+ myeloma cells.

Biological Example 8: HBCMA Engager of the Invention Bound to an hCD16a Binder Induces Immune Cell Activation in the Presence of Tumour Cells

Experiments were performed to evaluate the propensity of an anti-hBCMA engager of the invention attached to hCD16a binders to evoke an IFN□□ response in cocultures of human PBMCs and the BCMA positive multiple myeloma cells. Thawed and overnight rested PBMCs (4W-270, Lonza) were co-cultured with MM.1S cells (CRL-2974, ATCC) at effector to target ratio (E:T) of 25:1 in V shaped 96-well plate (249935, Thermo Scientific). Total 4×10⁵ cells in 200 μl/well were treated with 200 nM hBCMA×hCD16a dual engagers or 200 nM Elotuzumab (300 mg Empliciti, Bristol-Myers Squibb) for 4 hours at 37° C. in a humidified 5% CO₂ incubator. Supernatant was harvested followed by centrifugation 600 g for 5 min. IFN-γ level was determined by enzyme-linked immunoassay (ELISA) using Human IFN-γ ELISA MAX™ Deluxe Set (430104, BioLegend). Data was processed using Excel and plotted using GraphPad Prism 9.

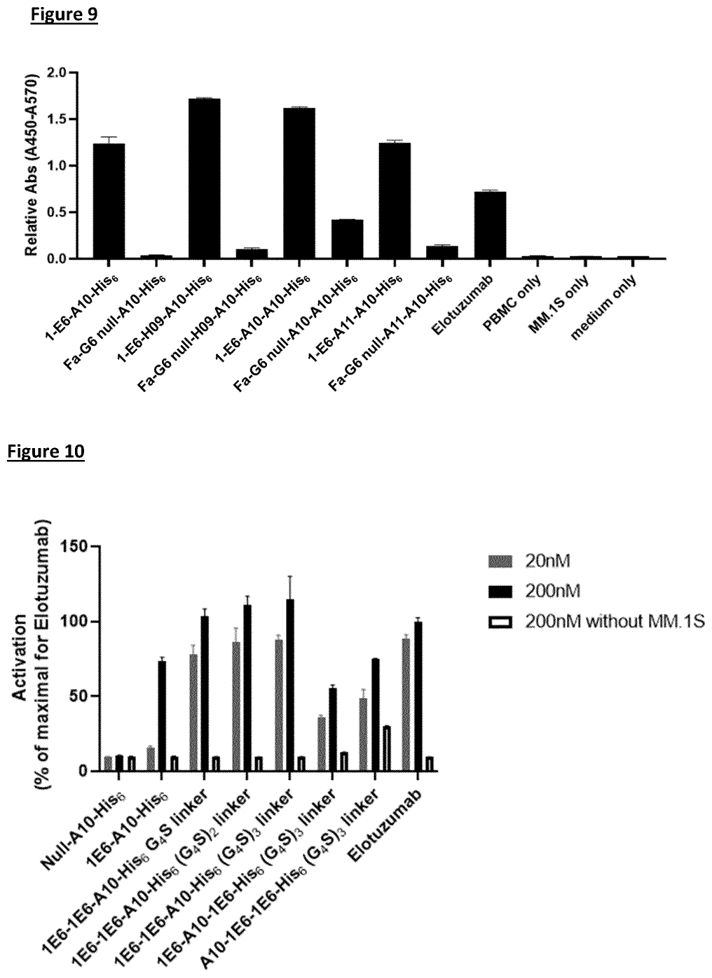

hBCMA×hCD16a dual engager constructs were investigated, containing an hBCMA-binding polypeptide of the invention (1-E6, SEQ ID 2) genetically fused to an hCD16a binding arm composed of a CD16a-binding polypeptide (referred to herein as A10 (SEQ ID 167), H09 (SEQ ID 276) and A11 (SEQ ID 277) in either monomeric or dimeric form. Dual engagers tested were: monomeric A10 (1-E6-A10-His₆ [SEQ ID NO. 130]); heterodimeric H09-A10 (1-E6-H09-A10-His₆ [SEQ ID NO. 131]); homodimeric A10 (1-E6-A10-A10-His₆ [SEQ ID NO. 132]; and heterodimeric A11-A10 (1-E6-A11-A10-His₆ [SEQ ID NO. 133]). Sequence information and SEQ ID references for each of these four dual engager constructs are given in Table 1 below. Corresponding constructs harbouring a null non-hBCMA-binding polypeptide (Fa-G6 null) were also evaluated. The anti-SLAMF7 monoclonal antibody Elotuzumab was used as a positive control and single cultures of PBMC and MM.1S served as negative controls. The dual engagers containing a BCMA-binding polypeptide all evoked an IFN□ response in cocultures of PBMC and MM. 1S cells, which for all engagers was larger than the response of the positive control Elotuzumab at 200 nM concentration. (FIG. 9). Dual engager constructs devoid of hBCMA binding showed no or limited IFN□ response in cocultures of PBMC and MM.1S cells. These experiments illustrate the hBCMA dependent activation of PBMC against the BCMA positive MM.1S myeloma cell line induced by the anti-hBCMA×CD16a NK cell engagers of the invention.

The CD16a-binding polypeptides referred to herein as A10, H09 and A11 have the sequences:

| | | SEQ |
|---|---|---|
| Name | Sequence | ID |
| A10 | VDNKFNKEVQMAQFEIRKLPNLNHHQSFAFIKS | 167 |
| | LMDDPSQSANLLAEAKKLNDAQAPK | |
| A11 | VDNKFNKEQFYARDEIDLLPNLNEDQKWAFYMS | 276 |
| | LIDDPSQSANLLAEAKKLNDAQAPK | |
| H09 | VDNKFNKEFWIAESEIESLPNLNIYQKWAFKYS | 277 |
| | LADDPSQSANLLAEAKKLNDAQAPK | |

Biological Example 9: HBCMA Engager of the Invention Bound to an hCD16a Binder Induces CD16 Mediated Activation in the Presence of Tumour Cells

Experiments were performed to evaluate the propensity of an anti-hBCMA engager of the invention attached to hCD16a binder A10 to evoke a CD16 dependent response in a reporter assay using the commercially available Jurkat-Lucia NFAT cell line from Invivogen. Culturing was according to manufacturer's specifications using 100,000 cells/mL of Jurkat-Lucia NFAT cell line and 50,000 cells/mL of MM.1S. Cells were allowed to incubate with engager constructs for 24 h.

Anti-hBCMA dual engager constructs containing either one or two hBCMA-binding polypeptides of the invention (1-E6) genetically fused to an hCD16a binding arm A10 were evaluated. Sequence information and SEQ ID references for these dual engager constructs are given in Table 1 below [SEQ ID NOs 130, 134-138]. A construct harbouring a null non-hBCMA-binding polypeptide (Fa-G6 null, denoted as “null” in FIG. 10) were also evaluated. The anti-SLAMF7 monoclonal antibody Elotuzumab was used as a positive control. The dual engagers containing a BCMA-binding polypeptide all evoked a CD16 mediated response in the presence of MM.1S cells. (FIG. 10). The dual engager construct devoid of hBCMA binding showed no response. These experiments illustrate the hBCMA dependent activation by the anti-hBCMA×CD16a NK cell engagers of the invention.

TABLE 1

| | | SEQ |
|---|---|---|
| Name | Sequence | ID |
| Fa-G6 | VDNKFNKEETFADLEISNLPNLNFYQKWAFIRSLMDDPSQ | 1 |
| | SANLLAEAKKLNDAQAPK | |
| 1-E6 | VDNKFNKENQFADEEIAALPNLNFYQKWAFIRKLMDDPS | 2 |
| | QSANLLAEAKKLNDAQAPK | |
| His₆-Fa-G6- | GSSHHHHHHYYLEVDNKFNKEETFADLEISNLPNLNFYQ | 3 |
| ABD_{WT} | KWAFIRSLMDDPSQSANLLAEAKKLNDAQAPKVDYVLA | |
| | EAKVLANRELDKYGVSDYYKNLINNAKTVEGVKALIDEI | |
| | LAALP | |
| Fa-G6-His₆ | VDNKFNKEETFADLEISNLPNLNFYQKWAFIRSLMDDPSQ | 4 |
| | SANLLAEAKKLNDAQAPKYYHHHHHH | |
| Fa-G6 [−2 aa]- | NKFNKEETFADLEISNLPNLNFYQKWAFIRSLMDDPSQSA | 5 |
| His₆ | NLLAEAKKLNDAQAPKYYHHHHHH | |
| Fa-G6 [−3 aa]- | KFNKEETFADLEISNLPNLNFYQKWAFIRSLMDDPSQSAN | 6 |
| His₆ | LLAEAKKLNDAQAPKYYHHHHHH | |
| Fa-G6 [−4 aa]- | FNKEETFADLEISNLPNLNFYQKWAFIRSLMDDPSQSANL | 7 |
| His₆ | LAEAKKLNDAQAPKYYHHHHHH | |
| Fa-G6 [−5 aa]- | NKEETFADLEISNLPNLNFYQKWAFIRSLMDDPSQSANLL | 8 |
| His₆ | AEAKKLNDAQAPKYYHHHHHH | |
| Fa-G6 E9A | GSSHHHHHHYYLEVDNKFNKEATFADLEISNLPNLNFYQ | 9 |
| | KWAFIRSLMDDPSQSANLLAEAKKLNDAQAPK | |
| Fa-G6 T10A | GSSHHHHHHYYLEVDNKFNKEEAFADLEISNLPNLNFYQ | 10 |
| | KWAFIRSLMDDPSQSANLLAEAKKLNDAQAPK | |
| Fa-G6 F11A | GSSHHHHHHYYLEVDNKFNKEETAADLEISNLPNLNFYQ | 11 |
| | KWAFIRSLMDDPSQSANLLAEAKKLNDAQAPK | |
| Fa-G6 D13A | GSSHHHHHHYYLEVDNKFNKEETFAALEISNLPNLNFYQ | 12 |
| | KWAFIRSLMDDPSQSANLLAEAKKLNDAQAPK | |
| Fa-G6 L14A | GSSHHHHHHYYLEVDNKFNKEETFADAEISNLPNLNFYQ | 13 |
| | KWAFIRSLMDDPSQSANLLAEAKKLNDAQAPK | |
| Fa-G6 S17A | GSSHHHHHHYYLEVDNKFNKEETFADLEIANLPNLNFYQ | 14 |
| | KWAFIRSLMDDPSQSANLLAEAKKLNDAQAPK | |
| Fa-G6 N18A | GSSHHHHHHYYLEVDNKFNKEETFADLEISALPNLNFYQ | 15 |
| | KWAFIRSLMDDPSQSANLLAEAKKLNDAQAPK | |
| Fa-G6 F24A | GSSHHHHHHYYLEVDNKFNKEETFADLEISNLPNLNAYQ | 16 |
| | KWAFIRSLMDDPSQSANLLAEAKKLNDAQAPK | |
| Fa-G6 Y25A | GSSHHHHHHYYLEVDNKFNKEETFADLEISNLPNLNFAQ | 17 |
| | KWAFIRSLMDDPSQSANLLAEAKKLNDAQAPK | |
| Fa-G6 K27A | GSSHHHHHHYYLEVDNKFNKEETFADLEISNLPNLNFYQ | 18 |
| | AWAFIRSLMDDPSQSANLLAEAKKLNDAQAPK | |
| Fa-G6 W28A | VDNKFNKEETFADLEISNLPNLNFYQKAAFIRSLMDDPSQ | 19 |
| | SANLLAEAKKLNDAQAPK | |
| Fa-G6 I31A | GSSHHHHHHYYLEVDNKFNKEETFADLEISNLPNLNFYQ | 20 |
| | KWAFARSLMDDPSQSANLLAEAKKLNDAQAPK | |
| Fa-G6 R32A | GSSHHHHHHYYLEVDNKFNKEETFADLEISNLPNLNFYQ | 21 |
| | KWAFIASLMDDPSQSANLLAEAKKLNDAQAPK | |
| Fa-G6 M35A | GSSHHHHHHYYLEVDNKFNKEETFADLEISNLPNLNFYQ | 22 |
| | KWAFIRSLADDPSQSANLLAEAKKLNDAQAPK | |
| 1-E1 | VDNKFNKEEIFADREIAFLPNLNFYQKWAFIRKLMDDPSQ | 23 |
| | SANLLAEAKKLNDAQAPK | |
| 1-A5 | VDNKFNKESQFADEEIEALPNLNFYQKWAFIRSLMDDPSQ | 24 |
| | SANLLAEAKKLNDAQAPK | |
| 1-A7 | VDNKFNKEQEFADYEIATLPNLNFYQKWAFIRSLMDDPS | 25 |
| | QSANLLAEAKKLNDAQAPK | |
| 1-F9 | VDNKFNKEDSFADTEIAKLPNLNFYQKWAFIRSLMDDPS | 26 |
| | QSANLLAEAKKLNDAQAPK | |
| 1-F10 | GDNKFNKEEIFADQEIEALPNLNFYQKWAFIRSLMDDPSQ | 27 |
| | SANLLAEAKKLNDAQAPK | |
| 2-B1 | VDNKFNKEDYFADYEIATLPNLNFYQKWAFIRSLMDDPS | 28 |
| | QSANLLAEAKKLNDAQAPK | |
| 2-C4 | VDNKFNKEEQFADEEIAHLPNLNFYQKWAFIRSLMDDPS | 29 |
| | QSANLLAEAKKLNDAQAPK | |
| 2-D3 | VDNKFNKESMFADIEITKLPNLNFYQKWAFIRKLMDDPSQ | 30 |
| | SANLLAEAKKLNDAQAPK | |
| 2-H2 | VDNKFNKEEVFADEEIASLPNLNFYQKWAFIRSLMDDPSQ | 31 |
| | SANLLAEAKKLNDAQAPK | |
| 2-A7 | VDNKFNKEVREADEEIAALPNLNFYQKWAFIRKLMDDPS | 32 |
| | QSANLLAEAKKLNDAQAPK | |
| 2-E5 | VDNKFNKEHQFADYEIAMLPNLNFYQKWAFIRSLMDDPS | 33 |
| | QSANLLAEAKKLNDAQAPK | |
| 2-E6 | VDNKFNKEHFFADKEIAKLPNLNFYQKWAFIRSLMDDPS | 34 |
| | QSANLLAEAKKLNDAQAPK | |
| 2-G5 | VDNKFNKEKAFADTEIVNLPNLNFYQKWAFIRSLMDDPS | 35 |
| | QSANLLAEAKKLNDAQAPK | |
| 2-A10 | VDNKFNKESEFADAEIANLPNLNFYQKWAFIRKLMDDPS | 36 |
| | QSANLLAEAKKLNDAQAPK | |
| 2-B10 | GDNKFNKEENFADHEIALLPNLNFYQKWAFIRKLMDDPS | 37 |
| | QSANLLAEAKKLNDAQAPK | |
| 2-C5 | VDNKFNKEDSHADEEIAALPNLNFYQKWAFIRKLMDDPS | 38 |
| | QSANLLAEAKKLNDAQAPK | |
| 2-D10 | VDNKFNKESKFADEEIAKLPNLNFYQKWAFIRSLMDDPS | 39 |
| | QSANLLAEAKKLNDAQAPK | |
| 1-E2 | VDNKFNKESSADLEIAHLPNLNFYQKWAFIRSLMDDPSQS | 40 |
| | ANLLAEAKKLNDAQAPK | |
| 1-A2 | VDNKFNKEQQFADEEIAMLPNLNFYQKWAFIRKLMDDPS | 41 |
| | QSANLLAEAKKLNDAQAPK | |
| 1-B1 | VDNKFNKEHHFADIEISHLPNLNFYQKWAFIRSLMDDPSQ | 42 |
| | SANLLAEAKKLNDAQAPK | |
| 1-B2 | VDNKFNKESQFADQEIAALPNLNFYQKWAFIRKLMDDPS | 43 |
| | QSANLLAEAKKLNDAQAPK | |
| 1-B3 | VDNKFNKEEYFADSEIAQLPNLNFYQKWAFIRKLMDDPS | 44 |
| | QSANLLAEAKKLNDAQAPK | |
| 1-C1 | VDNKFNKEEKFADVEIAYLPNLNFYQKWAFIRKLMDDPS | 45 |
| | QSANLLAEAKKLNDAQAPK | |
| 1-C2 | VDNKFNKETEFADSEIVRLPNLNFYQKWAFIRSLMDDPSQ | 46 |
| | SANLLAEAKKLNDAQAPK | |
| 1-C3 | VDNKFNKETTFADQEIASLPNLNFYQKWAFIRKLMDDPS | 47 |
| | QSANLLAEAKKLNDAQAPK | |
| 1-D1 | VDNKFNKEYKFADYEIVALPNLNFYQKWAFIRKLMDDPS | 48 |
| | QSANLLAEAKKLNDAQAPK | |
| 1-D2 | VDNKFNKEFQFADIEIAELPNLNFYQKWAFIRSLMDDPSQ | 49 |
| | SANLLAEAKKLNDAQAPK | |
| 1-D3 | VDNKFNKEQTFADQEIAELPNLNFYQKWAFIRKLMDDPS | 50 |
| | QSANLLAEAKKLNDAQAPK | |
| 1-E3 | VDNKFNKETSFADKEIALLPNLNFYQKWAFIRKLMDDPS | 51 |
| | QSANLLAEAKKLNDAQAPK | |
| 1-F1 | VDNKFNKERMFADIEIQQLPNLNFYQKWAFIRSLMDDPS | 52 |
| | QSANLLAEAKKLNDAQAPK | |
| 1-F2 | VDNKFNKEIMFADQEIVSLPNLNFYQKWAFIRSLMDDPSQ | 53 |
| | SANLLAEAKKLNDAQAPK | |
| 1-F3 | VDNKFNKETQFADQEIAFLPNLNFYQKWAFIRKLMDDPS | 54 |
| | QSANLLAEAKKLNDAQAPK | |
| 1-G1 | VDNKFNKELDFADQEIAYLPNLNFYQKWAFIRSLMDDPS | 55 |
| | QSANLLAEAKKLNDAQAPK | |
| 1-G2 | VDNKFNKELIFADEEIVALPNLNFYQKWAFIRSLMDDPSQ | 56 |
| | SANLLAEAKKLNDAQAPK | |
| 1-H1 | VDNKFNKEHFFADSEIAALPNLNFYQKWAFVRSLMDDPS | 57 |
| | QSANLLAEAKKLNDAQAPK | |
| 1-H2 | VDNKFNKEHNFADQEIANLPNLNFYQKWAFIRSLMDDPS | 58 |
| | QSANLLAEAKKLNDAQAPK | |
| 1-A4 | VDNKFNKEMEFADIEIQSLPNLNFYQKWAFIRSLMDDPSQ | 59 |
| | SANLLAEAKKLNDAQAPK | |
| 1-A6 | VDNKFNKETWFADSEIAQLPNLNFYQKWAFIRSLMDDPS | 60 |
| | QSANLLAEAKKLNDAQAPK | |
| 1-B4 | VDNKFNKEETFADKEIAELPNLNFYQKWAFIRKLMDDPS | 61 |
| | QSANLLAEAKKLNDAQAPK | |
| 1-B6 | VDNKFNKEQEFADVEIATLPNLNFYQKWAFIRKLMDDPS | 62 |
| | QSANLLAEAKKLNDAQAPK | |
| 1-C4 | VDNKFNKEEHFADIEIASLPNLNFYQKWAFIRSLMDDPSQ | 63 |
| | SANLLAEAKKLNDAQAPK | |
| 1-C5 | VDNKFNKEFQFADREIAVLPNLNFYQKWAFIRSLMDDPS | 64 |
| | QSANLLAEAKKLNDAQAPK | |
| 1-C6 | VDNKFNKEITFADSEIASLPNLNFYQKWAFIRSLMDDPSQ | 65 |
| | SANLLAEAKKLNDAQAPK | |
| 1-D4 | VDNKFNKEMEFADQEIVVLPNLNFYQKWAFIRKLMDDPS | 66 |
| | QSANLLAEAKKLNDAQAPK | |
| 1-D5 | VDNKFNKEKSFADNEIAELPNLNFYQKWAFIRKLMDDPS | 67 |
| | QSANLLAEAKKLNDAQAPK | |
| 1-E4 | VDNKFNKETHFADKEIANLPNLNFYQKWAFIRSLMDDPS | 68 |
| | QSANLLAEAKKLNDAQAPK | |
| 1-E5 | VDNKFNKEEQFADMEIASLPNLNFYQKWAFIRKLMDDPS | 69 |
| | QSANLLAEAKKLNDAQAPK | |
| 1-F4 | VDNKFNKEHSFADNEIAALPNLNFYQKWAFIRSLMDDPS | 70 |
| | QSANLLAEAKKLNDAQAPK | |
| 1-F5 | VDNKFNKEATFADLEIEALPNLNFYQKWAFIRKLMDDPS | 71 |
| | QSANLLAEAKKLNDAQAPK | |
| 1-G3 | VDNKFNKEENFADNEIASLPNLNFYQKWAFIRSLMDDPS | 72 |
| | QSANLLAEAKKLNDAQAPK | |
| 1-G4 | VDNKFNKESAFADIEIASLPNLNFYQKWAFIRKLMDDPSQ | 73 |
| | SANLLAEAKKLNDAQAPK | |
| 1-G5 | VDNKFNKEELFADQEIANLPNLNFYQKWAFMRSLMDDPS | 74 |
| | QSANLLAEAKKLNDAQAPK | |
| 1-H3 | VDNKFNKEAVFADEEITNLPNLNFYQKWAFIRSLMDDPS | 75 |
| | QSANLLAEAKKLNDAQAPK | |
| 1-H5 | VDNKFNKETLFADHEIANLPNLNFYQKWAFIRSLMDDPS | 76 |
| | QSANLLAEAKKLNDAQAPK | |
| 1-A8 | VDNKFNKEKHFADEEIAKLPNLNFYQKWAFIRKLMDDPS | 77 |
| | QSANLLAEAKKLNDAQAPK | |
| 1-B7 | VDNKFNKEERFADLEISVLPNLNFYQKWAFIRKLMDDPS | 78 |
| | QSANLLAEAKKLNDAQAPK | |
| 1-B8 | VDNKFNKEMTFADYEISVLPNLNFYQKWAFIRSLMDDPS | 79 |
| | QSANLLAEAKKLNDAQAPK | |
| 1-C7 | VDNKFNKEVQFADEEIAMLPNLNFYQKWAFIRKLMDDPS | 80 |
| | QSANLLAEAKKLNDAQAPK | |
| 1-C8 | VDNKFNKEELFADEEIQALPNLNFYQKWAFIRSLMDDPSQ | 81 |
| | SANLLAEAKKLNDAQAPK | |
| 1-D6 | VDNKFNKEDKFADEEIAALPNLNFYQKWAFIRSLMDDPS | 82 |
| | QSANLLAEAKKLNDAQAPK | |
| 1-D7 | VDNKFNKEEHFADEEIASLPNLNFYQKWAFIRKLMDDPS | 83 |
| | QSANLLAEAKKLNDAQAPK | |
| 1-D8 | VDNKFNKEERFADEEIASLPNLNFYQKWAFIRKLMDDPS | 84 |
| | QSANLLAEAKKLNDAQAPK | |
| 1-E7 | VDNKFNKEYRFADTEIADLPNLNFYQKWAFIRKLMDDPS | 85 |
| | QSANLLAEAKKLNDAQAPK | |
| 1-E8 | VDNKFNKEQSFADEEIAFLPNLNFYQKWAFIRSLMDDPSQ | 86 |
| | SANLLAEAKKLNDAQAPK | |
| 1-F8 | VDNKFNKEELFADTEIAQLPNLNFYQKWAFIRSLMDDPSQ | 87 |
| | SANLLAEAKKLNDAQAPK | |
| 1-G6 | VDNKFNKENQFADIEIA YLPNLNFYQKWAFIRSLMDDPSQ | 88 |
| | SANLLAEAKKLNDAQAPK | |
| 1-H6 | VDNKFNKEHFFADTEIAFLPNLNFYQKWAFIRKLMDDPS | 89 |
| | QSANLLAEAKKLNDAQAPK | |
| 1-H7 | VDNKFNKEAVFADHEIAFLPNLNFYQKWAFIRKLMDDPS | 90 |
| | QSANLLAEAKKLNDAQAPK | |
| 1-H8 | VDNKFNKENEFADEEIALLPNLNFYQKWAFIRSLMDDPSQ | 91 |
| | SANLLAEAKKLNDAQAPK | |
| 1-A9 | VDNKFNKEDDFADEEIAALPNLNFYQKWAFIRSLMDDPS | 92 |
| | QSANLLAEAKKLNDAQAPK | |
| 1-A10 | VDNKFNKETDFADVEIVALPNLNFYQKWAFIRKLMDDPS | 93 |
| | QSANLLAEAKKLNDAQAPK | |
| 1-C9 | VDNKFNKEERFADMEISNLPNLNFYQKWAFIRSLMDDPS | 94 |
| | QSANLLAEAKKLNDAQAPK | |
| 1-C11 | VDNKFNKEEAFADTEIAALPNLNFYQKWAFIRSLMDDPS | 95 |
| | QSANLLAEAKKLNDAQAPK | |
| 1-D10 | VDNKFNKEETEADMEISNLPNLNFYQKWAFIRSLMDDPS | 96 |
| | QSANLLAEAKKLNDAQAPK | |
| 1-D11 | VDNKFNKEQKFADDEIAALPNLNFYQKWAFIRSLMDDPS | 97 |
| | QSANLLAEAKKLNDAQAPK | |
| 1-E9 | VDNKFNKEEAFADMEIERLPNLNFYQKWAFIRSLMDDPS | 98 |
| | QSANLLAEAKKLNDAQAPK | |
| 1-E10 | VDNKFNKEINFADEEIAYLPNLNFYQKWAFIRSLMDDPSQ | 99 |
| | SANLLAEAKKLNDAQAPK | |
| 1-H10 | VDNKFNKEEQFADQEIALLPNLNFYQKWAFIRKLMDDPS | 100 |
| | QSANLLAEAKKLNDAQAPK | |
| 2-B2 | VDNKFNKEHAFADQEIAILPNLNFYQKWAFIRSLVDDPSQ | 101 |
| | SANLLAEAKKLNDAQAPK | |
| 1-B12 | VDNKFNKEDTFADNEIAFLPNLNFYQKWAFIRSLMDDPS | 102 |
| | QSANLLAEAKKLNDAQAPK | |
| 2-D1 | VDNKFNKEQYFADYEIAHLPNLNFYQKWAFIRKLMDDPS | 103 |
| | QSANLLAEAKKLNDAQAPK | |
| 1-D12 | VDNKFNKEEHFADLEISALPNLNFYQKWAFIRSLLDDPSQ | 104 |
| | SANLLAEAKKLNDAQAPK | |
| 1-F11 | VDNKFNKEISFADQEIATLPNLNFYQKWAFIRSLMDDPSQ | 105 |
| | SANLLAEAKKLNDAQAPK | |
| 2-G1 | VDNKFNKESNFADYEIAQLPNLNFYQKWAFIRKLIDDPSQ | 106 |
| | SANLLAEAKKLNDAQAPK | |
| 1-G11 | VDNKFNKEYDFADEEITALPNLNFYQKWAFIRKLLDDPSQ | 107 |
| | SANLLAEAKKLNDAQAPK | |
| 2-A4 | VDNKFNKEAQFADIEISFLPNLNFYQKWAFIRSL VDDPSQS | 108 |
| | ANLLAEAKKLNDAQAPK | |
| 2-B3 | VDNKFNKEVDFADEEIAQLPNLNFYQKWAFIRSLMDDPS | 109 |
| | QSANLLAEAKKLNDAQAPK | |
| 2-B4 | VDNKFNKEKEHADEEIAALPNLNFYQKWAFIRSLMDDPS | 110 |
| | QSANLLAEAKKLNDAQAPK | |
| 2-C3 | VDNKFNKEEWFADNEIAELPNLNFYQKWAFIRSLMDDPS | 111 |
| | QSANLLAEAKKLNDAQAPK | |
| 2-D4 | VDNKFNKEEQFADQEITNLPNLNFYQKWAFIRSLMDDPS | 112 |
| | QSANLLAEAKKLNDAQAPK | |
| 2-E3 | VDNKFNKEESFADTEIANLPNLNFYQKWAFIRSLMDDPSQ | 113 |
| | SANLLAEAKKLNDAQAPK | |
| 2-G2 | VDNKFNKEDVEADKEIAQLPNLNFYQKWAFIRKLMDDPS | 114 |
| | QSANLLAEAKKLNDAQAPK | |
| 2-G3 | VDNKFNKERYYADVEIAKLPNLNFFQKWAFIRKLMDDPS | 115 |
| | QSANLLAEAKKLNDAQAPK | |
| 2-H4 | VDNKFNKENEFADEEIAELPNLNFYQKWAFIRSLMDDPSQ | 116 |
| | SANLLAEAKKLNDAQAPK | |
| 2-A5 | VDNKFNKEQFQADMEIAQLPNLNFYQKWAFIRSLMDDPS | 117 |
| | QSANLLAEAKKLNDAQAPK | |
| 2-A6 | VDNKFNKEFAFADEEIAQLPNLNFYQKWAFIRSLMDDPS | 118 |
| | QSANLLAEAKKLNDAQAPK | |
| 2-B7 | VDNKFNKEYKFADVEITDLPNLNFYQKWAFIRSLMDDPS | 119 |
| | QSANLLAEAKKLNDAQAPK | |
| 2-D6 | VDNKFNKEFQADMEISHLPNLNFYQKWAFIRSLMDDPSQ | 120 |
| | SANLLAEAKKLNDAQAPK | |
| 2-F6 | VDNKFNKEEETADMEIARLPNLNFYQKWAFIRSLMDDPS | 121 |
| | QSANLLAEAKKLNDAQAPK | |
| 2-D9 | VDNKFNKEKQQADEEIAHLPNLNFYQKWAFIRSLMDDPS | 122 |
| | QSANLLAEAKKLNDAQAPK | |
| 2-H8 | GDNKFNKEDQFADTEIAALPNLNFYQKWAFIRSLMDDPS | 123 |
| | QSANLLAEAKKLNDAQAPK | |
| 2-E10 | VDNKFNKEVRFADTEIA YLPNLNFYQKWAFIRSLMDDPS | 124 |
| | QSANLLAEAKKLNDAQAPK | |
| 2-H10 | VDNKFNKEDMFADMEIATLPNLNFYQKWAFIRKLMDDPS | 125 |
| | QSANLLAEAKKLNDAQAPK | |
| 2-C11 | VDNKFNKEQSFADEEIAILPNLNFYQKWAFVRSLMDDPS | 126 |
| | QSANLLAEAKKLNDAQAPK | |
| 2-G12 | VDNKFNKEEQYADVEIAYLPNLNFYQKWAFIRKLMDDPS | 127 |
| | QSANLLAEAKKLNDAQAPK | |
| 2-F11 | VDNKFNKEESFADWEIQKLPNLNFYQKWAFIRKLMDDPS | 128 |
| | QSANLLAEAKKLNDAQAPK | |
| His₆-Fa-G6 | GSSHHHHHHYYLEVDNKFNKEETFADLEISNLPNLNFYQ | 129 |
| (“Fa-G6 wild | KWAFIRSLMDDPSQSANLLAEAKKLNDAQAPK | |
| type”) | | |
| 1-E6-A10-His₆ | VDNKFNKENQFADEEIAALPNLNFYQKWAFIRKLMDDPS | 130 |
| | QSANLLAEAKKLNDAQAPKGGGSGGGGSGGGGSGVDNK | |
| | FNKEVQMAQFEIRKLPNLNHHQSFAFIKSLMDDPSQSANL | |
| | LAEAKKLNDAQAPKYYHHHHHH | |
| 1-E6-H09- | VDNKFNKENQFADEEIAALPNLNFYQKWAFIRKLMDDPS | 131 |
| A10-His₆ | QSANLLAEAKKLNDAQAPKGGGSGGGGSGGGGSGVDNK | |
| | FNKEFWIAESEIESLPNLNIYQKWAFKYSLADDPSQSANLL | |
| | AEAKKLNDAQAPKGGGSGGGGSGGGGSGVDNKFNKEV | |
| | QMAQFEIRKLPNLNHHQSFAFIKSLMDDPSQSANLLAEAK | |
| | KLNDAQAPKYYHHHHHH | |
| 1-E6-A10- | VDNKFNKENQFADEEIAALPNLNFYQKWAFIRKLMDDPS | 132 |
| A10-His₆ | QSANLLAEAKKLNDAQAPKGGGSGGGGSGGGGSGVDNK | |
| | FNKEVQMAQFEIRKLPNLNHHQSFAFIKSLMDDPSQSANL | |
| | LAEAKKLNDAQAPKGGGSGGGGSGGGGSGVDNKFNKEV | |
| | QMAQFEIRKLPNLNHHQSFAFIKSLMDDPSQSANLLAEAK | |
| | KLNDAQAPKYYHHHHHH | |
| 1-E6-A11- | VDNKFNKENQFADEEIAALPNLNFYQKWAFIRKLMDDPS | 133 |
| A10-His₆ | QSANLLAEAKKLNDAQAPKGGGSGGGGSGGGGSGVDNK | |
| | FNKEQFYARDEIDLLPNLNEDQKWAFYMSLIDDPSQSANL | |
| | LAEAKKLNDAQAPKGGGSGGGGSGGGGSGVDNKFNKEV | |
| | QMAQFEIRKLPNLNHHQSFAFIKSLMDDPSQSANLLAEAK | |
| | KLNDAQAPKYYHHHHHH | |
| 1-E6-1-E6- | VDNKFNKENQFADEEIAALPNLNFYQKWAFIRKLMDDPS | 134 |
| A10- His₆ G₄S | QSANLLAEAKKLNDAQAPKGGGSGVDNKFNKENQFADE | |
| linker | EIAALPNLNFYQKWAFIRKLMDDPSQSANLLAEAKKLND | |
| | AQAPKGGGSGVDNKFNKEVQMAQFEIRKLPNLNHHQSF | |
| | AFIKSLMDDPSQSANLLAEAKKLNDAQAPKYYHHHHHH | |
| 1-E6-1-E6- | VDNKFNKENQFADEEIAALPNLNFYQKWAFIRKLMDDPS | 135 |
| A10-His₆ | QSANLLAEAKKLNDAQAPKGGGSGGGGSGVDNKFNKEN | |
| (G₄S)₂ linker | QFADEEIAALPNLNFYQKWAFIRKLMDDPSQSANLLAEA | |
| | KKLNDAQAPKGGGSGGGGSGVDNKFNKEVQMAQFEIRK | |
| | LPNLNHHQSFAFIKSLMDDPSQSANLLAEAKKLNDAQAP | |
| | KYYHHHHHH | |
| 1-E6-1-E6- | VDNKFNKENQFADEEIAALPNLNFYQKWAFIRKLMDDPS | 136 |
| A10 -His₆ | QSANLLAEAKKLNDAQAPKGGGSGGGGSGGGGSGVDNK | |
| (G₄S)₃ linker | FNKENQFADEEIAALPNLNFYQKWAFIRKLMDDPSQSAN | |
| | LLAEAKKLNDAQAPKGGGSGGGGSGGGGSGVDNKFNKE | |
| | VQMAQFEIRKLPNLNHHQSFAFIKSLMDDPSQSANLLAEA | |
| | KKLNDAQAPKYYHHHHHH | |
| 1-E6-A10-1- | VDNKFNKENQFADEEIAALPNLNFYQKWAFIRKLMDDPS | 137 |
| E6- His₆ | QSANLLAEAKKLNDAQAPKGGGSGGGGSGGGGSGVDNK | |
| (G₄S)₃ linker | FNKEVQMAQFEIRKLPNLNHHQSFAFIKSLMDDPSQSANL | |
| | LAEAKKLNDAQAPKGGGSGGGGSGGGGSGVDNKFNKEN | |
| | QFADEEIAALPNLNFYQKWAFIRKLMDDPSQSANLLAEA | |
| | KKLNDAQAPKYYHHHHHH | |
| A10-1-E6-1- | VDNKFNKEVQMAQFEIRKLPNLNHHQSFAFIKSLMDDPS | 138 |
| E6- His₆ | QSANLLAEAKKLNDAQAPKGGGSGGGGSGGGGSGVDNK | |
| (G₄S)₃ linker | FNKENQFADEEIAALPNLNFYQKWAFIRKLMDDPSQSAN | |
| | LLAEAKKLNDAQAPKGGGSGGGGSGGGGSGVDNKFNKE | |
| | NQFADEEIAALPNLNFYQKWAFIRKLMDDPSQSANLLAE | |
| | AKKLNDAQAPKYYHHHHHH | |
| 1-E6-A10 (no | VDNKFNKENQFADEEIAALPNLNFYQKWAFIRKLMDDPS | 139 |
| C terminal tag) | QSANLLAEAKKLNDAQAPKGGGSGGGGSGGGGSGVDNK | |
| | FNKEVQMAQFEIRKLPNLNHHQSFAFIKSLMDDPSQSANL | |
| | LAEAKKLNDAQAPK | |
| 1-E6-H09-A10 | VDNKFNKENQFADEEIAALPNLNFYQKWAFIRKLMDDPS | 140 |
| (no C terminal | QSANLLAEAKKLNDAQAPKGGGSGGGGSGGGGSGVDNK | |
| tag) | FNKEFWIAESEIESLPNLNIYQKWAFKYSLADDPSQSANLL | |
| | AEAKKLNDAQAPKGGGSGGGGSGGGGSGVDNKFNKEV | |
| | QMAQFEIRKLPNLNHHQSFAFIKSLMDDPSQSANLLAEAK | |
| | KLNDAQAPK | |
| 1-E6-A10-A10 | VDNKFNKENQFADEEIAALPNLNFYQKWAFIRKLMDDPS | 141 |
| (no C terminal | QSANLLAEAKKLNDAQAPKGGGSGGGGSGGGGSGVDNK | |
| tag) | FNKEVQMAQFEIRKLPNLNHHQSFAFIKSLMDDPSQSANL | |
| | LAEAKKLNDAQAPKGGGSGGGGSGGGGSGVDNKFNKEV | |
| | QMAQFEIRKLPNLNHHQSFAFIKSLMDDPSQSANLLAEAK | |
| | KLNDAQAPK | |
| 1-E6-A11-A10 | VDNKFNKENQFADEEIAALPNLNFYQKWAFIRKLMDDPS | 142 |
| (no C terminal | QSANLLAEAKKLNDAQAPKGGGSGGGGSGGGGSGVDNK | |
| tag) | FNKEQFYARDEIDLLPNLNEDQKWAFYMSLIDDPSQSANL | |
| | LAEAKKLNDAQAPKGGGSGGGGGGGGSGVDNKFNKEV | |
| | QMAQFEIRKLPNLNHHQSFAFIKSLMDDPSQSANLLAEAK | |
| | KLNDAQAPK | |
| 1-E6-1-E6- | VDNKFNKENQFADEEIAALPNLNFYQKWAFIRKLMDDPS | 143 |
| A10 G₄S linker | QSANLLAEAKKLNDAQAPKGGGSGVDNKFNKENQFADE | |
| (no C terminal | EIAALPNLNFYQKWAFIRKLMDDPSQSANLLAEAKKLND | |
| tag) | AQAPKGGGSGVDNKFNKEVQMAQFEIRKLPNLNHHQSF | |
| | AFIKSLMDDPSQSANLLAEAKKLNDAQAPK | |
| 1-E6-1-E6- | VDNKFNKENQFADEEIAALPNLNFYQKWAFIRKLMDDPS | 144 |
| A10 (G₄S)₂ | QSANLLAEAKKLNDAQAPKGGGSGGGGSGVDNKFNKEN | |
| linker (no C | QFADEEIAALPNLNFYQKWAFIRKLMDDPSQSANLLAEA | |
| terminal tag) | KKLNDAQAPKGGGSGGGGSGVDNKFNKEVQMAQFEIRK | |
| | LPNLNHHQSFAFIKSLMDDPSQSANLLAEAKKLNDAQAP | |
| | K | |
| 1-E6-1-E6- | VDNKFNKENQFADEEIAALPNLNFYQKWAFIRKLMDDPS | 145 |
| A10 (G₄S)₃ | QSANLLAEAKKLNDAQAPKGGGSGGGGSGGGGSGVDNK | |
| linker (no C | FNKENQFADEEIAALPNLNFYQKWAFIRKLMDDPSQSAN | |
| terminal tag) | LLAEAKKLNDAQAPKGGGSGGGGSGGGGSGVDNKFNKE | |
| | VQMAQFEIRKLPNLNHHQSFAFIKSLMDDPSQSANLLAEA | |
| | KKLNDAQAPK | |
| 1-E6-A10-1- | VDNKFNKENQFADEEIAALPNLNFYQKWAFIRKLMDDPS | 146 |
| E6 (G₄S)₃ | QSANLLAEAKKLNDAQAPKGGGSGGGGSGGGGSGVDNK | |
| linker (no C | FNKEVQMAQFEIRKLPNLNHHQSFAFIKSLMDDPSQSANL | |
| terminal tag) | LAEAKKLNDAQAPKGGGSGGGGSGGGGSGVDNKFNKEN | |
| | QFADEEIAALPNLNFYQKWAFIRKLMDDPSQSANLLAEA | |
| | KKLNDAQAPK | |
| A10-1-E6-1- | VDNKFNKEVQMAQFEIRKLPNLNHHQSFAFIKSLMDDPS | 147 |
| E6 (G₄S)₃ | QSANLLAEAKKLNDAQAPKGGGSGGGGSGGGGSGVDNK | |
| linker (no C | FNKENQFADEEIAALPNLNFYQKWAFIRKLMDDPSQSAN | |
| terminal tag) | LLAEAKKLNDAQAPKGGGSGGGGSGGGGSGVDNKFNKE | |
| | NQFADEEIAALPNLNFYQKWAFIRKLMDDPSQSANLLAE | |
| | AKKLNDAQAPK | |
| 1-E6-(V1G) | GDNKFNKENQFADEEIAALPNLNFYQKWAFIRKLMDDPS | 168 |
| | QSANLLAEAKKLNDAQAPK | |
| 1-E6-(6-55) | NKENQFADEEIAALPNLNFYQKWAFIRKLMDDPSQSANL | 169 |
| | LAEAKKLNDAQ | |

Table 1 shows the sequences of the polypeptides disclosed in the present application, including some of the dual engagers [SEQ ID Nos 130-147].

TABLE 2

| | | |
|---|---|---|
| Synthetic oligo | GCGCTTTGGCTTGGGTCATCXXXTAAACTXXXYYYGAA | X |
| (used in | GGCXXXXXXTTGXXXXXXGTTCAGGTTCGGCAGXXXX | |
| Preparative | XXGATCTCXXXXXXCGCXXXXXXXXTTCTTTGTTGAA | |
| Example 1) | TTTGTTGT-3 | |
| Primer forward | GATGAAGCCCTCGAGGTAGACAACAAATTCAACAAAG | X |
| (used in | AA | |
| Preparative | | |
| Examples 1 | | |
| and 5) | | |
| Primer reverse | TTAGCTTCTGCTAGCAAGTTAGCGCTTTGGCTTGGGTCA | X |
| (used in | TC | |
| Preparative | | |
| Examples 1 | | |
| and 5) | | |
| Synthetic oligo | ACAACAAATTCAACAAAGAA X01 X01 X03 GCG X04 X01 | X |
| (Oligo A, used | GAGATC X06 X06 CTGCCGAACCTGAAC X03 X09 CAA | |
| in Preparative | X10 X11 GCCTTC X12 X13 X14 TTA X15 | |
| Example 5) | GATGACCCAAGCCAAAGCGC | |
| Synthetic oligo | ACAACAAATTCAACAAAGAA X01 X01 Y03 GCG Y04 X01 | X |
| (Oligo B, used | GAGATC X06 X06 CTGCCGAACCTGAAC Y03 Y09 CAA | |
| in Preparative | Y10 Y11 GCCTTC Y12 Y13 X14 TTA Y15 | |
| Example 5) | GATGACCCAAGCCAAAGCGC | |

Table 2 shows sequences of primers and synthetic oligonucleotides used in the used in Preparative Examples of the present application.

### Biological Example 10:

hCD16a Activation by a hBCMA×hCD16a Dual Engager Harbouring an IL-15 Cytokine Polypeptide:

### Material and Methods

The cDNA coding for a polypeptide of the invention harboring a stop codon was synthesized and ligated into the Nde1 Xho1 restriction sites of the pET29 vector. E coli BL21 (DE3) was transformed with the vector under Kanamycin selection. The Compound, referred to herein as Example Compound 148 (of SEQ ID 148) was expressed by induction of IPTG at an OD of 0.6 and harvested 16 h later. Example Compound 148 was purified from inclusion bodies by dissolving in 1×PBS supplemented by 30 mM imidazole, 6 M Urea, 10 mM glutathione (reduced). Soluble material was purified by IMAC using 1×PBS supplemented by 500 mM imidazole, 6 M Urea as elution buffer. Example Compound 148 was thereafter refolded by dialysis against 1×PBS.

The propensity of the compound to stimulate CD16a activation was assessed in a Lucia Luciferase reporter assay Jurkat-Lucia™ NFAT-CD16 cells (InvivoGen) and compared to the antibody dependent cellular cytotoxicity (ADCC) CD16a activation of Elotuzumab (Empliciti, Bristol Myers Squibb, clinical grade a-SLAMF7 monoclonal antibody) according to method in Example 11.

### Results

A hBCMA×hCD16a dual engager harbouring an IL-15 cytokine sequence (Example Compound 148=SEQ ID 148) was constructed and expressed as soluble gene products in E. coli (DE3). The CD16a activation response of the engager construct in the presence and absence of target MM.1s cells is shown in FIG. 11.

It is seen in the Figure that the affibody-based IL-15 containing dual engager can activate CD16a in a target specific manner.

| | Descriptive ID | |
|---|---|---|
| SEQ | CD16a binder | |
| ID | SEQ ID | Sequence |
| 148 | H_Met2_aBCMA- | MMVDNKFNKENQFADEEIAALPNLNFYQK |
| | 1- | WAFIRKLMDDPSQSANLLAEAKKLNDAQA |
| | E6_15aa_IL15(49- | PKGGGSGGGGSGGGGSGNWVNVISDLKKI |
| | 162)_15aa_aCD16- | EDLIQSMHIDATLYTESDVHPSCKVTAMKC |
| | A10_His6c_OH | FLLELQVISLESGDASIHDTVENLIILANNSL |
| | (containing SEQ ID | SSNGNVTESGCKECEELEEKNIKEFLQSFVH |
| | 167 polypeptide) | IVQMFINTSGGGSGGGGSGGGGSGVDNKF |
| | | NKEVQMAQFEIRKLPNLNHHQSFAFIKSLM |
| | | DDPSQSANLLAEAKKLNDAQAPKYYHHHH |
| | | HH |

### Biological Example 11:

CD16a Activation and NK Cell Mediated Cell Killing of hBCMA×hCD16a Dual Engager Constructs with Different Domain Order and Linkers

Compounds with different distances and ordering of targeting domains in dual engager constructs were prepared. The propensity of the hBCMA×hCD16a dual engagers to stimulate CD16a activation and to enhance NK cell-mediated killing were assessed. The hCD16a binding polypeptide was, in each case, the polypeptide with SEQ ID 167. The hBCMA binding polypeptide was, in each case, the polypeptide with SEQ ID 2.

| SEQ | | |
|---|---|---|
| ID | Descriptive ID | Sequence |
| 149 | H_Met2_aBCMA- | MMVDNKFNKENQFADEEIAALPNLNFYQK |
| | 1-E6_5aa_aBCMA- | WAFIRKLMDDPSQSANLLAEAKKLNDAQA |
| | 1-E6_5aa_aCD16- | PKGGGSGVDNKFNKENQFADEEIAALPNLN |
| | A10_His6c_OH | FYQKWAFIRKLMDDPSQSANLLAEAKKLN |
| | | DAQAPKGGGSGVDNKFNKEVQMAQFEIRK |
| | | LPNLNHHQSFAFIKSLMDDPSQSANLLAEA |
| | | KKLNDAQAPKYYHHHHHH |
| 150 | H_Met2_aBCMA- | MMVDNKFNKENQFADEEIAALPNLNFYQK |
| | 1- | WAFIRKLMDDPSQSANLLAEAKKLNDAQA |
| | E6_10aa_aBCMA- | PKGGGSGGGGSGVDNKFNKENQFADEEIA |
| | 1-E6_10aa_aCD16- | ALPNLNFYQKWAFIRKLMDDPSQSANLLA |
| | A10_His6c_OH | EAKKLNDAQAPKGGGSGGGGSGVDNKFN |
| | | KEVQMAQFEIRKLPNLNHHQSFAFIKSLMD |
| | | DPSQSANLLAEAKKLNDAQAPKYYHHHHH |
| | | H |
| 151 | H_Met2_aBCMA- | MMVDNKFNKENQFADEEIAALPNLNFYQK |
| | 1- | WAFIRKLMDDPSQSANLLAEAKKLNDAQA |
| | E6_15aa_aBCMA- | PKGGGSGGGGSGGGGSGVDNKFNKENQFA |
| | 1-E6_15aa_aCD16- | DEEIAALPNLNFYQKWAFIRKLMDDPSQSA |
| | A10_His6c_OH | NLLAEAKKLNDAQAPKGGGSGGGGSGGG |
| | | GSGVDNKFNKEVQMAQFEIRKLPNLNHHQ |
| | | SFAFIKSLMDDPSQSANLLAEAKKLNDAQA |
| | | PKYYHHHHHH |
| 152 | H_Met2_aBCMA- | MMVDNKFNKENQFADEEIAALPNLNFYQK |
| | 1-E6_15aa_aCD16- | WAFIRKLMDDPSQSANLLAEAKKLNDAQA |
| | A10_15aa_aBCMA- | PKGGGSGGGGSGGGGSGVDNKFNKEVQM |
| | 1-E6_His6c_OH | AQFEIRKLPNLNHHQSFAFIKSLMDDPSQSA |
| | | NLLAEAKKLNDAQAPKGGGSGGGGSGGG |
| | | GSGVDNKFNKENQFADEEIAALPNLNFYQK |
| | | WAFIRKLMDDPSQSANLLAEAKKLNDAQA |
| | | PKYYHHHHHH |
| 153 | H_Met2_aCD16- | MMVDNKFNKEVQMAQFEIRKLPNLNHHQS |
| | A10_15aa_aBCMA- | FAFIKSLMDDPSQSANLLAEAKKLNDAQAP |
| | 1- | KGGGSGGGGSGGGGSGVDNKFNKENQFA |
| | E6_15aa_aBCMA- | DEEIAALPNLNFYQKWAFIRKLMDDPSQSA |
| | 1-E6_His6c_OH | NLLAEAKKLNDAQAPKGGGSGGGGSGGG |
| | | GSGVDNKFNKENQFADEEIAALPNLNFYQK |
| | | WAFIRKLMDDPSQSANLLAEAKKLNDAQA |
| | | PKYYHHHHHH |
| 130 | H_aBCMA-1- | VDNKFNKENQFADEEIAALPNLNFYQKWA |
| | E6_15aa_aCD16- | FIRKLMDDPSQSANLLAEAKKLNDAQAPK |
| | A10_His6c_OH | GGGSGGGGSGGGGSGVDNKFNKEVQMAQ |
| | | FEIRKLPNLNHHQSFAFIKSLMDDPSQSANL |
| | | LAEAKKLNDAQAPKYYHHHHHH |
| 154 | H_aBCMA-1- | VDNKFNKENQFADEEIAALPNLNFYQKWA |
| | E6_aCD16- | FIRKLMDDPSQSANLLAEAKKLNDAQAPK |
| | A10_His6c_OH | VDNKFNKEVQMAQFEIRKLPNLNHHQSFA |
| | | FIKSLMDDPSQSANLLAEAKKLNDAQAPKY |
| | | YHHHHHH |
| 155 | H_aCD16- | VDNKFNKEVQMAQFEIRKLPNLNHHQSFA |
| | A10_aBCMA-1- | FIKSLMDDPSQSANLLAEAKKLNDAQAPKV |
| | E6_His6c_OH | DNKFNKENQFADEEIAALPNLNFYQKWAFI |
| | | RKLMDDPSQSANLLAEAKKLNDAQAPKYY |
| | | HHHHHH |
| 156 | H_aBCMA-1- | VDNKFNKENQFADEEIAALPNLNFYQKWA |
| | E6_Gly_aCD16- | FIRKLMDDPSQSANLLAEAKKLNDAQAPK |
| | A10_His6c_OH | GVDNKFNKEVQMAQFEIRKLPNLNHHQSF |
| | | AFIKSLMDDPSQSANLLAEAKKLNDAQAPK |
| | | YYHHHHHH |
| 157 | H_aCD16- | VDNKFNKEVQMAQFEIRKLPNLNHHQSFA |
| | A10_Gly_aBCMA- | FIKSLMDDPSQSANLLAEAKKLNDAQAPKG |
| | 1-E6_His6c_OH | VDNKFNKENQFADEEIAALPNLNFYQKWA |
| | | FIRKLMDDPSQSANLLAEAKKLNDAQAPK |
| | | YYHHHHHH |

Design, Construction, Expression, and Protein Purification of hBCMA×hCD16a-Binding Polypeptide Engager Constructs.

hBCMA×hCD16a binding constructs were designed for expression and purification as follows: The cDNA coding for each hBCMA×hCD16a binding constructs harbouring a stop codon was synthesized and ligated into the Nde1 Xho1 restriction sites of the pET29 vector. E coli BL21 (DE3) was transformed with vector under Kanamycin selection and constructs were expressed by induction of IPTG at an OD of 0.6 and harvested 16 h later. Soluble cytosolic product was harvested and resuspended in 1×PBS supplemented by 30 mM imidazole. Purification was achieved by IMAC using 1×PBS supplemented by 500 mM imidazole as elution buffer. Further purification was achieved by RP-HPLC and identity/purity was confirmed by SDS-PAGE and analysis by LC-MS/MS.

Both heterodimeric hBCMA×hCD16a dual binding constructs (Example Compounds 130, 154, 155, 156 and 157) and heterotrimeric hBCMA×hCD16a binding constructs containing two BCMA-binding polypeptides were evaluated (Example Compounds 149, 150, 151, 152 and 153).

The propensity of the hBCMA×hCD16a dual engagers to stimulate CD16a activation and to enhance NK cell mediated killing were assessed:

### CD16 Activation Jurkat-Lucia™ Luciferase Reporter Assay

The propensity of the hBCMA×hCD16a dual engagers to stimulate CD16 activation was assessed in a Lucia Luciferase reporter assay and compared to the antibody dependent cellular cytotoxicity (ADCC)potency of Elotuzumab (clinical grade a-SLAMF7 monoclonal antibody) through evaluation of CD16a activation. Jurkat-Lucia™ NFAT-CD16 cells (Invivogen) were seeded with MM.1s cells at an effector to target (E:T) ratio of 2:1 in a 96-well flat-bottom plate. 3×10⁵ cells in 200 μl per well were treated with 20 or 200 nM of the reagent of interest for 24 h. The supernatant was harvested, and Lucia luciferase activity, which reflects the induced ADCC response, was assessed using QUANTI Luc™ Gold (InvivoGen). Responses were normalized to the maximal response of Elotuzumab.

### Calcein-Release-Based In Vitro Cytotoxicity Assay

NK cell cytotoxic activity was assessed in calcein-release-based cytotoxicity assay previously described by (Gauthier et al, 2023) PMID 36635380. In brief, MM1.S target cells were pre-labelled with Calcein-AM (Biolegend) and co-cultured with NK cells at a 1:1 ratio in 96-well v-bottom plate. Engagers were added last to the co-culture, after effector and target cells. After 4 h of co-culture, supernatant was collected and transferred to black flat-bottom 96-well plate. Fluorescent signal of calcein-released by dead MM1.S cells was quantified in SpectraMax i3x (Molecular Devices). Percent specific lysis is calculated in Microsoft Excel (Microsoft) using target cells alone for spontaneous release and target cells with detergent for maximum release. NK cell cytotoxic activity in presence of engagers is compared to antibody dependent cellular cytotoxicity activity of Belantamab biosimilar (purchased from IchorBio).

### Results

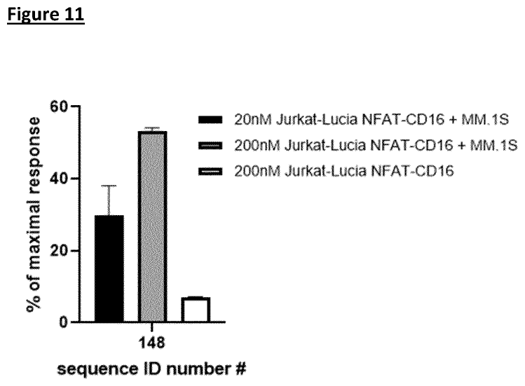

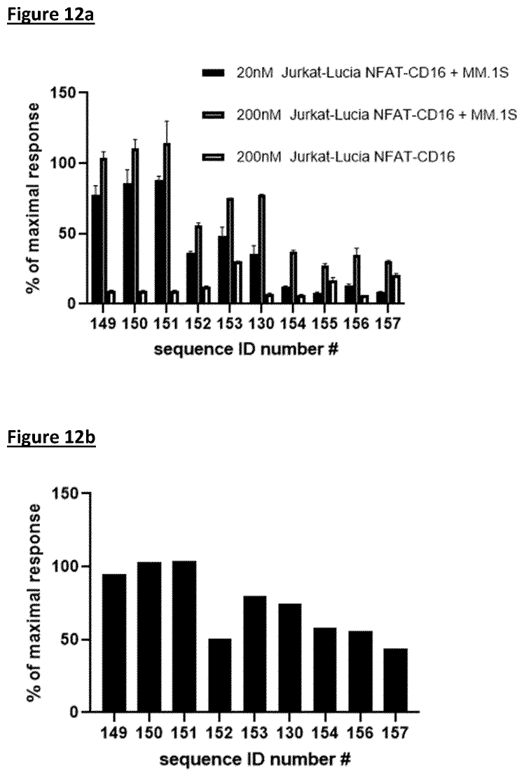

hBCMA×hCD16a dual engager constructs containing one or two hBCMA-binding polypeptides of SEQ ID 2 genetically fused to a hCD16a binding arm composed of the polypeptide of SEQ ID 167 and a C-terminal His₆ tag were constructed and expressed as soluble gene products in E. coli (DE3). FIGS. 12a and 12b show the hCD16a activation responses of the engager constructs in the presence or absence of target MM.1s cells.

The results show that the affibody-based heterodimeric hBCMA×hCD16a dual binding constructs and heterotrimeric hBCMA×hCD16a binding constructs, here composed of the polypeptides of SEQ ID 2 (for hBCMA) and SEQ ID 167 (for hCD16a), can activate hCD16a in a target specific manner when genetically fused together in either order. Good activation was observed for engagers with linker length varying from 0 and 15 amino acids.

### Biological Example 12:

Assessment of Domain Boundaries in hBCMA×hCD16a Dual Engager Constructs Using a Jurkat CD16a Activation Reporter Assay and a NK Medisted Cell Killing Assay

hBCMA×hCD16a dual binding constructs with truncations in both the N- and C-terminal of both the BCMA binding polypeptide and the CD16a binding A10 affibody were produced and evaluated in a hCD16a-mediated activation cell-based reporter assay and cell killing. This enabled the domain boundaries of the targeting domains in dual engager constructs to be assessed.

| SEQ | | |
|---|---|---|
| ID | Descriptive ID | Sequence |
| 156 | H_aBCMA-1- | VDNKFNKENQFADEEIAALPNLNFYQKWA |
| | E6_Gly_aCD16- | FIRKLMDDPSQSANLLAEAKKLNDAQAPK |
| | A10_His6c_OH | GVDNKFNKEVQMAQFEIRKLPNLNHHQSF |
| | | AFIKSLMDDPSQSANLLAEAKKLNDAQAPK |
| | | YYHHHHHH |
| 158 | H_Met_aBCMA-1- | MNKENQFADEEIAALPNLNFYQKWAFIRKL |
| | E6-(6- | MDDPSQSANLLAEAKKLNDAQGNKEVQM |
| | 55)_Gly_aCD16- | AQFEIRKLPNLNHHQSFAFIKSLMDDPSQSA |
| | A10-(6- | NLLAEAKKLNDAQAPKYYHHHHHH |
| | 58)_His6c_OH | |
| 159 | H_Met_aBCMA-1- | MNKENQFADEEIAALPNLNFYQKWAFIRKL |
| | E6-(6- | MDDPSQSANLLAEAKKLNDAQGVDNKFN |
| | 55)_Gly_aCD16- | KEVQMAQFEIRKLPNLNHHQSFAFIKSLMD |
| | A10-(1- | DPSQSANLLAEAKKLNDAQYYHHHHHH |
| | 55)_His6c_OH | |
| 160 | H_Met_aBCMA-1- | MNKENQFADEEIAALPNLNFYQKWAFIRKL |
| | E6-(6- | MDDPSQSANLLAEAKKLNDAQGVDNKFN |
| | 55)_Gly_aCD16- | KEVQMAQFEIRKLPNLNHHQSFAFIKSLMD |
| | A10_His6c_OH | DPSQSANLLAEAKKLNDAQAPKYYHHHHH |
| | | H |
| 161 | H_aBCMA-1-E6- | GDNKFNKENQFADEEIAALPNLNFYQKWA |
| | (V1G)_Gly_aCD16- | FIRKLMDDPSQSANLLAEAKKLNDAQAPK |
| | A10-(6- | GNKEVQMAQFEIRKLPNLNHHQSFAFIKSL |
| | 58)_His6c_OH | MDDPSQSANLLAEAKKLNDAQAPKYYHH |
| | | HHHH |
| 162 | H_aBCMA-1-E6- | GDNKFNKENQFADEEIAALPNLNFYQKWA |
| | (V1G)_Gly_aCD16- | FIRKLMDDPSQSANLLAEAKKLNDAQAPK |
| | A10-(1- | GVDNKFNKEVQMAQFEIRKLPNLNHHQSF |
| | 55)_His6c_OH | AFIKSLMDDPSQSANLLAEAKKLNDAQYY |
| | | HHHHHH |
| 163 | H_aBCMA-1-E6- | GDNKFNKENQFADEEIAALPNLNFYQKWA |
| | (V1G)_Gly_aCD16- | FIRKLMDDPSQSANLLAEAKKLNDAQAPK |
| | A10_His6c_OH | GVDNKFNKEVQMAQFEIRKLPNLNHHQSF |
| | | AFIKSLMDDPSQSANLLAEAKKLNDAQAPK |
| | | YYHHHHHH |

Design, Construction, Expression, and Protein Purification of hBCMA×hCD16a CD16a-Binding Polypeptide Dual Engager Constructs

hBCMA×hCD16a dual binding constructs were designed for expression and purification as described above in Biological Example 11. The heterodimeric hBCMA×hCD16a dual binding constructs that were prepared are listed above (SEQ IDs156 and 158-163).

The propensity of the hBCMA×hCD16a dual engagers to stimulate CD16a activation and to enhance NK cell mediated killing were assessed according to the protocols as described above in Biological Example 11.

### Results

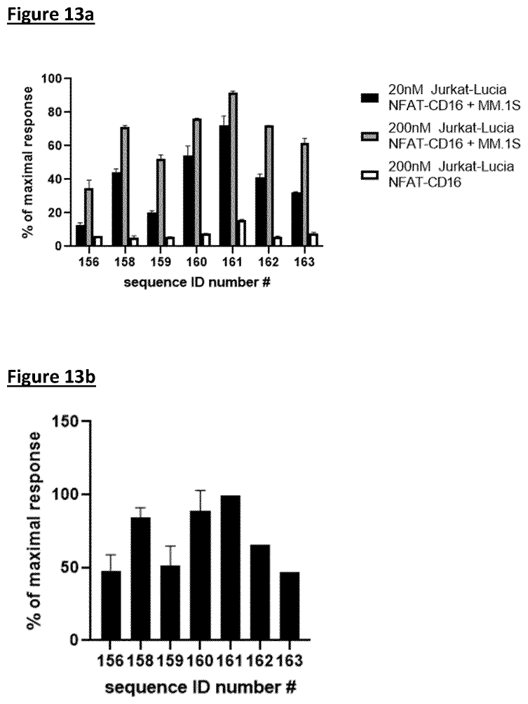

hBCMA×hCD16a dual engager constructs containing one BCMA-binding polypeptide of SEQ ID 2 genetically fused to a hCD16a binding arm composed of the polypeptide of SEQ ID 167 (in some instances with truncations) and a C-terminal His₆ tag were constructed and expressed as a soluble gene products in E. coli (DE3). FIG. 13a shows the CD16a activation responses of the engager constructs in the presence or absence of target MM. 1s cells. Moreover, the engagers induced NK cell mediated killing of MM.1S cells (FIG. 13b).

The results show that the affibody-based heterodimeric hBCMA×hCD16a dual binding constructs, here composed of the polypeptides of SEQ ID 2 (for hBCMA) and SEQ ID 167 (for hCD16a), can activate hCD16a in a target specific manner when genetically fused together, including with truncations in certain location.

### Biological Example 13:

### NK Cell Mediated Multiple Myeloma Cell Killing

The ability of a hBCMA×hCD16a dual engager to promote NK cell mediated lysis and cell killing was assessed by a flow cytometry assay and an Incucyte based assay. The compounds investigated were as shown in the table below:

| Ex. | | |
|---|---|---|
| SEQ | | |
| ID | Descriptive ID | Sequence |
| 131 | H_aBCMA-1- | VDNKFNKENQFADEEIAALPNLNFYQKWA |
| | E6_15aa_aCD16- | FIRKLMDDPSQSANLLAEAKKLNDAQAPK |
| | H09_15aa_aCD16- | GGGSGGGGSGGGGSGVDNKFNKEFWIAES |
| | A10_His6c_OH | EIESLPNLNIYQKWAFKYSLADDPSQSANLL |
| | | AEAKKLNDAQAPKGGGSGGGGSGGGGSG |
| | | VDNKFNKEVQMAQFEIRKLPNLNHHQSFA |
| | | FIKSLMDDPSQSANLLAEAKKLNDAQAPKY |
| | | YHHHHHH |
| 132 | H_aBCMA-1- | VDNKFNKENQFADEEIAALPNLNFYQKWA |
| | E6_15aa_aCD16- | FIRKLMDDPSQSANLLAEAKKLNDAQAPK |
| | A10_15aa_aCD16- | GGGSGGGGSGGGGSGVDNKFNKEVQMAQ |
| | A10_His6c_OH | FEIRKLPNLNHHQSFAFIKSLMDDPSQSANL |
| | | LAEAKKLNDAQAPKGGGSGGGGSGGGGS |
| | | GVDNKFNKEVQMAQFEIRKLPNLNHHQSF |
| | | AFIKSLMDDPSQSANLLAEAKKLNDAQAPK |
| | | YYHHHHHH |
| 130 | H_aBCMA-1- | VDNKFNKENQFADEEIAALPNLNFYQKWA |
| | E6_15aa_aCD16- | FIRKLMDDPSQSANLLAEAKKLNDAQAPK |
| | A10_His6c_OH | GGGSGGGGSGGGGSGVDNKFNKEVQMAQ |
| | | FEIRKLPNLNHHQSFAFIKSLMDDPSQSANL |
| | | LAEAKKLNDAQAPKYYHHHHHH |

Compounds were produced according to the preparation described in Biological Example 11.

### Flow Cytometry-Based In Vitro Assays.

NK cell function was assessed in flow cytometry-based assays described previously (Bryceson, Fauriat et al. 2010). In brief, NK cells were co-cultured with target cells for 6 hours at an E:T ratio of 1:1, followed by staining for degranulation marker CD107a and interferon-gamma. BD GolgiStop (BD Biosciences) was added after 1 hour at a concentration of 1/1500. For cytotoxicity assays, target cells were pre-stained with CellTrace Violet (Invitrogen) and co-incubated with NK cells at different E:T ratios for 8-12 hours, based on kinetics from live cell imaging assays. Samples were acquired on either a BD LSR II or a FACSymphony A5 (BD Biosciences). Data were analyzed with FlowJo version 10 (BD Biosciences).

### Incucyte-Based In Vitro Cytotoxicity Assays.

Real-time tumor cell killing was measured on the Incucyte S3 platform (Essen Biosciences). NK cells were co-cultured with MM. 1s cells labelled with the red fluorescent protein mCherry, on 96-well plates precoated with Poly-L-Ornithine 0.01% (Sigma-Aldrich) at various E:T ratios depending on the type of assay. For assays involving reagents (e.g. affibody constructs), these were added after the effector and target cells. Scanning was done every 60 minutes for at least 24 hours, using a 10X objective lens, and images were analyzed using Incucyte Controller v2020A (Essen Biosciences). The same masking was used for repeat analyses. In the experiments carried out, the cells were treated with 100 nM of dual engager, null variant (not binding hBCMA), or daratumumab.

### Results

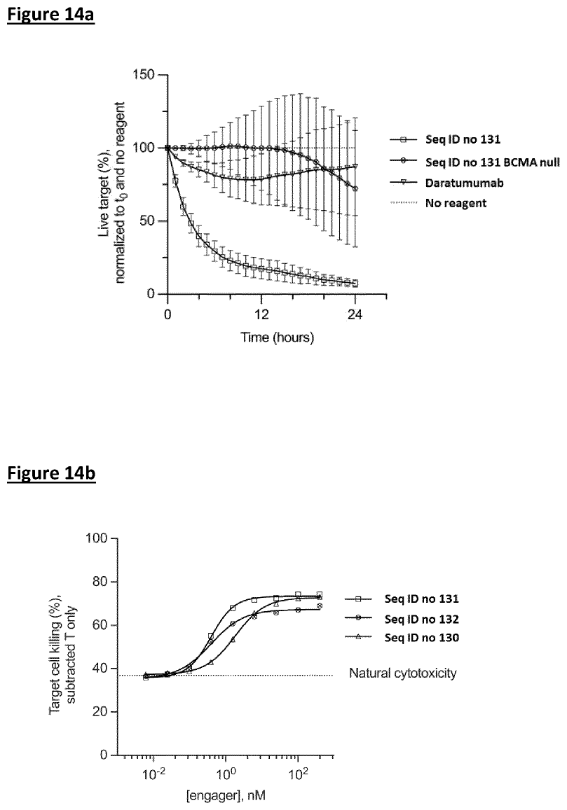

The capacity of the dual engager Example Compound 131 to induce lysis of MM.1s cells was examined. The results are shown in FIG. 14a. Results are from one representative experiment (n=4). In a 24-hour killing assay, Example compound 131 elicited rapid and superior lysis of MM.1s cells, compared to both daratumumab and the control null-variant anti-hBCMA-Null-H09-A10-His₆. The bulk cytotoxic effect of Example Compound 131 was seen within the first 8 to 12 hours after the start of the co-culture.

Finally, a flow cytometry-based killing assay was implemented to establish functional EC₅₀ and EC₉₀ values of the three constructs of Example Compounds 131, 132 and 130. The frequency of dead target cells was calculated based on the staining of dead cell marker on target cells pre-incubated with cell trace violet (CTV). The results are shown in FIG. 14b. Results are from two independent experiments (n=8), where spontaneous target cell death is subtracted. The plateau-level cytotoxicity was highest for Example Compound 131 and lowest for Example Compound 132, in agreement with the degranulation responses, which showed a higher fratricide effect for the latter construct. Compared with Example Compound 130, plateau cytotoxicity for Example Compound 131 was reached at a lower dose. EC₅₀ values were 0.4 nM for Example Compound 131 and Example Compound 132, and 1.8 nM for Example Compound 130, while EC₉₀ values were 2.0, 3.8, and 14.7 nM, respectively. The data demonstrate that these dual engagers elicit potent responses.

### Biological Example 14:

Enhancement of NK Cell Mediated Lysis of Multiple Myeloma Cells in a hBCMA Selective Manner.

The ability of a hBCMA×hCD16a dual engager to enhance NK cell mediated lysis of multiple myeloma cells in a BCMA selective manner was investigated. The compound that was tested was Example Compound 131.

### Genetic Cell Engineering

A BCMA knock-out variant of cell line MM.1s was made using CRISPR-Cas9. The BCMA overexpression variant of cell line MM.1s and variants expressing the red fluorescent protein mCherry was done through lentiviral transfection, with vectors designed and ordered from VectorBuilder (VectorBuilder Inc.). MM.1s expressing the nuclear red fluorescent protein mCherry: MM. 1s cells were transduced with 0.3 MOI lentiviral particles. After transduction, approximately 30% of cells appeared red in the fluorescence microscope. Cells were then sorted by fluorescence-activated cell sorting (FACS) on a Sony SH800 cell sorter (Sony Biotechnology). MM.1s with BCMA gene overexpressed: On day 1, 1 million MM. 1s cells were transduced with 10 MOI lentiviral particles (denoted SK-V93, and shown in Figure S6A) and seeded out on a 24-well plate. On day 4, cells were washed in PBS and seeded out at 0.5 million cells per milliliter in fresh media. After sufficient expansion, cells went through FACS. MM. 1s with BCMA gene knocked out: The following sgRNA sequences were used: sgRNA #0, gacgagtttaaaaacac (SEQ ID 287); sgRNA #1, gagcttaataatttctt (SEQ ID 288); and sgRNA #2, gtgaccaattcagtgaa (SEQ ID 289). sgRNA and Cas9 were assembled at a ratio of 9:1 on sterile 96-well U-bottom plates. 300,000 MM.1s cells were used per nucleofection. Knockout efficiency was confirmed after five days by PCR and Sanger sequencing, analyzed with Synthego Performance Analysis (ICE) (Synthego). After sufficient expansion, cells went through FACS.

### Flow Cytometry-Based In Vitro Assays.

NK cell function was assessed in flow cytometry-based assays described previously (Bryceson, Fauriat et al. 2010). In brief, NK cells were co-cultured with target cells for 6 hours at an E:T ratio of 1:1, followed by staining for degranulation marker CD107a and interferon-gamma. BD GolgiStop (BD Biosciences) was added after 1 hour at a concentration of 1/1500.

### Results

Using engineered MM.1s target cells in which BCMA was either knocked out or overexpressed, a clear correlation between the BCMA expression level and NK cell activation was observed (see FIG. 15). The data demonstrate that the dual engager Example Compound 131 a concentration of 100 nM elicits responses, with specificity to BCMA. In the figure, the results are shown for MM. 1s wild type (WT), BCMA knock-out (KO), and BCMA overexpression (OE). Results are from one representative experiment (n=4).

### Biological Example 15:

### A Further Anti-hBCMA Drug Conjugate is Cytotoxic to BCMA⁺ Multiple Myeloma Cells

An Example Compound with the Fa-G6 sequence (SEQ ID 1) harboring a c-terminal Cys was produced by conventional solid peptide synthesis. The Compound had SEQ ID 164:

| Seq | Descriptive | |
|---|---|---|
| ID | ID | Sequence |
| 164 | aBCMA- | VDNKFNKEETFADLEISNLPNLNFYQK |
| | Fa-G6-Cys | WAFIRSLMDDPSQSANLLAEAKKLNDAQAPKC |

MC-MMAF linker toxin was dissolved in DMF organic solvent and conjugated at a molar ratio 2:1. The product harboring one mc-MMAF was isolated by RP-HPLC and prepared in 10% IPA in PBS buffer.

By using a CellTiter-Blue® Cell Viability Assay, the anti-cancer activity of the affibody drug conjugate Fa-G6-MMAF was investigated in five multiple myeloma cancer cell lines, namely, EJM, MOLP-2, NCI-H929, OPM-2, and U-266. The Fa-G6-MMAF was evaluated in a concentration range of 1 ng/mL to 30 μg/mL.

### Results

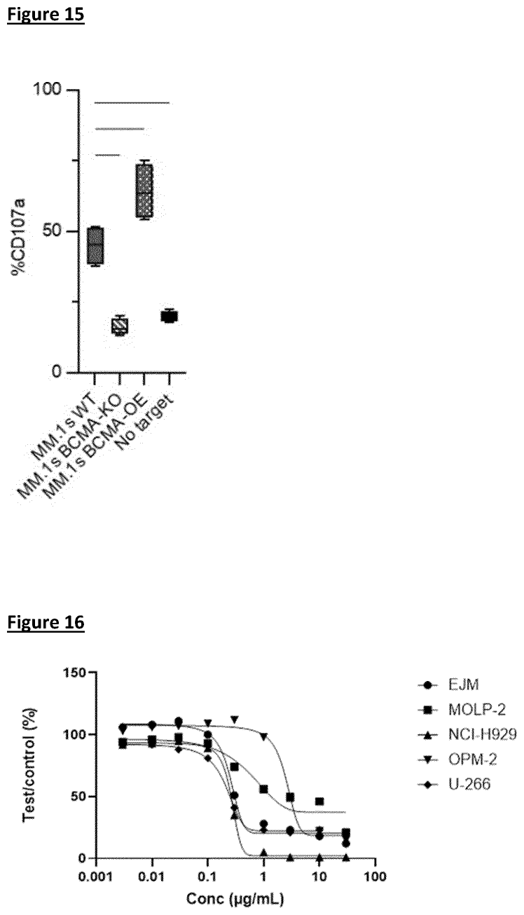

The results are shown in FIG. 16. The Fa-G6-MMAF affibody drug conjugate showed anti-cancer activity in all multiple myeloma cell lines investigated with EC₅₀ values ranging from 0.19 to 3.5 μg/mL. Thus, Fa-G6-MMAF show activity in several BCMA+ multiple myeloma cell lines.

### Biological Example 16A:

### Compound Containing Non-Natural Amino Acid Norleucine

### Experimental

The polypeptide with sequence of SEQ ID 165 (aBCMA-1-E6-(6-55)M35 nL) was synthesized by solid-phase peptide synthesis (SPSS), purified by RP-HPLC and lyophilized. Correct Mw was confirmed by LC-MS/MS.

| Seq | Descriptive | |
|---|---|---|
| ID | ID | Sequence |
| 165 | aBCMA- | GNKENQFADEEIAALPNLNFYQKWAFIRKLnLDD |
| | 1-E6-(6- | PSQSANLLAEAKKLNDAQK |
| | 55)M35n | |
| | L | |

Binding affinity to the extracellular domain of BCMA was studied by SPR in a Biacore T200. BCMA was immobilized as ligand onto a CM5 ship (Cytiva) at 1300 Ru. The Example Compound with SEQ ID 165 was used as analyte and responses were monitored in a concentration range of 1-100 nM. Data was fitted to a 1:1 binding mode.

### Results

It was observed that the Example Compound with SEQ ID 165 binds BCMA with an apparent dissociation constant of 10 nM.

### Biological Example 16B:

### Assessment of Domain Boundaries in BCMA×hCD16a Dual Engager Constructs Using a Jurkat CD16a Activation Reporter Assay and a NK Mediated Cell Killing Assay

hBCMA×hCD16a dual binding constructs were produced and evaluated in a hCD16a-mediated activation cell-based reporter assay and cell killing as described above in Biological Example 11. Activity was compared for an engager with a full length aBCMA 1E6 domain (SEQ ID 156) with an engager harbouring truncation in both the N- and C-terminal (SEQ ID 160), as well as an engager harbouring a 1E6 V1G point mutation (SEQ ID 163).

| SEQ | | |
|---|---|---|
| ID | Descriptive ID | Sequence |
| 156 | H_aBCMA-1- | VDNKFNKENQFADEEIAALPNLNFYQKWA |
| | E6_Gly_aCD16- | FIRKLMDDPSQSANLLAEAKKLNDAQAPK |
| | A10_His6c_OH | GVDNKFNKEVQMAQFEIRKLPNLNHHQSF |
| | | AFIKSLMDDPSQSANLLAEAKKLNDAQAPK |
| | | YYHHHHHH |
| 160 | H_Met_aBCMA-1- | MNKENQFADEEIAALPNLNFYQKWAFIRKL |
| | E6-(6- | MDDPSQSANLLAEAKKLNDAQGVDNKFN |
| | 55)_Gly_aCD16- | KEVQMAQFEIRKLPNLNHHQSFAFIKSLMD |
| | A10_His6c_OH | DPSQSANLLAEAKKLNDAQAPKYYHHHHH |
| | | H |
| 163 | H_aBCMA-1-E6- | GDNKFNKENQFADEEIAALPNLNFYQKWA |
| | (V1G)_Gly_aCD16- | FIRKLMDDPSQSANLLAEAKKLNDAQAPK |
| | A10_His6c_OH | GVDNKFNKEVQMAQFEIRKLPNLNHHQSF |
| | | AFIKSLMDDPSQSANLLAEAKKLNDAQAPK |
| | | YYHHHHHH |

Design, Construction, Expression, and Protein Purification of hBCMA×hCD16a CD16a-Binding Polypeptide Dual Engager Constructs.

hBCMA×hCD16a dual binding constructs were designed for expression and purification according to Biological Example 11. The heterodimeric hBCMA×hCD16a dual binding constructs that were prepared are listed above (SEQ IDs 156, 160 and 163).

The propensity of the hBCMA×hCD16a dual engagers to stimulate CD16a activation and to enhance NK cell mediated killing were assessed according to the protocols described in Biological Example 11.

### Results

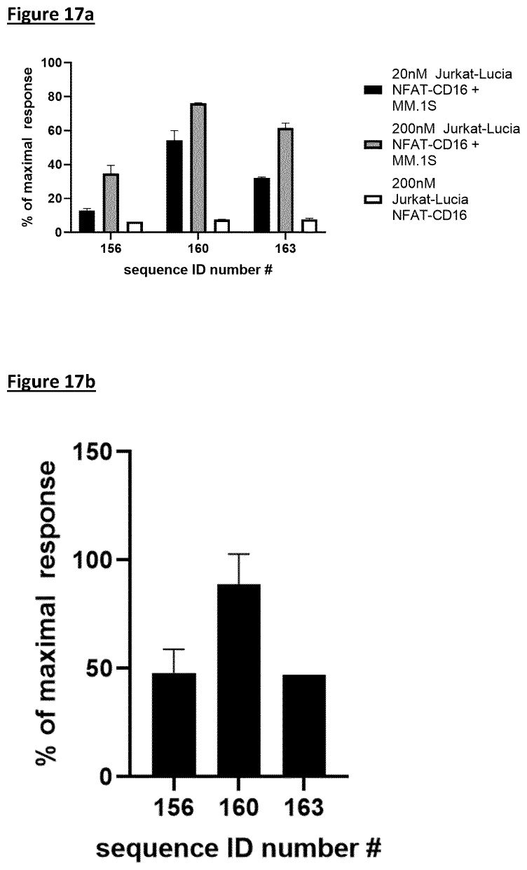

hBCMA×hCD16a dual engager constructs containing one BCMA-binding polypeptide genetically fused to a hCD16a binding arm and a C-terminal His₆ tag were constructed and expressed as a soluble gene products in E. coli (DE3). FIG. 17a shows the CD16a activation responses of the engager constructs in the presence or absence of target MM.1s cells. Moreover, the engagers induced NK cell mediated killing of MM.1S cells as shown in FIG. 17b.

The results show that the hBCMA binding arm, here composed of the polypeptide of SEQ ID 2 (1E6) can be shortened and still retain hCD16a activation. Moreover, Valine 1 can be substituted for Glycine without loss of activity.

### Biological Example 17:

### Anti-BCMA Engager Induce Immune Cell Activation in the Presence of Tumour Cells

Experiments were performed to evaluate the propensity of the anti-BCMA engager Example Compound with SEQ ID number 130 to evoke an IFN□□ response in cocultures of human PBMCs and the BCMA positive multiple myeloma cell line MM.1S. Thawed and overnight rested PBMCs (4W-270, Lonza) were co-cultured with MM.1S cells (CRL-2974, ATCC) at effector to target ratio of 25:1 in V shaped 96-well plate (249935, Thermo Scientific). Total 4×10⁵ cells in 200 μl/well were treated with 200 nM anti-BCMA engagers or 200 nM Elotuzumab (300 mg Empliciti, Bristol-Myers Squibb) for 4 hours at 37° C. in a humidified 5% CO₂ incubator. Supernatant was harvested followed by centrifugation 600 g for 5 min. IFN-γ level was determined by enzyme-linked immunoassay (ELISA) using Human IFN-γ ELISA MAX™ Deluxe Set (430104, BioLegend). Data was processed using Excel and plotted using GraphPad Prism 9.

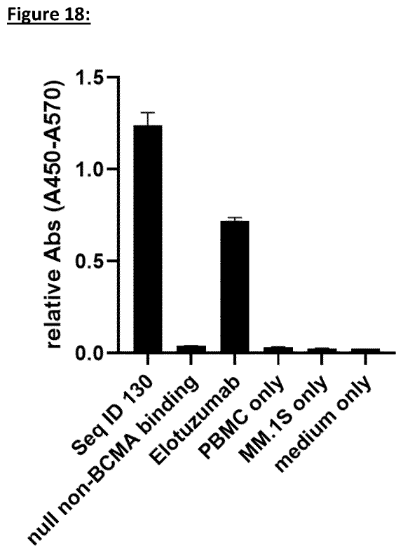

The anti-BCMA engager construct with SEQ ID 130 containing a BCMA-binding polypeptide (herein termed 1-E6 (SEQ ID 2) genetically fused to a hCD16a binding polypetide (herein termed A10 (SEQ Seq ID 167) was evaluated. The SLAMF7 monoclonal antibody Elotuzumab was used as a positive control and single cultures of PBMC and MM.1S served as negative controls. The dual engager containing a BCMA-binding polypeptide evoked an IFN□ response in cocultures of PBMC and MM.1S cells and the response was larger than of the positive control Elotuzumab (FIG. 18). The dual engager construct devoid of BCMA binding showed no IFN response in cocultures of PBMC and MM.1S cells. This experiment illustrates the BCMA dependent activation of PBMC against the BCMA positive MM.1S myeloma cell line induced by the anti-BCMA engager described herein.

### Biological Example 18:

CD16a Activation and NK Cell Mediated Cell Killing by hBCMA×hCD16a Dual Engagers with hCD16a Binding Polypeptide

The hBCMA×hCD16a dual engagers comprising hBCMA binding polypeptides with SEQ ID Numbers 166 and 144 were expressed in E. coli (DE3) and thereafter characterized for their CD16a activation properties.

The cDNA coding for each hBCMA×hCD16a dual engager harbouring a stop codon was synthesized and ligated into the Nde1 Xho1 restriction sites of the pET29 vector. E coli BL21 (DE3) was transformed with vector under Kanamycin selection and constructs were expressed by induction of IPTG at an OD of 0.6 and harvested 16 h later. Soluble cytosolic product was harvested and resuspended in 1×PBS. Heat denaturation for 7 min at 95° C. was applied and precipitated protein was pelleted by centrifugation at 20,000×g. Further purification was achieved by RP-HPLC and identity/purity was confirmed by SDS-PAGE and LC/MS/MS analysis. The propensity of the hBCMA×hCD16a dual engagers to stimulate CD16a activation and to enhance NK cell mediated killing were assessed according to the protocols described in Biological Example 11.

| SEQ | | |
|---|---|---|
| ID | Descriptive ID | Sequence |
| 166 | H_Met2_aBCMA- | MMVDNKFNKENQFADEEIAALPNLNFYQK |
| | 1-E6_15aa_aCD16- | WAFIRKLMDDPSQSANLLAEAKKLNDAQA |
| | A10_OH | PKGGGSGGGGSGGGGSGVDNKFNKEVQM |
| | | AQFEIRKLPNLNHHQSFAFIKSLMDDPSQSA |
| | | NLLAEAKKLNDAQAPK |
| 144 | H_aBCMA-1- | VDNKFNKENQFADEEIAALPNLNFYQKWA |
| | E6_10aa_aBCMA- | FIRKLMDDPSQSANLLAEAKKLNDAQAPK |
| | 1-E6_10aa_aCD16- | GGGSGGGGSGVDNKFNKENQFADEEIAAL |
| | A10_OH | PNLNFYQKWAFIRKLMDDPSQSANLLAEA |
| | | KKLNDAQAPKGGGSGGGGSGVDNKFNKE |
| | | VQMAQFEIRKLPNLNHHQSFAFIKSLMDDP |
| | | SQSANLLAEAKKLNDAQAPK |

### Results

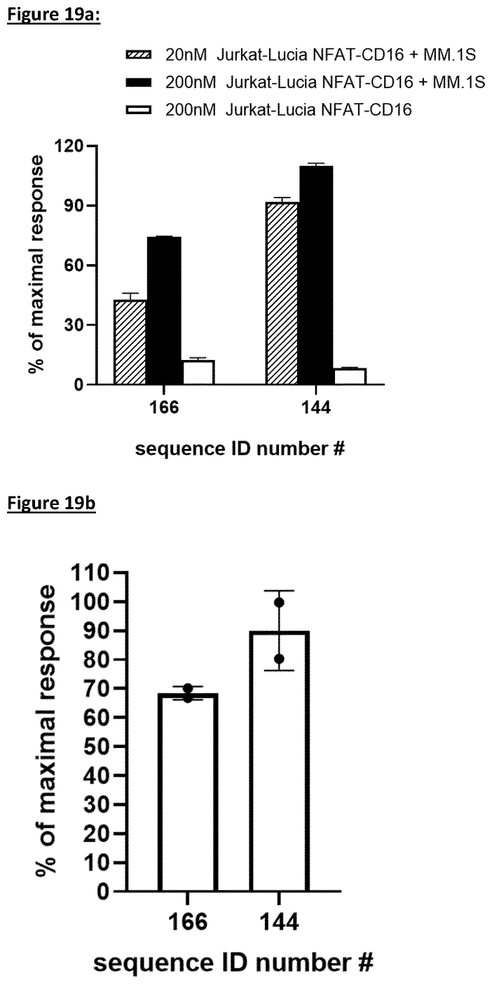

The results are shown in FIG. 19. It is seen that the hBCMA×hCD16a dual engagers (Examples 166 and 144) demonstrated CD16a activation in the Lucia Luciferase reporter assay in the presence of hBCMA+MM.1S cells, whereas no detectable activation was seen in the absence of target cells (FIG. 19a). Moreover, the engagers induced NK cell mediated killing of MM.1S cells (FIG. 19b). Thus, a firm and target specific activation leading to NK mediated cell killing was seen for the engagers investigated.

Whilst the present invention has been described and illustrated with reference to particular embodiments, it will be appreciated by those of ordinary skill in the art that the invention lends itself to many different variations not specifically illustrated herein.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

The following embodiments (Embodiments 1-32) additionally illustrate the invention:

Embodiment 1. An hBCMA-binding polypeptide which comprises at least one motif that binds to hBCMA, wherein said polypeptide comprises the following structure:
[N-terminal portion]-[Helix 1]-[Separating portion]-[Helix 2]-[C-terminal portion]
the hBCMA binding motif being the portion [Helix 1]-[Separating portion]-[Helix 2].

Embodiment 2. The hBCMA-binding polypeptide according to embodiment 1, wherein:
i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are any naturally occurring amino acid; X₁₁ is E, F, H, Q, T or Y; X₁₄ is any naturally occurring amino acid; X₁₇ is A, E, Q, S, T or V; X₁₈ is any naturally occurring amino acid; X₂₅ is F or Y; X₃₁ is I, M, or V; X₃₃ is K or S; X₃₅ is I, L, M, or V;
or ii) Helix 1 and Helix 2 are defined as in i), wherein within Helix 1 and Helix 2, at least 1 and no more than 5 of the Xₙ residues are replaced by an alternative residue, and/or at least 1 and no more than 5 of the residues not labelled as Xₙ are replaced by an alternative residue.

Embodiment 3. The hBCMA-binding polypeptide according to embodiment 2, wherein the hBCMA binding efficacy is at least 1% of SEQ ID NO: 2.

Embodiment 4. The hBCMA-binding polypeptide according to embodiment 1, wherein:
i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are any naturally occurring amino acid; X₁₁ is E, F, H, Q, T or Y; X₁₄ is any naturally occurring amino acid; X₁₇ is A, E, Q, S, T or V; X₁₈ is any naturally occurring amino acid; X₂₅ is F or Y; X₃₁ is I, M, or V; X₃₃ is K or S; X₃₅ is I, L, M, or V;
or ii) Helix 1 and Helix 2 are defined as in i), wherein within Helix 1 and Helix 2, at least 1 and no more than 5 of the Xₙ residues are replaced by an alternative residue, and/or at least 1 and no more than 5 of the residues not labelled as Xₙ are replaced by an alternative residue;
and wherein the hBCMA binding efficacy is at least 1% of SEQ ID NO: 2.

Embodiment 5. The hBCMA-binding polypeptide according to embodiment 2, wherein:
i) Helix 1 comprises the sequence X₉X₁₀X₁₁ADX₁₄EIX₁₇X₁₈ [SEQ ID NO. 278] and Helix 2 comprises the sequence FYQKWAFIRX₃₃LM, wherein, independently from each other,
X₉ is D, E, H, K, N, Q, S, or V; X₁₀ is A, E, F, I, K, M, N, Q, R, S, T, Y, or V; X₁₁ is E, F, or H; X₁₄ is A, E, H, I, K, L, Q, R, T, or Y; X₁₇ is A, E S, T, or V; X₁₈ is A, F, H, K, L, M, N, T, or S; X₃₃ is K or S;
or ii) Helix 1 and Helix 2 are defined as in i), wherein within Helix 1 and Helix 2, at least 1 and no more than 3 of the Xₙ residues are replaced by an alternative residue, and/or at least 1 and no more than 3 of the residues not labelled as Xₙ are replaced by an alternative residue.

Embodiment 6. The hBCMA-binding polypeptide according to any of embodiments 2 to 5, wherein:
i) Helix 1 comprises the sequence NKEETFADLEISNL and Helix 2 comprises the sequence NFYQKWAFIRSLMDD,
or ii) Helix 1 and Helix 2 are defined as in i), wherein within Helix 1 and Helix 2, at least 1 and no more than 2 (for example 1 or 2) residues are replaced by an alternative residue.

Embodiment 7. The hBCMA-engaging polypeptide according to any of embodiments 2 to 5, wherein:
i) Helix 1 comprises the sequence NKENQFADEEIAAL and Helix 2 comprises the sequence NFYQKWAFIRKLMDD,
or ii) Helix 1 and Helix 2 are defined as in i), wherein within Helix 1 and Helix 2, at least 1 and no more than 2 residues are replaced by an alternative residue.

Embodiment 8. The hBCMA-binding polypeptide according to any of embodiments 1 to 7, wherein:
(i) said separating portion has the sequence PNL and said hBCMA binding motif is flanked by an N-terminal portion X₁X₂X₃X₄X₅ and a C-terminal portion PSQSANLLAEAKKLNDAQAPK,
wherein in said N-terminal portion,
X₁ is G, V, or deleted; X₂ is D or deleted; X₃ is N or deleted; X₄ is K or deleted; X₅ is F or deleted;
or (ii) the separating portion, N-terminal portion, and C-terminal portion are as defined in (i), wherein within those portions taken together at least 1 and no more than 5 residues are replaced by an alternative residue.

Embodiment 9. The hBCMA-binding polypeptide according to embodiment 8, wherein said N-terminal portion has the sequence VDNKF

Embodiment 10. The hBCMA-binding polypeptide according to any of embodiments 5, 6, 7, 8 or 9 wherein the hBCMA binding efficacy is at least 1% of SEQ ID NO: 2.

Embodiment 11. The hBCMA-binding polypeptide according to embodiment 1, wherein the sequence of said at least one hBCMA-binding polypeptide is selected from:

[SEQ ID NO: 1]
VDNKFNKEETFADLEISNLPNLNFYQKWAFIRSLMDDPSQSANLLAEA
KKLNDAQAPK;
or
[SEQ ID NO: 2]
VDNKFNKENQFADEEIAALPNLNFYQKWAFIRKLMDDPSQSANLLAE
AKKLNDAQAPK

Embodiment 12. The hBCMA-binding polypeptide according to any of embodiments 1 to 11, which comprises at least two hBCMA-binding polypeptides.

Embodiment 13. The hBCMA-binding polypeptide according to embodiment 12, wherein the at least two hBCMA-binding polypeptides are separated by a linker.

Embodiment 14. The hBCMA-binding polypeptide according to any of embodiments 1 to 13, which further comprises one or more additional binding moiety(ies).

Embodiment 15. The hBCMA-binding polypeptide according to embodiment 14, wherein the one or more additional binding moiety(ies) is specific for an immune cell surface target, for example an NK cell activating receptor, for example CD16a.

Embodiment 16. The hBCMA-binding polypeptide according to embodiment 14 or embodiment 15, wherein the additional binding moiety(ies) is separated from the at least one hBCMA-binding polypeptide by a linker.

Embodiment 17. An hBCMA binder-drug conjugate comprising the hBCMA-binding polypeptide according to any of embodiments 1-13 and an additional therapeutic agent.

Embodiment 18. The hBCMA binder-drug conjugate according to embodiment 17, wherein the additional therapeutic agent is a cytotoxic drug, for example MMAF, MMAE MMAF, doxorubicin, pyrrolobenzodiazepine, amanitin, maytansinoids, duostatins, mitomycin C, desmethyltopotecan or SN-38.

Embodiment 19. The hBCMA binder-drug conjugate according to embodiment 17 or embodiment 18, wherein the hBCMA-binding polypeptide is connected to the additional therapeutic agent via a linker.

Embodiment 20. A nucleic acid molecule encoding the hBCMA-binding polypeptide according to any of embodiments 1 to 16.

Embodiment 21. An expression vector comprising the nucleic acid molecule according to embodiment 20.

Embodiment 22. A host cell comprising the nucleic acid molecule according to embodiment 19 or the expression vector according to embodiment 21.

Embodiment 23. A method of making the hBCMA-binding polypeptide according to any of embodiments 1 to 16, the method comprising maintaining the host cell according to embodiment 22 under optimal conditions for expression of the nucleic acid and isolating the hBCMA-binding polypeptide.

Embodiment 24. A pharmaceutical composition comprising the hBCMA-binding polypeptide according to any of embodiments 1 to 16, the hBCMA binder-drug conjugate according to any of embodiments 17 to 19, the nucleic acid molecule according to embodiment 20 or the expression vector according to embodiment 21.

Embodiment 25. The hBCMA-binding polypeptide according to any of embodiments 1 to 16, the hBCMA binder-drug conjugate according to any of embodiments 17 to 19, the nucleic acid molecule according to embodiment 20, the expression vector according to embodiment 21, and/or the pharmaceutical composition according to embodiment 24, for use in medicine.

Embodiment 26. The hBCMA-binding polypeptide according to any of embodiments 1 to 16, the hBCMA binder-drug conjugate according to any of embodiments 17 to 19, the nucleic acid molecule according to embodiment 20, the expression vector according to embodiment 21, and/or the pharmaceutical composition according to embodiment 24, for use in the treatment of cancer.

Embodiment 27. The hBCMA-binding polypeptide according to any of embodiments 1 to 16, the hBCMA binder-drug conjugate according to any of embodiments 17 to 19, the nucleic acid molecule according to embodiment 20, the expression vector according to embodiment 21, and/or the pharmaceutical composition according to embodiment 24, for use according to embodiment 26, wherein the cancer is multiple myeloma.

Embodiment 28. Use of the hBCMA-binding polypeptide according to any of embodiments 1 to 16, the hBCMA binder-drug conjugate according to any of embodiments 17 to 19, the nucleic acid molecule according to embodiment 20, the expression vector according to embodiment 21, and/or the pharmaceutical composition according to embodiment 24, for the manufacture of a medicament for the treatment of cancer.

Embodiment 29. A method of treating cancer, the method comprising administering to a patient in need thereof the hBCMA-binding polypeptide according to any of embodiments 1 to 16, the hBCMA binder-drug conjugate according to any of embodiments 17 to 19, the nucleic acid molecule according to embodiment 20, the expression vector according to embodiment 21, and/or the pharmaceutical composition according to embodiment 24.

Embodiment 30. A kit comprising the hBCMA-binding polypeptide according to any of embodiments 1 to 16, the hBCMA binder-drug conjugate according to any of embodiments 17 to 19, or the pharmaceutical composition according to embodiment 24 and, optionally, one or more further therapeutic agent(s).

Embodiment 31. The kit according to embodiment 30, wherein the one or more further therapeutic agent(s) is selected from a proteasome inhibitor (for example carlfizomib or bortezomib), an immunomodulatory agent (for example lenalidomide or thalidomide), an alkylator (for example melphalan), a steroid (for example dexamethasone or prednisone), an anti-CD38 agent (for example daratumumab), an immune checkpoint inhibitor (for example a CTLA-4 inhibitor, a PD-1 inhibitor, or a PD-L1 inhibitor), and an ADAM17 inhibitor.

Embodiment 32. The kit according to embodiment 30 or embodiment 31, for use in the treatment of cancer, for example multiple myeloma.

1. An hBCMA-binding polypeptide which comprises at least one motif that binds to hBCMA, wherein said polypeptide comprises the following structure:
[N-terminal portion]-[Helix 1]-[Separating portion]-[Helix 2]-[C-terminal portion]
the hBCMA binding motif being the portion [Helix 1]-[Separating portion]-[Helix 2].

2. The hBCMA-binding polypeptide as claimed in claim 1, wherein:
i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are any naturally occurring amino acid;
X₁₁ is E, F, H, Q, T or Y;
X₁₄ is any naturally occurring amino acid;
X₁₇ is A, E, Q, S, T or V;
X₁₈ is any naturally occurring amino acid;
X₂₅ is F or Y;
X₃₁ is I, M, or V;
X₃₃ is K or S;
X₃₅ is I, L, M, or V;
or ii) Helix 1 and Helix 2 are defined as in i), wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue.

3. The hBCMA-binding polypeptide as claimed in claim 2, wherein the hBCMA binding efficacy is at least 1% of SEQ ID NO: 2.

4. The hBCMA-binding polypeptide as claimed in claim 1, wherein:
i) Helix 1 comprises the sequence X₉X₁₀X₁₁ADX₁₄EIX₁₇X₁₈ [SEQ ID NO. 278] and Helix 2 comprises the sequence FX₂₅QKWAFX₃₁RX₃₃LX₃₅ [SEQ ID NO. 279], wherein, independently from each other,
X₉ and X₁₀ are any naturally occurring amino acid;
X₁₁ is E, F, H, Q, T or Y;
X₁₄ is any naturally occurring amino acid;
X₁₇ is A, E, Q, S, T or V;
X₁₈ is any naturally occurring amino acid;
X₂₅ is F or Y;
X₃₁ is I, M, or V;
X₃₃ is K or S;
X₃₅ is I, L, M, or V;
or ii) Helix 1 and Helix 2 are defined as in i), wherein within Helix 1 and Helix 2, at least 1 and no more than 5 (for example at least 1 and no more than 3) of the Xₙ residues are replaced by an alternative residue, and/or at least 1 and no more than 5 (for example at least 1 and no more than 3) of the residues not labelled as Xₙ are replaced by an alternative residue;
and wherein the hBCMA binding efficacy is at least 1% of SEQ ID NO: 2.

5. The hBCMA-binding polypeptide as claimed in claim 2, wherein:
i) Helix 1 comprises the sequence X₉X₁₀X₁₁ADX₁₄EIX₁₇X₁₈ [SEQ ID NO. 278] and Helix 2 comprises the sequence FYQKWAFIRX₃₃LM, wherein, independently from each other,
X₉ is D, E, H, K, N, Q, S, or V;
X₁₀ is A, E, F, I, K, M, N, Q, R, S, T, Y, or V;
X₁₁ is E, F, or H;
X₁₄ is A, E, H, I, K, L, Q, R, T, or Y;
X₁₇ is A, E S, T, or V;
X₁₈ is A, F, H, K, L, M, N, T, or S;
X₃₃ is K or S;
or ii) Helix 1 and Helix 2 are defined as in i), wherein within Helix 1 and Helix 2, at least 1 and no more than 3 (for example 1, 2 or 3) of the Xₙ residues are replaced by an alternative residue, and/or at least 1 and no more than 3 (for example 1, 2 or 3) of the residues not labelled as Xₙ are replaced by an alternative residue (for example replaced by an alternative residue that is a conservative replacement).

6. The hBCMA-binding polypeptide as claimed in claim 5, wherein the hBCMA binding efficacy is at least 1% of SEQ ID NO: 2.

7. The hBCMA-binding polypeptide as claimed in any preceding claim, wherein:
Helix 1 comprises the sequence X₆X₇X₈X₉X₁₀X₁₁ADX₁₄EIX₁₇X₁₈X₁₉ and/or Helix 2 comprises the sequence,
wherein, X₆ is any naturally occurring amino 164 acid (preferably D, E, N or Q; more preferably N) or is absent;
X₇ is any naturally occurring amino acid (preferably H, K or R; more preferably K) or is absent;
X₈ is any naturally occurring amino acid (preferably D, E, N or Q; more preferably E) or is absent;
X₁₉ is any naturally occurring amino acid (preferably G, A, V, L or I; more preferably L) or is absent;
X₂₃ is any naturally occurring amino acid (preferably D, E, N or Q; more preferably N) or is absent;
X₃₆ is any naturally occurring amino acid (preferably D, E, N or Q; more preferably D) or is absent; and
X₃₇ is any naturally occurring amino acid (preferably D, E, N or Q; more preferably D) or is absent;
and preferably wherein X₆ is N; X₇ is K; and X₈ is E; and/or wherein X₃₆ is D; and X₃₇ is D;
for example wherein X₆ is N; X₇ is K; X₈ is E; X₁₉ is L; X₂₃ is N; X₃₆ is D; and X₃₇ is D.

8. The hBCMA-binding polypeptide as claimed in claim 7, wherein:
i) Helix 1 comprises the sequence NKEETFADLEISNL and Helix 2 comprises the sequence NFYQKWAFIRSLMDD,
or ii) Helix 1 and Helix 2 are defined as in i), wherein within Helix 1 and Helix 2, at least 1 and no more than 2 (for example 1 or 2) residues are replaced by an alternative residue,
or (iii) at least 1 and no more than 3 (for example 1, 2, or 3) residues in the sequence of Helix 1 and/or Helix 2 are replaced by an alternative residue (for example replaced by an alternative residue that is a conservative replacement).

9. The hBCMA-binding polypeptide as claimed in claim 8, wherein the hBCMA binding efficacy is at least 1% of SEQ ID NO: 1.

10. The hBCMA-binding polypeptide as claimed in claim 7, wherein:
i) Helix 1 comprises the sequence NKENQFADEEIAAL and Helix 2 comprises the sequence NFYQKWAFIRKLMDD,
or ii) Helix 1 and Helix 2 are defined as in i), wherein within Helix 1 and Helix 2, at least 1 and no more than 2 (for example 1 or 2) residues are replaced by an alternative residue
or (iii) at least 1 and no more than 3 (for example 1, 2, or 3) residues in the sequence of Helix 1 and/or Helix 2 are replaced by an alternative residue (for example replaced by an alternative residue that is a conservative replacement).

11. The hBCMA-binding polypeptide as claimed in claim 10, wherein the hBCMA binding efficacy is at least 1% of SEQ ID NO: 2.

12. The hBCMA-binding polypeptide as claimed in any preceding claim wherein the separating portion is a sequence of 1 to 5 (preferably 2 to 5, for example 2, 3, 4 or 5; or for example 3 to 5, for example 3, 4 or 5, and more preferably 3) naturally occurring amino acids.

13. The hBCMA-binding polypeptide as claimed in any preceding claim wherein the separating portion has the sequence X₂₀X₂₁X₂₂, wherein
X₂₀ is any naturally occurring amino acid (preferably S, T, M, P, F, Y or W; more preferably P);
X₂₁ is any naturally occurring amino acid (preferably D, E, N or Q; more preferably N);
X₂₂ is any naturally occurring amino acid (preferably G, A, V, L or I; more preferably L); and wherein optionally one or two (for example optionally 1) of X₂₀, X₂₁ or X₂₂ are absent;
and preferably wherein X₂₀ is P; X₂₁ is N; and X₂₂ is L (for example, the separating portion has the sequence PNL).

14. The hBCMA-binding polypeptide as claimed in any preceding claim, wherein the N-terminal portion is
absent or
is a sequence of 1 to 15 (preferably 1 to 10 or 1 to 8, more preferably 1 to 5, for example 1, 2, 3 4 or 5) naturally occurring amino acids.

15. The hBCMA-binding polypeptide as claimed in any preceding claim wherein the N-terminal portion has the sequence XₐX_{b}X₁X₂X₃X₄X₅, wherein
Xₐ is M or is absent;
X_{b} is M or is absent;
X₁ is any naturally occurring amino acid (preferably G, A, V, L or I; more preferably V or G; most preferably V) or is absent;
X₂ is any naturally occurring amino acid (preferably D, E, N or Q; more preferably D) or is absent;
X₃ is any naturally occurring amino acid (preferably D, E, N or Q; more preferably N) or is absent;
X₄ is any naturally occurring amino acid (preferably H, K or R; more preferably K) or is absent; and
X₅ is any naturally occurring amino acid (preferably F, Y or W; more preferably F) or is absent;
preferably wherein Xₐ is M or is absent, X_{b} is M or is absent, X₁ is V, G or absent (preferably V or absent), X₂ is D or absent, X₃ is N or absent, X₄ is K or absent, and X₅ is F or absent.

16. The hBCMA-binding polypeptide as claimed in any preceding claim, wherein the N-terminal portion is absent, or the N-terminal portion has the sequence XₐX_{b}X₁X₂X₃X₄X₅ wherein
Xₐ is M, X_{b} is M, X₁ is V or G (preferably V), X₂ is D, X₃ is N, X₄ is K, and X₅ is F; or
Xₐ is absent, X_{b} is absent, X₁ is V or G (preferably V), X₂ is D, X₃ is N, X₄ is K, and X₅ is F; or
Xₐ is absent, X_{b} is absent, X₁ is absent, X₂ is D, X₃ is N, X₄ is K, and X₅ is F; or
Xₐ is absent, X_{b} is absent, X₁ is absent, X₂ is absent, X₃ is N, X₄ is K, and X₅ is F; or
Xₐ is absent, X_{b} is absent, X₁ is absent, X₂ is absent, X₃ is absent, X₄ is K, and X₅ is F; or
Xₐ is absent, X_{b} is absent, X₁ is absent, X₂ is absent, X₃ is absent, X₄ is absent, and X₅ is F.

17. The hBCMA-binding polypeptide as claimed in any preceding claim, wherein the N-terminal portion has the sequence X₁X₂X₃X₄X₅, wherein
X₁ is any naturally occurring amino acid (preferably G, A, V, L or I; more preferably V or G; and most preferably V) or is absent;
X₂ is any naturally occurring amino acid (preferably D, E, N or Q; more preferably D) or is absent;
X₃ is any naturally occurring amino acid (preferably D, E, N or Q; more preferably N) or is absent;
X₄ is any naturally occurring amino acid (preferably H, K or R; more preferably K) or is absent; and
X₅ is any naturally occurring amino acid (preferably F, Y or W; more preferably F) or is absent;
and preferably wherein X₁ is V, G or absent, X₂ is D or absent, X₃ is N or absent, X₄ is K or absent, and X₅ is F or absent.

18. The hBCMA-binding polypeptide as claimed in any preceding claim, wherein the N-terminal portion is absent, or the N-terminal portion has the sequence X₁X₂X₃X₄X₅ wherein
X₁ is V or G (preferably V), X₂ is D, X₃ is N, X₄ is K, and X₅ is F; or
X₁ is absent, X₂ is D, X₃ is N, X₄ is K, and X₅ is F; or
X₁ is absent, X₂ is absent, X₃ is N, X₄ is K, and X₅ is F; or
X₁ is absent, X₂ is absent, X₃ is absent, X₄ is K, and X₅ is F; or
X₁ is absent, X₂ is absent, X₃ is absent, X₄ is absent, and X₅ is F.

19. The hBCMA-binding polypeptide as claimed in any preceding claim, wherein the C-terminal portion is
absent or
is a sequence of 1 to 50 (for example 1 to 35, 1 to 30, 15 to 25 or 18 to 22) naturally occurring amino acids.

20. The hBCMA-binding polypeptide as claimed in claim 19 wherein the C-terminal portion has the sequence X₃₈X₃₉QSANLLAEAKKLNDAQX₅₆X₅₇X₅₈, wherein
X₃₈ is a sequence of 1 to 14 (preferably 1 to 9 or 1 to 7, more preferably 1 to 4, for example 1, 2, 3 or 4) naturally occurring amino acids (and preferably X₃₈ is any naturally occurring amino acid (most preferably X₃₈ is P)),
X₃₉ is any naturally occurring amino acid (preferably S, T, M, P, F, Y or W; more preferably S),
X₅₆ is any naturally occurring amino acid (preferably G, A, V, L or I; more preferably A) or is absent,
X₅₇ is any naturally occurring amino acid (preferably P) or is absent; and
X₅₈ is any naturally occurring amino acid (preferably H, K or R; more preferably K) or is absent;
(for example X₃₈ is P; X₃₉ is S; X₅₆ is A or absent; X₅₇ is P or absent; X₅₈ is K or absent)
and optionally wherein, at least 1 and no more than 5 (for example 1, 2, 3, 4 or 5) (preferably at least 1 and no more than 3 (for example 1, 2, or 3)) of the residues in the sequence QSANLLAEAKKLNDAQ are replaced by an alternative residue;
and preferably optionally wherein, at least 1 and no more than 5 (for example 1, 2, 3, 4 or 5) (preferably at least 1 and no more than 3 (for example 1, 2, or 3)) of the residues in the sequence QSANLLAEAKKLNDAQ are replaced by an alternative residue that is a conservative replacement.

21. The hBCMA-binding polypeptide as claimed in claim 20 wherein the C-terminal portion has the sequence X₃₈X₃₉QSANLLAEAKKLNDAQX₅₆X₅₇X₅₈, wherein
X₅₆ is A, X₅₇ is P, and X₅₈ is K;
X₅₆ is A; or X₅₇ is P, and X₅₈ is absent;
X₅₆ is A, X₅₇ is absent; and X₅₈ is absent; or
X₅₆ is absent, X₅₇ is absent, and X₅₈ is absent.
and optionally wherein, at least 1 and no more than 3 (for example 1, 2, or 3) of the residues in the sequence QSANLLAEAKKLNDAQ are replaced by an alternative residue;
and preferably optionally wherein, at least 1 and no more than 3 (for example 1, 2, or 3) of the residues in the sequence QSANLLAEAKKLNDAQ are replaced by an alternative residue that is a conservative replacement; and
preferably wherein X₃₈ is P and X₃₉ is S.

22. The hBCMA-binding polypeptide as claimed in any preceding claim, wherein:
(i) the separating portion has the sequence X₂₀X₂₁X₂₂; and/or
the N-terminal portion has the sequence XₐX_{b}X₁X₂X₃X₄X₅; and/or
the C-terminal portion has the sequence
PSQSANLLAEAKKLNDAQX₅₆X₅₇X₅₈;
wherein, in said separating portion,
X₂₀ is P; X₂₁ is N; X₂₂ is L;
wherein in said N-terminal portion,
Xₐ is M or absent; X_{b} is M or absent; X₁ is V or G (preferably V), or absent; X₂ is D or G, or absent; X₃ is G or N, or absent; X₄ is K or absent; X₅ is F or absent;
and wherein in said C-terminal portion,
X₅₆ is A or absent; X₅₇ is P or absent; X₅₈ is K or absent;
or (ii) the separating portion, N-terminal portion, and C-terminal portion are as defined in (i), wherein optionally
(a) within each portion 1, 2 or 3 residues are replaced by an alternative residue; or
(b) within those portions taken together at least 1 and no more than 10 (for example, not more than 5, for example 1, 2, 3, 4, or 5) residues are replaced by an alternative residue.

23. The hBCMA-binding polypeptide as claimed in any preceding claim, wherein said separating portion has the sequence PNL.

24. The hBCMA-binding polypeptide as claimed in any preceding claim, wherein:
(i) said separating portion has the sequence PNL and said hBCMA binding motif is flanked by an N-terminal portion X₁X₂X₃X₄X₅ and a C-terminal portion PSQSANLLAEAKKLNDAQAPK,
wherein in said N-terminal portion,
X₁ is G, V, or deleted; X₂ is D or deleted; X₃ is N or deleted; X₄ is K or deleted; X₅ is F or deleted;
or (ii) the separating portion, N-terminal portion, and C-terminal portion are as defined in (i), wherein within those portions taken together at least 1 and no more than 5 residues are replaced by an alternative residue.

25. The hBCMA-binding polypeptide as claimed in any preceding claim, wherein said N-terminal portion has the sequence VDNKF.

26. The hBCMA-binding polypeptide as claimed in any of claims 12-25 wherein the hBCMA binding efficacy is at least 1% of SEQ ID NO: 2.

27. The hBCMA-binding polypeptide as claimed in any preceding claim, wherein the hBCMA binding motif sequence is selected from SEQ ID NOs. 170 to 275;
preferably from SEQ ID NOs. 170-188;
and wherein optionally from 1 to 5 (preferably 1, 2 or 3) residues in the sequence are replaced by an alternative residue, and preferably optionally from 1 to 5 (preferably 1, 2 or 3) residues in the sequence are replaced by an alternative residue that is a conservative replacement.

28. The hBCMA-binding polypeptide as claimed in claim 1 or claim 2, which comprises a sequence selected from SEQ ID NOs. 1, 2, 23-39, 41-119, or 121-128 and wherein optionally from 1 to 5 (preferably 1, 2 or 3) residues in the sequence are replaced by an alternative residue, and preferably optionally from 1 to 5 (preferably 1, 2 or 3) residues in the sequence are replaced by an alternative residue that is a conservative replacement; or
the hBCMA-binding polypeptide as claimed in claim 1 or claim 2, which comprises a sequence selected from SEQ ID NOs. 2 and 23-39, and wherein optionally from 1 to 5 (preferably 1, 2 or 3) residues in the sequence are replaced by an alternative residue, and preferably optionally from 1 to 5 (preferably 1, 2 or 3) residues in the sequence are replaced by an alternative residue that is a conservative replacement.

29. The hBCMA-binding polypeptide as claimed in claim 1 or claim 2, wherein the sequence of the hBCMA-binding polypeptide is selected from:
[SEQ ID NO: 1]
VDNKFNKEETFADLEISNLPNLNFYQKWAFIRSLMDDPSQSANLLAEA
KKLNDAQAPK;
or
[SEQ ID NO: 2]
VDNKFNKENQFADEEIAALPNLNFYQKWAFIRKLMDDPSQSANLLAE
AKKLNDAQAPK;
or
[SEQ ID NO: 23]
VDNKFNKEEIFADREIAFLPNLNFYQKWAFIRKLMDDPSQSANLLAEA
KKLNDAQAPK;
or
[SEQ ID NO: 33]
VDNKFNKEHQFADYEIAMLPNLNFYQKWAFIRSLMDDPSQSANLLAE
AKKLNDAQAPK.

30. The hBCMA-binding polypeptide as claimed in claim 1 or claim 2 wherein the sequence of the hBCMA-binding polypeptide is selected from SEQ ID NOs. 23-39.

31. The hBCMA-binding polypeptide according to any preceding claim, wherein the hBCMA-binding polypeptide does not comprise methionine.

32. The hBCMA-binding polypeptide with the sequence according to any preceding claim, wherein, at a position at which a methionine residue is recited, the polypeptide has the sequence with the methionine residue independently substituted for a different naturally occurring amino acid or an unnatural amino acid (for example a different naturally occurring amino acid or norleucine); and preferably each methionine residue is independently substituted for an amino acid selected from isoleucine (I), leucine (L), glutamine (Q) and norleucine; and more preferably each methionine residue is independently substituted for a norleucine or isoleucine.

33. The hBCMA-binding polypeptide according to any preceding claim,
wherein one or more residues (for example 1 to 5 residues, for example 1, 2, 3, 4 or 5) of the hBCMA-binding polypeptide is/are substituted for an unnatural amino acid, for example norleucine;
and/or wherein one or more methionine residues, when present (for example 1 or 2 methionine residue(s) when present), is/are substituted for an unnatural amino acid, for example norleucine;
and/or wherein one or more leucine residues, when present (for example 1 to 5 leucine residues, when present), is/are substituted for norleucine;
and/or wherein one or more methionine residues, when present (for example 1 or 2 methionine residue(s) when present), is/are oxidised (for example is/are Met(O)).

34. An hBCMA-binding oligomer, which comprises at least two hBCMA-binding polypeptides as defined in any one of claims 1 to 33, for example 2, 3, 4, or 5 hBCMA binding polypeptides as defined in any one of claims 1 to 33.

35. The hBCMA-binding oligomer as claimed in claim 34, which comprises at least two hBCMA-binding polypeptides, wherein a first hBCMA-binding polypeptide is as defined in any of claims 2-33, and a second hBCMA-binding polypeptide is as defined in any of claims 2-33.

36. The hBCMA-binding oligomer as claimed in claim 35, which comprises at least two hBCMA-binding polypeptides, wherein the first and second hBCMA-binding polypeptide have the same sequence.

37. The hBCMA-binding oligomer as claimed in claim 35, which comprises at least two hBCMA-binding polypeptides, wherein the first and second hBCMA-binding polypeptide have a different sequence.

38. The hBCMA-binding oligomer according to claim 34, which comprises at least two hBCMA-binding polypeptides, wherein:
a first hBCMA-binding polypeptide comprises a first binding motif selected from SEQ ID NOs. 170 to 275 (preferably from SEQ ID NOs. 170-188) or comprises a first sequence selected from SEQ ID NOs. 1, 2, 23-39, 41-119, or 121-128;
and a second hBCMA-binding polypeptide comprises a second binding motif selected from SEQ ID NOs. 170 to 275 (preferably from SEQ ID NOs. 170-188) or comprises a second sequence selected from SEQ ID NOs. 1, 2, 23-39, 41-119, or 121-128.

39. The hBCMA-binding oligomer according to claim 38, which comprises at least two hBCMA-binding polypeptides, wherein the first and second hBCMA-binding polypeptides have the same sequence, or the first and second binding motifs selected have the same sequence.

40. The hBCMA-binding oligomer according to claim 38, which comprises at least two hBCMA-binding polypeptides, wherein the first and second binding motif selected or first and second hBCMA-binding polypeptide have a different sequence.

41. The hBCMA-binding oligomer as claimed in any of claims 34-40, wherein the hBCMA-binding polypeptides are each separated by a linker.

42. The hBCMA-binding oligomer as claimed in claim 41, wherein the linker is a sequence of 1 to 50 (for example 1 to 25) naturally occurring amino acids; preferably 1 to 25 (for example 1 to 20) naturally occurring amino acids selected from the group consisting of G, S and T (preferably G and S).

43. The hBCMA-binding oligomer as claimed in claim 41 or 42, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG, GGSGGGGSGGGGSGGGGSGG, GGGGS, GGGGSGGGGS, GGGGSGGGGSGGGGS, or GGGGSGGGGSGGGGSGGGGS.

44. The hBCMA-binding oligomer as claimed in any of claims 41 to 43, wherein the hBCMA-binding oligomer comprises at least 2 (for example 2) hBCMA-binding polypeptides, and the hBCMA-binding oligomer comprises the following structure:
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion];
wherein the linker is a sequence of 1 to 50 naturally occurring amino acids, preferably 1 to 25 (for example 1-20) naturally occurring amino acids selected from the group consisting of G, S and T (preferably G and S) (for example, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG, GGSGGGGSGGGGSGGGGSGG, GGGGS, GGGGSGGGGS, GGGGSGGGGSGGGGS, or GGGGSGGGGSGGGGSGGGGS;
wherein each N-terminal portion in the oligomer may have the same sequence or have different sequences;
each C-terminal portion in the oligomer may have the same sequence or have different sequences;
each separating portion in the oligomer may have the same sequence or have different sequences;
each Helix 1 portion in the oligomer may have the same sequence or have different sequences; and
each Helix 2 portion in the oligomer may have the same sequence or have different sequences.

45. The hBCMA-binding oligomer as claimed in claim 44, wherein the hBCMA-binding oligomer comprises at least 3 (for example 3) hBCMA-binding polypeptides, and the hBCMA-binding oligomer comprises the following structure
[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion]-[Linker]-[N-terminal portion]-[Helix 1]-[Separating portion]-[Helix 2]-[C-terminal portion];
wherein the linker is a sequence of 1 to 50 naturally occurring amino acids, preferably 1 to 25 (for example 1-20) naturally occurring amino acids selected from the group consisting of G, S and T (preferably G and S) (for example, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG, GGSGGGGSGGGGSGGGGSGG, GGGGS, GGGGSGGGGS, GGGGSGGGGSGGGGS, or GGGGSGGGGSGGGGSGGGGS;
and wherein each linker portion in the oligomer may have the same sequence or have different sequences;
each N-terminal portion in the oligomer may have the same sequence, have different sequences, or two may have the same sequence and one may have a different sequence;
each C-terminal portion may have the same sequence, have different sequences, or two may have the same sequence and one may have a different sequence;
each separating portion in the oligomer may have the same sequence, have different sequences, or two may have the same sequence and one may have a different sequence;
each Helix 1 portion in the oligomer may have the same sequence, have different sequences, or two may have the same sequence and one may have a different sequence; and
each Helix 2 portion in the oligomer may have the same sequence, have different sequences, or two may have the same sequence and one may have a different sequence.

46. The hBCMA-binding polypeptide as claimed in one of claims 2 to 33, or hBCMA-binding oligomer as claimed in any one of claims 34 to 45, which further comprises an additional functional portion (for example at least one, at least two, or at least three; for example 1, 2, 3, 4 or 5 additional functional portions).

47. The hBCMA-binding polypeptide or hBCMA-binding oligomer as claimed in claim 46, wherein the additional functional portion comprises an immune signalling molecule or derivative thereof, for example a cytokine or a derivative thereof, for example IL-15 or a derivative thereof.

48. The hBCMA-binding polypeptide or hBCMA-binding oligomer as claimed in claim 46, wherein the additional functional portion comprises an additional binding moiety.

49. The hBCMA-binding polypeptide or hBCMA-binding oligomer as claimed in claim 48, wherein the additional binding moiety is specific for an immune cell surface target, for example an NK cell activating receptor, for example CD16a.

50. The hBCMA-binding polypeptide or hBCMA-binding oligomer as claimed in claim 46, wherein the hBCMA-binding polypeptide or hBCMA-binding oligomer comprises at least two (for example 2, 3, 4 or 5) additional functional portions, wherein each additional functional portion may be the same or may be different.

51. The hBCMA-binding polypeptide or hBCMA-binding oligomer as claimed in claim 50, wherein the hBCMA-binding polypeptide or hBCMA-binding oligomer comprises at least two (for example 2, 3, 4 or 5) additional functional portions,
wherein a first additional functional portion comprises an additional binding moiety (for example an additional binding moiety specific for an immune cell surface target, for example an NK cell activating receptor, for example CD16a); and wherein the second additional functional portion comprises an immune signalling molecule or derivative thereof, for example a cytokine or a derivative thereof, for example IL-15 or a derivative thereof; or
wherein a first additional functional portion comprises an additional binding moiety (for example an additional binding moiety specific for an immune cell surface target, for example an NK cell activating receptor, for example CD16a); and wherein the second additional functional portion comprises an additional binding moiety (for example an additional binding moiety specific for an immune cell surface target, for example an NK cell activating receptor, for example CD16a).

52. The hBCMA-binding polypeptide or hBCMA-binding oligomer as claimed in claim 51 comprising an additional functional portion, wherein the hBCMA-binding polypeptide or hBCMA-binding oligomer comprises at least 3 (for example 3, 4 or 5) additional functional portions, wherein a third additional functional portion comprises an additional binding moiety (specific for an immune cell surface target, for example an NK cell activating receptor, for example CD16a), or comprises an immune signalling molecule or derivative thereof, for example a cytokine or a derivative thereof, for example IL-15 or a derivative thereof.

53. The hBCMA-binding polypeptide or hBCMA-binding oligomer as claimed in any of claims 46-52, wherein the additional functional portion(s) is/are separated from the hBCMA-binding polypeptide or the or hBCMA-binding oligomer by a linker,
for example wherein the linker is a sequence of 1 to 50 naturally occurring amino acids, preferably 1 to 25 (for example 1 to 20), naturally occurring amino acids selected from the group consisting of G, S and T (preferably G and S).

54. The hBCMA-binding polypeptide or hBCMA-binding oligomer as claimed in claim 65, wherein the linker wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGGGGGSG, GGGSGGGGSGGGGSGGGGSG, GGSGG, GGSGGGGSGG, GGSGGGGSGGGGSGG, GGSGGGGSGGGGSGGGGSGG, GGGGS, GGGGSGGGGS, GGGGSGGGGSGGGGS, or GGGGSGGGGSGGGGSGGGGS.

55. The hBCMA-binding polypeptide or hBCMA-binding oligomer as claimed in claim 53 or 54 comprising an additional functional portion, wherein the hBCMA-binding polypeptide comprises the following structure:
[N-terminal portion]-[Helix 1]-[Separating portion]-[Helix 2]-[C-terminal portion]-[linker]-[additional functional portion]; or
[additional functional portion]-[linker]-[N-terminal portion]-[Helix 1]-[Separating portion]-[Helix 2]-[C-terminal portion]; or
[N-terminal portion]-[Helix 1]-[Separating portion]-[Helix 2]-[C-terminal portion]-[linker]-[additional functional portion]-[additional functional portion];
[additional functional portion]-[additional functional portion]-[linker]-[N-terminal portion]-[Helix 1]-[Separating portion]-[Helix 2]-[C-terminal portion]; or
[additional functional portion]-[linker]-[N-terminal portion]-[Helix 1]-[Separating portion]-[Helix 2]-[C-terminal portion]-[additional functional portion];
wherein the linker is a sequence of 1 to 50 naturally occurring amino acids, preferably 1 to 25 (for example 1-20) naturally occurring amino acids selected from the group consisting of G, S and T (preferably G and S) (for example, wherein the linker is G or comprises the sequence GGGSG, GGGGS, GGSGG, GSGGG and/or SGGGG; for example wherein the linker is G or comprises or has the sequence GGGSG, GGGSGGGGSG, GGGSGGGGSGGGGSG, GGGSGGGGSGGGGSGGGGSG, GGSGG, GGSGGGGSGG, GGSGGGGSGGGGSGG, GGSGGGGGGGGSGGGGSGG, GGGGS, GGGGSGGGGS, GGGGSGGGGSGGGGS, or GGGGSGGGGSGGGGSGGGGS).
and
wherein, when more than one additional functional portion is present, each additional functional may be the same, or be different.

56. The hBCMA-binding polypeptide or hBCMA-binding oligomer as claimed in claims 53 to 55, which comprises the sequence
[SEQ ID NO. 139]
VDNKFNKENQFADEEIAALPNLNFYQKWAFIRKLMDDPSQSANLLAE
AKKLNDAQAPKGGGSGGGGSGGGGSGVDNKFNKEVQMAQFEIRKLP
NLNHHQSFAFIKSLMDDPSQSANLLAEAKKLNDAQAPK;
[SEQ ID NO. 140]
VDNKFNKENQFADEEIAALPNLNFYQKWAFIRKLMDDPSQSANLLAE
AKKLNDAQAPKGGGSGGGGSGGGGSGVDNKFNKEFWIAESEIESLPN
LNIYQKWAFKYSLADDPSQSANLLAEAKKLNDAQAPKGGGSGGGGS
GGGGSGVDNKFNKEVQMAQFEIRKLPNLNHHQSFAFIKSLMDDPSQS
ANLLAEAKKLNDAQAPK;
[SEQ ID NO. 141]
VDNKFNKENQFADEEIAALPNLNFYQKWAFIRKLMDDPSQSANLLAE
AKKLNDAQAPKGGGSGGGGSGGGGSGVDNKFNKEVQMAQFEIRKLP
NLNHHQSFAFIKSLMDDPSQSANLLAEAKKLNDAQAPKGGGSGGGGS
GGGGSGVDNKFNKEVQMAQFEIRKLPNLNHHQSFAFIKSLMDDPSQS
ANLLAEAKKLNDAQAPK;
[SEQ ID NO. 142]
VDNKFNKENQFADEEIAALPNLNFYQKWAFIRKLMDDPSQSANLLAE
AKKLNDAQAPKGGGSGGGGSGGGGSGVDNKFNKEQFYARDEIDLLP
NLNEDQKWAFYMSLIDDPSQSANLLAEAKKLNDAQAPKGGGSGGGG
SGGGGSGVDNKFNKEVQMAQFEIRKLPNLNHHQSFAFIKSLMDDPSQS
ANLLAEAKKLNDAQAPK;
[SEQ ID NO. 143]
VDNKFNKENQFADEEIAALPNLNFYQKWAFIRKLMDDPSQSANLLAE
AKKLNDAQAPKGGGSGVDNKFNKENQFADEEIAALPNLNFYQKWAFI
RKLMDDPSQSANLLAEAKKLNDAQAPKGGGSGVDNKFNKEVQMAQ
FEIRKLPNLNHHQSFAFIKSLMDDPSQSANLLAEAKKLNDAQAPK;
[SEQ ID NO. 144]
VDNKFNKENQFADEEIAALPNLNFYQKWAFIRKLMDDPSQSANLLAE
AKKLNDAQAPKGGGSGGGGSGVDNKFNKENQFADEEIAALPNLNFY
QKWAFIRKLMDDPSQSANLLAEAKKLNDAQAPKGGGSGGGGSGVDN
KFNKEVQMAQFEIRKLPNLNHHQSFAFIKSLMDDPSQSANLLAEAKKL
NDAQAPK;
or
[SEQ ID NO. 145]
VDNKFNKENQFADEEIAALPNLNFYQKWAFIRKLMDDPSQSANLLAE
AKKLNDAQAPKGGGSGGGGSGGGGSGVDNKFNKENQFADEEIAALP
NLNFYQKWAFIRKLMDDPSQSANLLAEAKKLNDAQAPKGGGSGGGG
SGGGGSGVDNKFNKEVQMAQFEIRKLPNLNHHQSFAFIKSLMDDPSQS
ANLLAEAKKLNDAQAPK.

57. An hBCMA binder-drug conjugate comprising the hBCMA-binding polypeptide or hBCMA binding oligomer as claimed in any of claims 1-56 and an additional therapeutic agent.

58. The hBCMA binder-drug conjugate as claimed in claim 57, wherein the additional therapeutic agent is a cytotoxic drug, for example MMAF, MMAE, doxorubicin, pyrrolobenzodiazepine, amanitin, maytansinoids, duostatins, mitomycin C, desmethyltopotecan or SN-38.

59. The hBCMA binder-drug conjugate as claimed in claim 57 or claim 58, wherein the hBCMA-binding polypeptide is connected to the additional therapeutic agent via a linker.

60. A nucleic acid molecule encoding the hBCMA-binding polypeptide as claimed in any one of claims 1 to 33 or 46 to 56; or encoding the hBCMA-binding oligomer as claimed in any of claims 34 to 56.

61. An expression vector comprising the nucleic acid molecule as claimed in claim 60.

62. A host cell comprising the nucleic acid molecule as claimed in claim 60 or the expression vector as claimed in claim 61.

63. A method of making the hBCMA-binding polypeptide as claimed in any one of claims 1 to 33 or 46 to 56, the hBCMA binding oligomer as claimed in any of claims 34 to 56, the method comprising maintaining the host cell of claim 62 under optimal conditions for expression of the nucleic acid and isolating the hBCMA-binding polypeptide or hBCMA binding oligomer.

64. A pharmaceutical composition comprising the hBCMA-binding polypeptide as claimed in any one of claims 1 to 33 or 46 to 56, the hBCMA binding oligomer as claimed in any of claims 34 to 56, the hBCMA binder-drug conjugate as claimed in any of claims 57 to 59, the nucleic acid molecule as claimed in claim 60 or the expression vector as claimed in claim 61.

65. The hBCMA-binding polypeptide as claimed in any one of claims 1 to 33 or 46 to 56, the hBCMA binding oligomer as claimed in any of claims 34 to 56, the hBCMA binder-drug conjugate as claimed in claims 57 to 59, the nucleic acid molecule as claimed in claim 60, the expression vector as claimed in claim 61, and/or the pharmaceutical composition as claimed in claim 64, for use in medicine.

66. The hBCMA-binding polypeptide as claimed in any one of claims 1 to 33 or 46 to 56, the hBCMA binding oligomer as claimed in any of claims 34 to 56, the hBCMA binder-drug conjugate as claimed in claims 57 to 59, the nucleic acid molecule as claimed in claim 60, the expression vector as claimed in claim 61, and/or the pharmaceutical composition as claimed in claim 64, for use in the treatment of cancer.

67. The hBCMA-binding polypeptide as claimed in any one of claims 1 to 33 or 46 to 56, the hBCMA binding oligomer as claimed in any of claims 34 to 56, the hBCMA binder-drug conjugate as claimed in claims 57 to 59, the nucleic acid molecule as claimed in claim 60, the expression vector as claimed in claim 61, and/or the pharmaceutical composition as claimed in claim 64, for use as claimed in claim 66, wherein the cancer is multiple myeloma.

68. Use of the hBCMA-binding polypeptide as claimed in any one of claims 1 to 33 or 46 to 56, the hBCMA binding oligomer as claimed in any of claims 34 to 56, the hBCMA binder-drug conjugate as claimed in claims 57-59, the nucleic acid molecule as claimed in claim 60, the expression vector as claimed in claim 61, and/or the pharmaceutical composition as claimed in claim 64, for the manufacture of a medicament for the treatment of cancer.

69. A method of treating cancer, the method comprising administering to a patient in need thereof the hBCMA-binding polypeptide as claimed in any one of claims 1 to 33 or 46 to 56, the hBCMA binding oligomer as claimed in any of claims 34 to 56, the hBCMA binder-drug conjugate as claimed in claims 57-59, the nucleic acid molecule as claimed in claim 60, the expression vector as claimed in claim 61, and/or the pharmaceutical composition as claimed in claim 64.

70. A kit comprising the hBCMA-binding polypeptide as claimed in any one of claims 1 to 33 or 46 to 56, the hBCMA binding oligomer as claimed in any of claims 34 to 56, the hBCMA binder-drug conjugate as claimed in claims 57-59, or the pharmaceutical composition as claimed in claim 64 and, optionally, one or more further therapeutic agent(s).

71. The kit as claimed in claim 70, wherein the one or more further therapeutic agent(s) is selected from a proteasome inhibitor (for example carlfizomib or bortezomib), an immunomodulatory agent (for example lenalidomide or thalidomide), an alkylator (for example melphalan or melflufen), a steroid (for example dexamethasone or prednisone), an anti-CD38 agent (for example daratumumab), an immune checkpoint inhibitor (for example a CTLA-4 inhibitor, a PD-1 inhibitor, or a PD-L1 inhibitor), and an ADAM17 inhibitor.

72. The kit as claimed in claim 70 or claim 71, for use in the treatment of cancer, for example multiple myeloma
